SEQUENCE LISTING (191 sequences)

SEQ ID NO: 1 - protein, 7 aa, Artificial Sequence
  Recognition Sequence
  MISC_FEATURE (1)..(1): Asp, Ala or Gly
  MISC_FEATURE (4)..(4): Val, Asp, Glu, Ala or Gly
  MISC_FEATURE (5)..(5): Leu or Arg
  MISC_FEATURE (6)..(6): Thr or Lys
  MISC_FEATURE (7)..(7): Arg, Lys or Gly
XSSXXXX

SEQ ID NO: 2 - protein, 7 aa, Artificial Sequence
  Recognition Sequence
  MISC_FEATURE (1)..(1): Asp, Ala, Gly, Thr, Val or Ser
  MISC_FEATURE (2)..(2): Ser, Asn, Arg, Ala or Gly
  MISC_FEATURE (3)..(3): Ser, Arg, Glu, Ala or Gly
  MISC_FEATURE (4)..(4): Val, Asp, Glu, Ala, Gly, Ile, Leu, Ser or Thr
  MISC_FEATURE (5)..(5): Leu or Arg
  MISC_FEATURE (6)..(6): Thr or Lys
  MISC_FEATURE (7)..(7): Arg, Lys or Gly
XXXXXXX

SEQ ID NO: 3 - protein, 7 aa, Artificial Sequence
  Recognition Sequence
  MISC_FEATURE (3)..(3): Asp, Ala or Gly
  MISC_FEATURE (6)..(6): Thr, Ser or Ala
  MISC_FEATURE (7)..(7): Arg, Lys or Gly
RSXHLXX

SEQ ID NO: 4 - protein, 7 aa, Artificial Sequence
  Recognition Sequence
  MISC_FEATURE (1)..(1): Asp, Ala or Gly
  MISC_FEATURE (4)..(4): Val, Asp, Glu, Ala or Gly
  MISC_FEATURE (7)..(7): Arg, Lys or Gly
XSSXLTX

SEQ ID NO: 5 - protein, 7 aa, Artificial Sequence
  Recognition Sequence
  MISC_FEATURE (1)..(1): Asp, Ala, Gly, Thr, Val or Ser
  MISC_FEATURE (2)..(2): Ser, Asn or Arg
  MISC_FEATURE (3)..(3): Ser, Arg, Glu, Ala or Gly
  MISC_FEATURE (4)..(4): Val, Asp, Glu, Ala, Gly, Ile, Leu, Ser or Thr
  MISC_FEATURE (7)..(7): Arg, Lys or Gly
XXXXLTX

SEQ ID NO: 6 - protein, 7 aa, Artificial Sequence
  Recognition Sequence
  MISC_FEATURE (1)..(1): Asp, Ala or Gly
  MISC_FEATURE (4)..(4): Val, Asp, Glu, Ala or Gly
  MISC_FEATURE (7)..(7): Arg, Lys or Gly
XSSXRKX

SEQ ID NO: 7 - protein, 7 aa, Artificial Sequence
  Recognition Sequence
  MISC_FEATURE (1)..(1): Asp, Ala, Gly, Thr, Val or Ser
  MISC_FEATURE (2)..(2): Ser, Asn or Arg
  MISC_FEATURE (3)..(3): Ser, Arg, Glu, Ala or Gly
  MISC_FEATURE (4)..(4): Val, Asp, Glu, Ala, Gly, Ile, Leu, Ser or Thr
  MISC_FEATURE (7)..(7): Arg, Lys or Gly
XXXXRKX

SEQ ID NO: 8 - protein, 7 aa, Artificial Sequence
  Recognition Sequence
  MISC_FEATURE (1)..(1): Asp or Ala
  MISC_FEATURE (4)..(4): Val or Glu
  MISC_FEATURE (7)..(7): Arg or Lys
XSSXLTX

SEQ ID NO: 9 - protein, 7 aa, Artificial Sequence
  Recognition Sequence
  MISC_FEATURE (1)..(1): Asp, Ala or Thr
  MISC_FEATURE (2)..(2): Ser, Asn or Arg
  MISC_FEATURE (3)..(3): Ser, Arg or Glu
  MISC_FEATURE (4)..(4): Val, Glu or Asp
XXXXLTR

SEQ ID NO: 10 - protein, 7 aa, Artificial Sequence
  Recognition Sequence
  MISC_FEATURE (1)..(1): Asp or Ala
  MISC_FEATURE (4)..(4): Val or Glu
  MISC_FEATURE (7)..(7): Arg or Lys
XSSXRKX

SEQ ID NO: 11 - protein, 7 aa, Artificial Sequence
  Recognition Sequence
  MISC_FEATURE (1)..(1): Asp, Ala or Thr
  MISC_FEATURE (2)..(2): Ser, Asn or Arg
  MISC_FEATURE (3)..(3): Ser, Arg or Glu
  MISC_FEATURE (4)..(4): Val, Glu or Asp
XXXXRKR

SEQ ID NO: 12 - protein, 7 aa, Artificial Sequence
  Recognition Sequence
  MISC_FEATURE (3)..(3): Asp or Gly
  MISC_FEATURE (7)..(7): Arg or Lys
RSXHLTX

SEQ ID NO: 13 - protein, 7 aa, Artificial Sequence
  Recognition Sequence
DSSVLTR

SEQ ID NO: 14 - protein, 7 aa, Artificial Sequence
  Recognition Sequence
DSSVRKR

SEQ ID NO: 15 - protein, 7 aa, Artificial Sequence
  Recognition Sequence
ASSVLTR

SEQ ID NO: 16 - protein, 7 aa, Artificial Sequence
  Recognition Sequence
ASSVRKR

SEQ ID NO: 17 - protein, 7 aa, Artificial Sequence
  Recognition Sequence
DSSELTR

SEQ ID NO: 18 - protein, 7 aa, Artificial Sequence
  Recognition Sequence
DSSERKR

SEQ ID NO: 19 - protein, 7 aa, Artificial Sequence
  Recognition Sequence
ASSELTR

SEQ ID NO: 20 - protein, 7 aa, Artificial Sequence
  Recognition Sequence
ASSERKR

SEQ ID NO: 21 - protein, 7 aa, Artificial Sequence
  Recognition Sequence
GSSVLTR

SEQ ID NO: 22 - protein, 7 aa, Artificial Sequence
  Recognition Sequence
GSSVRKR

SEQ ID NO: 23 - protein, 7 aa, Artificial Sequence
  Recognition Sequence
GSSELTR

SEQ ID NO: 24 - protein, 7 aa, Artificial Sequence
  Recognition Sequence
GSSERKR

SEQ ID NO: 25 - protein, 7 aa, Artificial Sequence
  Recognition Sequence
DSSGLTR

SEQ ID NO: 26 - protein, 7 aa, Artificial Sequence
  Recognition Sequence
DSSGRKR

SEQ ID NO: 27 - protein, 7 aa, Artificial Sequence
  Recognition Sequence
ASSGLTR

SEQ ID NO: 28 - protein, 7 aa, Artificial Sequence
  Recognition Sequence
ASSGRKR

SEQ ID NO: 29 - protein, 7 aa, Artificial Sequence
  Recognition Sequence
GSSGLTR

SEQ ID NO: 30 - protein, 7 aa, Artificial Sequence
  Recognition Sequence
GSSGRKR

SEQ ID NO: 31 - protein, 7 aa, Artificial Sequence
  Recognition Sequence
DNRDLTR

SEQ ID NO: 32 - protein, 7 aa, Artificial Sequence
  Recognition Sequence
DNRDRKR

SEQ ID NO: 33 - protein, 7 aa, Artificial Sequence
  Recognition Sequence
TREDLTR

SEQ ID NO: 34 - protein, 7 aa, Artificial Sequence
  Recognition Sequence
TREDRKR

SEQ ID NO: 35 - protein, 7 aa, Artificial Sequence
  Recognition Sequence
DNRELTR

SEQ ID NO: 36 - protein, 7 aa, Artificial Sequence
  Recognition Sequence
DNRERKR

SEQ ID NO: 37 - protein, 7 aa, Artificial Sequence
  Recognition Sequence
ANRELTR

SEQ ID NO: 38 - protein, 7 aa, Artificial Sequence
  Recognition Sequence
ANRERKR

SEQ ID NO: 39 - protein, 7 aa, Artificial Sequence
  Recognition Sequence
DREELTR

SEQ ID NO: 40 - protein, 7 aa, Artificial Sequence
  Recognition Sequence
DREERKR

SEQ ID NO: 41 - protein, 7 aa, Artificial Sequence
  Recognition Sequence
AREELTR

SEQ ID NO: 42 - protein, 7 aa, Artificial Sequence
  Recognition Sequence
AREERKR

SEQ ID NO: 43 - protein, 7 aa, Artificial Sequence
  Recognition Sequence
TNRELTR

SEQ ID NO: 44 - protein, 7 aa, Artificial Sequence
  Recognition Sequence
TNRERKR

SEQ ID NO: 45 - protein, 7 aa, Artificial Sequence
  Recognition Sequence
TREELTR

SEQ ID NO: 46 - protein, 7 aa, Artificial Sequence
  Recognition Sequence
TREERKR

SEQ ID NO: 47 - protein, 7 aa, Artificial Sequence
  Recognition Sequence
GNRELTR

SEQ ID NO: 48 - protein, 7 aa, Artificial Sequence
  Recognition Sequence
GNRERKR

SEQ ID NO: 49 - protein, 7 aa, Artificial Sequence
  Recognition Sequence
GREELTR

SEQ ID NO: 50 - protein, 7 aa, Artificial Sequence
  Recognition Sequence
GREERKR

SEQ ID NO: 51 - protein, 7 aa, Artificial Sequence
  Recognition Sequence
DNRGLTR

SEQ ID NO: 52 - protein, 7 aa, Artificial Sequence
  Recognition Sequence
DNRGRKR

SEQ ID NO: 53 - protein, 7 aa, Artificial Sequence
  Recognition Sequence
ANRGLTR

SEQ ID NO: 54 - protein, 7 aa, Artificial Sequence
  Recognition Sequence
ANRGRKR

SEQ ID NO: 55 - protein, 7 aa, Artificial Sequence
  Recognition Sequence
DREGLTR

SEQ ID NO: 56 - protein, 7 aa, Artificial Sequence
  Recognition Sequence
DREGRKR

SEQ ID NO: 57 - protein, 7 aa, Artificial Sequence
  Recognition Sequence
AREGLTR

SEQ ID NO: 58 - protein, 7 aa, Artificial Sequence
  Recognition Sequence
AREGRKR

SEQ ID NO: 59 - protein, 7 aa, Artificial Sequence
  Recognition Sequence
TNRGLTR

SEQ ID NO: 60 - protein, 7 aa, Artificial Sequence
  Recognition Sequence
TNRGRKR

SEQ ID NO: 61 - protein, 7 aa, Artificial Sequence
  Recognition Sequence
TREGLTR

SEQ ID NO: 62 - protein, 7 aa, Artificial Sequence
  Recognition Sequence
TREGRKR

SEQ ID NO: 63 - protein, 7 aa, Artificial Sequence
  Recognition Sequence
DNRELTG

SEQ ID NO: 64 - protein, 7 aa, Artificial Sequence
  Recognition Sequence
DNRERKG

SEQ ID NO: 65 - protein, 7 aa, Artificial Sequence
  Recognition Sequence
TREELTG

SEQ ID NO: 66 - protein, 7 aa, Artificial Sequence
  Recognition Sequence
TREERKG

SEQ ID NO: 67 - protein, 7 aa, Artificial Sequence
  Recognition Sequence
GNRDLTR

SEQ ID NO: 68 - protein, 7 aa, Artificial Sequence
  Recognition Sequence
GNRDRKR

SEQ ID NO: 69 - protein, 7 aa, Artificial Sequence
  Recognition Sequence
GREDLTR

SEQ ID NO: 70 - protein, 7 aa, Artificial Sequence
  Recognition Sequence
GREDRKR

SEQ ID NO: 71 - protein, 7 aa, Artificial Sequence
  Recognition Sequence
GNRGLTR

SEQ ID NO: 72 - protein, 7 aa, Artificial Sequence
  Recognition Sequence
GNRGRKR

SEQ ID NO: 73 - protein, 7 aa, Artificial Sequence
  Recognition Sequence
GREGLTR

SEQ ID NO: 74 - protein, 7 aa, Artificial Sequence
  Recognition Sequence
GREGRKR

SEQ ID NO: 75 - protein, 7 aa, Artificial Sequence
  Recognition Sequence
RSDHLTR

SEQ ID NO: 76 - protein, 7 aa, Artificial Sequence
  Recognition Sequence
RSDHLTK

SEQ ID NO: 77 - protein, 7 aa, Artificial Sequence
  Recognition Sequence
RSDHLTG

SEQ ID NO: 78 - protein, 7 aa, Artificial Sequence
  Recognition Sequence
RSAHLTR

SEQ ID NO: 79 - protein, 7 aa, Artificial Sequence
  Recognition Sequence
RSAHLTK

SEQ ID NO: 80 - protein, 7 aa, Artificial Sequence
  Recognition Sequence
RSAHLTG

SEQ ID NO: 81 - protein, 7 aa, Artificial Sequence
  Recognition Sequence
RSGHLTR

SEQ ID NO: 82 - protein, 7 aa, Artificial Sequence
  Recognition Sequence
RSGHLTK

SEQ ID NO: 83 - protein, 7 aa, Artificial Sequence
  Recognition Sequence
RSGHLTG

SEQ ID NO: 84 - DNA, 63 bp, Artificial Sequence
  BMP10 secretion signal
atgggctctctggtcctgacactgtgcgctcttttctgcctggcagcttacttggtttctggc

SEQ ID NO: 85 - protein, 4 aa, Artificial Sequence
  Peptide cleavage sequence
RIRR

SEQ ID NO: 86 - DNA, 12 bp, Artificial Sequence
  Peptide cleavage sequence
cgaatcagaagg

SEQ ID NO: 87 - DNA, 42 bp, Artificial Sequence
  Double-NLS
ccgaagaaaaaacgtaaagtgccgaagaaaaaacgtaaagtg

SEQ ID NO: 88 - DNA, 1518 bp, Artificial Sequence
  Active delivery construct (dna) pCMV-SS- 2xNLS-ZFP-KOX-IRES-GFP
atgggctctctggtcctgacactgtgcgctcttttctgcctggcagcttacttggtttctggccgaatcagaagggatatgggaccgaagaaaaaacgtaaagtgccgaagaaaaaacgtaaagtgggcgaaagaccatacgcatgtcccgtcgaaagttgcgatagaaggtttagtcagtctggcgacctgaccaggcacatccgcatccacacaggctcccagaagccattccagtgcaggatctgtatgcgcaacttttctcagagcggcgatctgacccggcacatcagaacccacacaggcgagaagcccttcgcctgcgacatctgtggcaggaagtttgcccagtccggcgatcggaagagacacaccaagatccacacaggctctcagaagcctttccagtgccggatctgtatgagaaatttttcccagtctggcgacctgactagacacatccgcactcatacaggcgagaagccattcgcctgtgatatctgtggccggaagtttgcccagagcggcgataggaagcgccacacaaagatccacctgagacagaaggatggaggaggaggctctggaggaggaggcagcggaggaggaggctcccagaagccctttcagtgtagaatttgtatgcgcaactttagccagtctggcgatctgactagacatattaggactcataccggcgagaagcctttcgcctgtgatatttgtggccggaaattcgcccagagtggcgatcggaaaaggcatactaaaattcataccggctcccagaaaccatttcagtgtagaatctgcatgagaaatttttctcagagcggcgacctgactcgccacatcaggactcatactggagaaaaacccttcgcctgtgacatctgtggcagaaagtttgcccagtctggcgataggaaaagacatactaagatccacacaggcagccagaaaccattccagtgtagaatctgtatgagaaacttttcccagtccggcgatctgactagacacattaggactcacaccggagagaagccattcgcctgcgacatctgcggcaggaaattcgctcagagcggcgatcggaaaaggcacaccaaaatccacctgcgccagaaagatggaggaggaggatccggcggaggaggcagctccctgagcccccagcactccgccgtgacccagggctctatcatcaagaacaaggagggcatggatgccaagtctctgacagcctggagcaggaccctggtgacattcaaggacgtgttcgtggacttcacccgggaggagtggaagctgctggacacagcccagcagatcgtgtacagaaatgtgatgctggagaactataagaatctggtgagcctgggctaccagctgaccaagcccgatgtgatcctgcggctggagaagggcgaggagccttggctggtggagagagagattcatcaggaaactcatcccgatagcgaaaccgcattcgagattaagtcatccgtgtga

SEQ ID NO: 89 - protein, 14 aa, Artificial Sequence
  Mouse ZFP leader sequence
MGRIRKKLRLAERP

SEQ ID NO: 90 - protein, 14 aa, Artificial Sequence
  Human ZFP leader sequence
MGPKKRRKVTGERP

SEQ ID NO: 91 - protein, 35 aa, Artificial Sequence
  Human p65- RelA activation domain
PLGAPGLPNGLLSGDEDFSSIADMDFSALLSQISS

SEQ ID NO: 92 - protein, 34 aa, Artificial Sequence
  Mouse p65-RelA activation domain
PLGTSGLPNGLSGDEDFSSIADMDFSALLSQISS

SEQ ID NO: 93 - protein, 19 aa, Artificial Sequence
  Herpes simplex virus VP16 domain
PADALDDFDLDMLGDGDSP

SEQ ID NO: 94 - protein, 58 aa, Artificial Sequence
  VP64 domain
TSDALDDFDLDMLGSDALDDFDLDMLGSDALDDFDLDMLGSDALDDFDLDMLSSQLSQ

SEQ ID NO: 95 - protein, 15 aa, Artificial Sequence
  Linker Sequence
LRQKDGGGGSGGGGS

SEQ ID NO: 96 - protein, 472 aa, Artificial Sequence
  ZF11xALS1-Kox
MGRIRKKLRLAERPYACPVESCDRRFSDSSVLTRHIRIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHLRQKDGGGGSGGGGSGGGGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHLRQKDGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 97 - protein, 482 aa, Artificial Sequence
  ZF11xALS2-Kox
MGRIRKKLRLAERPYACPVESCDRRFSDNRDLTRHIRIHTGSQKPFQCRICMRNFSRSDHLTRHIRTHTGEKPYACPVESCDRRFSDNRDLTRHIRIHTGSQKPFQCRICMRNFSRSDHLTRHIRTHTGEKPYACPVESCDRRFSDNRDLTRHIRIHTGQKPGGGGSGGGGSGGGGSQKPFQCRICMRNFSRSDHLTRHIRTHTGEKPYACPVESCDRRFSDNRDLTRHIRIHTGSQKPFQCRICMRNFSRSDHLTRHIRTHTGEKPYACPVESCDRRFSDNRDLTRHIRIHTGSQKPFQCRICMRNFSRSDHLTRHIRTHTGEKPYACPVESCDRRFSDNRDLTRHIRIHTGQKPGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 98 - protein, 477 aa, Artificial Sequence
  ZF11xALS3-Kox
MGRIRKKLRLAERPYACPVESCDRRFSTREDLTRHIRIHTGSQKPFQCRICMRNFSRSDHLTRHIRTHTGEKPFACDICGRKFATREDRKRHTKIHTGSQKPFQCRICMRNFSRSDHLTRHIRTHTGEKPFACDICGRKFATREDRKRHTKIHLRQKDGGGGSGGGGSGGGGSQKPFQCRICMRNFSRSDHLTRHIRTHTGEKPFACDICGRKFATREDRKRHTKIHTGSQKPFQCRICMRNFSRSDHLTRHIRTHTGEKPFACDICGRKFATREDRKRHTKIHTGSQKPFQCRICMRNFSRSDHLTRHIRTHTGEKPFACDICGRKFATREDRKRHTKIHLRQKDLRQKDGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 99 - protein, 482 aa, Artificial Sequence
  ZF11xALS-TV8-Kox
MGRIRKKLRLAERPYACPVESCDRRFSDNGDLTRHIRIHTGSQKPFQCRICMRNFSRSAHLTRHIRTHTGEKPYACPVESCDRRFSDNGDLTRHIRIHTGSQKPFQCRICMRNFSRSAHLTRHIRTHTGEKPYACPVESCDRRFSDNGDLTRHIRIHTGQKPGGGGSGGGGSGGGGSQKPFQCRICMRNFSRSAHLTRHIRTHTGEKPYACPVESCDRRFSDNGDLTRHIRIHTGSQKPFQCRICMRNFSRSAHLTRHIRTHTGEKPYACPVESCDRRFSDNGDLTRHIRIHTGSQKPFQCRICMRNFSRSAHLTRHIRTHTGEKPYACPVESCDRRFSDNGDLTRHIRIHTGQKPGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 100 - protein, 482 aa, Artificial Sequence
  ZF11xALS-TV9-Kox
MGRIRKKLRLAERPYACPVESCDRRFSDGADLTRHIRIHTGSQKPFQCRICMRNFSRSAHLTRHIRTHTGEKPYACPVESCDRRFSDGADLTRHIRIHTGSQKPFQCRICMRNFSRSAHLTRHIRTHTGEKPYACPVESCDRRFSDGADLTRHIRIHTGQKPGGGGSGGGGSGGGGSQKPFQCRICMRNFSRSAHLTRHIRTHTGEKPYACPVESCDRRFSDGADLTRHIRIHTGSQKPFQCRICMRNFSRSAHLTRHIRTHTGEKPYACPVESCDRRFSDGADLTRHIRIHTGSQKPFQCRICMRNFSRSAHLTRHIRTHTGEKPYACPVESCDRRFSDGADLTRHIRIHTGQKPGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 101 - protein, 482 aa, Artificial Sequence
  ZF11xALS-TV10-Kox
MGRIRKKLRLAERPYACPVESCDRRFSAGADLTRHIRIHTGSQKPFQCRICMRNFSRSAHLTRHIRTHTGEKPYACPVESCDRRFSAGADLTRHIRIHTGSQKPFQCRICMRNFSRSAHLTRHIRTHTGEKPYACPVESCDRRFSAGADLTRHIRIHTGQKPGGGGSGGGGSGGGGSQKPFQCRICMRNFSRSAHLTRHIRTHTGEKPYACPVESCDRRFSAGADLTRHIRIHTGSQKPFQCRICMRNFSRSAHLTRHIRTHTGEKPYACPVESCDRRFSAGADLTRHIRIHTGSQKPFQCRICMRNFSRSAHLTRHIRTHTGEKPYACPVESCDRRFSAGADLTRHIRIHTGQKPGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 102 - protein, 505 aa, Artificial Sequence
  Active delivery construct (prt) SS-2xNLS-ZFP- KOX
MGSLVLTLCALFCLAAYLVSGRIRRDMGPKKKRKVPKKKRKVGERPYACPVESCDRRFSQSGDLTRHIRIHTGSQKPFQCRICMRNFSQSGDLTRHIRTHTGEKPFACDICGRKFAQSGDRKRHTKIHTGSQKPFQCRICMRNFSQSGDLTRHIRTHTGEKPFACDICGRKFAQSGDRKRHTKIHLRQKDGGGGSGGGGSGGGGSQKPFQCRICMRNFSQSGDLTRHIRTHTGEKPFACDICGRKFAQSGDRKRHTKIHTGSQKPFQCRICMRNFSQSGDLTRHIRTHTGEKPFACDICGRKFAQSGDRKRHTKIHTGSQKPFQCRICMRNFSQSGDLTRHIRTHTGEKPFACDICGRKFAQSGDRKRHTKIHLRQKDGGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 103 - protein, 505 aa, Artificial Sequence
  Active delivery construct (prt) SS-2xNLS-ZFP- ALS1-KOX
MGSLVLTLCALFCLAAYLVSGRIRRDMGPKKKRKVPKKKRKVGERPYACPVESCDRRFSDSSVLTRHIRIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHLRQKDGGGGSGGGGSGGGGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHLRQKDGGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 104 - protein, 505 aa, Artificial Sequence
  Active delivery construct (prt) SS-2xNLS-ZFP- FXTAS1-KOX
MGSLVLTLCALFCLAAYLVSGRIRRDMGPKKKRKVPKKKRKVGERPYACPVESCDRRFSRSDELTRHIRIHTGSQKPFQCRICMRNFSRSDELTRHIRTHTGEKPFACDICGRKFARSDERKRHTKIHTGSQKPFQCRICMRNFSRSDELTRHIRTHTGEKPFACDICGRKFARSDERKRHTKIHLRQKDGGGGSGGGGSGGGGSQKPFQCRICMRNFSRSDELTRHIRTHTGEKPFACDICGRKFARSDERKRHTKIHTGSQKPFQCRICMRNFSRSDELTRHIRTHTGEKPFACDICGRKFARSDERKRHTKIHTGSQKPFQCRICMRNFSRSDELTRHIRTHTGEKPFACDICGRKFARSDERKRHTKIHLRQKDGGGGSGGGGSSLSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 105 - DNA, 1292 bp, Artificial Sequence
  CMV promoter
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaaattggatctccattcgccattcaggctgcgcaactgctgggaaggacgatcagagcgggcctcttcgctattacgccagctggcgaaagggacgtggcaagcaaggcgattaagttgagttacgccaggattttcccagtcacgacgttgtaaaacgacggccagagaattataatacgactcactatagggcgaattcggatccttgctagcctcgagacgcgtgattcaccatg

SEQ ID NO: 106 - DNA, 819 bp, Artificial Sequence
  CBh promoter
gcggccgcacgcgtcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggggatcc

SEQ ID NO: 107 - protein, 7 aa, Artificial Sequence
  Recognition Sequence
  MISC_FEATURE (1)..(1): Asp, Glu, Thr, Val or Ser
  MISC_FEATURE (2)..(2): Ser, Asn or Arg
  MISC_FEATURE (3)..(3): Ser, Arg or Glu
  MISC_FEATURE (4)..(4): Val, Asp, Glu, Ile, Leu, Ser or Thr
  MISC_FEATURE (5)..(5): Leu or Arg
  MISC_FEATURE (6)..(6): Thr or Lys
  MISC_FEATURE (7)..(7): Arg or Lys
XXXXXXX

SEQ ID NO: 108 - protein, 7 aa, Artificial Sequence
  Recognition Sequence
  MISC_FEATURE (6)..(6): Thr, Ser or Ala
DSSVLXR

SEQ ID NO: 109 - protein, 7 aa, Artificial Sequence
  Recognition Sequence
  MISC_FEATURE (5)..(5): Leu or Arg
  MISC_FEATURE (6)..(6): Thr or Lys
  MISC_FEATURE (7)..(7): Arg or Lys
RSDHXXX

SEQ ID NO: 110 - protein, 7 aa, Artificial Sequence
  Recognition Sequence
  MISC_FEATURE (6)..(6): Thr, Ser or Ala
  MISC_FEATURE (7)..(7): Arg or Lys
RSDHLXX

SEQ ID NO: 111 - protein, 7 aa, Artificial Sequence
  Recognition Sequence
RSDHRKR

SEQ ID NO: 112 - protein, 5 aa, Artificial Sequence
  Linker Sequence
TGEKP

SEQ ID NO: 113 - protein, 5 aa, Artificial Sequence
  Linker Sequence
  MISC_FEATURE (3)..(3): Glu or Gln
  MISC_FEATURE (4)..(4): Lys or Arg
TGXXP

SEQ ID NO: 114 - protein, 5 aa, Artificial Sequence
  Linker Sequence
TGQKP

SEQ ID NO: 115 - protein, 6 aa, Artificial Sequence
  Linker Sequence
  MISC_FEATURE (3)..(3): Gly or Ser
  MISC_FEATURE (4)..(4): Glu or Gln
  MISC_FEATURE (5)..(5): Lys or Arg
TGXXXP

SEQ ID NO: 116 - protein, 6 aa, Artificial Sequence
  Linker Sequence
TGGERP

SEQ ID NO: 117 - protein, 6 aa, Artificial Sequence
  Linker Sequence
TGSERP

SEQ ID NO: 118 - protein, 6 aa, Artificial Sequence
  Linker Sequence
TGGQRP

SEQ ID NO: 119 - protein, 6 aa, Artificial Sequence
  Linker Sequence
TGSQRP

SEQ ID NO: 120 - protein, 6 aa, Artificial Sequence
  Linker Sequence
TGGEKP

SEQ ID NO: 121 - protein, 6 aa, Artificial Sequence
  Linker Sequence
TGSEKP

SEQ ID NO: 122 - protein, 6 aa, Artificial Sequence
  Linker Sequence
TGGQKP

SEQ ID NO: 123 - protein, 6 aa, Artificial Sequence
  Linker Sequence
TGSQKP

SEQ ID NO: 124 - protein, 7 aa, Artificial Sequence
  Linker Sequence
  MISC_FEATURE (3)..(4): Gly or Ser
  MISC_FEATURE (3)..(4): This region may encompass 0, 1 or 2 residues
  MISC_FEATURE (5)..(5): Glu or Gln
  MISC_FEATURE (6)..(6): Lys or Arg
TGXXXXP

SEQ ID NO: 125 - protein, 7 aa, Artificial Sequence
  Linker Sequence
  MISC_FEATURE (2)..(3): Gly or Ser
  MISC_FEATURE (2)..(3): This region may encompass 0, 1 or 2 residues
  MISC_FEATURE (5)..(5): Glu or Gln
  MISC_FEATURE (6)..(6): Lys or Arg
TXXGXXP

SEQ ID NO: 126 - protein, 8 aa, Artificial Sequence
  Linker Sequence
  MISC_FEATURE (3)..(5): Gly or Ser
  MISC_FEATURE (6)..(6): Glu or Gln
  MISC_FEATURE (7)..(7): Lys or Arg
TGXXXXXP

SEQ ID NO: 127 - protein, 8 aa, Artificial Sequence
  Linker Sequence
  MISC_FEATURE (2)..(4): Gly or Ser
  MISC_FEATURE (6)..(6): Glu or Gln
  MISC_FEATURE (7)..(7): Lys or Arg
TXXXGXXP

SEQ ID NO: 128 - protein, 19 aa, Artificial Sequence
  Linker Sequence
  MISC_FEATURE (6)..(10): This region may include 1, 2, 3 or 4 repeat sequences of Gly Gly Gly Gly Ser
LRQKDGGGGSQLVGTAERP

SEQ ID NO: 129 - protein, 13 aa, Artificial Sequence
  Linker Sequence
  MISC_FEATURE (6)..(10): This region may include 1, 2, 3 or 4 repeat sequences of Gly Gly Gly Gly Ser
LRQKDGGGGSQKP

SEQ ID NO: 130 - protein, 29 aa, Artificial Sequence
  Linker Sequence
LRQKDGGGGSGGGGSGGGGSQLVGTAERP

SEQ ID NO: 131 - protein, 23 aa, Artificial Sequence
  Linker Sequence
LRQKDGGGGSGGGGSGGGGSQKP

SEQ ID NO: 132 - protein, 5 aa, Artificial Sequence
  Linker Sequence
TGERP

SEQ ID NO: 133 - protein, 7 aa, Artificial Sequence
  Recognition Sequence
  MISC_FEATURE (1)..(1): Arg or Gly
  MISC_FEATURE (3)..(3): Asp or Gly
  MISC_FEATURE (6)..(6): Thr, Ser or Ala
  MISC_FEATURE (7)..(7): Arg, Lys or Gly
XSXHLXX

SEQ ID NO: 134 - protein, 7 aa, Artificial Sequence
  Recognition Sequence
  MISC_FEATURE (1)..(1): Arg or Gly
  MISC_FEATURE (3)..(3): Asp, Ser or Gly
  MISC_FEATURE (6)..(6): Lys, Ile or Ala
  MISC_FEATURE (7)..(7): Arg, Lys or Gly
XGXHRXX

SEQ ID NO: 135 - protein, 7 aa, Artificial Sequence
  Recognition Sequence
  MISC_FEATURE (1)..(1): Arg or Gly
  MISC_FEATURE (7)..(7): Arg or Lys
XSDHLTX

SEQ ID NO: 136 - protein, 7 aa, Artificial Sequence
  Recognition Sequence
  MISC_FEATURE (3)..(3): Asp or Ser
  MISC_FEATURE (7)..(7): Arg or Lys
RGXHRKX

SEQ ID NO: 137 - protein, 7 aa, Artificial Sequence
  Recognition Sequence
DSSVLTG

SEQ ID NO: 138 - protein, 7 aa, Artificial Sequence
  Recognition Sequence
DSSVRKG

SEQ ID NO: 139 - protein, 7 aa, Artificial Sequence
  Recognition Sequence
DSSELTG

SEQ ID NO: 140 - protein, 7 aa, Artificial Sequence
  Recognition Sequence
DSSERKG

SEQ ID NO: 141 - protein, 7 aa, Artificial Sequence
  Recognition Sequence
RGDHLTR

SEQ ID NO: 142 - protein, 7 aa, Artificial Sequence
  Recognition Sequence
GSDHLTR

SEQ ID NO: 143 - protein, 7 aa, Artificial Sequence
  Recognition Sequence
RGDHLTK

SEQ ID NO: 144 - protein, 7 aa, Artificial Sequence
  Recognition Sequence
GSDHLTK

SEQ ID NO: 145 - protein, 7 aa, Artificial Sequence
  Recognition Sequence
DNGDLTR

SEQ ID NO: 146 - protein, 7 aa, Artificial Sequence
  Recognition Sequence
DGADLTR

SEQ ID NO: 147 - protein, 7 aa, Artificial Sequence
  Recognition Sequence
AGADLTR

SEQ ID NO: 148 - protein, 7 aa, Artificial Sequence
  SV40 NLS
PKKKRKV

SEQ ID NO: 149 - protein, 8 aa, Artificial Sequence
  Human KIAA2022 NLS
PKKRRKVT

SEQ ID NO: 150 - protein, 7 aa, Artificial Sequence
  Mouse primase p58 NLS9
RIRKKLR

SEQ ID NO: 151 - protein, 116 aa, Artificial Sequence
  Human Kox-1 KRAB domain
LSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 152 - protein, 75 aa, Artificial Sequence
  Mouse ZF87 KRAB domain
EEMLSFRDVAIDFSAEEWECLEPAQWNLYRDVMLENYSHLVFLGLASCKPYLVTFLEQRQEPSVVKRPAAATVHP

SEQ ID NO: 153 - protein, 25 aa, Artificial Sequence
  Linker Sequence
LRQKDGGGGSGGGGSGGGGSQLVSS

SEQ ID NO: 154 - protein, 16 aa, Artificial Sequence
  Linker Sequence
LRQKDGGGGSGGGGSS

SEQ ID NO: 155 - protein, 14 aa, Artificial Sequence
  Linker Sequence
LRQKDGGGSGGGGS

SEQ ID NO: 156 - protein, 21 aa, Artificial Sequence
  BMP10 secretion signal
MGSLVLTLCALFCLAAYLVSG

SEQ ID NO: 157 - protein, 14 aa, Artificial Sequence
  Human N-terminal leader
MGPKKRRKVTGERP

SEQ ID NO: 158 - protein, 15 aa, Artificial Sequence
  Human N-terminal leader
MGPKKRRKVTLAERP

SEQ ID NO: 159 - protein, 14 aa, Artificial Sequence
  Mouse N-terminal leader
MGRIRKKLRLAERP

SEQ ID NO: 160 - protein, 14 aa, Artificial Sequence
  Double SV40 NLS
PKKKRKVPKKKRKV

SEQ ID NO: 161 - DNA, 1188 bp, Artificial Sequence
  Human EF1alpha-1 promoter - 1188bp (GenBank J04617.1)
gaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaa

SEQ ID NO: 162 - DNA, 507 bp, Artificial Sequence
  Human synapsin promoter
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatgg

SEQ ID NO: 163 - DNA, 1815 bp, Artificial Sequence
  pCAG-promoter
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcctcgagaccatg

SEQ ID NO: 164 - protein, 89 aa, Artificial Sequence
  Mouse Zif268
ERPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 165 - protein, 89 aa, Artificial Sequence
  Human Zif268
ERPYACPVESCDRRFSRSDELTRHIRIHTGQKPFQCRICMRNFSRSDHLTTHIRTHTGEKPFACDICGRKFARSDERKGHTKIHLRQKD

SEQ ID NO: 166 - protein, 327 aa, Artificial Sequence
  11-zinc finger peptide - ALS-binding 1
YACPVESCDRRFSDSSVLTRHIRIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHLRQKDGGGGSGGGGSGGGGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIH

SEQ ID NO: 167 - protein, 337 aa, Artificial Sequence
  11-zinc finger peptide - ALS-binding 2
YACPVESCDRRFSDNRDLTRHIRIHTGSQKPFQCRICMRNFSRSDHLTRHIRTHTGEKPYACPVESCDRRFSDNRDLTRHIRIHTGSQKPFQCRICMRNFSRSDHLTRHIRTHTGEKPYACPVESCDRRFSDNRDLTRHIRIHTGQKPGGGGSGGGGSGGGGSQKPFQCRICMRNFSRSDHLTRHIRTHTGEKPYACPVESCDRRFSDNRDLTRHIRIHTGSQKPFQCRICMRNFSRSDHLTRHIRTHTGEKPYACPVESCDRRFSDNRDLTRHIRIHTGSQKPFQCRICMRNFSRSDHLTRHIRTHTGEKPYACPVESCDRRFSDNRDLTRHIRIH

SEQ ID NO: 168 - protein, 327 aa, Artificial Sequence
  11-zinc finger peptide - ALS-binding 3
YACPVESCDRRFSTREDLTRHIRIHTGSQKPFQCRICMRNFSRSDHLTRHIRTHTGEKPFACDICGRKFATREDRKRHTKIHTGSQKPFQCRICMRNFSRSDHLTRHIRTHTGEKPFACDICGRKFATREDRKRHTKIHLRQKDGGGGSGGGGSGGGGSQKPFQCRICMRNFSRSDHLTRHIRTHTGEKPFACDICGRKFATREDRKRHTKIHTGSQKPFQCRICMRNFSRSDHLTRHIRTHTGEKPFACDICGRKFATREDRKRHTKIHTGSQKPFQCRICMRNFSRSDHLTRHIRTHTGEKPFACDICGRKFATREDRKRHTKIH

SEQ ID NO: 169 - protein, 139 aa, Artificial Sequence
  5-zinc finger peptide - ALS-binding 1
YACPVESCDRRFSDSSVLTRHIRIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIH

SEQ ID NO: 170 - protein, 165 aa, Artificial Sequence
  6-zinc finger peptide - ALS-binding 1
FQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIHTGSQKPFQCRICMRNFSRSDHLTRHIRTHTGEKPFACDICGRKFADSSVRKRHTKIH

SEQ ID NO: 171 - protein, 327 aa, Artificial Sequence
  11-zinc finger peptide - ALS-binding variant 1 (TV1)
YACPVESCDRRFSDSSVLTRHIRIHTGSQKPFQCRICMRNFSRSGHLTRHIRTHTGEKPFACDICGRKFADSSVRKRHTKIHTGSQKPFQCRICMRNFSRSGHLTRHIRTHTGEKPFACDICGRKFADSSVRKRHTKIHLRQKDGGGGSGGGGSGGGGSQKPFQCRICMRNFSRSGHLTRHIRTHTGEKPFACDICGRKFADSSVRKRHTKIHTGSQKPFQCRICMRNFSRSGHLTRHIRTHTGEKPFACDICGRKFADSSVRKRHTKIHTGSQKPFQCRICMRNFSRSGHLTRHIRTHTGEKPFACDICGRKFADSSVRKRHTKIH

SEQ ID NO: 172 - protein, 327 aa, Artificial Sequence
  11-zinc finger peptide - ALS-binding variant 2 (TV2)
YACPVESCDRRFSASSELTRHIRIHTGSQKPFQCRICMRNFSRSGHLTRHIRTHTGEKPFACDICGRKFAASSERKRHTKIHTGSQKPFQCRICMRNFSRSGHLTRHIRTHTGEKPFACDICGRKFAASSERKRHTKIHLRQKDGGGGSGGGGSGGGGSQKPFQCRICMRNFSRSGHLTRHIRTHTGEKPFACDICGRKFAASSERKRHTKIHTGSQKPFQCRICMRNFSRSGHLTRHIRTHTGEKPFACDICGRKFAASSERKRHTKIHTGSQKPFQCRICMRNFSRSGHLTRHIRTHTGEKPFACDICGRKFAASSERKRHTKIH

SEQ ID NO: 173 - protein, 327 aa, Artificial Sequence
  11-zinc finger peptide - ALS-binding variant 3 (TV3)
YACPVESCDRRFSASSELTRHIRIHTGSQKPFQCRICMRNFSRSGHLTKHIRTHTGEKPFACDICGRKFAASSERKRHTKIHTGSQKPFQCRICMRNFSRSGHLTKHIRTHTGEKPFACDICGRKFAASSERKRHTKIHLRQKDGGGGSGGGGSGGGGSQKPFQCRICMRNFSRSGHLTKHIRTHTGEKPFACDICGRKFAASSERKRHTKIHTGSQKPFQCRICMRNFSRSGHLTKHIRTHTGEKPFACDICGRKFAASSERKRHTKIHTGSQKPFQCRICMRNFSRSGHLTKHIRTHTGEKPFACDICGRKFAASSERKRHTKIH

SEQ ID NO: 174 - protein, 327 aa, Artificial Sequence
  11-zinc finger peptide - ALS-binding variant 4 (TV4)
YACPVESCDRRFSDSGDLTRHIRIHTGSQKPFQCRICMRNFSRSGHLTKHIRTHTGEKPFACDICGRKFADSGDRKRHTKIHTGSQKPFQCRICMRNFSRSGHLTKHIRTHTGEKPFACDICGRKFADSGDRKRHTKIHLRQKDGGGGSGGGGSGGGGSQKPFQCRICMRNFSRSGHLTKHIRTHTGEKPFACDICGRKFADSGDRKRHTKIHTGSQKPFQCRICMRNFSRSGHLTKHIRTHTGEKPFACDICGRKFADSGDRKRHTKIHTGSQKPFQCRICMRNFSRSGHLTKHIRTHTGEKPFACDICGRKFADSGDRKRHTKIH

SEQ ID NO: 175 - protein, 327 aa, Artificial Sequence
  11-zinc finger peptide - ALS-binding variant 5 (TV5)
YACPVESCDRRFSDSGELTRHIRIHTGSQKPFQCRICMRNFSRSGHLTGHIRTHTGEKPFACDICGRKFADSGERKRHTKIHTGSQKPFQCRICMRNFSRSGHLTGHIRTHTGEKPFACDICGRKFADSGERKRHTKIHLRQKDGGGGSGGGGSGGGGSQKPFQCRICMRNFSRSGHLTGHIRTHTGEKPFACDICGRKFADSGERKRHTKIHTGSQKPFQCRICMRNFSRSGLTKGHIRTHTGEKPFACDICGRKFADSGERKRHTKIHTGSQKPFQCRICMRNFSRSGHLTGHIRTHTGEKPFACDICGRKFADSGERKRHTKIH

SEQ ID NO: 176 - protein, 327 aa, Artificial Sequence
  11-zinc finger peptide - ALS-binding variant 6 (TV6)
YACPVESCDRRFSGSSELTRHIRIHTGSQKPFQCRICMRNFSRSDHLTRHIRTHTGEKPFACDICGRKFAGSSERKRHTKIHTGSQKPFQCRICMRNFSRSDHLTRHIRTHTGEKPFACDICGRKFAGSSERKRHTKIHLRQKDGGGGSGGGGSGGGGSQKPFQCRICMRNFSRSDHLTRHIRTHTGEKPFACDICGRKFAGSSERKRHTKIHTGSQKPFQCRICMRNFSRSDHLTRHIRTHTGEKPFACDICGRKFAGSSERKRHTKIHTGSQKPFQCRICMRNFSRSDHLTRHIRTHTGEKPFACDICGRKFAGSSERKRHTKIH

SEQ ID NO: 177 - protein, 327 aa, Artificial Sequence
  11-zinc finger peptide - ALS-binding variant 7 (TV7)
YACPVESCDRRFSGSSELTKHIRIHTGSQKPFQCRICMRNFSRSGHLTKHIRTHTGEKPFACDICGRKFAGSSELTKHTKIHTGSQKPFQCRICMRNFSRSGHLTKHIRTHTGEKPFACDICGRKFAGSSELTKHTKIHLRQKDGGGGSGGGGSGGGGSQKPFQCRICMRNFSRSGHLTKHIRTHTGEKPFACDICGRKFAGSSELTKHTKIHTGSQKPFQCRICMRNFSRSGHLTKHIRTHTGEKPFACDICGRKFAGSSELTKHTKIHTGSQKPFQCRICMRNFSRSGHLTKHIRTHTGEKPFACDICGRKFAGSSELTKHTKIH

SEQ ID NO: 178 - protein, 337 aa, Artificial Sequence
  11-zinc finger peptide - ALS-binding variant 8 (TV8)
YACPVESCDRRFSDNGDLTRHIRIHTGSQKPFQCRICMRNFSRSAHLTRHIRTHTGEKPYACPVESCDRRFSDNGDLTRHIRIHTGSQKPFQCRICMRNFSRSAHLTRHIRTHTGEKPYACPVESCDRRFSDNGDLTRHIRIHTGQKPGGGGSGGGGSGGGGSQKPFQCRICMRNFSRSAHLTRHIRTHTGEKPYACPVESCDRRFSDNGDLTRHIRIHTGSQKPFQCRICMRNFSRSAHLTRHIRTHTGEKPYACPVESCDRRFSDNGDLTRHIRIHTGSQKPFQCRICMRNFSRSAHLTRHIRTHTGEKPYACPVESCDRRFSDNGDLTRHIRIH

SEQ ID NO: 179 - protein, 337 aa, Artificial Sequence
  11-zinc finger peptide - ALS-binding variant 9 (TV9)
YACPVESCDRRFSDGADLTRHIRIHTGSQKPFQCRICMRNFSRSAHLTRHIRTHTGEKPYACPVESCDRRFSDGADLTRHIRIHTGSQKPFQCRICMRNFSRSAHLTRHIRTHTGEKPYACPVESCDRRFSDGADLTRHIRIHTGQKPGGGGSGGGGSGGGGSQKPFQCRICMRNFSRSAHLTRHIRTHTGEKPYACPVESCDRRFSDGADLTRHIRIHTGSQKPFQCRICMRNFSRSAHLTRHIRTHTGEKPYACPVESCDRRFSDGADLTRHIRIHTGSQKPFQCRICMRNFSRSAHLTRHIRTHTGEKPYACPVESCDRRFSDGADLTRHIRIH

SEQ ID NO: 180 - protein, 337 aa, Artificial Sequence
  11-zinc finger peptide - ALS-binding variant 10 (TV10)
YACPVESCDRRFSAGADLTRHIRIHTGSQKPFQCRICMRNFSRSAHLTRHIRTHTGEKPYACPVESCDRRFSAGADLTRHIRIHTGSQKPFQCRICMRNFSRSAHLTRHIRTHTGEKPYACPVESCDRRFSAGADLTRHIRIHTGQKPGGGGSGGGGSGGGGSQKPFQCRICMRNFSRSAHLTRHIRTHTGEKPYACPVESCDRRFSAGADLTRHIRIHTGSQKPFQCRICMRNFSRSAHLTRHIRTHTGEKPYACPVESCDRRFSAGADLTRHIRIHTGSQKPFQCRICMRNFSRSAHLTRHIRTHTGEKPYACPVESCDRRFSAGADLTRHIRIH

SEQ ID NO: 181 - protein, 7 aa, Artificial Sequence
  Recognition Sequence
  MISC_FEATURE (1)..(1): Gly or Asp
  MISC_FEATURE (3)..(3): Ser or Gly
  MISC_FEATURE (4)..(4): Glu or Asp
  MISC_FEATURE (5)..(5): Leu or Arg
  MISC_FEATURE (6)..(6): Thr or Lys
  MISC_FEATURE (7)..(7): Arg or Lys
XSXXXXX

SEQ ID NO: 182 - protein, 7 aa, Artificial Sequence
  Recognition Sequence
  MISC_FEATURE (1)..(1): Gly or Asp
  MISC_FEATURE (3)..(3): Ser or Gly
  MISC_FEATURE (4)..(4): Glu or Asp
  MISC_FEATURE (7)..(7): Arg or Lys
XSXXLTX

SEQ ID NO: 183 - protein, 7 aa, Artificial Sequence
  Recognition Sequence
  MISC_FEATURE (1)..(1): Gly or Asp
  MISC_FEATURE (3)..(3): Ser or Gly
  MISC_FEATURE (4)..(4): Glu or Asp
  MISC_FEATURE (7)..(7): Arg or Lys
XSXXRKX

SEQ ID NO: 184 - protein, 7 aa, Artificial Sequence
  Recognition Sequence
  MISC_FEATURE (1)..(1): Asp or Ala
  MISC_FEATURE (2)..(2): Asn or Gly
  MISC_FEATURE (3)..(3): Gly or Ala
  MISC_FEATURE (4)..(4): Val or Asp
  MISC_FEATURE (5)..(5): Leu or Arg
  MISC_FEATURE (6)..(6): Thr or Lys
  MISC_FEATURE (7)..(7): Arg or Lys
XXXXXXX

SEQ ID NO: 185 - protein, 7 aa, Artificial Sequence
  Recognition Sequence
  MISC_FEATURE (1)..(1): Asp or Ala
  MISC_FEATURE (2)..(2): Asn or Gly
  MISC_FEATURE (3)..(3): Gly or Ala
  MISC_FEATURE (4)..(4): Val or Asp
  MISC_FEATURE (7)..(7): Arg or Lys
XXXXLTX

SEQ ID NO: 186 - protein, 7 aa, Artificial Sequence
  Recognition Sequence
  MISC_FEATURE (1)..(1): Asp or Ala
  MISC_FEATURE (2)..(2): Asn or Gly
  MISC_FEATURE (3)..(3): Gly or Ala
  MISC_FEATURE (4)..(4): Val or Asp
  MISC_FEATURE (7)..(7): Arg or Lys
XXXXRKX

SEQ ID NO: 187 - protein, 7 aa, Artificial Sequence
  Recognition Sequence
DSGDLTR

SEQ ID NO: 188 - protein, 7 aa, Artificial Sequence
  Recognition Sequence
DSGDRKR

SEQ ID NO: 189 - protein, 7 aa, Artificial Sequence
  Recognition Sequence
DSGELTR

SEQ ID NO: 190 - protein, 7 aa, Artificial Sequence
  Recognition Sequence
DSGERKR

SEQ ID NO: 191 - protein, 7 aa, Artificial Sequence
  Recognition Sequence
GSSELTK

(19) United States
(12) Patent Application Publication
(10) Pub. No.: US 2025/0188137 A1
(43) Pub. Date: Jun. 12, 2025

(54) THERAPEUTIC NUCLEIC ACIDS, PEPTIDES AND USES I

(71) Applicant: IMPERIAL COLLEGE INNOVATIONS LIMITED, London (GB)

(72) Inventors: Mark ISALAN, London (GB); Michal MIELCAREK, London (GB)

(21) Appl. No.: 18/004,122

(22) PCT Filed: Jul. 1, 2021
(86) PCT No.: PCT/GB2021/051677

§ 371 Date: Jan. 3, 2023

(30) Foreign Application Priority Data
Jul. 1, 2020 (GB) 2010075.6

(51) Int. Cl. C07K 14/47; A61K 48/00; C12N 15/86

(52) U.S. Cl. CPC C07K 14/4702; A61K 48/005; C12N 15/86; C07K 2319/09; C12N 2750/14143

(57) ABSTRACT

Disclosed herein are polypeptides for use in treating diseases associated with pathogenic genomic repeat sequences, such as neurological disorders. Also disclosed are nucleic acid molecules and vectors that encode such polypeptides. Therapeutic uses and methods for treating such diseases are also disclosed; in particular, therapeutic uses and methods comprising complementary pairs and combinations of therapeutic polypeptides, nucleic acids or vectors. Also disclosed is a method and associated peptides and nucleic acids for active, long-term delivery of therapeutic molecules to target cells in vivo or in vitro.

### FIELD OF THE INVENTION

This invention relates to novel zinc finger peptides and nucleic acids having desirable properties, and to methods and uses for such peptides and nucleic acids. In particular, the invention relates to novel combinations of nucleic acids or zinc finger peptides for therapeutic uses. More particularly, the invention relates to zinc finger peptide or nucleic acid combinations for the treatment of conditions characterised by overexpression of undesirable gene alleles and underexpression of desirable gene alleles.

### Sequence Listing

A CRF sequence listing having file name IMP0001 PA Sequence.txt (133,209 bytes) created on Jul. 18, 2023 is incorporated herein by reference. The nucleic acid and/or amino acid sequences listed in the accompanying sequence listing are shown using standard abbreviations as defined in 37 C.R.F. 1.822.

### BACKGROUND OF THE INVENTION

Neurological disorders are diseases that affect the central nervous system (brain and spinal cord), the peripheral nervous system (peripheral nerves and cranial nerves), and the autonomic nervous system (parts of which are located in both central and peripheral nervous systems). More than 600 neurological diseases have been identified in humans, which together affect all functions of the body, including coordination, communication, memory, learning, eating, and in some cases mortality.

Although many tissues and organs in animals are capable of self-repair, generally the neurological system is not. Therefore, neurological disorders are often characterised by a progressive worsening of symptoms, beginning with minor problems that allow detection and diagnosis, but becoming steadily more severe—often resulting in the death of the affected individual. While the exact causes or triggers of many neurological disorders are still unknown, for others the causes are well documented and researched. For some of these diseases there are ‘effective’ treatments, which alleviate symptoms and/or prolong survival. However, despite intense research efforts, for most neurological disorders, and particularly for the most serious diseases, there are still no cures. Hence, there is a clear need for new therapeutics and treatments for neurological disorders.

Current knowledge of neurological disorders suggests that they can be caused by many different factors, including (but not limited to): inherited genetic abnormalities, problems in the immune system, injury to the brain or nervous system, or diabetes. One known cause of neurological disorder is a genetic abnormality leading to the pathological expansion of nucleic acid repeats sequences, such as CAG repeats in the htt gene that leads to Huntington's disease (HD) (Walker (2007) Lancet 369(9557): 218-228; and Kumar et al. Pharmacol. Rep. 62(1): 1-14), and GGGGCC repeats in the C9ORF72 gene in Amyotrophic lateral sclerosis (ALS) or Frontotemporal dementia (FTD) (DeJesus-Hernandez et al. (2011), Neuron, 72: 245-56). The GGGGCC repeat expansion in C9ORF72 appears to cause errors in splicing transcript formation that leads to an overall downregulation of correctly-spliced C9ORF72 expression. Moreover, there is abberant Repeat-Associated Non-AUG (RAN) dependent translation of the expanded C9ORF72 transcript, leading to toxic peptide production that is thought to be important in the pathogenesis of ALS. This is also true in another repeat-expansion disease, Fragile X-Associated Tremor/Ataxia Syndrome (FXTAS), that is associated with CGG repeats and RAN translation toxicity (Kong et al., (2017) Frontiers in Cellular Neuroscience, 11, 128).

Amyotrophic lateral sclerosis (ALS) is a devastating neurological disease that belongs to the wider group of disorders known as ‘motor neuron diseases’, which are characterised by the gradual and progressive deterioration (degeneration) of the nerve cells (motor neurons) that control muscle movements. The disease, which is the most common motor neuron disease among adults, affects about 1 in 50,000 people and is currently without a cure. ALS tends to appear in mid-life (between the ages of 40 and 60), and affects men more frequently than women. In most cases, it appears to occur at random with no family history of the disease.

Frontotemporal dementia (FTD) is a relatively rare from of dementia, which occurs when nerve cells in the frontal and/or temporal lobes of the brain die, and the pathways that connect the lobes change as a result. Some of the chemical messengers that transmit signals between nerve cells are also lost. Over time, as more and more nerve cells die, the brain tissue in the frontal and temporal lobes shrinks, resulting in changes in personality and behaviour, and difficulties with language. These symptoms are initially different from the memory loss often associated with more common types of dementia, such as Alzheimer's disease, but as the disease progresses more of the brain becomes damaged and symptoms are often similar to those of the later stages of Alzheimer's disease. About 10 to 20% of people with FTD develop a motor neuron disorder.

The study by DeJesus-Hernandez et al. (2011) found the C9ORF72 repeat expansion to be the most common genetic abnormality in both familial ALS (22.5%) and familial FTD (11.7%). The repeat expansion appears to cause errors in splicing transcript formation that leads to an overall downregulation of correctly-spliced C9ORF72 expression.

To date, treatments for these and similar diseases, have generally focused on trying to control the symptoms of rather than the causes of illness. The U.S. Food and Drug Administration (FDA) has approved the drugs riluzole (Rilutek™) and edaravone (Radicava™) to treat ALS. Riluzole is believed to reduce damage to motor neurons by decreasing levels of glutamate, which transports messages between nerve cells and motor neurons. Clinical trials in people with ALS showed that riluzole may prolong survival by a few months, but does not reverse the damage already done to motor neurons. Edaravone has also been shown to slow the decline in clinical assessment of daily functioning in persons with ALS. Other medications often prescribed to treat immediate symptoms of the disease include drugs such as baclofen or diazepam to help control spasticity; gabapentin to help control pain; and trihexyphenidyl or amitriptyline to help patients swallow. There are no recognised treatments to specifically target FTD and any treatments focus on the symptoms; for example, patients may be prescribed behavioural modification drugs. In some cases, patients may be prescribed drugs that are used to treat Alzheimer's disease, but results are variable/unpredictable.

Therefore, it would be highly desirable to have alternative and/or more effective therapeutic molecules and treatments for diseases such as ALS and FTD and related disorders caused by expanded GGGGCC repeats.

It is thought that the treatment of most neurodegenerative diseases may require the correction of mutation(s) in vivo, directly in the affected tissue, or the sustained expression of therapeutic factors (Agustin-Pavón & Isalan (2014) BioEssays 36: 979-990), e.g. to alter gene expression levels. Since the brain has limited regenerative capacity and complex connectivity, the tissue cannot simply be removed, repaired and re-implanted.

Given that many genetic neurodegenerative diseases lead to the progressive physical and mental decline of the affected individual over months and typically years, unless a treatment is capable of fully reversing the cause of disease, it is likely that ongoing treatment will be required over a period of months or, more likely, years. Current therapeutic treatments (e.g. by gene therapy) reduce in efficacy over the days, weeks and months following a course of treatment/administration: for example, as the expression of a therapeutic transgene declines. In previous studies (WO2017077329) we demonstrated that an AAV vector containing a zinc finger therapeutic peptide expression cassette could be used to cause repression of the htt gene in an in vivo mouse model of Huntington's disease (HD) for at least 6 months after a single administration. However, by 6 months it was found that only approximately 25% of mouse brain cells expressed the therapeutic peptide.

Therefore, it would also be desirable to have an improved system and therapeutic genes and peptides for the expansion and maintenance of therapeutic peptide exposure to and activity in diseased cells.

The present invention seeks to overcome or at least alleviate one or more of the problems found in the prior art.

### SUMMARY OF THE INVENTION

The present inventors have identified that by down-regulating/repressing mutant gene alleles responsible to onset of disease symptoms, and/or by up-regulating/activating wild-type (WT) gene alleles, the normal/WT function may be restored.

Thus, in general terms, the present invention provides new zinc finger peptides and encoding nucleic acid molecules that can be used for the modulation of gene expression in vitro and/or in vivo. The new zinc finger peptides of the invention may be particularly useful in the modulation of target genes associated with expanded GGGGCC hexanucleotide repeats, and more specifically the targeted repression of such genes.

In first aspects and embodiments of the invention, the new zinc finger peptides (ZFPs) of the invention beneficially bind to expanded GGGGCC hexanucleotide repeats associated with mutated pathogenic genes more effectively/efficaciously (e.g. with greater specificity and affinity) than to wild-type hexanucleotide repeat sequences associated with non-pathogenic, normal genes. As a consequence, the possibility of more specific gene targeting is envisaged, which may be particularly useful for the modulation of gene expression within the genome and/or for distinguishing between similar nucleic acid sequences of differing lengths. Such ZFPs may particularly down-regulate/repress the expression of target pathogenic genes. Beneficially, non-target non-pathogenic (WT) genes are not down-regulated/repressed or are repressed to a much lesser extent than the mutant pathogenic genes.

In other aspects and embodiments, the new zinc finger peptides (ZFPs) of the invention, respectively, beneficially bind to wild-type/non-pathogenic genes associated with GGGGCC hexanucleotide repeats of shorter length than mutated, pathogenic allele repeat hexanucleotide sequences. Such ZFPs may particularly up-regulate/activate the expression of target WT genes. Beneficially, non-target pathogenic (mutant) genes are not up-regulated/activated or are activated to a much lesser extent than the target WT genes.

Furthermore, the invention relates to therapeutic molecules, molecular combinations and compositions for use in methods for treating neurological diseases, such as—in first aspects-Amyotrophic lateral sclerosis (ALS) and Frontotemporal dementia (FTD). In some aspects and embodiments, the invention is directed to methods and therapeutic treatment regimes for treating patients affected by or diagnosed with ALS and/or FTD and other diseases characterised by expanded nucleotide repeat sequences. For example, the therapeutic molecules of the invention may be used in medical treatments in isolation, in combination with other medicaments and in combination with each other. In particular, aspects and embodiments of the invention relate to combination therapies comprising one or more ZFP that down-regulates/represses the expression of target pathogenic genes (a ZFP repressor) in conjunction/in combination with one or more ZFP that up-regulates/activates the expression of target WT genes (a ZFP activator). According to some aspects and embodiments of the invention, both ZFP repressor and ZFP activator proteins may bind to and target the same hexanucleotide repeat sequence—particularly the repeat sequence GGGGCC. Suitably, ZFP repressor proteins (respectively) preferentially target expanded hexanucleotide repeat sequences associated with pathogenic alleles, whereas ZFP activator proteins preferentially target normal (short) hexanucleotide repeat sequences associated with WT gene alleles.

In embodiments of any aspect of the invention, ZFP repressor proteins bind with lower affinity to their respective hexanucleotide repeat sequences than their corresponding ZFP activator protein partner. In some embodiments, ZFP activator proteins bind to their respective hexanucleotide repeat sequences with higher affinity than their corresponding ZFP repressor protein partner. In some embodiments, ZFP repressor proteins may comprise more nucleotide-binding zinc finger domains than their corresponding ZFP activator protein partner.

The peptides/proteins of the invention may be useful in vitro and/or in vivo. In particular, the peptides of the invention may be useful in disease therapy, such as gene therapy; e.g. for delaying the onset of symptoms, and/or for treating or alleviating the symptoms of a disease or diseases; and/or for reducing the severity of or preventing the progression of a disease or diseases. Particular diseases include ALS and/or FTD.

In aspects and embodiments of the methods and therapeutic uses of the invention, the binding affinity and expression of ZFP combinations comprising a ZFP repressor and ZFP activator are ‘tuned’ so as to repress desired target pathogenic gene alleles and activate desired target WT gene alleles simultaneously in the same cells. ‘Tuning’ of complementary pairs/partners (or groups) of ZFPs may be achieved through a combination of deliberate weakening or strengthening of binding interactions between zinc finger domains and target nucleic acid sequences; differences in the number of zinc finger domains in the therapeutic ZFPs; and differences in the relative expression levels of the therapeutic ZFPs.

In aspects and embodiments, the invention is directed towards novel zinc finger peptides (ZFP) that may exhibit prolonged, mid- to long-term, expression in target organisms in vivo, so as to be useful in medical treatments that may require long-term activity of the therapeutic agent. The ZFP sequences of the invention, in some embodiments, are adapted/optimised to closely match endogenous/wild-type peptide sequences expressed in the target organism so as to have reduced toxity and immunogenicity. Cells expressing the zinc finger peptides of the invention may therefore be protected from the immune response of the target organism so as to prolong expression of the heterologous peptide in these cells.

In the present invention, the inventors have designed zinc finger peptides (ZFPs) to target the GGGGCC-expansion, which may be useful for targeting both ALS and FTD therapeutically. Zinc fingers are DNA-binding proteins that may be reengineered to bind to user-defined DNA-sequences (Nat. Biotechnol., (2001) 19, 656-60). Moreover, the presence of essentially identical nucleic acid sequences that are associated with wild-type genes that may be associated with an already evident haploinsufficiency makes such genomic targeting of pathogenic genes particularly challenging. Although the GGGGCC sequence has been targeted before (WO 2019/084140A1) the design presented here has significant differences and advantages over the prior art. First, the GGGGCC-targeting ZF sequences of the present invention are designed to function in a long single-chain poly-zinc finger protein that is tuned to bind longer expansions, preferentially using designed binding-destabilising mutations and/or linkers. Second, these contraints are applied within the further constraint of minimising potential epitopes and non-host (mouse, human) residues, in order to increase immunocompatibility in a therapeutic application. The inventors have accordingly devised a formula to define the design space for this challenging multi-objective optimisation.

Furthermore, the present inventors have determined that, in order to mitigate against the risk of further reducing the expression of any wild-type gene products, the ZFP repressors of the invention may desirably be optimised with novel binding-destabilising mutations to target binding preferentially to longer nucleotide repeat sequences of pathogenic genes, (i.e. higher repeat number), which in ALS and FTD may comprise between 700 and 1,600 repeats, rather than normal gene sequences which may have between 2 and 23 repeats (Neuron (2011) 72, 245-56). The present invention describes the engineering of zinc finger peptides to discriminate between alleles having long or short hexanucleotide repeat sequences in a therapeutic manner.

In a first aspect, therefore, the invention provides a polypeptide comprising a zinc finger peptide having from 8 to 32 zinc finger domains (F1 to F32) according to Formula 2: X0-2 C X1-5 C X2-7 X−1 X+1 X+2 X+3 X+4 X+5 X+6 H X3-6 H/C where X is any amino acid, the numbers in subscript indicate the possible numbers of residues represented by X, and the numbers in superscript indicate the position of the amino acid in the α-helix; wherein the polypeptide binds to a 5′-GGGGCC-3′ nucleic acid repeat sequence. Suitably, at least 8 adjacent zinc finger domains, F1 to F8, have a recognition sequence X−1 X+1 X+2 X+3 X+4 X+5 X+6 according to the sequence patterns disclosed herein for repressor peptides of the invention. In some embodiments, the sequences of the adjacent zinc finger domains may be defined by the following pattern:

| | | F2, F4, F6, | F3, F5, F7, |
|---|---|---|---|
| | F1 | F8, F10 etc | F9, F11 etc |
| ZFP I: | SEQ ID NO: 8 | SEQ ID NO: 3 | SEQ ID NO: 8 |
| ZFP J: | SEQ ID NO: 8 | SEQ ID NO: 3 | SEQ ID NO: 10 |
| ZFP K: | SEQ ID NO: 10 | SEQ ID NO: 3 | SEQ ID NO: 10 |
| ZFP L: | SEQ ID NO: 9 | SEQ ID NO: 3 | SEQ ID NO: 9 |
| ZFP M: | SEQ ID NO: 9 | SEQ ID NO: 3 | SEQ ID NO: 11 |
| ZFP N: | SEQ ID NO: 11 | SEQ ID NO: 3 | SEQ ID NO: 11 |
| ZFP W: | SEQ ID NO: 8 | SEQ ID NO: 12 | SEQ ID NO: 8 |
| ZFP X: | SEQ ID NO: 8 | SEQ ID NO: 12 | SEQ ID NO: 10 |
| ZFP Y: | SEQ ID NO: 10 | SEQ ID NO: 12 | SEQ ID NO: 10 |
| ZFP Z: | SEQ ID NO: 9 | SEQ ID NO: 12 | SEQ ID NO: 9 |
| ZFP AA: | SEQ ID NO: 9 | SEQ ID NO: 12 | SEQ ID NO: 11 |
| ZFP AB: | SEQ ID NO: 11 | SEQ ID NO: 12 | SEQ ID NO: 11 |
| ZFP AI: | SEQ ID NO: 3 | SEQ ID NO: 8 | SEQ ID NO: 3 |
| ZFP AJ: | SEQ ID NO: 3 | SEQ ID NO: 9 | SEQ ID NO: 3 |
| ZFP AK: | SEQ ID NO: 3 | SEQ ID NO: 10 | SEQ ID NO: 3 |
| ZFP AL: | SEQ ID NO: 3 | SEQ ID NO: 11 | SEQ ID NO: 3 |
| ZFP AS: | SEQ ID NO: 12 | SEQ ID NO: 8 | SEQ ID NO: 12 |
| ZFP AT: | SEQ ID NO: 12 | SEQ ID NO: 9 | SEQ ID NO: 12 |
| ZFP AU: | SEQ ID NO: 12 | SEQ ID NO: 10 | SEQ ID NO: 12 |
| ZFP AV: | SEQ ID NO: 12 | SEQ ID NO: 11 | SEQ ID NO: 12. |

In any of the above sequence patterns, SEQ ID NO: 12 may be replaced with SEQ ID NO: 135 and/or SEQ ID NO: 136.

In some embodiments, the sequences of the adjacent zinc finger domains may be defined by the following pattern:

| | | F2, F4, F6, | F3, F5, F7, |
|---|---|---|---|
| | F1 | F8, F10 etc | F9, F11 etc |
| ZFP JX: | SEQ ID NO: 181 | SEQ ID NO: 12 | SEQ ID NO: 181 |
| ZFP JY: | SEQ ID NO: 182 | SEQ ID NO: 12 | SEQ ID NO: 183 |
| ZFP JZ: | SEQ ID NO: 182 | SEQ ID NO: 12 | SEQ ID NO: 183 |
| ZFP KA: | SEQ ID NO: 181 | SEQ ID NO: 133 | SEQ ID NO: 181 |
| ZFP KB: | SEQ ID NO: 182 | SEQ ID NO: 133 | SEQ ID NO: 182 |
| ZFP KC: | SEQ ID NO: 182 | SEQ ID NO: 133 | SEQ ID NO: 183 |
| ZFP KD: | SEQ ID NO: 181 | SEQ ID NO: 134 | SEQ ID NO: 181 |
| ZFP KE: | SEQ ID NO: 182 | SEQ ID NO: 134 | SEQ ID NO: 182 |
| ZFP KF: | SEQ ID NO: 182 | SEQ ID NO: 134 | SEQ ID NO: 183 |
| ZFP KG: | SEQ ID NO: 12 | SEQ ID NO: 181 | SEQ ID NO: 134 |
| ZFP KH: | SEQ ID NO: 12 | SEQ ID NO: 182 | SEQ ID NO: 134 |
| ZFP KI: | SEQ ID NO: 12 | SEQ ID NO: 183 | SEQ ID NO: 134 |
| ZFP KJ: | SEQ ID NO: 133 | SEQ ID NO: 181 | SEQ ID NO: 134 |
| ZFP KK: | SEQ ID NO: 133 | SEQ ID NO: 182 | SEQ ID NO: 134 |
| ZFP KL: | SEQ ID NO: 133 | SEQ ID NO: 183 | SEQ ID NO: 134 |
| ZFP LP: | SEQ ID NO: 184 | SEQ ID NO: 3 | SEQ ID NO: 184 |
| ZFP LQ: | SEQ ID NO: 185 | SEQ ID NO: 3 | SEQ ID NO: 185 |
| ZFP LR: | SEQ ID NO: 186 | SEQ ID NO: 3 | SEQ ID NO: 186 |
| ZFP LS: | SEQ ID NO: 184 | SEQ ID NO: 12 | SEQ ID NO: 184 |
| ZFP LT: | SEQ ID NO: 185 | SEQ ID NO: 12 | SEQ ID NO: 185 |
| ZFP LU: | SEQ ID NO: 186 | SEQ ID NO: 12 | SEQ ID NO: 186. |

In any of the above sequence patterns, SEQ ID NO: 12 may be replaced with SEQ ID NO: 135 and/or SEQ ID NO: 136.

In another embodiment/aspect of this first aspect, the invention provides a polypeptide comprising a zinc finger peptide having from 5 to 7 zinc finger domains (F1 to F7) according to Formula 2: X0-2 C X1-5 C X2-7 X−1 X+1 X+2 X+3 X+4 X+5 X+6 H X3-6 H/C where X is any amino acid, the numbers in subscript indicate the possible numbers of residues represented by X, and the numbers in superscript indicate the position of the amino acid in the α-helix; wherein the polypeptide binds to a 5′-GGGGCC-3′ nucleic acid repeat sequence. Beneficially, the zinc finger domains have a recognition sequence X−1 X+1 X+2 X+3 X+4 X+5 X+6 according to the sequence patterns disclosed herein for activator peptides of the invention. In some embodiments, the sequences of the adjacent zinc finger domains may be defined by the following pattern:

| | F1 | F2, F4, F6 | F3, F5, F7 |
|---|---|---|---|
| ZFP GO: | SEQ ID NO: 109 | SEQ ID NO: 108 | SEQ ID NO: 109 |
| ZFP GN: | SEQ ID NO: 109 | SEQ ID NO: 108 | SEQ ID NO: 110 |
| ZFP FW: | SEQ ID NO: 107 | SEQ ID NO: 109 | SEQ ID NO: 107 |
| ZFP FV: | SEQ ID NO: 107 | SEQ ID NO: 109 | SEQ ID NO: 108. |

In another aspect, the invention provides a combination of a repressor peptide and an activator peptide of the invention, both of which target the same polynucleotide-repeat sequences (i.e. 5′-GGGGCC-3′ nucleic acid repeat sequences), as well as combinations of corresponding polynucleotides, expression constructs (such as vectors) and pharmaceutical compositions; or polynucleotides, expression constructs (such as vectors) and pharmaceutical compositions that encode/deliver both the activator and the repressor peptide to a target cell. In such combinations, the zinc finger activator peptide beneficially has fewer zinc finger-nucleic acid binding domains than the zinc finger repressor peptide. In this way, such activator peptides may be more suitably adapted to target the shorter nucleic acid-repeat sequences associated with wild-type (non-pathogenic) target genes in vivo; whereas such repressor peptides may be more suited to target expanded nucleic acid-repeat sequences associated with pathogenic gene constructs. Advantageously, the binding affinity of such zinc finger activator peptides for the repeat nucleic acid sequence is higher (on average) per zinc finger domain than for the corresponding zinc finger repressor peptide (i.e. if compared over the same number of zinc finger domains, such a zinc finger activator would have higher binding affinity than the zinc finger repressor). In other embodiments, the zinc finger activator has higher affinity for the nucleic acid repeat sequence than the zinc finger repressor. In this way, the zinc finger activator peptide may bind more preferentially and more strongly to the shorter nucleic acid-repeat sequences associated with wild-type (non-pathogenic) target genes than to expanded nucleic acid-repeat sequences associated with pathogenic gene constructs. Suitably, therefore, a zinc finger repressor peptide of the invention will not outcompete a zinc finger activator peptide for a target wild-type repeat sequence. Suitably, in use, such zinc finger activator peptides of the invention are expressed at a lower concentration than corresponding zinc finger repressor peptides, and expression constructs are suitably adapted to achieve higher expression levels of zinc finger repressor peptides of the invention compared to zinc finger activator peptides. In this way, the repressor peptides of the invention may preferably target and bind to expanded nucleic acid repeat sequences associated with pathogenic gene constructs over wild-type repeat sequences; and the zinc finger activator peptides of the invention may preferably target wild-type repeat sequences associated with beneficial gene constructs. In such embodiments, the nucleic acid repeat sequences may be 5′-GGGGCC-3′ repeat sequences. The invention also encompasses any such polypeptides, polynucleotides, vectors and compositions in methods of therapeutic treatments and for use in such methods.

Such methods and therapeutic uses may comprise administering to a subject the polypeptide, nucleic acid or vector according to these aspects and embodiments of the invention, such that the same target cell is exposed to or expresses both a repressor peptide and an activator peptide of the invention. Administration of the repressor and activator peptides may be simultaneously, sequentially or separate, provided both effector peptides are expressed in the same cell. Surprisingly, in this way, the expression of WT target genes may be beneficially upregulated while the expression of pathogenic target genes may be beneficially down-regulated through transcription activator and repressor peptides that target/bind to the same nucleic acid repeat sequences.

Polypeptides of the invention may comprise sequences having at least 90%, at least 91%, at least 92%, at least 93%, at least 94%, at least 95%, at least 96%, at least 97%, at least 98%, or at least 99% identity to any of the polypeptides of SEQ ID NOs: 166 to 180, 96 to 101 and 102 to 104.

As indicated above, the invention is directed to polynucleotide (or nucleic acid) molecules that encode the zinc finger peptides and polypeptides of the invention. Particularly, isolated polynucleotides are encompassed. In addition, the polynucleotides (or nucleic acid molecules) of the invention may be expression constructs for the expression of the peptide or polypeptide of the invention in vitro and/or in vivo. The nucleic acids of the invention may be adapted for expression in any desired system or organism, but preferred organisms are mouse—in which therapeutic effects for diseases targeted by the therapeutic polypeptides of the invention may be tested, and humans—which will likely be the ultimate recipients or any potential therapy.

For expression of polypeptides, nucleic acid molecules are conveniently inserted into a vector or plasmid. Vectors and plasmids may be adapted for replication (e.g. to produce large quantities of its own nucleic acid sequence in host cells), or may be adapted for protein expression (e.g. to produce large or suitable quantities of zinc finger-containing protein in host cells). Any vector may be used, but preferred are polypeptide expression vectors so that the encoded polypeptide is expressed in host cells (e.g. for purposes of therapeutic treatment). Advantageously, the vector comprises a beneficial long acting, tissue specific and/or (very) strong promoter/enhancer sequence such as pNSE, pHsp90, CBh, EF1α-1 or synapsin, as described herein.

Viral vectors are particularly useful for potential use in therapeutic applications due to their ability to target and/or infect specific cells types. Suitable viral vectors may include those derived from retroviruses (such as influenza, SIV, HIV, lentivirus, and Moloney murine leukaemia); adenoviruses; adeno-associated viruses (AAV); herpes simplex virus (HSV); and chimeric viruses. Adeno-associated virus (AAV) vectors are considered particularly useful for targeting therapeutic peptides to the central and peripheral nervous systems and to the brain. A preferred viral vector delivery system is based on the AAV2/1 and AAV2/9 viral subtypes.

Thus, the invention is particularly directed to an adeno-associated virus (AAV) vector comprising a nucleic acid expression construct capable of expressing at least one polypeptide comprising a zinc finger peptide, wherein the polypeptide and the zinc finger peptide are defined as disclosed herein. The invention is also, therefore, directed to a gene therapy method; as well as to methods for treating diseases; particularly neurological diseases, such as ALS and/or FTD.

In some embodiments of the methods and therapeutic uses of the invention more than one (e.g. two) nucleic acid construct may be administered sequentially, simultaneously or separately to a cell or patient to be treated. Each nucleic acid construct may encode one or more ZFP according to the invention, so as to cause two or more complementary ZFPs to be expressed, advantageously within the same cell.

The invention relates to polypeptides comprising zinc finger peptides as defined herein. Typically, the polypeptides of the invention include a zinc finger portion comprising a plurality of zinc finger domains and one or more beneficial auxiliary sequences, such as effector domains. Effector domains include nuclear localisation sequences and transcriptional repressor domains or transcriptional activation domains as described elsewhere herein. It will be appreciated that the invention encompasses any polypeptides that may be encoded by the nucleic acid molecules defined herein; and any nucleic acid molecules capable of expressing a polypeptide as defined herein. The at least one effector domain may be selected from transcriptional repressor domains, transcriptional activator domains, transcriptional insulator domains, chromatin remodelling, condensation or decondensation domains, nucleic acid or protein cleavage domains, dimerisation domains, enzymatic domains, signalling/targeting sequences or domains. Preferred effector domains are transcriptional repressor domains and transcriptional activator domains. Embodiments of the invention relate to pairs of different (complementary) ZFPs, one or which comprises a transcriptional repressor domains and one or which comprises a transcriptional activator domain.

Conveniently, the ZFPs according to first aspects of the invention bind double-stranded hexanucleotide repeat sequences comprising GGGGCC-repeat, GGGCCG-repeat, GGCCGG-repeat, GCCGGG-repeat, and/or CCGGGG-repeat sequences. In preferred embodiments, the ZFPs of the invention target and bind to GGGGCC-repeat sequences.

In some aspects and embodiments, ZFPs according to the invention bind double-stranded ALS/FTD hexanucleotide repeat sequences containing at least 30 hexanucleotide repeats, at least 100 hexanucleotide repeats or at least 200 hexanucleotide repeats. In embodiments, ZFPs according to these aspects and embodiments of the invention preferentially bind double-stranded hexanucleotide repeat sequences containing between about 30 and 2,000 hexanucleotide repeats, between about 100 and 1,600 hexanucleotide repeats, or between about 700 and 1,600 hexanucleotide repeats. Suitably, ZFPs according to these embodiments of the invention bind to such double-stranded hexanucleotide repeat sequences preferentially over double-stranded hexanucleotide repeat sequences containing less than 30 hexanucleotide repeats, less than 20 hexanucleotide repeats, and particularly over double-stranded hexanucleotide repeat sequences containing up to 10 hexanucleotide repeats. Such nucleic acid sequences are beneficially bound with a binding dissociation constant (Kd) of less than about 1 μM, less than about 100 nM, less than about 10 nM, or less than about 1 nM. ZFPs according to such aspects and embodiments on the invention are suitably ZFP repressors, which down-regulate or otherwise repress the expression of a target gene, particularly a pathogenic gene associated with the expanded hexanucleotide repeat sequence. In some embodiments, ZFPs according to the invention bind double-stranded hexanucleotide repeat sequences containing up to 30 hexanucleotide repeats, or up to 10 hexanucleotide repeats. In some such embodiments, ZFPs according to the invention bind double-stranded hexanucleotide repeat sequences containing between about 2 and 30 hexanucleotide repeats, or between about 2 and 8 hexanucleotide repeats. Suitably, ZFPs according to such embodiments of the invention may bind to double-stranded hexanucleotide repeat sequences with a binding dissociation constant of less than about 10 nM, less than about 1 nM, less than 100 pM or less than 10 pM. ZFPs according to such aspects and embodiments of the invention are suitably ZFP activators, which up-regulate or otherwise activate the expression of a target gene, particularly a wild-type gene associated with the hexanucleotide repeat sequence.

Polypeptides of the invention may also be administered to an individual or patient in need thereof. Suitably, the polypeptides of the invention are to treat neurodegenerative diseases; particularly diseases associated with expanded hexanucleotide repeat sequences, such as ALS and/or FTD.

A gene therapy method according to the invention may comprise administering to a person in need thereof or to cells previously removed from a person, a nucleic acid encoding a ZFP of the invention, and causing the polypeptide to be expressed in cells of the person/subject. In this way, the gene therapy method may be useful for treating a neurodegenerative disease; and particularly diseases associated with expanded hexanucleotide repeat sequences, such as ALS and/or FTD. Suitably, the ZFP is a ZFP repressor protein. In embodiments of these aspects of the invention, the method comprises administering more than one nucleic acid expression construct, each encoding a ZFP of the invention, and causing the ZFPs to be expressed in cells of the subject to be treated. The ZFPs may comprise a complementary pair of ZFPs, one of which is a ZFP repressor and one of which is a ZFP activator. In such embodiments, the ZFP repressor and ZFP activator proteins of the complementary pair preferably bind to the same nucleotide repeat sequence, but with a different binding dissociate content. In such embodiments, the ZFP repressor and ZFP activator proteins of the complementary pair may have different numbers of zinc finger domains, preferably where the ZFP repressor comprises a longer array of adjacent zinc finger domains than the ZFP activator. In some embodiments, the method comprises administering one nucleic acid encoding two (or more) ZFPs according to the invention; suitably, wherein the ZFPs comprise a complementary pair of ZFPs, one of which is a ZFP repressor and one of which is a ZFP activator. Where more than one nucleic acid/expression construct of the invention is used, such nucleic acids may be administered simultaneous, sequentially or separately.

Pharmaceutical composition of the invention may comprise nucleic acid molecules (such as vectors) and/or polypeptides as defined herein. It is envisaged that the pharmaceutical compositions of the invention may be used in a method of combination therapy with one or more additional therapeutic agent, may be used on their own, or may be used in combination with other compositions of the invention and optionally one or more additional therapeutic agent.

In aspects and embodiments, the invention relates to chimeric or fusion proteins comprising the zinc finger peptides of the invention conjugated to one or more non-zinc finger domain, such as effector domains as described elsewhere herein.

Some aspects and embodiments of the invention include formulations, medicaments and pharmaceutical compositions comprising the zinc finger peptides. In some embodiments, the invention relates to a zinc finger peptide for use in medicine. More specifically, the zinc finger peptides and therapeutics of the invention may be used for modulating the expression of a target gene in a cell. In some embodiments the target gene is the C9ORF72 gene in Amyotrophic lateral sclerosis (ALS) and Frontotemporal dementia (FTD). Particularly, in these aspects and embodiments the invention relates to the treatment of diseases or conditions associated with the expanded GGGGCC hexanucleotide repeat and/or expression of gene products encoded by such repeat sequences. Treatment may also include preventative as well as therapeutic treatments and alleviation of a disease or condition.

Beneficially, nucleic acid expression constructs according to the invention are suitable for sustained constitutive expression of ZFPs. Accordingly, nucleic acid sequences encoding ZFPs may be operably linked/associated with promoter sequences suitable for such sustained expression in vivo. Sustained expression is beneficially for a period of at least 3 weeks, at least 6 weeks, at least 12 weeks or at least 24 weeks. In the context of this invention, ‘promoter’ sequences may encompass both transcriptional promoter and enhancer elements within a nucleic acid sequence which have the effect of enabling, causing and/or enhancing transcription of an associated gene/nucleic acid construct. In other words, the use of the term ‘promoter’ does not exclude the possibility that the nucleic acid sequence concerned may also encompass other elements associated with transcription, such as enhancer elements.

Gene therapy methods are also disclosed, comprising administering to a subject in need thereof or to cells previously removed from the subject, a nucleic acid encoding one or more ZFP under the control of natural or synthetic promoter-enhancer sequences, and causing the polypeptide to be expressed in cells of the subject.

Thus, in embodiments there is provided a gene therapy method comprising administering to a subject in need thereof, or to cells previously removed from the subject, a vector comprising a pNSE, pHsp90, CBh, EF1α-1 or synapsin promoter-enhancer construct. In embodiments, the methods comprise administering to the subject to be treated (or to cells of the subject) a vector according to the invention with neuronal targeting specificity in combination with a promiscuous vector according to the invention. The method may comprise administering to the subject to be treated an AAV2/1 subtype adeno-associated virus (AAV) vector according to the invention in combination with an AAV2/9 subtype adeno-associated virus (AAV) vector according to the invention. The administering ‘in combination’ may be simultaneous, separate or sequential, as appropriate. Therapeutic uses of the constructs and viral vectors of the invention are also encompassed. The methods and constructs of the invention may be for treating a neurological disease or condition; particularly a disease or condition selected from the group consisting of Amyotrophic lateral sclerosis (ALS) and Frontotemporal dementia (FTD).

In second aspects of the invention, there are provided constructs and methods for enhanced expression and delivery of therapeutic molecules of the invention to target cells in vivo or in vitro.

In embodiments of these aspects of the invention, the therapeutic molecule is a polypeptide that comprises an active/therapeutic agent, a secretory sequence (SS)/signal peptide (SP), and at least one nuclear localisation sequence (NLS) (as described herein). Suitably the active agent is a transcription factor such as a zinc finger peptide. The active agent may comprise an ‘effector’ domain, such as a restriction endonuclease or a transcriptional repressor or activator domain. Beneficially, a protease cleavage site is provided between the secretory sequence and the active agent, so that the secretory sequence may be removed once the therapeutic molecule enters a target cell.

In embodiments on the third aspect, there is provided an isolated polynucleotide encoding a polypeptide for delivery of an effector peptide to a target cell or a second population of cells; the polynucleotide comprising: (a) sequence encoding a polypeptide, the polypeptide comprising: (i) the effector peptide sequence; (ii) a cell secretion peptide sequence operably linked to the effector peptide sequence; (iii) a cell penetration and/or cell localisation peptide sequence operably linked to the effector peptide sequence; and (b) a polypeptide expression element operable to cause the polypeptide to be expressed in a source cell or first population of cells. Beneficially, in accordance with these aspects and embodiments, the first population of cells comprises different cells to the second population of cells; or the target cell is a different cell to the source cell, such that the effector peptide is expressed in a different cell or cells to the cell or cells to which it is intended to be delivered.

Corresponding methods in this aspect of the invention relate to a method (e.g. in vitro or in vivo) for delivery of a biological effector moiety to a target cell, the method comprising: (i) providing a nucleic acid expression construct encoding an expressible biological effector peptide, the biological effector peptide adapted for (a) cell secretion from a first cell or population of cells, and (b) cell penetration of a second cell or population of cells, wherein the first and second target cells may be of the same type or of different types; (ii) delivering the nucleic acid expression construct to the first cell or population of cells; (iii) expressing the expressible biological effector peptide in the first cell or population of cells, and allowing it to be secreted from the first cell or population of cells; (iv) bringing the secreted biological effector peptide into contact with the second cell or population of cells under conditions that allow the biological effector peptide to penetrate the second cell or population of cells; thereby to deliver the biological effector moiety to the target (second) cell or population of cells. Advantageously, according to these aspects and embodiments of the invention the second (target) cell or population of cells is/are different to the first cell or population of cells in which the biological effector peptide was expressed.

Preferably the therapeutic peptide is a ZFP according to the invention.

The invention also encompasses nucleic acid molecules encoding these therapeutic peptides of the invention.

It will be appreciated that any features of one aspect or embodiment of the invention may be combined with any combination of features in any other aspect or embodiment of the invention, unless otherwise stated, and such combinations fall within the scope of the claimed invention.

### BRIEF DESCRIPTION OF THE DRAWINGS

The invention is further illustrated by the accompanying drawings in which:

FIG. 1 A schematic illustration of an optimal design for a 2-zinc finger peptide array that recognises the nucleic acid sequence 5′-GGG GCC-3. 2-zinc finger subunits can be linked by wild-type of modified linkers to create zinc finger arrays of a desired length. In some embodiments, e.g. in zinc finger repressor proteins of the invention, the DNA-binding residues at the circled positions may be substituted, for example, with residues that bind their respective DNA nucleotides with less strength, so as to achieve long allele preferential binding of the repressor proteins of the invention. Depending on the position, amino acid substitutions may include K, D, E, A and G, wherein increasing the % of G or A provides the weakest overall binding interaction between the zinc finger peptide and the target polynucleotide.

FIG. 2 (A) A schematic illustration of an 11-zinc finger repressor protein according to the invention, showing recognition helices from adjacent pairs of zinc finger domains contacting 5′-GGG GCC-3′ bases on the lower DNA strand. Similar arrays comprising from 8 to 32 zinc fingers, for example, 8, 10, 12 and 18 zinc finger domains can be built. A nuclear localisation signal (NLS) is provided at the N-terminus and a transcription repressor domain is located at the C-terminus. For optimal use in mouse the NLS is from mouse p58 and the transcriptional repressor domain is from mouse KRAB. (B) A schematic illustration of a 6-zinc finger activator protein according to the invention, showing recognition helices from adjacent pairs of zinc finger domains contacting 5′-GGG GCC-3′ bases on the lower DNA strand. Similar arrays comprising from 3 to 8 zinc fingers, for example, 5 and 7 zinc finger domains can be built. A nuclear localisation signal (NLS) is provided at the N-terminus and a transcription activator domain is located at the C-terminus. For optimal use in mouse the NLS is from mouse p58 and the transcriptional activator domain is from mouse p65-RelA. For purposes of illustration, the sequences of representative DNA recognition helices from fingers 1 and 2 (F1, F2) are displayed below the zinc finger arrays, with foreign sequences in bold font and natural host sequences in normal font.

FIG. 3 Graph showing zinc finger repressor protein mediated silencing of the c9orf72 locus in the lymphoblastoid cell line (LCL; ND10966). Columns as follows: ‘control’=negative control; ‘A’=ZF11xALS1-Kox repressor peptide (SEQ ID NO: 96); ‘B’=ZF11xALS2-Kox repressor peptide (SEQ ID NO: 97); ‘C’=ZF11xALS-TV8-Kox repressor peptide (SEQ ID NO: 99); ‘D’=ZF11xALS-TV9-Kox repressor peptide (SEQ ID NO: 100); ‘E’=ZF11xALS-TV10-Kox repressor peptide (SEQ ID NO: 101) and ‘F’=ZF11xALS3-Kox repressor peptide (SEQ ID NO: 98). Transcript levels of c9orf72 were assessed in cell extracts. All Taqman qPCR values were normalised to the geometric mean of three housekeeping genes Gapdh, 18S and Hprt. Error bars are ±SEM (n=7). Student's t-test: *p<0.05, **p<0.01; ***p<0.001. The zinc finger repressor proteins of the invention are ‘tuned’ to alter their binding affinity for the target nucleic acid sequence and the results demonstrate that ‘tuning’ can alter the relative repression of the target gene, as desired.

FIG. 4 Graph showing zinc finger repressor protein mediated silencing of the c9orf72 locus in the human induced pluripotent stem cell line (hiPSC; RCFB60c7, RCi177). Columns as follows: ‘control’=negative control; ‘A’=ZF11xALS1-Kox repressor peptide (SEQ ID NO: 96); ‘B’=ZF11xALS2-Kox repressor peptide (SEQ ID NO: 97); ‘C’=ZF11xALS-TV8-Kox repressor peptide (SEQ ID NO: 99); ‘D’=ZF11xALS-TV9-Kox repressor peptide (SEQ ID NO: 100); ‘E’=ZF11xALS-TV10-Kox repressor peptide (SEQ ID NO: 101) and ‘F’=ZF11xALS3-Kox repressor peptide (SEQ ID NO: 98). Transcript levels of c9orf72 were assessed in cell extracts. All Taqman qPCR values were normalized to the geometric mean of three housekeeping genes Gapdh, 18S and Hprt. Error bars are ±SEM (n=4). Student's t-test: *p<0.05, **p<0.01; ***p<0.001. The zinc finger repressor proteins of the invention are ‘tuned’ to alter their binding affinity for the target nucleic acid sequence and the results demonstrate that ‘tuning’ can alter the relative repression of the target gene, as desired.

FIG. 5 (A) Schematic representation showing the model of active delivery in an in vivo system—e.g. in the target brain 1: therapeutic peptide is delivered to a first population of target cells 2 using a suitable delivery system (e.g. such as a viral delivery vector); therapeutic peptide is expressed and secreted from the first population of target cells; and secreted therapeutic peptide diffuses within the in vivo system coming into contact with a second population of target cells 3; cell penetration of the secreted therapeutic peptide allows the therapeutic effect to take effect in both the first 2 and second 4 populations of target cells, such that delivery of therapeutic peptide to a relatively small first population of target cells 2, 4 can enable therapeutic effect in a relatively larger population of target cells 3, 5 (Key: 1=target brain; 2=therapeutic viral delivery site A: therapeutic peptide expressed in viral-infected cells; 3=diffusion volume of secretable therapeutic peptide expressed at site A; 4=therapeutic viral delivery site B: secretable therapeutic peptide expressed in viral-infected cells; and 5=diffusion volume of secretable therapeutic peptide expressed at site B); and (B) schematic illustration showing hypothetical deliver of therapeutic peptide via ‘active delivery’ in neuronal cells: step (1) infection with AAV-ZF; step (2) ZFG secretion; (3) ZF cell penetration (Key: 6=microglia; 7=oligodendrocytes; 8=myelin sheath; 9=neuron; 10=dendrite; 11=synapse; 12=axon; 13=astrocyte).

FIG. 6 Graph showing repression of mutant gene target but not wild-type gene by cell-penetrating zinc finger peptides according to the invention, in engineered 293T and human fibroblast cells. Stable 293T cell lines, carrying either wild-type target gene (‘WT target’—panel (A)) or mutant target gene (‘Mutant target’—panel (B)), and a human fibroblast cell line carrying both wild-type and mutant target genes were grown in serum free (SF) medium. Zinc finger peptide (ZFP)-enriched SF medium (at 0%, 50% or 100% v/v ZFP medium) was added to the target cell population and incubated for 96h. Zinc finger peptides were designed to preferentially bind and repress the mutant target genes. Wild-type and mutant target mRNAs were analysed by Taqman qPCR. Values were normalised to the housekeeping gene human 18S. Error bars are SEM (n=3). Student's t-test: *p<0.05; **p<0.01. Data showing that zinc finger peptides are expressed and secreted and that secreted zinc finger peptides are capable of penetrating target cells and repressing a desired mutant target gene in vitro in both mouse and human cells. Y-axis ‘Normalised to serum free (SF) treated cells’; X-axis: column 1, 293WT SF; column 2, 293WT 50% ZFP; column 3, 293WT 100% ZFP; column 4, 293Mutant SF; column 5, 293Mutant 50% ZFP; column 6, 293Mutant 100% ZFP; column 7, Fibroblasts WT/Mutant SF; column 8, Fibroblasts WT/Mutant 50% ZFP; column 9, Fibroblasts WT/Mutant 100% ZFP.

FIG. 7 Secreted cell-penetrating TFs repress specifically in vivo, in mice. Hela cells were transfected with a plasmid carrying a zinc finger repressor peptide having 11-zinc finger domains (ZFP-SP) or empty control plasmid. 12 hours post transfection, media were replaced. Supernatant (spt) fractions of medium were harvested after 72 hours and were dialyzed (against 20 mm HEPES buffer (pH 8.0) containing 135 mm NaCl). Y-axis ‘qRT-PCR normalized to housekeeping genes’: (A) Newborn mice at p0 were injected intraventricularly with 2 μl of dialysed ZFP spt (column 2), control spt (column 1) or 20 mm HEPES buffer (column 3). Tissues (whole brain) were harvested at 96h and were analysed by qRT-PCR (n=7 per group). TNF-alpha was used as an inflammation control. X-axis: panel (A)=control allele; panel (B)=target allele; panel (C)=TNF-alpha. (B) 8 wks old mice were injected with dialysed spt or buffer into Tibialis Anterior (TA) muscles. Muscles were harvested 96h after injection (n=5 per group). One-way ANOVA with Bonferroni correction, *p<0.05, **p<0.001. X-axis: column 1, 10 μl HEPES control; column 2, 50 μl HEPES control; column 3, 10 μl control spt; column 4, 50 μl control spt; column 5, 10 μl ZFP spt; column 6, 50 μl ZFP spt: panel (A)=control allele; panel (B)=target allele; panel (C)=TNF-alpha. Therefore, non-concentrated cell supernatant is sufficient to repress a target in vivo.

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs (e.g. in cell culture, molecular genetics, nucleic acid chemistry and biochemistry).

Unless otherwise indicated, the practice of the present invention employs conventional techniques of chemistry, molecular biology, microbiology, recombinant DNA technology, chemical methods, pharmaceutical formulations and delivery and treatment of animals, which are within the capabilities of a person of ordinary skill in the art. Such techniques are also explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

In order to assist with the understanding of the invention several terms are defined herein.

The term ‘amino acid’ in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The term ‘peptide’ as used herein (e.g. in the context of a zinc finger peptide (ZFP) or framework) refers to a plurality of amino acids joined together in a linear or circular chain. term oligopeptide is typically used to describe peptides having between 2 and about 50 or more amino acids. Peptides larger than about 50 amino acids are often referred to as polypeptides or proteins. For purposes of the present invention, however, the term ‘peptide’ is not limited to any particular number of amino acids, and is used interchangeably with the terms ‘polypeptide’ and ‘protein’.

As used herein, the term ‘zinc finger domain’ refers to an individual ‘finger’, which comprises a ββα-fold stabilised by a zinc ion (as described elsewhere herein). Each zinc finger domain typically includes approximately 30 amino acids. The term ‘domain’ (or ‘module’), according to its ordinary usage in the art, refers to a discrete continuous part of the amino acid sequence of a polypeptide that can be equated with a particular function. Zinc finger domains are largely structurally independent and may retain their structure and function in different environments. Typically, a zinc finger domain binds a triplet or (overlapping) quadruplet nucleotide sequence. Adjacent zinc finger domains arranged in tandem are joined together by linker sequences. A zinc finger peptide of the invention is composed of a plurality of ‘zinc finger domains’, which in combination do not exist in nature. Therefore, they may be considered to be artificial or synthetic zinc finger peptides.

The terms ‘nucleic acid’, ‘polynucleotide’, and ‘oligonucleotide’ are used interchangeably and refer to a deoxyribonucleotide (DNA) or ribonucleotide (RNA) polymer, in linear or circular conformation, and in either single- or double-stranded form. For the purposes of the present invention such DNA or RNA polymers may include natural nucleotides, non-natural or synthetic nucleotides, and mixtures thereof. Non-natural nucleotides may include analogues of natural nucleotides, as well as nucleotides that are modified in the base, sugar and/or phosphate moieties (e.g. phosphorothioate backbones). Examples of modified nucleic acids are PNAs and morpholino nucleic acids. Generally, an analogue of a particular nucleotide has the same base-pairing specificity, i.e. an analogue of G will base-pair with C. For the purposes of the invention, these terms are not to be considered limiting with respect to the length of a polymer.

A ‘gene’, as used herein, is the segment of nucleic acid (typically DNA) that is involved in producing a polypeptide or ribonucleic acid gene product. It includes regions preceding and following the coding region (leader and trailer) as well as intervening sequences (introns) between individual coding segments (exons). Conveniently, this term also includes the necessary control sequences for gene expression (e.g. enhancers, silencers, promoters, terminators etc.), which may be adjacent to or distant to the relevant coding sequence, as well as the coding and/or transcribed regions encoding the gene product. Preferred genes in accordance with the present invention are those associated with neurological disease conditions; particularly those exhibiting aberrant hexanucleotide repeat sequences, such as mutant C9Orf72 genes.

As used herein the term ‘modulation’, in relation to the expression of a gene refers to a change in the gene's activity. Modulation includes both activation (i.e. increase in activity or expression level) and repression (i.e. reduction or inhibition) of gene activity. In some embodiments of the invention, the therapeutic molecules (e.g. peptides) of the invention are repressors of gene expression or activity; in some embodiments of the invention, the therapeutic molecules (e.g. peptides) of the invention are activators of gene expression or activity.

A nucleic acid ‘target’, ‘target site’ or ‘target sequence’, as used herein, is a nucleic acid sequence to which a zinc finger peptide of the invention will bind, provided that conditions of the binding reaction are not prohibitive. A target site may be a nucleic acid molecule or a portion of a larger polynucleotide. Particularly suitable target sites comprise repetitive nucleic acid sequences; especially hexanucleotide or trinucleotide repeat sequences. Preferred target sequences in accordance with the invention include those defined by GGGGCC-repeat sequences (e.g. GGGGCC . . . ; GGGCCG . . . ; GGCCGG . . . ; GCCGGG; and CCGGGG . . . ), and their complementary sequences. In accordance with the invention, a target sequence for a poly-zinc finger peptide of the invention may comprise a single contiguous nucleic acid sequence, or more than one non-contiguous nucleic acid sequence (e.g. two separate contiguous sequences, each representing a partial target site), which are interspersed by one or more intervening nucleotide or sequence of nucleotides. These terms may also be substituted or supplemented with the terms ‘binding site’, ‘binding sequence’, or ‘recognition site’, which are used interchangeably.

As used herein, ‘binding’ in the context of the present invention refers to a non-covalent interaction between macromolecules (e.g. between a zinc finger peptide and a nucleic acid molecule containing an appropriate target site). In some cases, binding will be sequence-specific, such as between one or more specific nucleotides (or base pairs) and one or more specific amino acids. It will be appreciated, however, that not all components of a binding interaction need be sequence-specific (e.g. non-covalent interactions with phosphate residues in a DNA backbone). Binding interactions between a nucleic acid sequence and a zinc finger peptide of the invention may be characterised by binding affinity and/or dissociation constant (Kd). A suitable dissociation constant for a zinc finger peptide of the invention binding to its target site may be in the order of 1 μM or lower, 1 nM or lower, or 1 pM or lower, as described elsewhere herein. ‘Affinity’ refers to the strength of binding, such that increased binding affinity correlates with a lower Kd value. Zinc finger peptides may have DNA-binding activity, RNA-binding activity, and/or even protein-binding activity. Generally, the zinc finger peptides of the invention are designed or selected to have sequence specific nucleic acid-binding activity, especially to dsDNA. Typically, the target site for a particular zinc finger peptide is a sequence to which the zinc finger peptide concerned is capable of nucleotide-specific binding. It will be appreciated, however, that depending on the amino acid sequence of a zinc finger peptide it may bind to or recognise more than one target sequence, although typically one sequence will be bound in preference to any other recognised sequences, depending on the relative specificity of the individual non-covalent interactions. Generally, specific binding is preferably achieved with a dissociation constant (Kd) of 1 μm or lower, 1 nM or lower, 100 pM or lower; or 10 pM or lower. In some embodiments, particularly as regards ZFP repressor proteins of the invention, binding affinity for a target site may be deliberated weakened (reduced) such that a zinc finger repressor protein of the invention may bind preferentially to expanded, pathogenic-repeat sequences, e.g. in ALS/FTD comprising 30 or more, 100 or more or 200 or more repeat sequences as compared to shorter hexanucleotide repeat sequences, e.g. comprising less than 30, less than 20, less than 10 or between 2 and 8 hexanucleotide repeat sequences. In some embodiments, therefore, a zinc finger peptide of the invention may bind a target sequence with a dissociation constant that is weaker than about 100 pM, weaker than 1 nM, weaker than 10 nm, or weaker than 100 nM.

By ‘non-target’ it is meant that the nucleic acid sequence concerned is not appreciably bound by the relevant zinc finger peptide. In some embodiments, it may be considered that, where a zinc finger peptide of the invention has a known sequence-specific target sequence, essentially all other nucleic acid sequences may be considered to be non-target. From a practical perspective it can be convenient to define an interaction between a non-target sequence and a particular zinc finger peptide as being sub-physiological (i.e. not capable of creating a physiological response under physiological target sequence/zinc finger peptide concentrations). For example, if any binding can be measured between the zinc finger peptide and the non-target sequence, the dissociation constant (Kd) is typically weaker than 1 μM, such as 10 μM or weaker, 100 μM or weaker, or at least 1 mM.

### Zinc Finger Peptides

A ‘zinc finger’ is a relatively small polypeptide domain comprising approximately 30 amino acids, which folds to form a secondary structure including an α-helix adjacent an antiparallel β-sheet (known as a ββα-fold). The fold is stabilised by the co-ordination of a zinc ion between four largely invariant (depending on zinc finger framework type) Cys and/or His residues, as described further below. Natural zinc finger domains have been well studied and described in the literature, see for example, Miller et al., (1985) EMBO J. 4: 1609-1614; Berg (1988) Proc. Natl. Acad. Sci. USA 85: 99-102; and Lee et al., (1989) Science 245: 635-637. A zinc finger domain typically recognises and binds to a nucleic acid triplet, or an overlapping quadruplet (as explained below), in a double-stranded DNA target sequence. However, zinc fingers are also known to bind RNA and proteins (Clemens, K. R. et al. (1993) Science 260: 530-533; Bogenhagen, D. F. (1993) Mol. Cell. Biol. 13: 5149-5158; Searles, M. A. et al. (2000) J. Mol. Biol. 301: 47-60; Mackay, J. P. & Crossley, M. (1998) Trends Biochem. Sci. 23: 1-4). Zinc finger proteins generally contain strings or chains of zinc finger domains (or modules). Thus, a natural zinc finger protein may include two or more zinc finger domains, which may be directly adjacent one another, e.g. separated by a short (canonical) or canonical-like linker sequence; or a longer, flexible or structured polypeptide sequence. Adjacent zinc finger domains linked by short canonical or canonical-like linker sequences of 5, 6 to 7 amino acids are expected to bind to contiguous nucleic acid sequences, i.e. they typically bind to adjacent trinucleotides/triplets; or protein structures. In some cases, cross-binding may also occur between adjacent zinc fingers and their respective target triplets, which helps to strengthen or enhance the recognition of the target sequence, and leads to the binding of overlapping quadruplet sequences (Isalan et al., (1997) Proc. Natl. Acad. Sci. USA, 94: 5617-5621). By comparison, distant zinc finger domains within the same poly-zinc finger protein may recognise (or bind to) non-contiguous nucleic acid sequences or even to different molecules (e.g. protein rather than nucleic acid). Indeed, naturally occurring zinc finger-containing proteins may include both zinc finger domains for binding to protein structures as well as zinc finger domains for binding to nucleic acid sequences.

In accordance with the invention, some pairs of adjacent zinc finger domains of the same polypeptide may be separated by relatively long, flexible linker sequences. Such adjacent zinc fingers can readily bind to non-contiguous nucleic acid sequences, although it is also possible for them to bind to contiguous sequences. In such embodiments, the relative binding location of the pairs of zinc finger domains separated by long linker sequences may be determined by the sequence context, i.e. by dominant binding interactions from other zinc finger domains within the peptide.

The majority of the amino acid side chains in a zinc finger domain that are important for dsDNA base recognition are located on the α-helix of the finger. Conveniently, therefore, the amino acid positions in a zinc finger domain are numbered from the first residue in the α-helix, which is given the number (+)1; and the helix is generally considered to end at the final zinc-coordinating Cys or His residue, which is typically position +11. Thus, “−1” refers to the residue in the framework structure immediately preceding the first residue of the α-helix. As used herein, residues referred to as “++” are located in the immediately adjacent (C-terminal) zinc finger domain. Generally, nucleic acid recognition by a zinc finger module is achieved primarily by the amino acid side chains at positions −1, +3, +6 and ++2; although other amino acid positions (especially of the α-helix) may sometimes contribute to binding between the zinc finger and the target molecule. Since the vast majority of base-specific interactions between dsDNA and a zinc finger domain come from this relatively short stretch of amino acids, it is convenient to define the sequence of the zinc finger domain from −1 to +6 (i.e. residues −1, 1, 2, 3, 4, 5 and 6) as a zinc finger ‘recognition sequence’. For ease of understanding, it is worth noting that the first invariant histidine residue that coordinates the zinc ion is position (+)7 of the zinc finger domain.

When binding to a nucleic acid sequence, the zinc finger recognition sequence primarily interacts with one strand of a double-stranded nucleic acid molecule (the primary strand or sequence). However, there can be subsidiary interactions between amino acids of a zinc finger domain and the complementary (or secondary) strand of the double-stranded nucleic acid molecule. For example, the amino acid residue at the ++2 position typically may interact with a nucleic acid residue in the secondary strand.

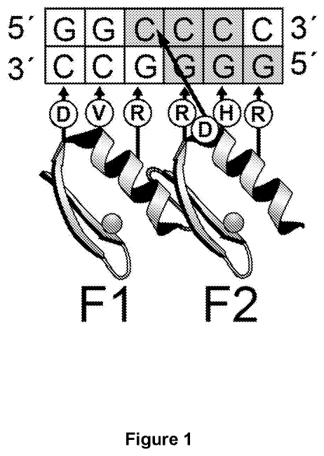

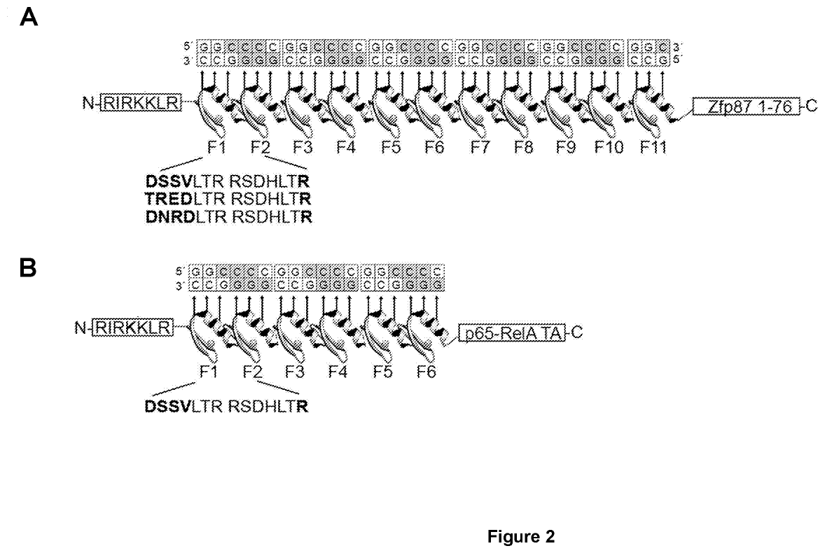

During binding, the α-helix of the zinc finger domain almost invariably lies within the major groove of dsDNA and aligns antiparallel to the target nucleic acid strand. Accordingly, the primary nucleic acid sequence is arranged 3′ to 5′ in order to correspond with the N-terminal to C-terminal sequence of the zinc finger peptide. Since nucleic acid sequences are conventionally written 5′ to 3, and amino acid sequences N-terminus to C-terminus, when a target nucleic acid sequence and a zinc finger peptide are aligned according to convention, the primary interaction of the zinc finger peptide is with the complementary (or minus) strand of the nucleic acid sequence, since it is this strand which is aligned 3′ to 5′ (see also FIGS. 1 and 2). These conventions are followed in the nomenclature used herein.

Zinc finger peptides according to the invention are non-natural and suitably contain 3 or more, for example, 4, 5, 6, 7, 8, 9, 10, 11, 12, 13, 14, 15, 16, 17, 18, 24 or more (e.g. up to approximately 30 or 32) zinc finger domains arranged adjacent one another in tandem. Such peptides may also be referred to herein as ‘poly-zinc finger peptides’.

In aspects and embodiments, zinc finger peptides of the invention include at least 6 zinc finger domains, preferably at least 8, at least 11, at least 12 or at least 18 zinc finger domains; and in some cases at least 24 zinc finger domains. Preferably, the zinc finger peptides in these aspects and embodiments of the invention have from 8 to 18, from 10 to 18 or from 11 to 18 zinc finger domains arranged in tandem (e.g. 8, 9, 10, 11, 12, 13, 14, 15, 16, 17 or 18). Particularly beneficial zinc finger peptides have 10, 11 or 12 zinc finger domains arranged in tandem; and especially 11 zinc finger domains.

In other aspects and embodiments, zinc finger peptides of the invention include no more than 8 zinc finger domains; such as between 3 and 8 zinc finger domains, or between 4 and 7 zinc finger domains. Preferably, in these aspects and embodiments, the zinc finger peptide has 5, 6 or 7 zinc finger domains, and more preferably has 6 zinc finger domains arranged in tandem.

Particularly beneficial aspects and embodiments comprise two poly-zinc finger peptides which differ in the number of zinc finger domains arranged in tandem. For example, one poly-zinc finger peptide in these aspects and embodiments has 8 or fewer zinc finger domains arranged in tandem and the other poly-zinc finger peptide has 8 or more zinc finger domains arranged in tandem. For example, one zinc finger peptide may have from 3 to 8, from 3 to 7, from 4 to 7, or from 4 to 6 (e.g. 4, 5 or 6) zinc finger domains arranged in tandem; and the other zinc finger peptide of the pair has from 8 to 32, from 8 to 24, from 8 to 18 or from 10 to 18 (e.g. 10, 11, 12, 13, 14, 15, 16, 17 or 18) zinc finger domains arranged in tandem. In one particular embodiment one zinc finger peptide of the pair has 6 zinc finger domains in tandem and the other zinc finger peptide has 11 zinc finger domains in tandem.

As already noted, the zinc finger peptides of the invention may bind to non-contiguous or contiguous nucleic acid binding sites. When targeted to non-contiguous binding sites, each sub-site (or half-site where there are two non-contiguous sequences) is suitably at least approximately 18 bases long, but may alternatively be approximately 12, 15 or 24 bases long. Preferred 11 zinc finger peptides of the invention bind to full-length nucleic acid sequences which are approximately 33 nucleotides long, but which may contain two subsites of 18 and 15 nucleotides arranged directly adjacent to one another to form a contiguous sequence, or which subsites are separated by intervening nucleotides to create a non-contiguous target site. Preferred 12 zinc finger peptides of the invention bind to full-length nucleic acid sequences which are approximately 36 nucleotides long, but which may contain two subsites of 18 nucleotides that are arranged directly adjacent to one another to form a contiguous sequence, or may be separated by intervening nucleotides as in the case of a non-contiguous target site. Preferred 6 zinc finger peptides of the invention bind to full-length nucleic acid sequences which are approximately 18 nucleotides long, but which may contain two subsites of 9 nucleotides arranged directly adjacent to one another to form a contiguous sequence, or which are separated by intervening nucleotides to create a non-contiguous target site.

In (poly-)zinc finger peptides of the present invention, adjacent zinc finger domains are joined to one another by ‘linker sequences’ that may be canonical, canonical-like, flexible or structured, as described, for example, in WO 01/53480 (Moore et al., (2001) Proc. Natl. Acad. Sci. USA 98: 1437-1441). Generally, a natural zinc finger linker sequence lacks secondary structure in the free form of the peptide. However, when the protein is bound to its target site a canonical linker is typically in an extended, linear conformation, and amino acid side chains within the linker may form local interactions with the adjacent nucleic acid. In a tandem array of zinc finger domains, the linker sequence is the amino acid sequence that lies between the last residue of the α-helix in an N-terminal zinc finger and the first residue of the β-sheet in the next (i.e. C-terminal adjacent) zinc finger. For the purposes of the present invention, the last amino acid of the α-helix in a zinc finger is considered to be the final zinc coordinating histidine (or cysteine) residue, while the first amino acid of the following finger is generally a tyrosine, phenylalanine or other hydrophobic residue.

It is desirable that the zinc finger peptides of the invention bind relatively specifically to their target sequence. It will be appreciated, however, that ‘specificity’ to a highly repetitive sequence is not a straightforward concept in the sense that relatively shorter and relatively longer repetitive sequences may both be targeted and bound with good affinity. In accordance with some embodiments of the invention (and as described elsewhere herein), the zinc finger peptides of the invention may beneficially exhibit preferential binding to relatively longer repeat sequences over relatively shorter repeat sequences.

Binding affinity (e.g. dissociation constant, Kd) is one way to assess the binding interaction between a zinc finger peptide of the invention and a potential target nucleic acid sequence. The binding affinity of a zinc finger peptide for its selected/potential target sequence can be measured using techniques known to the person of skill in the art, such as surface plasmon resonance, or biolayer interferometry. Biosensor approaches are reviewed by Rich et al. (2009), “A global benchmark study using affinity-based biosensors”, Anal. Biochem., 386:194-216. Alternatively, real-time binding assays between a zinc finger peptide and target site may be performed using biolayer interferometry with an Octet Red system (Fortebio, Menlo Park, CA). It can be useful to measure binding affinity of the zinc finger peptides of the invention to ensure that each achieves the desired binding strength; especially in aspects and embodiments comprising pairs of complementary zinc finger peptide, wherein the relative binding strength may be relevant to the performance of the invention. In addition, where zinc finger peptides of the invention are modified, e.g. to lower potential immunogenicity for host-optimisation, it can be useful to measure the binding affinity so ensure that those modifications—especially those in the recognition sequence region—have not adversely affected nucleic acid binding affinity.

Zinc finger peptides of the invention typically have μM or higher binding affinity for a target nucleic acid sequence. Suitably, in some embodiments a zinc finger peptide of the invention has nM or sub-nM binding affinity for its specific target sequence; for example, 10⁻⁹ M, 10⁻¹⁰ M, 10⁻¹¹ M, or 10⁻¹² M or less. In some particularly preferred embodiments the affinity of a zinc finger peptide of the invention for its target sequence is in the μM range or below, for example, in the range of 10⁻¹³ M, 10⁻¹⁴ M, or 10⁻¹⁵ M or less. In other embodiments a zinc finger peptide of the invention has weaker than nM or sub-nM binding affinity for its specific target sequence; for example, 10⁻⁹ M, 10⁻⁸ M, 10⁻⁷ M, or 10⁻⁶ M or less.

Binding affinity between a zinc finger peptide of the invention and a target nucleic acid sequence can conveniently be assessed using an ELISA assay, as is know to the person of skill in the art.

The present invention relates to non-naturally occurring poly-zinc finger peptides for binding to repetitive nucleic acid sequences, such as hexanucleotide repeat sequences (particularly to GGGGCC-repeats) or any off-frame repeat variants, as may be found in naturally-occurring genomic DNA sequences. The invention also relates to the use of such poly-zinc finger peptides as therapeutic molecules and to related methods of treatment: for example, for treating diseases associated with expanded GGGGCC-repeat sequences such as ALS and FTD. Desirably, in some embodiments poly-zinc finger peptides of the invention bind to expanded GGGGCC-repeats (or any of the other 5, respectively, related frame variations based on the double stranded repeat sequence) associated with mutated gene sequences in preference to and/or selectively over the shorter GGGGCC-repeat sequences, respectively, of normal, non-pathogenic genes. For example, the binding affinity of a zinc finger peptide of the invention for a pathogenetic nucleotide repeat sequence may be at least 2-fold higher, at least 10-fold higher, or at least 100-fold higher than for a wild-type/non-pathogenic nucleotide repeat sequence for the respective gene. In ALS/FTD embodiments, the binding affinity of zinc finger peptides of the invention for sequences of 30 or more GGGGCC repeats may be at least 2-fold higher, at least 5-fold, or at least 10-fold higher than for sequences of 8 or less GGGGCC repeats. Suitably, the affinity of such zinc finger peptides of the invention for DNA sequences having at least 100 GGGGCC repeats is at least 5-fold, at least 10-fold or at least 20-fold higher than for sequences having 8 or less GGGGCC repeats. In some particularly advantageous embodiments, the affinity of zinc finger peptides of the invention for DNA sequences having at least 700 GGGGCC repeats is at least 5-fold, at least 10-fold or at least 20-fold higher than for sequences having less than 30 GGGGCC repeats.

In some particularly advantageous embodiments, the invention comprises two (also termed herein a complementary pair of) poly-zinc finger peptides according to the invention. In embodiments of one aspect of the invention, one of a pair binds to GGGGCC repeat sequences with greater affinity than the other of the pair of zinc finger peptides. For example, the dissociation constant for sequences comprising 30 or more GGGGCC repeats may be at least 2-fold, at least 5-fold, at least 10-fold or at least 100-fold higher for one of the pair of zinc finger peptides than for the other of the pair. In embodiments, the dissociation constant for dsDNA sequences comprising between 2 and 8 GGGGCC repeats may be at least 2-fold, at least 5-fold, at least 10-fold or at least 100-fold higher for one of the pair of zinc finger peptides than for the other of the pair.

Zinc Finger Peptide Frameworks and Derivatives Zinc finger peptides have proven to be extremely versatile scaffolds for engineering novel DNA-binding domains (e.g. Rebar & Pabo (1994) Science 263: 671-673; Jamieson et al., (1994) Biochemistry 33: 5689-5695; Choo & Klug (1994) Proc. Natl. Acad. Sci. USA. 91: 11163-11167; Choo et al., (1994) Nature 372: 642-645; Isalan & Choo (2000) J. Mol. Biol. 295: 471-477; and many others).

There are a number of natural zinc finger frameworks known in the art, and any of these frameworks may be suitable for use in the zinc finger peptide frameworks of the invention. In general, a natural zinc finger framework has the sequence, Formula 1: X₀₋₂ C X₁₋₅ C X₉₋₁₄ H X₃₋₆H/c; or Formula 2: X₀₋₂ C X₁₋₅ C X₂₋₇ X⁻¹ X⁺¹ X⁺² X⁺³ X⁺⁴ X⁺⁵ X⁺⁶ H X₃₋₆ H/c where X is any amino acid, the numbers in subscript indicate the possible numbers of residues represented by X, and the numbers in superscript indicate the position of the amino acid in the α-helix. In embodiments of the invention, the zinc finger peptide framework is based on an array of zinc finger domains of Formula 1 or 2. Alternatively, the zinc finger motif may be represented by the general sequence, Formula 3: X₂ C X_{2,4} C X₁₂ H X_{3,4,5} H/c; or Formula 4: X₂ C X_{2,4} C X₅ X⁻¹ X⁻¹ X⁺² X⁺³ X⁺⁴ X⁺⁵ X⁺⁶ H X_{3,4,5} H/c. Still more preferably the zinc finger motif may be represented by the general sequence, Formula 5: X₂ C X₂ C X₁₂ H X₃ H; or Formula 6: X₂ C X₂ C X₅ X⁻¹ X⁺¹ X⁺² X⁺³ X⁺⁴ X⁺⁵ X⁺⁶ H X₃ H. Accordingly, an extended zinc finger peptide framework of the invention may be based on zinc finger domains of Formulas 1 to 6, or combinations of Formulas 1 to 6, joined together in an array using the linker sequences described herein.

In these formulas, the fixed C and H residues coordinate the zinc ion to stabilise the zinc finger structure: the first H residue is position +7 of the α-helix. Particularly preferred positions for diversification within the zinc finger domain frameworks of the invention, in order to direct binding to a desired target, are those within or adjacent the α-helix, for example, positions −1, 2, 3 and 6. It can be beneficial to minimise these diversifications, particularly with respect to residues of the α-helix outside of these positions, where the zinc finger framework is otherwise native to the biological system in which the zinc finger peptides of the invention may be used in vivo, so as to reduce host-immune reactions.

Preferred zinc finger peptide arrays of the invention have a sequence and framework (excluding the recognition sequences, which are described elsewhere herein) according to one or more of Structures I, II, III and IV as defined in our earlier patent applications, WO 2012/049332 and WO 2017/077329, which teaching of said zinc finger peptide frameworks (i.e. Structures I, II, III and IV) is explicitly incorporated herein by reference in its entirely, including any preferred and optional features thereof.

In some aspects and embodiments of the invention, the extended zinc finger peptide framework comprises at least 8 zinc finger domains of one of Formulas 1 to 6, joined together by linker sequences, i.e. Structure V: [(Formula 1-6)-linker]ₙ-(Formula 1-6)], where n is ≥10, such as between 10 and 31. As indicated, in Structure V any combination of Formulas 1 to 6 may be used. In another embodiment the extended zinc finger peptide framework comprises between 10 and 18 (e.g. 11 to 18) zinc finger domains of the above formulae. Suitably, therefore, n is 9 to 17 (e.g. 10 to 17); more suitably n is 9, 10, 11, 13, 14, 15 or 17; preferably n is 9, 10, 11 or 17; most preferably n is 10.

As already described, adjacent zinc finger domains are joined together by linker sequences. In a natural zinc finger protein, threonine is often the first residue in the linker, and proline is often the last residue of the linker. On the basis of sequence homology, the canonical natural linker sequence is considered to be -TGEKP- (Linker 1 or L1; SEQ ID NO: 112). However, natural linkers can vary greatly in terms of amino acid sequence and length. Therefore, a common consensus sequence based on natural linker sequences may be represented by -TGE/QK/RP-(Linker 2 or L2; SEQ ID NO: 113), and this sequence is preferred for use as a ‘canonical’ (or ‘canonical-like’) linker in accordance with the invention. Thus, another useful canonical linker sequence is -TGQKP- (SEQ ID NO: 114).

However, in extended zinc finger arrays of e.g. 4 or more zinc finger domains, it has been shown that it can be beneficial to periodically disrupt the canonical linker sequence, when used between adjacent zinc fingers in an array, by adding one or more amino acid residue (e.g. Gly and/or Ser), to create groups of 2 or 3 zinc finger domains within the array (Moore et al., (2001) Proc. Natl. Acad. Sci. USA 98: 1437-1441; and WO 01/53480). Therefore, suitable linker sequences for use in accordance with the invention include canonical linker sequences of 5 amino acids (e.g. Linker 1 or Linker 2, above), and related canonical-like linker sequences of 6 or 7 amino acids.

Canonical-like linkers for use in accordance with the invention may suitably be based on the sequence, -TGG/SE/QK/RP- (Linker 3 or L3; SEQ ID NO: 115). Preferred canonical-like linkers thus include the specific sequences: TGGERP (SEQ ID NO: 116), TGSERP (SEQ ID NO: 117), TGGQRP (SEQ ID NO: 118), TGSQRP (SEQ ID NO: 119), TGGEKP (SEQ ID NO: 120), TGSEKP (SEQ ID NO: 121), TGGQKP (SEQ ID NO: 122), or TGSQKP (SEQ ID NO: 123). A particularly preferred canonical-like linker is TGSERP (Linker 4 or L4; SEQ ID NO: 117). Another particularly preferred canonical-like linker is TGSQKP (Linker 5 or L5; SEQ ID NO: 123). However, other linker sequences may also be used between one or more pairs of zinc finger domains, for example, linkers of the sequence -TG(G/S)₀₋₂E/QK/RP- (SEQ ID NO: 124) or -T(G/S)₀₋₂GE/QK/RP- (Linker 6 or L6; SEQ ID NO: 125).

In some embodiments still longer flexible linkers of 8 or more amino acids may be used, as previously described. Linkers of 8 amino acids include the sequences -TG(G/S)₃E/QK/RP- (SEQ ID NO: 126) and -T(G/S)₃GE/QK/RP- (L12; SEQ ID NO: 127). Alternative long flexible linkers are: LRQKD(GGGGS)₁₋₄QLVGTAERP (Linker 7 or L7; SEQ ID NO: 128) and LRQKD(GGGGS)₁₋₄QKP (Linker 8 or L8; SEQ ID NO: 129). Preferred long flexible linkers for use in the zinc finger peptides of the invention are, LRQKDGGGGSGGGGSGGGGSQLVGTAERP (Linker 9 or L9; SEQ ID NO: 130), and LRQKDGGGGSGGGGSGGGGSQKP (Linker 10 or L10; SEQ ID NO: 131).

### A. Extended Poly-Zinc Finger Proteins

For specific biological functionality and therapeutic use, particularly in vivo (e.g. in gene therapy and transgenic animals), it is generally desirable that a poly-zinc finger peptide of the invention is able to target unique or virtually unique sites (or clusters) within any genome. For complex genomes, such as in humans, it is generally considered that an address of at least 16 bps is required to specify a potentially unique DNA sequence. Shorter DNA sequences have a significant probability of appearing several times in a genome, which increases the possibility of obtaining undesirable non-specific gene targeting and biological effects. Since individual zinc fingers generally bind to three consecutive nucleotides, 6-zinc finger domains with an 18 bp binding site could, in theory, be used for the specific recognition of a unique target sequence within any genome. Accordingly, a great deal of research has been carried out into so-called ‘designer transcription factors’ for targeted gene regulation, which typically involve 4 or 6-zinc finger domains that may be arranged in tandem or in dimerisable groups (e.g. of three-finger units).

The present invention relates to targeting of long arrays of nucleotide (hexa-) repeat sequences, and so there will be considerably more than one identical target site within the genome. Nevertheless, effective targeting (e.g. for therapy) of a desired sequence can be difficult taking into account the potential for yet more identical sequences associated with non-pathogenice, wild-type genes.

The inventors have previously shown (WO 2012/049332 and WO 2017/077329) that by selecting appropriate linker sequences and suitable combinations of linker sequences within an array of zinc fingers, extended arrays of zinc finger peptides of at least 8 or 10 zinc fingers (such as 10, 11, 12 or 18) can be synthesised, expressed and can have selective gene targeting activity. The extended arrays of zinc finger peptides of the invention are conveniently arranged in tandem. By way of example, such 11- or 12-zinc finger peptides can recognise and specifically bind 33 or 36 nucleic acid residues, respectively, and longer arrays (such as 18-zinc finger peptides) recognise still longer nucleic acid sequences. In this way, the extended zinc finger peptides of the invention can be targeted to preferred genomic sequences, e.g. expanded GGGGCC repeat sequences.

In the zinc finger frameworks above (e.g. selected from Structures I to V), the total number of zinc finger domains is preferably from 10 to 18, especially 10, 11, 12 or 18. Particularly preferred zinc finger peptides have 11 or 12 zinc finger domains, each of which has a recognition sequence as set out above. In accordance with preferred aspects and embodiments of the invention, these recognition sequences are selected as described elsewhere herein such that the poly-zinc finger peptide binds effectively to target nucleic acid sequences, such as pathogenic GGGGCC-repeat nucleic acid sequences while reducing, minimising or preventing binding to non-pathogenic (off-target), wild-type GGGGCC-repeat sequences in the preferred expression host (e.g. mouse or human).

The inventor's earlier work (e.g. WO 2012/049332; WO 2017/077329, each of which are incorporated herein by reference in their entirety) was the first to demonstrate that tandem arrays of more than 6 zinc finger domains, such as 8, 9, 10, 11, 12, 18 or more zinc fingers can be synthesised and expressed; and, more significantly, that such long arrays of non-natural zinc finger domains can have in vitro or in vivo (specific) nucleic acid binding activity. In this earlier work we also reported that such extended arrays of zinc finger peptides were capable of targeting genomic DNA sequences and have gene modulation activity in vitro and/or in vivo. We have also demonstrated that such extended zinc finger peptide frameworks comprising at least 8, at least 10, at least 11, at least 12, or at least 18 zinc finger domains can preferentially target expanded nucleic acid repeat sequences—e.g. as associated with pathogenic phenotypes preferentially over wild-type shorter repeat sequences.

In embodiments, suitable extended poly-zinc finger peptide frameworks of the invention comprise from 8 to 32 zinc finger domains, from 8 to 28 zinc finger peptides, from 8 to 24 zinc finger peptides, or from 8 to 18 zinc finger peptides. Preferred zinc finger peptides according to aspects and embodiments of the invention comprise 8, 10, 11, 12 or 18 zinc finger domains; and particularly preferred zinc finger peptides of the invention comprise 10, 11 or 12 zinc finger domains.

The zinc finger peptide frameworks of the invention may comprise directly adjacent zinc finger domains having canonical (or canonical-like) linker sequences between adjacent zinc finger domains, such that they preferentially bind to contiguous nucleic acid sequences. Accordingly, a 6-zinc finger peptide (framework) of the invention is particularly suitable for binding to contiguous stretches of approximately 18 nucleic acid bases or more, particularly of the minus nucleic acid strand. Particularly preferred zinc finger peptides of the invention comprise more than 6 zinc finger domains, such as 8, 10, 11, 12, 18, 24 or 32 zinc finger domains. Typically, such extended poly-zinc finger peptides, according to the invention are designed to bind nucleic acid sequences which may be arranged as a contiguous stretch or as a non-contiguous stretch comprising two or three subsites. For example, an 8-zinc finger peptide is particularly suitable for binding a target sequence of approximately 24 nucleotides; a 10-zinc finger peptide is suitable for binding approximately 30 nucleotides; an 11-zinc finger peptide is suitable for binding approximately 33 nucleotides; a 12-zinc finger peptide is capable of binding approximately 36 nucleotides; and an 18-zinc finger peptide of the invention is particularly suitable for binding to approximately 54 nucleic acid bases or more. As already described, such target sequences may be arranged contiguously or in non-contiguous subsites especially arranged in subsites of e.g. 12, 15 or 18 nucleotide lengths.

The extended arrays of zinc finger domains in the peptides and polypeptides of the invention typically comprise canonical linker sequences, short flexible (canonical-like) linker sequences and long flexible linker sequences. Thus, in some embodiments, one or more pairs of adjacent zinc finger domains of a zinc finger peptide according to the invention may be separated by short canonical linker sequences (e.g. TGERP, SEQ ID NO: 132; TGEKP, SEQ ID NO: 112; etc.). In some embodiments, one or more pairs of adjacent zinc finger domains of a zinc finger peptide according to the invention may be separated by short flexible linker sequences (e.g. of 6 or 7 amino acids), ‘canonical-like’ linker sequences, which preferably comprise the amino acid residues of a canonical linker with an additional one or two amino acid residues within, before or after the canonical sequence (preferably within). Adjacent zinc finger domains separated by canonical and short flexible linker sequences (i.e. which are between 5 and 7 amino acids long) typically bind to contiguous nucleic acid target sites. In accordance with the invention, however, one or more pairs of adjacent zinc finger domains of a zinc finger peptide may be separated by long flexible linker sequences, for example, comprising 8 or more amino acids, such as between 8 and 50 amino acids. Particularly suitable long flexible linkers have between approximately 10 and 40 amino acids, between 15 and 35 amino acids, or between about 20 and 30 amino acids. Preferred long flexible linkers may have 18, 23 or 29 amino acids. Adjacent zinc finger domains separated by long flexible linkers have the capacity to bind to non-contiguous binding sites in addition to the capacity to bind to contiguous binding sites. The length of the flexible linker may influence the length of intervening DNA that may lie between such non-contiguous binding sub-sites. This can be a particular advantage in accordance with the invention, since poly-zinc finger peptides that target extended hexanucleotide repeat sequences may then have a number of options for binding to contiguous as well as discontiguous target sequences.

Suitably, the zinc finger peptides/frameworks of the invention may comprise two or more (e.g. 2, 3 or 4) arrays of 4, 5, 6 or 8 directly adjacent zinc finger domains (or any combination thereof) separated by long flexible (or structured) linkers. Preferably, such extended (poly-)zinc finger peptides are arranged in multiple arrays of 5 and/or 6-finger units separated by long flexible linkers.

The inventors have previously shown that such extended zinc finger peptides of more than 6 zinc fingers in total can exhibit specific and high affinity binding to desired target sequences, both in vitro and in vivo. For example, whereas a 3-finger peptide (with a 9 bp recognition sequence) may bind DNA with nanomolar affinity, a 6-finger peptide might be expected to bind an 18 bp sequence with an affinity of between 10⁻⁹ and 10⁻¹⁸ M, depending on the arrangement and sequence of zinc finger peptides. To optimise both the affinity and specificity of 6-finger peptides, a fusion of three 2-finger domains has been shown to be advantageous (Moore et al., (2001) Proc. Natl. Acad. Sci. USA 98: 1437-1441; and WO 01/53480). Therefore, in some embodiments, the zinc finger peptides of the invention comprise a series of 2-finger units arranged in tandem. Zinc finger peptides of the invention may alternatively include or comprise a series of 3-finger units.

However, in accordance with the present invention, the inventors have found that extended poly-zinc finger peptides can be ‘tuned’ to moderate binding affinity for nucleic acid-repeat sequences according to the presence of both pathogenic and non-pathogenic (WT) target sequences within the same target cells. In aspects and embodiments of the invention, therefore, zinc finger repressor proteins are tuned to bind preferentially to extended, pathogenic repeat sequences, and zinc finger activator proteins are tuned to bind with greater affinity than repressor proteins to non-pathogenic repeat sequences. In this way, expression of wild-type, desirable gene products may be upregulated, whereas expression of pathogenic, non-desirable gene products may be downregulated.

Furthermore, it has been demonstrated that the extended zinc finger peptides of the invention can be stably expressed within a target cell, can be non-toxic to the target cell, and can have a specific and desired gene modulation activity. In particular, it has been shown that the zinc finger repressor proteins of the invention can have prolonged expression in target cells in vivo, without causing toxic side-effects that are often associated with the expression of heterologous/foreign protein sequences in vivo.

As noted above, the extended zinc finger peptides of the invention are adapted for binding to repeat sequences (i.e. hexanucleotide repeats) in target genes. According to embodiments of first aspects of the invention, suitable target sequences in pathogenic ALS/FTD genome sequences may comprise at least 30 hexanucleotide repeats, at least 100 hexanucleotide repeats, or at least 700 hexanucleotide repeats; for example, up to 1,600 hexanucleotide repeats. In embodiments of the invention, suitable target sequences in non-pathogenic, wild-type genome sequences may have less than 30 hexanucleotide repeats; for example, up to 23 hexanucleotide repeats, up to 20 hexanucleotide repeats, or up to 10 hexanucleotide repeats.

The extended zinc finger peptides of the invention—particularly the zinc finger repressor peptides of the invention—preferably bind to sequences within expanded nucleotide-repeat sequences in double-stranded DNA e.g. DNA molecules, fragments, gene sequences or chromatin. Suitably, for targeting a pathogethic gene such as in ALS/FTD the binding site comprises repeats of 5′-GGG GCC-3′. However, it is envisaged that suitable binding sites may also or alternatively comprise repeats of 5′-GGG CCG-3′, 5′-GGC CGG-3′, 5′-GCC GGG-3′ or 5′-CCG GGG-3′. Desirably, target sequences for the extended zinc finger peptides of the invention comprise 30 or more contiguous 5′-GGG GCC-3′ repeats, such as at least 100 contiguous 5′-GGG GCC-3′ repeats, at least 200 contiguous 5′-GGG GCC-3′ repeats, or at least 700 contiguous 5′-GGG GCC-3′ repeats. In some embodiments of these aspects, target sequences for zinc finger peptides of the invention—preferably for zinc finger activator peptides of the invention—comprise less than 30 contiguous 5′-GGG GCC-3′ repeats, such as 20 or less contiguous 5′-GGG GCC-3′ repeats, 10 or less contiguous 5′-GGG GCC-3′ repeats, or 8 or less contiguous 5′-GGG GCC-3′ repeats.

In some aspects and embodiments, a particular advantage of the zinc finger peptides of the invention is that they bind to longer arrays of GGGGCC- repeat sequences in preference to shorter arrays. Accordingly, the GGGGCC-targeting extended zinc finger peptides of the invention bind more effectively (e.g. with higher affinity or greater gene modulation ability) to expanded, pathogenic nucleotide-repeat sequences compared to wild-type nucleotide-repeat sequences. For targeting/treatment of ALS/FTD, GGGGCC-targeting extended zinc finger peptides of the invention bind with higher affinity to expanded GGGGCC-repeat sequences containing at least 30 repeats, compared to sequences containing e.g. 8 or less repeats. Similarly, sequences containing at least 100 GGGGCC repeats may be bound preferentially over sequences containing 10 or less repeats (including 8 or less); sequences containing at least 200 or 700 GGGGCC repeats may be bound preferentially over sequences containing 20 or less repeats (as well as sequences including 10 or less or 8 or less). Similarly, sequences containing at least 55 GGGGCC repeats may be bound preferentially over sequences containing 20 or less repeats (including 10 or less).

### B. Poly Zinc Finger Repressor Proteins for Targeting GGGGCC-Repeats

As the skilled person will appreciate, the amino acid sequence of the zinc finger recognition sequence of each zinc finger domain of a poly-zinc finger peptide of the invention is determined by the nucleic acid sequence of the target nucleic acid triplet (or staggered quadrulplet). According to first aspects and embodiments of the invention, the zinc finger peptides are designed to target alternating GGG and GCC triplets. Accordingly, the recognition sequences of adjacent zinc finger domains of a poly-zinc finger peptide of the invention may generally alternate along the length of the zinc finger array. It may, therefore, be convenient to consider the zinc finger domains of a zinc finger peptide of these aspects and embodiments of the invention to belong to one of two sequence types, e.g. a ‘first type’ zinc finger domain for binding to a GCC triplet and a ‘second type’ zinc finger domain for binding to a GGG triplet. In embodiments the recognition sequences of the ‘first type’ represents the odd-numbered zinc finger domains of the relevant portion of the zinc finger array (e.g. fingers 1, 3, 5, 7, 9, 11, 13 etc. when read in a direction from N to C terminals), and the ‘second type’ represents the even-numbered zinc finger domains of the relevant portion of the zinc finger array (e.g. fingers 2, 4, 6, 8, 10, 12 etc. in N to C terminal direction), or vice versa, such that the ‘first type’ are located at fingers 2, 4, 6, 8, 10, 12 etc., whereas the ‘second type’ are located at the odd finger positions.

In alternative embodiments, a long flexible linker within the zinc finger array may be used to ‘reset’ the zinc finger ‘type’ as may be desired—e.g. so that each sub-array (i.e. a group of zinc fingers linked in tandem via short linker sequences of 5, 6 or 7 amino acids within a larger zinc finger peptide array comprising at least one long, flexible linker) of zinc finger domains may begin with the most N-terminal domain of a particular desired ‘type’. A long flexible linker can allow extended zinc finger peptides to target discontinuous sub-sites where the long flexible linker is able to span one or more, typically 3 or more nucleotides of a double-stranded polynucleic acid. Adding 6- and/or 7-amino acid linkers and long flexible linkers can help with ‘tuning’ of or otherwise customising the zinc finger-nucleic acid binding interaction as desired.

By way of example and not limitation, therefore, where a long flexible linker sequence is used between the fifth and sixth zinc finger domains of an array, the ‘first type’ of zinc finger recognition sequence may encompass fingers 1, 3, 5, 6, 8, 10 etc., and a ‘second type’ of zinc finger recognition sequence may encompass fingers 2, 4, 7, 9, 11 etc. (in N to C terminal direction), or vice versa.

Suitably, the recognition sequences of the zinc finger peptides of the invention may be selected from two general formulae, which alternate along the zinc finger array of the inventive zinc finger peptides. As noted above, where an extended zinc finger peptide of the invention comprises so-called ‘long/flexible linkers’ (as described herein), the two general formulae alternate within each zinc finger sub-array, which alternation may be in phase with, or out of phase with the alternation of each adjacent sub-array.

According to the invention, zinc finger recognition sequences (i.e. positions X⁻¹, X⁺¹, X⁺², X⁺³, X⁺⁴, X⁺⁵ and X⁺⁶ in Formulas 2, 4 and 6 above) may be of a first type represented by the amino acid sequence of:

SEQ ID NO: 1:
(D/A/G)SS(V/D/E/A/G)(L/R)(T/K)(R/K/G)
SEQ ID NO: 2:
(D/A/G/T/V/S)(S/N/R/A/G)(S/R/E/A/G)(V/D/E/A/G/I/L/S/T)(L/R)(T/K)(R/K/G);
and may be of a second type represented by the amino acid sequence of:
SEQ ID NO: 3:
RS(D/A/G)HL(T/S/A)(R/K/G);
SEQ ID NO: 133:
(R/G)S(D/G)HL(T/S/A)(R/K/G);
or
SEQ ID NO: 134:
(R/G)G(D/S/G)HR(K/I/A)(R/K/G).

The first type recognition sequences may further be represented by the amino acid sequences of:

SEQ ID NO: 4:
(D/A/G)SS(V/D/E/A/G)LT(R/K/G)
SEQ ID NO: 5:
(D/A/G/T/V/S)(S/N/R)(S/R/E/A/G)(V/D/E/A/G/I/L/S/T)LT(R/K/G)
SEQ ID NO: 6:
(D/A/G)SS(V/D/E/A/G)RK(R/K/G)
and
SEQ ID NO: 7:
(D/A/G/T/V/S)(S/N/R)(S/R/E/A/G)(V/D/E/A/G/I/L/S/T)RK(R/K/G);
or
SEQ ID NO: 8:
(D/A)SS(V/E)LT(R/K)
SEQ ID NO: 9:
D/A/T)(S/N/R)(S/R/E)(V/E/D)LTR
SEQ ID NO: 10:
(D/A)SS(V/E)RK(R/K)
and
SEQ ID NO: 11:
(D/A/T)(S/N/R)(S/R/E)(V/E/D)RKR;
or
SEQ ID NO: 181:
(G/D)S(S/G)(E/D)(L/R)(T/K)(R/K);
SEQ ID NO: 182:
(G/D)S(S/G)(E/D)LT(R/K);
SEQ ID NO: 183:
(G/D)S(S/G)(E/D)RK(R/K);
or
SEQ ID NO: 184:
(D/A)(N/G)(G/A)(V/D)(L/R)(T/K)(R/K);
SEQ ID NO: 185:
(D/A)(N/G)(G/A)(V/D)LT(R/K);
and
SEQ ID NO: 186:
(D/A)(N/G)(G/A)(V/D)RK(R/K).

The second type recognition sequences may further be represented by the amino acid sequences of:

SEQ ID NO: 12:
RS(D/G)HLT(R/K/G);
SEQ ID NO: 135:
(R/G)SDHLT(R/K);
or
SEQ ID NO: 136:
RG(D/S)HRK(R/K).

In some embodiments, at least 2—for example, 2, 3, 4 or 5 of the variable positions in each of SEQ ID NOs: 1 to 12, 133 to 136 and 181 to 186 are selected to be the first residue within each set of parentheses “( . . . )”. In some embodiments at least 1—for example, 1, 2, 3 or 4—of the variable positions in each of SEQ ID NOs: 1 to 12, 133 to 136 and 181 to 186 are selected to be other than the first residue within each set of parentheses “( . . . )”.

As noted above, beneficially, recognition sequences of the first type are adapted/tuned to bind the triplet 5′-GCC-3′ and recognition sequences of the second type are adapted/tuned to bind the triplet 5′-GGG-3′. Hence, in embodiments, odd-numbered zinc finger domains are suitably of the first type and even-numbered zinc finger domains are suitably of the second type. In other embodiments, odd-numbered zinc finger domains in a first zinc finger sub-array are suitably of the first type and odd-numbered zinc finger domains of a second, adjacent zinc finger sub-array are suitably of the second type, such that the recognition sequence of SEQ ID NO: 1 or 2 alternates with the recognition sequence of SEQ ID NO: 3, 133 or 134 within each zinc finger sub-array, and so on.

Thus, in embodiments, there is provided an engineered zinc finger (DNA-binding) peptide comprising at least 8, such as from 8 to 32, or more specifically 8, 10, 11, 12 or 18 zinc finger domains having the zinc finger recognition sequences of SEQ ID NO: 1 and/or 2 and SEQ ID NO: 3, 133 and/or 134.

In embodiments of the extended poly-zinc finger peptides of the invention the zinc finger domain recognition sequences alternate along the length of the zinc finger peptide array between any one or more of the first type and any one or more of the second type recognition sequences defined herein. Suitably, odd numbered zinc fingers of the zinc finger array are of the first type sequence and even number zinc fingers of the array are of the second type sequence. In some alternative embodiments, odd numbered zinc fingers of each sub-array within a poly-zinc finger peptide of the invention have the first type sequence and even number zinc fingers of each sub-array have the second type sequence.

A preferred extended zinc finger peptide of the invention has 11 zinc finger modules, wherein fingers F1, F3, F5, F7, F9 and F11 have recognition sequences according to the first type sequences set out herein, and fingers F2, F4, F6, F8 and F10 have recognition sequences according to the second type sequences set out herein. In another embodiment an extended zinc finger peptide of the invention has 11 zinc finger modules, wherein fingers F1, F3, F5, F6, F8 and F10 have recognition sequences according to the first type sequences set out herein, and fingers F2, F4, F7, F9 and F11 have recognition sequences according to the second type sequences set out herein. In some particularly beneficial embodiments, the recognition sequence of the first zinc finger of the zinc finger peptide array is selected from the sequence encompassed by SEQ ID NOs: 4 or 5, or 8 or 9. In some particularly beneficial embodiments, the recognition sequence of the first zinc finger in each zinc finger peptide array is selected from the sequence encompassed by SEQ ID NOs: 4 or 5, or 8 or 9, and all further first type zinc finger domains have a recognition sequence encompassed by SEQ ID NOs: 6 or 7, or 10 or 11. It will be understood, therefore, that within the scope of the invention, one or more recognition sequence of SEQ ID NO: 4 may be replaced with the sequence of SEQ ID NO: 5 and vice versa, and one or more recognition sequence of SEQ ID NO: 8 may be replaced with the sequence of SEQ ID NO: 9 and vice versa. Similarly, one or more recognition sequence of SEQ ID NO: 6 may be replaced with the sequence of SEQ ID NO: 7 and vice versa, and one or more recognition sequence of SEQ ID NO: 10 may be replaced with the sequence of SEQ ID NO: 11 and vice versa in order to tune the zinc finger peptide to have the desired binding characteristics. It is also noted that the features of the 11-zinc finger peptide embodiments set out above apply equally with appropriate, logical adjustments according to the number of zinc finger domains, to all other extended poly-zinc finger peptides of the invention, such as those containing 8, 10, 12 or 18 zinc finger domains.

Beneficially, therefore, the engineered zinc finger peptides of the invention comprise at least 10, 11, 12 or 18 adjacent zinc finger modules. In some embodiments, the zinc finger peptides of the invention comprise more than 10, 11, 12 or 18 zinc finger domains—such as any number between 11 and 32 zinc finger domains, provided that at least 8, 10, 11, 12 or 18 adjacent domains have the specified recognition sequence. In some embodiments all zinc finger domains of a zinc finger peptide of the invention are the recognition sequences as set out herein.

Table 1 below summarises preferred recognition sequence arrangements of the extended poly-zinc finger peptides (e.g. repressor peptides) of these aspects and embodiments of the invention. In this table, one or more sequences of SEQ ID NO: 3 may be substituted with the sequences of SEQ ID NO: 133 or 134; for example, all sequences of SEQ ID NO: 3 may be replaced with the sequences of SEQ ID NO: 133 or SEQ ID NO: 134, or mixtures thereof. Similarly, in the table below, one or more sequences of SEQ ID NO: 12 may be substituted with the sequences of SEQ ID NO: 135 or 136; for example, all sequences of SEQ ID NO: 12 may be replaced with the sequences of SEQ ID NO: 135 or SEQ ID NO: 136, or mixtures thereof.

TABLE 1
Exemplary zinc finger recognition helix arrangements of zinc finger peptides according
to the invention for binding GGGGCC repeat sequences, e.g. for treating Amyotrophic
lateral sclerosis (ALS) and Frontotemporal dementia (FTD). The zinc finger peptide
recognition sequence patterns of this table apply to an entire zinc finger peptide,
or to each sub-array of a zinc finger peptide of the invention. Zinc finger peptides
disclosed in this table may have from 8 to 32 fingers, for example, 8, 9, 10,
11, 12, 13, 14, 15, 16, 17 or 18 zinc finger domains.

| Finger number of zinc | Zinc Finger Number (N to C terminal direction) | | | |
|---|---|---|---|---|
| finger peptide or of | | F2, F4, F6, F8, | F3, F5, F7, F9, | |
| each sub-array of a | F1 | F10 etc. | F11 etc. | ZFP |
| zinc finger peptide | (GCC-binding) | (GGG-binding) | (GCC-binding) | Array |
| Recognition Sequence | SEQ ID NO: 1 | SEQ ID NO: 3 | SEQ ID NO: 1 | A |
| | SEQ ID NO: 2 | SEQ ID NO: 3 | SEQ ID NO: 2 | B |
| | SEQ ID NO: 4 | SEQ ID NO: 3 | SEQ ID NO: 4 | C |
| | SEQ ID NO: 4 | SEQ ID NO: 3 | SEQ ID NO: 6 | D |
| | SEQ ID NO: 6 | SEQ ID NO: 3 | SEQ ID NO: 6 | E |
| | SEQ ID NO: 5 | SEQ ID NO: 3 | SEQ ID NO: 5 | F |
| | SEQ ID NO: 5 | SEQ ID NO: 3 | SEQ ID NO: 7 | G |
| | SEQ ID NO: 7 | SEQ ID NO: 3 | SEQ ID NO: 7 | H |
| | SEQ ID NO: 8 | SEQ ID NO: 3 | SEQ ID NO: 8 | I |
| | SEQ ID NO: 8 | SEQ ID NO: 3 | SEQ ID NO: 10 | J |
| | SEQ ID NO: 10 | SEQ ID NO: 3 | SEQ ID NO: 10 | K |
| | SEQ ID NO: 9 | SEQ ID NO: 3 | SEQ ID NO: 9 | L |
| | SEQ ID NO: 9 | SEQ ID NO: 3 | SEQ ID NO: 11 | M |
| | SEQ ID NO: 11 | SEQ ID NO: 3 | SEQ ID NO: 11 | N |
| | SEQ ID NO: 1 | SEQ ID NO: 12 | SEQ ID NO: 1 | O |
| | SEQ ID NO: 2 | SEQ ID NO: 12 | SEQ ID NO: 2 | P |
| | SEQ ID NO: 4 | SEQ ID NO: 12 | SEQ ID NO: 4 | Q |
| | SEQ ID NO: 4 | SEQ ID NO: 12 | SEQ ID NO: 6 | R |
| | SEQ ID NO: 6 | SEQ ID NO: 12 | SEQ ID NO: 6 | S |
| | SEQ ID NO: 5 | SEQ ID NO: 12 | SEQ ID NO: 5 | T |
| | SEQ ID NO: 5 | SEQ ID NO: 12 | SEQ ID NO: 7 | U |
| | SEQ ID NO: 7 | SEQ ID NO: 12 | SEQ ID NO: 7 | V |
| | SEQ ID NO: 8 | SEQ ID NO: 12 | SEQ ID NO: 8 | W |
| | SEQ ID NO: 8 | SEQ ID NO: 12 | SEQ ID NO: 10 | X |
| | SEQ ID NO: 10 | SEQ ID NO: 12 | SEQ ID NO: 10 | Y |
| | SEQ ID NO: 9 | SEQ ID NO: 12 | SEQ ID NO: 9 | Z |
| | SEQ ID NO: 9 | SEQ ID NO: 12 | SEQ ID NO: 11 | AA |
| | SEQ ID NO: 11 | SEQ ID NO: 12 | SEQ ID NO: 11 | AB |
| | SEQ ID NO: 181 | SEQ ID NO: 12 | SEQ ID NO: 181 | JX |
| | SEQ ID NO: 182 | SEQ ID NO: 12 | SEQ ID NO: 183 | JY |
| | SEQ ID NO: 182 | SEQ ID NO: 12 | SEQ ID NO: 183 | JZ |
| | SEQ ID NO: 181 | SEQ ID NO: 133 | SEQ ID NO: 181 | KA |
| | SEQ ID NO: 182 | SEQ ID NO: 133 | SEQ ID NO: 182 | KB |
| | SEQ ID NO: 182 | SEQ ID NO: 133 | SEQ ID NO: 183 | KC |
| | SEQ ID NO: 181 | SEQ ID NO: 134 | SEQ ID NO: 181 | KD |
| | SEQ ID NO: 182 | SEQ ID NO: 134 | SEQ ID NO: 182 | KE |
| | SEQ ID NO: 182 | SEQ ID NO: 134 | SEQ ID NO: 183 | KF |
| | SEQ ID NO: 184 | SEQ ID NO: 3 | SEQ ID NO: 184 | LP |
| | SEQ ID NO: 185 | SEQ ID NO: 3 | SEQ ID NO: 185 | LQ |
| | SEQ ID NO: 186 | SEQ ID NO: 3 | SEQ ID NO: 186 | LR |
| | SEQ ID NO: 184 | SEQ ID NO: 12 | SEQ ID NO: 184 | LS |
| | SEQ ID NO: 185 | SEQ ID NO: 12 | SEQ ID NO: 185 | LT |
| | SEQ ID NO: 186 | SEQ ID NO: 12 | SEQ ID NO: 186 | LU |
| Finger number of zinc | | | | |
| finger peptide or of | | F2, F4, F6, F8, | F3, F5, F7, F9, | |
| each sub-array of a | F1 | F10 etc. | F11 etc. | |
| zinc finger peptide | (GGG-binding) | (GCC-binding) | (GGG-binding) | |
| Recognition Sequence | SEQ ID NO: 3 | SEQ ID NO: 1 | SEQ ID NO: 3 | AC |
| | SEQ ID NO: 3 | SEQ ID NO: 2 | SEQ ID NO: 3 | AD |
| | SEQ ID NO: 3 | SEQ ID NO: 4 | SEQ ID NO: 3 | AE |
| | SEQ ID NO: 3 | SEQ ID NO: 5 | SEQ ID NO: 3 | AF |
| | SEQ ID NO: 3 | SEQ ID NO: 6 | SEQ ID NO: 3 | AG |
| | SEQ ID NO: 3 | SEQ ID NO: 7 | SEQ ID NO: 3 | AH |
| | SEQ ID NO: 3 | SEQ ID NO: 8 | SEQ ID NO: 3 | AI |
| | SEQ ID NO: 3 | SEQ ID NO: 9 | SEQ ID NO: 3 | AJ |
| | SEQ ID NO: 3 | SEQ ID NO: 10 | SEQ ID NO: 3 | AK |
| | SEQ ID NO: 3 | SEQ ID NO: 11 | SEQ ID NO: 3 | AL |
| | SEQ ID NO: 12 | SEQ ID NO: 1 | SEQ ID NO: 12 | AM |
| | SEQ ID NO: 12 | SEQ ID NO: 2 | SEQ ID NO: 12 | AN |
| | SEQ ID NO: 12 | SEQ ID NO: 4 | SEQ ID NO: 12 | AO |
| | SEQ ID NO: 12 | SEQ ID NO: 5 | SEQ ID NO: 12 | AP |
| | SEQ ID NO: 12 | SEQ ID NO: 6 | SEQ ID NO: 12 | AQ |
| | SEQ ID NO: 12 | SEQ ID NO: 7 | SEQ ID NO: 12 | AR |
| | SEQ ID NO: 12 | SEQ ID NO: 8 | SEQ ID NO: 12 | AS |
| | SEQ ID NO: 12 | SEQ ID NO: 9 | SEQ ID NO: 12 | AT |
| | SEQ ID NO: 12 | SEQ ID NO: 10 | SEQ ID NO: 12 | AU |
| | SEQ ID NO: 12 | SEQ ID NO: 11 | SEQ ID NO: 12 | AV |
| | SEQ ID NO: 12 | SEQ ID NO: 181 | SEQ ID NO: 134 | KG |
| | SEQ ID NO: 12 | SEQ ID NO: 182 | SEQ ID NO: 134 | KH |
| | SEQ ID NO: 12 | SEQ ID NO: 183 | SEQ ID NO: 134 | KI |
| | SEQ ID NO: 133 | SEQ ID NO: 181 | SEQ ID NO: 134 | KJ |
| | SEQ ID NO: 133 | SEQ ID NO: 182 | SEQ ID NO: 134 | KK |
| | SEQ ID NO: 133 | SEQ ID NO: 183 | SEQ ID NO: 134 | KL |

Extended poly-zinc finger repressors of the invention may have 2, 3, 4, 5 or 6 sub-arrays, generally 2 or 3 sub-arrays and preferably 2 sub-arrays within each of which the zinc finger recognition sequence pattern may be selected from any of the combinations disclosed in Table 1 above.

In embodiments, the first type of recognition sequence is selected from (D/A/G)SS(V/D/E/A/G)LT(R/K/G), (D/A/G)SS(V/D/E/A/G)RK(R/K/G), (D/A/G/T/V/S)(S/N/R)(S/R/E/A/G)(V/D/E/A/G/I/L/S/T)LT(R/K/G), or (D/A/G/T/V/S)(S/N/R)(S/R/E/A/G)(V/D/E/A/G/I/L/S/T)RK(R/K/G). In some preferred embodiments, the recognition sequences of the first type of zinc finger domain within a poly-zinc zinc finger peptide of the invention includes recognition sequences of both (D/A/G)SS(V/D/E/A/G)LT(R/K/G) and (D/A/G)SS(V/D/E/A/G)RK(R/K/G); or both (D/A/G/T/V/S)(S/N/R)(S/R/E/A/G)(V/D/E/A/G/I/L/S/T)LT(R/K/G) and (D/A/G/T/V/S)(S/N/R)(S/R/E/A/G)(V/D/E/A/G/I/L/S/T)RK(R/K/G). Suitably, the first finger, F1 of the poly-zinc finger peptide has a recognition sequence according to (D/A/G)SS(V/D/E/A/G)LT(R/K/G) or (D/A/G/T/V/S)(S/N/R)(S/R/E/A/G)(V/D/E/A/G/I/L/S/T)LT(R/K/G), which sequences have most sequence similarity to the F1 recognition sequence of Zif268, thereby increasing host-matching of the sequences and reducing potential toxicity or immunogenic reactions; and the remaining recognition sequences of the first type of zinc finger domain may have a sequence according to (D/A/G)SS(V/D/E/A/G)RK(R/K/G) or (D/A/G/T/V/S)(S/N/R)(S/R/E/A/G)(V/D/E/A/G/I/L/S/T)RK(R/K/G), respectively, so as to increase the proportion of host-matched residues. In some embodiments, the first finger of each zinc finger sub-array within an extended poly-zinc finger of the invention has a recognition sequence according to (D/A/G)SS(V/D/E/A/G)LT(R/K/G) or (D/A/G/T/V/S)(S/N/R)(S/R/E/A/G)(V/D/E/A/G/I/L/S/T)LT(R/K/G); and the remaining recognition sequences of the first type of zinc finger domain within the extended poly-zinc finger peptide have a sequence according to (D/A/G)SS(V/D/E/A/G)RK(R/K/G) or (D/A/G/T/V/S)(S/N/R)(S/R/E/A/G)(V/D/E/A/G/I/L/S/T)RK(R/K/G), respectively.

In embodiments, in order to tune the binding affinity of the extended poly-zinc fingers of the invention against the GGGGCC repeat sequence, and particularly against the GCC triplet of the hexapeptide, at least one residue of SEQ ID NO: 1 or 2 is A or G; and is suitably G. In some such embodiments the residue at position −1 is G; in some embodiments the residue at position 3 is G; in some embodiments the residue at position 2 is G and in some embodiments the residue at position 6 is G. Preferably, the residue at position −1 is G or the residue at position 3 is G. Suitably, the residue at position −1 may be A or G and the residue at position 3 may be E or G. In this way, the binding affinity of extended poly-zinc finger peptides of the invention for GGGGCC repeat sequences may be advantageously reduced—particularly with respect to zinc finger repressor proteins—such that undesirable repression of wild-type alleles (i.e. those having less than 30 GGGGCC repeats) is reduced, minimised or substantially prevented. In embodiments, the proportion of G residues is increased as the number of zinc finger domains increases. Therefore, in an 11-zinc finger peptide there may be one G residue per zinc finger pair (i.e. for binding to each GGGGCC hexanucleotide). For an 18-zinc finger peptide there may be two G residues for each adjacent pair of zinc fingers.

In some embodiments, the recognition sequence of one or more zinc finger domains of the first type is selected from a sequence of SEQ ID NO: 1, e.g. selected from: SEQ ID NO: 13 (DSSVLTR), SEQ ID NO: 14 (DSSVRKR), SEQ ID NO: 15 (ASSVLTR), SEQ ID NO: 16 (ASSVRKR), SEQ ID NO: 17 (DSSELTR), SEQ ID NO: 18 (DSSERKR), SEQ ID NO: 19 (ASSELTR), SEQ ID NO: 20 (ASSERKR), SEQ ID NO: 21 (GSSVLTR), SEQ ID NO: 22 (GSSVRKR), SEQ ID NO: 23 (GSSELTR), SEQ ID NO: 24 (GSSERKR), SEQ ID NO: 25 (DSSGLTR), SEQ ID NO: 26 (DSSGRKR), SEQ ID NO: 27 (ASSGLTR), SEQ ID NO: 28 (ASSGRKR), SEQ ID NO: 29 (GSSGLTR), SEQ ID NO: 30 (GSSGRKR), SEQ ID NO: 137 (DSSVLTG), SEQ ID NO: 138 (DSSVRKG), SEQ ID NO: 139 (DSSELTG), SEQ ID NO: 140 (DSSERKG), SEQ ID NO: 187 (DSGDLTR), SEQ ID NO: 188 (DSGDRKR), SEQ ID NO: 189 (DSGELTR), SEQ ID NO: 190 (DSGERKR), SEQ ID NO: 191 (GSSELTK) individually or any combination of two or more thereof. In some beneficial embodiments, the recognition sequences of each zinc finger domain of the first type is selected from the group consisting of: SEQ ID NO: 13 (DSSVLTR), SEQ ID NO: 14 (DSSVRKR), SEQ ID NO: 19 (ASSELTR), SEQ ID NO: 20 (ASSERKR), SEQ ID NO: 21 (GSSVLTR), SEQ ID NO: 22 (GSSVRKR), SEQ ID NO: 23 (GSSELTR), SEQ ID NO: 24 (GSSERKR), SEQ ID NO: 25 (DSSGLTR), SEQ ID NO: 26 (DSSGRKR), SEQ ID NO: 27 (ASSGLTR), SEQ ID NO: 28 ASSGRKR), SEQ ID NO: 29 (GSSGLTR), SEQ ID NO: 30 (GSSGRKR), SEQ ID NO: 137 (DSSVLTG), SEQ ID NO: 138 (DSSVRKG), SEQ ID NO: 187 (DSGDLTR), SEQ ID NO: 188 (DSGDRKR), SEQ ID NO: 189 (DSGELTR), SEQ ID NO: 190 (DSGERKR), SEQ ID NO: 191 (GSSELTK) individually or any combination or two or more thereof. In some beneficial embodiments, the recognition sequences of each zinc finger domain of the first type is selected from the group consisting of: SEQ ID NO: 13 (DSSVLTR), SEQ ID NO: 21 (GSSVLTR), SEQ ID NO: 22 (GSSVRKR), SEQ ID NO: 23 (GSSELTR), SEQ ID NO: 24 (GSSERKR), SEQ ID NO: 25 (DSSGLTR), SEQ ID NO: 26 (DSSGRKR), SEQ ID NO: 27 (ASSGLTR), SEQ ID NO: 28 (ASSGRKR), SEQ ID NO: 29 (GSSGLTR), SEQ ID NO: 30 (GSSGRKR), SEQ ID NO: 187 (DSGDLTR), SEQ ID NO: 188 (DSGDRKR) individually or any combination or two or more thereof. In some beneficial embodiments, the recognition sequences of each zinc finger domain of the first type is selected from the group consisting of: SEQ ID NO: 13 (DSSVLTR), SEQ ID NO: 14 (DSSVRKR), SEQ ID NO: 19 (ASSELTR), SEQ ID NO: 20 (ASSERKR), SEQ ID NO: 23 (GSSELTR), SEQ ID NO: 24 (GSSERKR) individually or any combination or two or more thereof.

In some embodiments, the recognition sequence of one or more zinc finger domains of the first type is selected from a sequence of SEQ ID NO: 2 selected from: SEQ ID NO: 31 (DNRDLTR), SEQ ID NO: 145 (DNGDLTR), SEQ ID NO: 32 (DNRDRKR), SEQ ID NO: 33 (TREDLTR), SEQ ID NO: 34 (TREDRKR), SEQ ID NO: 35 (DNRELTR), SEQ ID NO: 36 (DNRERKR), SEQ ID NO: 37 (ANRELTR), SEQ ID NO: 38 (ANRERKR) SEQ ID NO: 39 (DREELTR), SEQ ID NO: 40 (DREERKR), SEQ ID NO: 41 (AREELTR), SEQ ID NO: 42 (AREERKR), SEQ ID NO: 43 (TNRELTR), SEQ ID NO: 44 (TNRERKR), SEQ ID NO: 45 (TREELTR), SEQ ID NO: 46 (TREERKR), SEQ ID NO: 47 (GNRELTR), SEQ ID NO: 48 (GNRERKR), SEQ ID NO: 49 (GREELTR), SEQ ID NO: 50 (GREERKR), SEQ ID NO: 51 (DNRGLTR), SEQ ID NO: 52 (DNRGRKR), SEQ ID NO: 53 (ANRGLTR), SEQ ID NO: 54 (ANRGRKR), SEQ ID NO: 55 (DREGLTR), SEQ ID NO: 56 (DREGRKR), SEQ ID NO: 57 (AREGLTR), SEQ ID NO: 58 (AREGRKR), SEQ ID NO: 59 (TNRGLTR), SEQ ID NO: 60 (TNRGRKR), SEQ ID NO: 61 (TREGLTR), SEQ ID NO: 62 (TREGRKR), SEQ ID NO: 63 (DNRELTG), SEQ ID NO: 64 (DNRERKG), SEQ ID NO: 65 (TREELTG), SEQ ID NO: 66 (TREERKG), SEQ ID NO: 67 (GNRDLTR), SEQ ID NO: 68 (GNRDRKR), SEQ ID NO: 69 (GREDLTR), SEQ ID NO: 70 (GREDRKR), SEQ ID NO: 71 (GNRGLTR), SEQ ID NO: 72 (GNRGRKR), SEQ ID NO: 73 (GREGLTR), SEQ ID NO: 74 (GREGRKR), SEQ ID NO: 146 (DGADLTR), SEQ ID NO: 147 (AGADLTR) individually or any combination or two or more thereof. In some beneficial embodiments, the recognition sequences of each zinc finger domain of the first type is selected from the group consisting of: SEQ ID NO: 31 (DNRDLTR), SEQ ID NO: 32 (DNRDRKR), SEQ ID NO: 33 (TREDLTR), SEQ ID NO: 34 (TREDRKR), SEQ ID NO: 51 (DNRGLTR), SEQ ID NO: 52 (DNRGRKR), SEQ ID NO: 61 (TREGLTR), SEQ ID NO: 62 (TREGRKR), SEQ ID NO: 63 (DNRELTG), SEQ ID NO: 64 (DNRERKG), SEQ ID NO: 67 (GNRDLTR), SEQ ID NO: 68 (GNRDRKR), SEQ ID NO: 69 (GREDLTR), SEQ ID NO: 70 (GREDRKR), SEQ ID NO: 71 (GNRGLTR), SEQ ID NO: 72 (GNRGRKR), SEQ ID NO: 73 (GREGLTR), SEQ ID NO: 74 (GREGRKR), SEQ ID NO: 145 (DNGDLTR), SEQ ID NO: 146 (DGADLTR), SEQ ID NO: 147 (AGADLTR) individually or any combination or two or more thereof. In some beneficial embodiments, the recognition sequences of each zinc finger domain of the first type is selected from the group consisting of: SEQ ID NO: 51 (DNRGLTR), SEQ ID NO: 52 (DNRGRKR), SEQ ID NO: 61 (TREGLTR), SEQ ID NO: 62 (TREGRKR), SEQ ID NO: 67 (GNRDLTR), SEQ ID NO: 68 (GNRDRKR), SEQ ID NO: 69 (GREDLTR), SEQ ID NO: 70 (GREDRKR), SEQ ID NO: 71 (GNRGLTR), SEQ ID NO: 72 (GNRGRKR), SEQ ID NO: 73 (GREGLTR), SEQ ID NO: 74 (GREGRKR), SEQ ID NO: 146 (DGADLTR), SEQ ID NO: 147 (AGADLTR) individually or any combination or two or more thereof. In some beneficial embodiments, the recognition sequences of each zinc finger domain of the first type is selected from the group consisting of: SEQ ID NO: 31 (DNRDLTR), SEQ ID NO: 32 (DNRDRKR), SEQ ID NO: 33 (TREDLTR), SEQ ID NO: 34 (TREDRKR), SEQ ID NO: 147 (AGADLTR) individually or any combination or two or more thereof.

Advantageously, in any of the embodiments of the invention, the recognition sequence of the first zinc finger domain of a zinc finger peptide (F1) may be a sequence wherein the residues at positions +4 and +5 are, respectively, L and T. In this way host matching for in vivo mouse or human applications may be improved. In such embodiments all remaining recognition sequences of the first type may preferably have the residues R and K, respectively, in the 4 and 5 positions. In other embodiments, all remaining recognition sequences of the first type may have the residues L and T, respectively, in the 4 and 5 positions; or may include a mixture of R and K or L and T residues in the 4 and 5 positions, respectively—beneficially selected to best match a corresponding host sequence. Thus, in some embodiments, as described above, the first zinc finger of each sub-array (wherein sub-arrays are separated from each other by long, flexible linkers in accordance with the invention) has a recognition sequence wherein the residues at positions 4 and 5 are, respectively, L and T. In such embodiments, all remaining recognition sequences of the first type in that sub-array preferably have the residues R and K, respectively, in the 4 and 5 positions; or may have the residues L and T, respectively, in the 4 and 5 positions; or may include a mixture of R and K or L and T residues in the 4 and 5 positions, respectively.

Further, in embodiments, in order to tune the binding affinity of the extended poly-zinc fingers of the invention against the GGGGCC repeat sequence, and specifically against the GGG triplet of the hexapeptide, at least one residue of SEQ ID NO: 3 is G; in some embodiments the residue at position 2 is G; in some embodiments the residue at position 6 is G; in some embodiments the residues at positions 2 and 6 are G; suitably, the residue at position 2 is G and the residue at position 6 is K. In this way, the binding affinity of extended poly-zinc finger peptides of the invention for GGGGCC repeat sequences may be advantageously and controllably reduced-particularly for a zinc finger repressor protein of the invention—such that undesirable repression of wild-type alleles (i.e. those having less than 30 GGGGCC repeats) is reduced, minimised or substantially prevented. In some embodiments, the recognition sequence of one or more zinc finger domains of the second type is selected from: SEQ ID NO: 75 (RSDHLTR), SEQ ID NO: 76 (RSDHLTK), SEQ ID NO: 77 (RSDHLTG), SEQ ID NO: 78 (RSAHLTR), SEQ ID NO: 79 (RSAHLTK), SEQ ID NO: 80 (RSAHLTG), SEQ ID NO: 81 (RSGHLTR), SEQ ID NO: 82 (RSGHLTK), SEQ ID NO: 83 (RSGHLTG), SEQ ID NO: 141 (RGDHLTR), SEQ ID NO: 143 (RGDHLTK), SEQ ID NO: 142 (GSDHLTR), SEQ ID NO: 144 (GSDHLTK) individually or any combination or two or more thereof. Beneficially, the recognition sequences of each zinc finger domain of the second type is selected from the group consisting of: SEQ ID NO: 75 (RSDHLTR), SEQ ID NO: 76 (RSDHLTK), SEQ ID NO: 77 (RSDHLTG), SEQ ID NO: 78 (RSAHLTR), SEQ ID NO: 81 (RSGHLTR), SEQ ID NO: 82 (RSGHLTK), SEQ ID NO: 83 (RSGHLTG), SEQ ID NO: 142 (GSDHLTR), SEQ ID NO: 144 (GSDHLTK) individually or any combination or two or more thereof. Advantageously, the recognition sequences of each zinc finger domain of the second type is selected from the group consisting of: SEQ ID NO: 75 (RSDHLTR), SEQ ID NO: 76 (RSDHLTK), SEQ ID NO: 78 (RSAHLTR), SEQ ID NO: 81 (RSGHLTR), SEQ ID NO: 82 (RSGHLTK) individually or any combination or two or more thereof. Preferably, the recognition sequences of each zinc finger domain of the second type is selected from the group consisting of: SEQ ID NO: 78 (RSAHLTR), SEQ ID NO: 81 (RSGHLTR) and SEQ ID NO: 82 (RSGHLTK), individually or in combination.

In some embodiments, the zinc finger domains of the second type have recognition sequences that comprise more than one of the sequences of SEQ ID NO: 3,133,134 or 12, 135,136 for example, 2 or 3 different recognition sequences. Conveniently, all of the recognition sequences of the second type within a single zinc finger peptide array of the invention include only 1 or 2 sequences selected from SEQ ID NOs: 3, 133, 134 or 12, 135, 136 or from each of the subgroups of SEQ ID NO: 3 and SEQ ID NO: 12 listed above. Suitably, the recognition sequences of the second type of zinc finger domain are selected from RSGHLTR (SEQ ID NO: 81) and RSGHLTK (SEQ ID NO: 82); for example, there may be 1,2,3,4,5 or 6 recognition sequences of RSGHLTR and 1, 2, 3, 4, 5 or 6 recognition sequences of RSGHLTK as appropriate. In the embodiments described herein, one or more—up to all of the recognition sequences of the second type of zinc finger domain may be RSGHLTG (SEQ ID NO: 83) or RSDHLTG (SEQ ID NO: 77); and particularly may be RSGHLTG. In other embodiments, all recognition sequences of the second type are the same, and more suitably, all are RSGHLTR or RSGHLTK.

Table 2 below summarises preferred recognition sequence arrangements of the extended poly-zinc finger peptides of these aspects and embodiments of the invention.

TABLE 2
Exemplary zinc finger recognition helix arrangements of zinc finger peptides
according to the invention. The zinc finger peptide recognition sequence patterns
of this table apply to an entire zinc finger peptide, or to each sub-array of a
zinc finger peptide of the invention. Zinc finger peptides disclosed in this table
may have from 8 to 32 fingers, for example, 8, 9, 10, 11, 12, 13, 14, 15, 16, 17
or 18 zinc finger domains. #All F1 sequences may be exchanged for the corresponding
sequence with RK or RI at positions +4 and +5 in place of LT and all such
combinations are disclosed herein. In addition, all of the TREDLTR sequences (SEQ
ID NO: 33) in the F1 and/or F3, F5, F7, F9, F11 etc. positions above (see sequences
DQ to EB) can be substituted for the TREGLTR sequence (SEQ ID NO: 61).

| Finger number of zinc | | | | |
|---|---|---|---|---|
| finger peptide or of | Zinc Finger Number (N to C terminal direction) | | | |
| each sub-array of a | | F2, F4, F6, | F3, F5, F7, | ZFP |
| zinc finger peptide | F1# | F8, F10 etc. | F9, F11 etc. | Array |
| Recognition Sequence | DSSVLTR | RSDHLTR | DSSVLTR | AW |
| | DSSVLTR | RSDHLTK | DSSVLTR | AX |
| | DSSVLTR | RSDHLTG | DSSVLTR | AY |
| | DSSVLTR | RSGHLTR | DSSVLTR | AZ |
| | DSSVLTR | RSGHLTK | DSSVLTR | BA |
| | DSSVLTR | RSGHLTG | DSSVLTR | BB |
| | DSSVLTR | RSDHLTR | DSSVRKR | BC |
| | DSSVLTR | RSDHLTK | DSSVRKR | BD |
| | DSSVLTR | RSDHLTG | DSSVRKR | BE |
| | DSSVLTR | RSGHLTR | DSSVRKR | BF |
| | DSSVLTR | RSGHLTK | DSSVRKR | BG |
| | DSSVLTR | RSGHLTG | DSSVRKR | BH |
| | GSSVLTR | RSDHLTR | GSSVLTR | BI |
| | GSSVLTR | RSDHLTK | GSSVLTR | BJ |
| | GSSVLTR | RSDHLTG | GSSVLTR | BK |
| | GSSVLTR | RSGHLTR | GSSVLTR | BL |
| | GSSVLTR | RSGHLTK | GSSVLTR | BM |
| | GSSVLTR | RSGHLTG | GSSVLTR | BN |
| | GSSVLTR | RSDHLTR | GSSVRKR | BO |
| | GSSVLTR | RSDHLTK | GSSVRKR | BP |
| | GSSVLTR | RSDHLTG | GSSVRKR | BQ |
| | GSSVLTR | RSGHLTR | GSSVRKR | BR |
| | GSSVLTR | RSGHLTK | GSSVRKR | BS |
| | GSSVLTR | RSGHLTG | GSSVRKR | BT |
| | ASSVLTR | RSDHLTR | ASSVLTR | BU |
| | ASSVLTR | RSDHLTK | ASSVLTR | BV |
| | ASSVLTR | RSDHLTG | ASSVLTR | BW |
| | ASSVLTR | RSGHLTR | ASSVLTR | BX |
| | ASSVLTR | RSGHLTK | ASSVLTR | BY |
| | ASSVLTR | RSGHLTG | ASSVLTR | BZ |
| | ASSVLTR | RSDHLTR | ASSVRKR | CA |
| | ASSVLTR | RSDHLTK | ASSVRKR | CB |
| | ASSVLTR | RSDHLTG | ASSVRKR | CC |
| | ASSVLTR | RSGHLTR | ASSVRKR | CD |
| | ASSVLTR | RSGHLTK | ASSVRKR | CE |
| | ASSVLTR | RSGHLTG | ASSVRKR | CF |
| | ASSELTR | RSDHLTR | ASSELTR | CG |
| | ASSELTR | RSDHLTK | ASSELTR | CH |
| | ASSELTR | RSDHLTG | ASSELTR | CI |
| | ASSELTR | RSGHLTR | ASSELTR | CJ |
| | ASSELTR | RSGHLTK | ASSELTR | CK |
| | ASSELTR | RSGHLTG | ASSELTR | CL |
| | ASSELTR | RSDHLTR | ASSERKR | CM |
| | ASSELTR | RSDHLTK | ASSERKR | CN |
| | ASSELTR | RSDHLTG | ASSERKR | CO |
| | ASSELTR | RSGHLTR | ASSERKR | CP |
| | ASSELTR | RSGHLTK | ASSERKR | CQ |
| | ASSELTR | RSGHLTG | ASSERKR | CR |
| | GSSGLTR | RSDHLTR | GSSGLTR | CS |
| | GSSGLTR | RSDHLTK | GSSGLTR | CT |
| | GSSGLTR | RSDHLTG | GSSGLTR | CU |
| | GSSGLTR | RSGHLTR | GSSGLTR | CV |
| | GSSGLTR | RSGHLTK | GSSGLTR | CW |
| | GSSGLTR | RSGHLTG | GSSGLTR | CX |
| | GSSGLTR | RSDHLTR | GSSGRKR | CY |
| | GSSGLTR | RSDHLTK | GSSGRKR | CZ |
| | GSSGLTR | RSDHLTG | GSSGRKR | DA |
| | GSSGLTR | RSGHLTR | GSSGRKR | DB |
| | GSSGLTR | RSGHLTK | GSSGRKR | DC |
| | GSSGLTR | RSGHLTG | GSSGRKR | DD |
| | DNRDLTR | RSDHLTR | DNRDLTR | DE |
| | DNRDLTR | RSDHLTK | DNRDLTR | DF |
| | DNRDLTR | RSDHLTG | DNRDLTR | DG |
| | DNRDLTR | RSGHLTR | DNRDLTR | DH |
| | DNRDLTR | RSGHLTK | DNRDLTR | DI |
| | DNRDLTR | RSGHLTG | DNRDLTR | DJ |
| | DNRDLTR | RSDHLTR | DNRDRKR | DK |
| | DNRDLTR | RSDHLTK | DNRDRKR | DL |
| | DNRDLTR | RSDHLTG | DNRDRKR | DM |
| | DNRDLTR | RSGHLTR | DNRDRKR | DN |
| | DNRDLTR | RSGHLTK | DNRDRKR | DO |
| | DNRDLTR | RSGHLTG | DNRDRKR | DP |
| | TREDLTR | RSDHLTR | TREDLTR | DQ |
| | TREDLTR | RSDHLTK | TREDLTR | DR |
| | TREDLTR | RSDHLTG | TREDLTR | DS |
| | TREDLTR | RSGHLTR | TREDLTR | DT |
| | TREDLTR | RSGHLTK | TREDLTR | DU |
| | TREDLTR | RSGHLTG | TREDLTR | DV |
| | TREDLTR | RSDHLTR | TREDRKR | DW |
| | TREDLTR | RSDHLTK | TREDRKR | DX |
| | TREDLTR | RSDHLTG | TREDRKR | DY |
| | TREDLTR | RSGHLTR | TREDRKR | DZ |
| | TREDLTR | RSGHLTK | TREDRKR | EA |
| | TREDLTR | RSGHLTG | TREDRKR | EB |
| | DSGDLTR | RSGHLTK | DSGDRKR | KM |
| | DSGELTR | RSGHLTK | DSGERKR | KN |
| | GSSELTR | RSGHLTK | GSSERKR | KO |
| | GSSELTK | RSGHLTK | GSSELTK | KP |
| | DSGDLTR | RSGHLTG | DSGDRKR | KQ |
| | DSGELTR | RSGHLTG | DSGERKR | KR |
| | GSSELTR | RSGHLTG | GSSERKR | KS |
| | GSSELTK | RSGHLTG | GSSELTK | KT |
| | DSGDLTR | RSDHLTR | DSGDRKR | KU |
| | DSGELTR | RSDHLTR | DSGERKR | KV |
| | GSSELTR | RSDHLTR | GSSERKR | KW |
| | GSSELTK | RSDHLTR | GSSELTK | KX |
| | DSGDLTR | RSGHLTK | DSGDRKR | KY |
| | DSGELTR | RSGHLTK | DSGERKR | KZ |
| | GSSELTR | RSGHLTK | GSSERKR | LA |
| | GSSELTK | RSGHLTK | GSSELTK | LB |
| | DNGDLTR | RSAHLTR | DNGDLTR | LM |
| | DGADLTR | RSAHLTR | DGADLTR | LN |
| | AGADLTR | RSAHLTR | AGADLTR | LO |

Preferably the zinc finger repressor peptides of the invention comprise (or have only) 11-zinc finger domains which are arranged in tandem. Such 11-zinc finger peptide sequences of the invention comprise the sequences having 90% or more, 95% or more, such as 96%, 97%, 98%, 99% or 100% sequence identity to the amino acid sequences of SEQ ID NO: 166, SEQ ID NO: 167, SEQ ID NO: 168 or SEQ ID NO: 171, SEQ ID NO: 172, SEQ ID NO: 173, SEQ ID NO: 174 or SEQ ID NO: 175, SEQ ID NO: 176, SEQ ID NO: 177, SEQ ID NO: 178, SEQ ID NO: 179 or SEQ ID NO: 180 (see Table 7). Thus, suitable zinc finger repressor proteins according to the invention may comprise sequences having 90% or more, 95% or more, such as 96%, 97%, 98%, 99% or 100% sequence identity to the amino acid sequences of SEQ ID NO: 96 to 101.

The invention also encompasses nucleic acid molecules that encode the peptide sequences of the invention. In view of codon redundancy, it will be appreciated that many slightly different nucleic acid sequences may accurately code for each of the zinc finger peptides of the invention, and each of these variants is encompassed within the scope of the present invention. The skilled person can readily determine suitable nucleic acid sequences for encoding each of the zinc finger peptides of the invention, and may select appropriate codon codes according to the system in which the zinc finger peptide is to be expressed (e.g. mouse or human). Any nucleic acid sequences that encode for the peptides of SEQ ID NOs: 166 to 180, SEQ ID NOs: 96 to 101 and SEQ ID NOs: 102 to 104 are encompassed within the invention.

### C. Poly Zinc Finger Activator Proteins

Zinc finger peptide frameworks of the invention may also comprise from 3 to 8 zinc finger domains, from 3 to 7 zinc finger domains, from 4 to 8 zinc finger domains, from 4 to 7 zinc finger domains, or from 4 to 6 zinc finger domains. Preferred zinc finger peptide activators according to aspects and embodiments of the invention comprise 5, 6 or 7 zinc finger domains; and particularly preferred zinc finger peptides of these aspects and embodiments of the invention comprise 6 zinc finger domains. In some embodiments a 6-finger binding unit may be provided by two 3-zinc finger peptides each of which is provided with a complementary dimerisation domain to form a 6-zinc finger binding unit.

In various embodiments, zinc finger peptide activators according to the invention may be based on the frameworks of Structures I to V as defined above and in our previous publications, WO 2012/049332; WO 2017/077329). Alternatively, such zinc finger peptides may be constructed from 2-finger building blocks, as described, for example, in Moore et al. (2001), Proc. Natl. Acad. Sci. USA, 98: 1437-1441. Zinc finger activator proteins of the invention may also be constructed from 3-finger building blocks, as is known in the art (Moore et al. (2001) Proc. Natl. Acad. Sci. USA 98(4): 1437-1441; and Kim & Pabo (1998) Proc. Natl. Acad. Sci. USA 95(6): 2812-2817), or from a combination of 2 and 3 finger building blocks, as desired.

The arrays of zinc finger domains in the zinc finger activator proteins of the invention typically comprise canonical linker sequences, short flexible (canonical-like) linker sequences and, in some embodiments long flexible linker sequences. Thus, as described in relation to the extended poly-zinc peptides (repressor proteins) of the invention, in some embodiments one or more pairs of adjacent zinc finger domains of a zinc finger peptide according to the invention may be separated by short canonical linker sequences; or one or more pairs of adjacent zinc finger domains may be separated by short flexible linker sequences (e.g. of 6 or 7 amino acids), ‘canonical-like’ linker sequences. In some embodiments, however, one or more pairs of adjacent zinc finger domains of a zinc finger peptide may be separated by long flexible linker sequences, for example, comprising 8 or more amino acids, such as between 8 and 50 amino acids as described elsewhere herein. Preferably, the zinc finger activator proteins of the invention—having less zinc finger domains arranged in tandem in comparison to the zinc finger repressor proteins of the invention—comprise zinc finger domains arranged in tandem and linked to each other by canonical or canonical-like linker sequences only.

In some embodiments, the zinc finger activator proteins of the invention may comprise two sub-arrays of 2, 3 or 4 directly adjacent zinc finger domains (or any combination thereof) separated by long flexible (or structured) linkers. Preferably, such poly-zinc finger peptides are arranged in two sub-arrays of 3 or 4-finger units separated by long flexible linkers (to provide a 6- or 8-finger peptide, respectively).

Poly zinc finger peptides of 4 to 8, e.g. 5, 6 or 7 tandem zinc finger domains can exhibit specific and high affinity binding to desired target sequences, both in vitro and in vivo. The inventors' previous studies, see e.g. WO 2012/049332, were the first to report on the systematic exploration of the binding modes of different-length ZFP to long repetitive DNA tracts. In particular, it has been demonstrated that whereas all poly-zinc finger peptides may bind to expanded (e.g. pathogenic) nucleic acid repeat sequences in preference over shorter (e.g. wild-type) repeat sequences; it appears that longer arrays of zinc fingers may demonstrate more pronounced preference for expanded repeat sequences. It is believed that this may, in part, be due to steric reasons, whereby long arrays of zinc fingers may interfere with each other when trying to bind shorter repeat sequences.

In accordance with the invention, it is desirable that zinc finger activator proteins preferentially target native, wild-type repeat sequences within the host genome so as to increase the expression of under-produced wild-type gene products, rather than the pathogenic gene products of abberant genes associated with expanded repeat sequences that present multiple copies of the same target binding sites. Without wishing to be bound by theory, the inventors have hypothesised that shorter arrays of zinc finger domains—for example, tandem arrays of 4 to 8 zinc finger domains of the zinc finger activator proteins of the invention, may show less preference for expanded nucleic acid repeat sequences (i.e. GGGGCC-repeat sequences) than the extended poly-zinc finger repressor proteins of the invention; for example, because they are less susceptible to steric hindrance and competition at the shorter target sequences. Further, it is hypothesised that to outcompete the extended poly-zinc finger repressor proteins at wild-type nucleic acid repeat sequences associated with wild-type genes, the zinc finger activator proteins of the invention should bind the wild-type nucleic acid repeat sequences with high affinity: preferably, with higher affinity (lower dissociation constant) than their corresponding or complementary repressor protein. Accordingly, as described elsewhere herein, in accordance with aspects and embodiments of the invention, preferred GGGGCC targeting sequences for zinc finger activator proteins of the invention comprise less than 30 GGGGCC repeat sequences, e.g. up to 20 hexanucleotide repeats, up to 10 hexanucleotide repeats, or between 2 and 8 hexanucleotide repeats.

### C(i) Poly Zinc Finger Activator Proteins Targeting GGGGCC-Repeat Sequences

The zinc finger activator peptides of the invention preferably bind to sequences within GGGGCC-repeat sequences in double-stranded DNA e.g. DNA molecules, fragments, gene sequences or chromatin. Suitably, the binding site comprises repeats of 5′-GGG GCC-3′. However, it is envisaged that suitable binding sites may also or alternatively comprise repeats of 5′-GGG CCG-3′, 5′- GGC CGG-3′, 5′- GCC GGG-3′ or 5′-CCG GGG-3′.

As described above in regard to the extended poly-zinc finger peptides of the invention, the amino acid sequence of the recognition sequence of each zinc finger domain of a poly-zinc finger activator peptide of the invention is suitably determined by the nucleic acid sequence of the target nucleic acid triplet (or staggered quadrulplet). According to preferred aspects and embodiments of the invention, the zinc finger peptides are designed to target alternating GGG and GCC triplets. Accordingly, the recognition sequences of adjacent zinc finger domains of a poly-zinc finger peptide of the invention may generally alternate along the length of the zinc finger array. Again, therefore, it may be convenient to consider the zinc finger domains of a zinc finger activator peptide of the invention to belong to one of two sequence types, e.g. a ‘first type’ zinc finger domain for binding to a GCC triplet and a ‘second type’ zinc finger domain for binding to a GGG triplet.

In embodiments the recognition sequences of the ‘first type’ represents the odd-numbered zinc finger domains of the zinc finger array (e.g. fingers 1, 3, 5, 7, when read in a direction from N to C terminal), and the ‘second type’ represents the even-numbered zinc finger domains of the zinc finger array (e.g. fingers 2, 4, 6, 8 when read in the N to C terminal direction). Alternatively, the ‘first type’ zinc finger domains may represent the even-numbered fingers of the array (fingers 2, 4, 6, 8), whereas the ‘second type’ zinc finger domains may represent the odd-numbered fingers of the array (fingers 1, 3, 5, 7). For example, the selection of which of the first or second type of domain should be positioned as the first finger of the zinc finger peptide array may be determined by the length of the array. Conveniently, when a zinc finger activator peptide of the invention has 6 or 8 zinc finger domains, the first finger of the array may be of the second type domain, binding GGG, such that the target site of the zinc finger peptide can be considered to be 5′- . . . GGGGCCGGG-3′. However, when a zinc finger activator peptide of the invention has e.g. 5 or 7 zinc finger domains, the first finger of the array may have a first type finger domain, such that the target site would be represented as 5′- . . . GCCGGGGCC-3′.

As described elsewhere herein, advantageously, in embodiments of the invention, the recognition sequence of the first zinc finger domain of a zinc finger activator peptide (F1) may be a sequence wherein the residues at positions +4 and +5 are, respectively, L and T. In this way host matching for in vivo mouse or human applications may be improved. In such embodiments all remaining recognition sequences of the first type may have the residues R and K, respectively, in the 4 and 5 positions; may have the residues L and T, respectively, in the 4 and 5 positions; or may include a mixture of R and K or L and T residues in the 4 and 5 positions, respectively. In some embodiments, as described above, the first zinc finger of each sub-array (wherein sub-arrays are separated from each other by long, flexible linkers in accordance with the invention) has a recognition sequence wherein the residues at positions 4 and 5 are, respectively, L and T. In such embodiments, all remaining recognition sequences of the same type to that of the first finger of the sub-array (first or second type) may have the residues R and K, respectively, in the 4 and 5 positions; may have the residues L and T, respectively, in the 4 and 5 positions; or may include a mixture of R and K or L and T residues in the 4 and 5 positions, respectively.

Further, in embodiments, in order to tune the binding affinity of the poly-zinc fingers of an activator peptide of the invention against the GGGGCC repeat sequence, and specifically against the GGG triplet of the hexapeptide, the residue at the −1 position is preferably R; in some embodiments the residue at position 3 is preferably H; in some embodiments the residue at position 6 is preferably R; in some embodiments the residues at position 2 is preferably D.

Furthermore, in embodiments, in order to tune the binding affinity of the poly-zinc fingers of an activator peptide of the invention specifically against the GCC triplet of the GGGGCC hexapeptide, the residue at the −1 position is preferably D; in some embodiments the residue at position 3 is preferably V; in some embodiments the residue at position 6 is preferably R; in some embodiments the residues at position 2 is preferably S.

Thus, as described above, the recognition sequences of the zinc finger (activator) peptides of the invention may be selected from two general formulae, which alternate along the zinc finger array of the inventive zinc finger peptides according to the nucleic acid binding site.

According to the invention, zinc finger recognition sequences (i.e. positions X⁻¹, X⁻¹, X⁺², X⁺³, X⁺⁴, X⁺⁵ and X⁺⁶) in zinc finger activator proteins of the invention (e.g. as defined by Formulas 2, 4 and 6 above) may be of a first type represented by the amino acid sequence of:

SEQ ID NO: 107:
(D/E/T/V/S)(S/N/R)(S/R/E)(V/D/E/I/L/S/T)(L/R)
(T/K)(R/K)
SEQ ID NO: 108:
DSSVL(T/S/A)R
SEQ ID NO: 13:
DSSVLTR
or
SEQ ID NO: 14:
DSSVRKR;

and may be of a second type represented by the amino acid sequence of:

SEQ ID NO: 109:
RSDH(L/R)(T/K)(R/K)
SEQ ID NO: 110:
RSDHL(T/S/A)(R/K)
SEQ ID NO: 75:
RSDHLTR
or
SEQ ID NO: 111:
RSDHRKR.

As noted above, beneficially, recognition sequences of the first type are adapted/tuned to bind the triplet 5′-GCC-3′ and recognition sequences of the second type are adapted/tuned to bind the triplet 5′-GGG-3′, such that the recognition sequence of the first type alternates with the recognition sequence of the second type within each zinc finger array or sub-array.

Thus, in embodiments, there is provided an engineered zinc finger (DNA-binding) peptide comprising from 3 to 8, such as from 4 to 8, or more specifically 5, 6 or 7 zinc finger domains having the zinc finger recognition sequences of SEQ ID NO: 107 and/or 108 alternating with the zinc finger recognition sequences of SEQ ID NO: 109 and/or 110. Preferably, zinc finger domains having the zinc finger recognition sequences of SEQ ID NO: 14 and/or 15 alternate with zinc finger domains having the zinc finger recognition sequences of SEQ ID NO: 75 and/or 111 along the length of the zinc finger activators according to these aspects and embodiments.

Suitably, when the zinc finger activator peptide has an odd number or zinc fingers, the odd numbered zinc fingers of the zinc finger array are of the first type sequence and the even number zinc fingers of the array are of the second type sequence. Alternatively, when the zinc finger activator peptide has an odd number or zinc fingers, the odd numbered zinc fingers of the zinc finger array are of the second type sequence and the even number zinc fingers of the array are of the first type sequence. In this way, a zinc finger peptide having an odd number of zinc finger domains can be designed to have a larger number of GCC-binding fingers than GGG-binding fingers, or vice versa.

In alternative embodiments, when the zinc finger activator peptide has an even number or zinc fingers, the odd numbered zinc fingers of the zinc finger array are of the second type sequence and the even number zinc fingers of the array are of the first type sequence. In other embodiments, when the zinc finger activator peptide has an even number or zinc fingers, the odd numbered zinc fingers of the zinc finger array are of the first type sequence and the even number zinc fingers of the array are of the second type sequence.

A preferred poly-zinc finger activator peptide of the invention has 6 zinc finger modules, wherein fingers F1, F3 and F5 have recognition sequences according to the second type sequences set out in this section, and fingers F2, F4 and F6 have recognition sequences according to the first type sequences as set out in this section. In another embodiment a poly-zinc finger activator peptide of the invention has 5 zinc finger modules, wherein fingers F1, F3 and F5 have recognition sequences according to the first type sequences set out in this section, and fingers F2 and F4 have recognition sequences according to the second type sequences set out in this section.

The table below summarising preferred recognition sequence arrangements of the poly-zinc finger activator peptides of these aspects and embodiments of the invention.

TABLE 3
Exemplary zinc finger recognition helix arrangements of zinc finger activator peptides
according to the invention. The zinc finger peptide recognition sequence patterns
of this table apply to an entire zinc finger peptide, or to each sub-array of a zinc
finger peptide of the invention. Zinc finger peptides disclosed in this table may
have from 3 to 8 fingers, for example, 3, 4, 5, 6, 7 or 8 zinc finger domains.

Finger number of

| zinc finger peptide or | Zinc Finger Number (N to C terminal direction) | | | |
|---|---|---|---|---|
| of each sub-array of | F1 | F2, F4, F6, F8 | F3, F5, F7 | ZFP |
| a zinc finger peptide | (GCC-binding) | (GGG-binding) | (GCC-binding) | Array |
| Recognition | SEQ ID NO: 107 | SEQ ID NO: 109 | SEQ ID NO: 108 | FV |
| Sequence | SEQ ID NO: 107 | SEQ ID NO: 109 | SEQ ID NO: 107 | FW |
| | SEQ ID NO: 107 | SEQ ID NO: 110 | SEQ ID NO: 108 | FX |
| | SEQ ID NO: 107 | SEQ ID NO: 110 | SEQ ID NO: 107 | FY |
| | SEQ ID NO: 108 | SEQ ID NO: 109 | SEQ ID NO: 108 | FZ |
| | SEQ ID NO: 108 | SEQ ID NO: 109 | SEQ ID NO: 107 | GA |
| | SEQ ID NO: 108 | SEQ ID NO: 110 | SEQ ID NO: 108 | GB |
| | SEQ ID NO: 108 | SEQ ID NO: 110 | SEQ ID NO: 107 | GC |
| | DSSVLTR | RSDHLTR | DSSVRKR | GD |
| | DSSVLTR | RSDHLTR | DSSVLTR | GE |
| | DSSVLTR | RSDHRKR | DSSVRKR | GF |
| | DSSVLTR | RSDHRKR | DSSVLTR | GG |
| | DSSVRKR | RSDHLTR | DSSVRKR | GH |
| | DSSVRKR | RSDHLTR | DSSVLTR | GI |
| | DSSVRKR | RSDHRKR | DSSVRKR | GJ |
| | DSSVRKR | RSDHRKR | DSSVLTR | GK |
| Finger number of | | | | |
| zinc finger peptide or | | | | |
| of each sub-array of | F1 | F2, F4, F6, F8 | F3, F5, F7 | |
| a zinc finger peptide | (GGG-binding) | (GCC-binding) | (GGG-binding) | |
| Recognition | SEQ ID NO: 109 | SEQ ID NO: 107 | SEQ ID NO: 110 | GL |
| Sequence | SEQ ID NO: 109 | SEQ ID NO: 107 | SEQ ID NO: 109 | GM |
| | SEQ ID NO: 109 | SEQ ID NO: 108 | SEQ ID NO: 110 | GN |
| | SEQ ID NO: 109 | SEQ ID NO: 108 | SEQ ID NO: 109 | GO |
| | SEQ ID NO: 110 | SEQ ID NO: 107 | SEQ ID NO: 110 | GP |
| | SEQ ID NO: 110 | SEQ ID NO: 107 | SEQ ID NO: 109 | GQ |
| | SEQ ID NO: 110 | SEQ ID NO: 108 | SEQ ID NO: 110 | GR |
| | SEQ ID NO: 110 | SEQ ID NO: 108 | SEQ ID NO: 111 | GS |
| | RSDHLTR | DSSVLTR | RSDHRKR | GT |
| | RSDHLTR | DSSVLTR | RSDHLTR | GU |
| | RSDHLTR | DSSVRKR | RSDHRKR | GV |
| | RSDHLTR | DSSVRKR | RSDHLTR | GW |
| | RSDHRKR | DSSVLTR | RSDHRKR | GX |
| | RSDHRKR | DSSVLTR | RSDHLTR | GY |
| | RSDHRKR | DSSVRKR | RSDHRKR | GZ |
| | RSDHRKR | DSSVRKR | RSDHLTR | HA |

Poly-zinc finger repressors of the invention may have 2 sub-arrays (e.g. of 3 or 4 zinc finger domains each) within each of which the zinc finger recognition sequence pattern may be selected from any of the combinations disclosed in Table 3 above.

The table below summarises preferred recognition sequence arrangements of the poly-zinc finger activator peptides of these aspects and embodiments of the invention.

TABLE 4
Exemplary zinc finger recognition helix arrangements of zinc finger activator
peptides according to the invention for binding to a GGGGCC repeat sequence.
The zinc finger peptide recognition sequence patterns of this table apply to
an entire zinc finger peptide, or to each sub-array of a zinc finger peptide
of the invention. Zinc finger peptides disclosed in this table suitably have
from 3 to 8 fingers, for example, 3, 4, 5, 6, 7 or 8 zinc finger domains:
preferably 5- or 6- zinc finger domains. In addition to the unique
sequence combinations indicated in the table above, all other combinations
of these sequences are also envisaged and disclosed herein.

| Finger | | | | | | | |
|---|---|---|---|---|---|---|---|
| number of | | | | | | | |
| zinc finger | Zinc Finger Number (N to C terminal direction) | | | | | | ZFP |
| peptide | F1 | F2 | F3 | F4 | F5 | F6 | Array |
| Recognition | RSDHLTR | DSSVRKR | RSDHLTR | DSSVRKR | RSDHLTR | DSSVRKR | HB |
| Sequence | RSDHLTR | DSSVLTR | RSDHLTR | DSSVLTR | RSDHLTR | DSSVLTR | HC |
| | RSDHLTR | DSSVRKR | RSDHRKR | DSSVRKR | RSDHRKR | DSSVRKR | HD |
| | RSDHLTR | DSSVLTR | RSDHRKR | DSSVLTR | RSDHRKR | DSSVLTR | HE |
| | RSDHLTR | DSSVRKR | RSDHLTR | DSSVRKR | RSDHRKR | DSSVRKR | HF |
| | RSDHLTR | DSSVLTR | RSDHLTR | DSSVLTR | RSDHRKR | DSSVLTR | HG |
| | RSDHLTR | DSSVRKR | RSDHRKR | DSSVLTR | RSDHLTR | DSSVRKR | HH |
| | RSDHLTR | DSSVLTR | RSDHLTR | DSSVRKR | RSDHLTR | DSSVLTR | HI |
| | RSDHLTR | DSSVRKR | RSDHRKR | DSSVLTR | RSDHLTR | DSSVLTR | HJ |
| | RSDHLTR | DSSVLTR | RSDHLTR | DSSVRKR | RSDHLTR | DSSVRKR | HK |
| | RSDHRKR | DSSVRKR | RSDHLTR | DSSVRKR | RSDHLTR | DSSVRKR | HL |
| | RSDHRKR | DSSVLTR | RSDHLTR | DSSVLTR | RSDHLTR | DSSVLTR | HM |
| | RSDHRKR | DSSVRKR | RSDHRKR | DSSVRKR | RSDHRKR | DSSVRKR | HN |
| | RSDHRKR | DSSVLTR | RSDHRKR | DSSVLTR | RSDHRKR | DSSVLTR | HO |
| | RSDHRKR | DSSVRKR | RSDHLTR | DSSVRKR | RSDHRKR | DSSVRKR | HP |
| | RSDHRKR | DSSVLTR | RSDHLTR | DSSVLTR | RSDHRKR | DSSVLTR | HQ |
| | RSDHRKR | DSSVRKR | RSDHRKR | DSSVLTR | RSDHLTR | DSSVRKR | HR |
| | RSDHRKR | DSSVLTR | RSDHLTR | DSSVRKR | RSDHLTR | DSSVLTR | HS |
| | RSDHRKR | DSSVRKR | RSDHRKR | DSSVLTR | RSDHLTR | DSSVLTR | HT |
| | RSDHRKR | DSSVLTR | RSDHLTR | DSSVRKR | RSDHLTR | DSSVRKR | HU |
| | RSDHLTR | DSSVRKR | RSDHLTR | DSSVRKR | RSDHLTR | | HV |
| | RSDHLTR | DSSVLTR | RSDHLTR | DSSVLTR | RSDHLTR | | HW |
| | RSDHLTR | DSSVRKR | RSDHRKR | DSSVRKR | RSDHRKR | | HX |
| | RSDHLTR | DSSVLTR | RSDHRKR | DSSVLTR | RSDHRKR | | HY |
| | RSDHLTR | DSSVRKR | RSDHLTR | DSSVRKR | RSDHRKR | | HZ |
| | RSDHLTR | DSSVLTR | RSDHLTR | DSSVLTR | RSDHRKR | | IA |
| | RSDHLTR | DSSVRKR | RSDHRKR | DSSVLTR | RSDHLTR | | IB |
| | RSDHLTR | DSSVLTR | RSDHLTR | DSSVRKR | RSDHLTR | | IC |
| | RSDHRKR | DSSVRKR | RSDHLTR | DSSVRKR | RSDHLTR | | ID |
| | RSDHRKR | DSSVLTR | RSDHLTR | DSSVLTR | RSDHLTR | | IE |
| | RSDHRKR | DSSVRKR | RSDHRKR | DSSVRKR | RSDHRKR | | IF |
| | RSDHRKR | DSSVLTR | RSDHRKR | DSSVLTR | RSDHRKR | | IG |
| | RSDHRKR | DSSVRKR | RSDHLTR | DSSVRKR | RSDHRKR | | IH |
| | RSDHRKR | DSSVLTR | RSDHLTR | DSSVLTR | RSDHRKR | | II |
| | RSDHRKR | DSSVRKR | RSDHRKR | DSSVLTR | RSDHLTR | | IJ |
| | RSDHRKR | DSSVLTR | RSDHLTR | DSSVRKR | RSDHLTR | | IK |
| | DSSVLTR | RSDHLTR | DSSVLTR | RSDHLTR | DSSVLTR | RSDHRKR | IL |
| | DSSVLTR | RSDHRKR | DSSVLTR | RSDHRKR | DSSVLTR | RSDHLTR | IM |
| | DSSVLTR | RSDHLTR | DSSVLTR | RSDHRKR | DSSVLTR | RSDHRKR | IN |
| | DSSVLTR | RSDHLTR | DSSVRKR | RSDHLTR | DSSVLTR | RSDHLTR | IO |
| | DSSVLTR | RSDHLTR | DSSVRKR | RSDHLTR | DSSVRKR | RSDHRKR | IP |
| | DSSVLTR | RSDHLTR | DSSVLTR | RSDHLTR | DSSVLTR | RSDHLTR | IQ |
| | DSSVLTR | RSDHRKR | DSSVLTR | RSDHRKR | DSSVLTR | RSDHRKR | IR |
| | DSSVLTR | RSDHLTR | DSSVLTR | RSDHRKR | DSSVLTR | RSDHLTR | IS |
| | DSSVLTR | RSDHLTR | DSSVRKR | RSDHLTR | DSSVLTR | RSDHRKR | IT |
| | DSSVLTR | RSDHLTR | DSSVRKR | RSDHLTR | DSSVRKR | RSDHLTR | IU |
| | DSSVRKR | RSDHLTR | DSSVRKR | RSDHLTR | DSSVRKR | RSDHRKR | IV |
| | DSSVRKR | RSDHRKR | DSSVRKR | RSDHRKR | DSSVRKR | RSDHLTR | IW |
| | DSSVRKR | RSDHLTR | DSSVRKR | RSDHRKR | DSSVRKR | RSDHRKR | IX |
| | DSSVRKR | RSDHRKR | DSSVLTR | RSDHLTR | DSSVRKR | RSDHLTR | IY |
| | DSSVRKR | RSDHRKR | DSSVLTR | RSDHLTR | DSSVLTR | RSDHRKR | IZ |
| | DSSVRKR | RSDHLTR | DSSVRKR | RSDHLTR | DSSVRKR | RSDHLTR | JA |
| | DSSVRKR | RSDHRKR | DSSVRKR | RSDHRKR | DSSVRKR | RSDHRKR | JB |
| | DSSVRKR | RSDHLTR | DSSVRKR | RSDHRKR | DSSVRKR | RSDHLTR | JC |
| | DSSVRKR | RSDHRKR | DSSVLTR | RSDHLTR | DSSVRKR | RSDHRKR | JD |
| | DSSVRKR | RSDHRKR | DSSVLTR | RSDHLTR | DSSVLTR | RSDHLTR | JE |
| | DSSVLTR | RSDHLTR | DSSVLTR | RSDHLTR | DSSVLTR | | JF |
| | DSSVLTR | RSDHRKR | DSSVLTR | RSDHRKR | DSSVLTR | | JG |
| | DSSVLTR | RSDHLTR | DSSVLTR | RSDHRKR | DSSVLTR | | JH |
| | DSSVLTR | RSDHLTR | DSSVRKR | RSDHLTR | DSSVLTR | | JI |
| | DSSVLTR | RSDHLTR | DSSVRKR | RSDHLTR | DSSVRKR | | JJ |
| | DSSVRKR | RSDHLTR | DSSVRKR | RSDHLTR | DSSVRKR | | JK |
| | DSSVRKR | RSDHRKR | DSSVRKR | RSDHRKR | DSSVRKR | | JL |
| | DSSVRKR | RSDHLTR | DSSVRKR | RSDHRKR | DSSVRKR | | JM |
| | DSSVRKR | RSDHRKR | DSSVLTR | RSDHLTR | DSSVRKR | | JN |
| | DSSVRKR | RSDHRKR | DSSVLTR | RSDHLTR | DSSVLTR | | JO |

The invention also encompasses nucleic acid molecules that encode the peptide sequences of the invention. In view of codon redundancy, it will be appreciated that many slightly different nucleic acid sequences may accurately code for each of the zinc finger peptides of the invention, and each of these variants is encompassed within the scope of the present invention. The skilled person can readily determine suitable nucleic acid sequences for encoding each of the zinc finger peptides of the invention, and may select appropriate codon codes according to the system in which the zinc finger peptide is to be expressed (e.g. mouse or human). Any nucleic acid sequences that encode the above peptides, such as the peptides of SEQ ID NOs: 169 and 170 are also encompassed within the invention.

### D. Zinc Finger Derivatives and Associated Sequences

The invention also encompasses derivatives of the zinc finger peptides of the invention. In this regard, it will be appreciated that modifications, such as amino acid substitutions may be made at one or more positions in the peptide without adversely affecting its physical properties (such as binding specificity or affinity). By ‘derivative’ of a zinc finger peptide it is meant a peptide sequence that has the desired activity (e.g. binding affinity for a selected target sequence, especially poly GGGGCC-repeat sequences), but that includes one or more mutations or modifications to the primary amino acid sequence having the desired activity. Thus, a derivative of the invention may have one or more (e.g. 1, 2, 3, 4, 5 or more) chemically modified amino acid side chains, such as pegylation, sialylation and glycosylation modifications. In addition, or alternatively, a derivative may contain one or more (e.g. 1, 2, 3, 4, 5 or more) amino acid mutations, substitutions, deletions or combinations thereof to the primary sequence of a selected poly-zinc finger peptide. Accordingly, the invention encompasses the results of maturation experiments conducted on a selected zinc finger peptide or a zinc finger peptide framework to improve or change one or more characteristics of the initially identified peptide. By way of example, one or more amino acid residues of a selected zinc finger domain may be randomly or specifically mutated (or substituted) using procedures known in the art (e.g. by modifying the encoding DNA or RNA sequence). The resultant library or population of derivatised peptides may further be selected—by any known method in the art—according to predetermined requirements: such as improved specificity against particular target sites; or improved drug properties (e.g. solubility, bioavailability, immunogenicity etc.). A particular benefit of the invention is improved compatibility with the host/target organism as assessed by sequence similarity to known host peptide sequences and/or immunogenicity/adverse immune response to the heterologous peptide when expressed. Peptides selected to exhibit such additional or improved characteristics and that display the activity for which the peptide was initially selected are derivatives of the zinc finger peptides of the invention and also fall within the scope of the invention.

Zinc finger frameworks of the invention may be diversified at one or more positions in order to improve their compatibility with the host system in which it is intended to express the proteins. In particular, specific amino acid substitutions may be made within the zinc finger peptide sequences and in any additional peptide sequences (such as effector domains) to reduce or eliminate possible immunological responses to the expression of these heterologous peptides in vivo. Target amino acid residues for modification or diversification are particularly those that create non-host amino acid sequences or epitopes that might not be recognised by the host organism and, consequently, might elicit an undesirable immune response. In some embodiments the framework is diversified or modified at one or more of amino acids positions −1, 1, 2, 3, 4, 5 and 6 of the recognition sequence. The polypeptide sequence changes may conveniently be achieved by diversifying or mutating the nucleic acid sequence encoding the zinc finger peptide frameworks at the codons for at least one of those positions, so as to encode one or more polypeptide variant. All such nucleic acid and polypeptide variants are encompassed within the scope of the invention.

The amino acid residues at each of the selected positions may be non-selectively randomised, i.e. by allowing the amino acid at the position concerned to be any of the 20 common naturally occurring amino acids; or may be selectively randomised or modified, i.e. by allowing the specified amino acid to be any one or more amino acids from a defined sub-group of the 20 naturally occurring amino acids. It will be appreciated that one way of creating a library of mutant peptides with modified amino acids at each selected location, is to specifically mutate or randomize the nucleic acid codon of the corresponding nucleic acid sequence that encodes the selected amino acid. On the other hand, given the knowledge that has now accumulated in relation to the sequence specific binding of zinc finger domains to nucleic acids, in some embodiments it may be convenient to select a specific amino acid (or small sub-group of amino acids) at one or more chosen positions in the zinc finger domain, for example, where it is known that a specific amino acid provides optimal binding to a particular nucleotide residue in a specific target sequence. In accordance with the invention, a predicted optimal interaction may be introduced when not already present (e.g. to optimise binding affinity in the case of a zinc finger peptide activator); or a predicted optimal interaction may be removed when it is already present and it is desired to reduce the binding affinity of the zinc finger peptide for the target sequence (e.g. in the case of a zinc finger repressor according to the invention). The resultant peptides or frameworks may be considered to be the result of rational or ‘intelligent’ design. Conveniently the whole of the zinc finger recognition sequence may be selected by intelligent design and inserted/incorporated into an appropriate zinc finger framework both of which, ideally, are derived from the intended host organism, such as mouse or human. The person of skill in the art is well aware of the codon sequences that may be used in order to specify one or more than one particular amino acid residue within a library. Preferably all amino acid positions in each zinc finger domain and in any additional peptide sequences (such as effector domains and leader sequences) are chosen from known wild-type sequences from the host organism in which the protein is intended to be used.

Taking into account that minor modifications to the primary sequence of the peptides/proteins of the invention can be made without substantially altering the scope of the claimed invention, the invention should be considered to encompass, in addition, any polypeptide sequences that are substantially the same as the specific amino acid sequences disclosed herein. For example, the claimed invention encompasses polypeptide sequences that have at least 80% identity to the SEQ ID NOs of the polypeptide sequences disclosed herein; at least 85% identity, at least 90% identity, at least 95% identity, at least 98% identity, at least 99% identity or approx. 100% identity to the polypeptide sequences of the SEQ ID NOs explicitly disclosed herein.

Similarly, the claimed invention encompasses polynucleotide sequences that have at least 70% identity to the polynucleotide SEQ ID NOs disclosed herein; at least 80% identity, at least 85% identity, at least 90% identity, at least 95% identity, at least 98% identity, at least 99% identity or approx. 100% identity to the polynucleotide sequences encoding the SEQ ID NOs explicitly disclosed herein.

### Zinc Finger Peptide Modulators and Effectors

It will be appreciated that the zinc finger peptide framework sequences of the invention may further include optional (N-terminal) leader sequences, such as: amino acids to aid expression (e.g. N-terminal Met-Ala or Met-Gly dipeptide); purification tags (e.g. FLAG-tags); and localisation/targeting sequences (e.g. nuclear localisation sequences (NLS), such as PKKKRKV (SV40 NLS, SEQ ID NO: 148); PKKRRKVT (human protein KIAA2022, SEQ ID NO: 149); or RIRKKLR (mouse primase p58 NLS9, SEQ ID NO: 150). Thus, a suitable leader sequence for use in conjunction with zinc finger peptide sequences of the invention includes MGRIRKKLRLAERP for expression and cellular localisation in mouse (SEQ ID NO: 89) and MGPKKRRKVTGERP for expression and cellular localisation in human cells (SEQ ID NO: 90) Also, the peptides of the invention may optionally include additional C-terminal sequences, such as: linker sequences for fusing zinc finger domains to effector molecules; and the effector molecules themselves. Other sequences may be employed for cloning purposes. The sequences of any N- or C-terminal sequences may be varied, typically without altering the binding activity of the zinc finger peptide framework, and such variants are encompassed within the scope of the invention. Preferred host-compatible additional sequences are Met-Gly dipeptide for protein expression in humans and mice; human (PKKRRKVT, SEQ ID NO: 149) or mouse (RIRKKLR, SEQ ID NO: 150) nuclear localisation sequences for expression in human or mouse respectively; and host-derived effector domain sequences as discussed below.

Suitably a zinc finger peptide of the invention for expression and use in mouse or human respectively, does not include purification tags where it is not intended to purify the zinc finger-containing peptide, e.g. where gene regulatory and/or therapeutic activities are intended. Thus, for reason of improved host-matching (reduced toxicity and reduced immunogenicity) the peptides and polypeptides of the invention are preferably devoid of peptide purification tags and the like, which are not found in endogenous, wild-type proteins of a host organism.

Particularly preferred polypeptides of the invention comprise an appropriate nuclear localisation sequence arranged N-terminal of a poly-zinc finger peptide, which is itself arranged N-terminal to an effector domain that may repress expression of a target gene. Effector domains are conveniently attached to the poly-zinc finger peptide covalently, such as by a peptide linker sequence as disclosed elsewhere herein.

While the zinc finger peptides of the invention may have useful biological properties in isolation, they can also be given useful biological functions by the addition of effector domains. Therefore, in some cases it is desirable to conjugate a zinc finger peptide of the invention to one or more non-zinc finger domain, thus creating chimeric or fusion zinc finger peptides. It may also be desirable, in some instances, to create a multimer (e.g. a dimer), of a zinc finger peptide of the invention—for example, to bind more than one target sequence simultaneously, which target sequences may be the same or different.

Thus, having identified a desirable zinc finger peptide, an appropriate effector or functional group may then be attached, conjugated or fused to the zinc finger peptide. The resultant protein of the invention, which comprises at least a zinc finger portion (of more than one zinc finger domain) and a non-zinc finger effector domain, portion or moiety may be termed a ‘fusion’, ‘chimeric’ or ‘composite’ zinc finger peptide. Beneficially, the zinc finger peptide will be linked to the other moiety at a position and/or via a linker that does not interfere with the activity of either moiety.

A ‘non-zinc finger domain’ (or moiety) as used herein, refers to an entity that does not contain a zinc finger (ββα-) fold. Thus, non-zinc finger moieties include nucleic acids and other polymers, peptides, proteins, peptide nucleic acids (PNAs), antibodies, antibody fragments, and small molecules, amongst others.

Chimeric zinc finger peptides or fusion proteins of the invention may in accordance with the invention be used to up- or down-regulate desired target genes, in vitro or in vivo. Thus, potential effector domains include transcriptional repressor domains, transcriptional activator domains, transcriptional insulator domains, chromatin remodelling, condensation or decondensation domains, nucleic acid or protein cleavage domains, dimerisation domains, enzymatic domains, signalling/targeting sequences or domains, or any other appropriate biologically functional domain. Other domains that may also be appended to zinc finger peptides of the invention (and which have biological functionality) include peptide sequences involved in protein transport, localisation sequences (e.g. subcellular localisation sequences, nuclear localisation, protein targeting) or signal sequences. Zinc finger peptides can also be fused to epitope tags (e.g. for use to signal the presence or location of a target nucleotide sequence recognised by the zinc finger peptide. Functional fragments of any such domain may also be used.

Beneficially, zinc finger peptides and fusion proteins/polypeptides of the invention have transcriptional modulatory activity and, therefore, preferred biological effector domains include transcriptional modulation domains such as transcriptional activators and transcriptional repressors, as well as their functional fragments. The effector domain can be directly derived from a basal or regulated transcription factor such as, for example, transactivators, repressors, and proteins that bind to insulator or silencer sequences (see Choo & Klug (1995) Curr. Opin. Biotech. 6: 431-436; Choo & Klug (1997) Curr. Opin. Str. Biol. 7:117-125; and Goodrich et al. (1996) Cell 84: 825-830); or from receptors such as nuclear hormone receptors (Kumar & Thompson (1999) Steroids 64: 310-319); or co-activators and co-repressors (Ugai et al. (1999) J. Mol. Med. 77: 481-494).

Other useful functional domains for control of gene expression include, for example, protein-modifying domains such as histone acetyltransferases, kinases, methylases and phosphatases, which can silence or activate genes by modifying DNA structure or the proteins that associate with nucleic acids (Wolffe (1996) Science 272: 371-372; and Hassig et al., (1998) Proc. Natl. Acad. Sci. USA 95: 3519-3524). Additional useful effector domains include those that modify or rearrange nucleic acid molecules such as methyltransferases, endonucleases, ligases, recombinases, and nucleic acid cleavage domains (see for example, Smith et al. (2000) Nucleic Acids Res., 17: 3361-9; WO 2007/139982 and references cited therein), such as the Fokl endonuclease domain, which in conjunction with zinc finger peptides of the invention may be used to truncate poly-CAG repeat genome sequences.

In embodiments, suitable transcriptional/gene activation domains for fusing to zinc finger peptides in order to produce a zinc finger activator protein of the invention include: the VP64 domain, SEQ ID NO: 94 (see Seipel et al., (1996) EMBO J. 11: 4961-4968) and the herpes simplex virus (HSV) VP16 domain, SEQ ID NO: 93 (Hagmann et al. (1997) J. Virol. 71: 5952-5962; Sadowski et al. (1988) Nature 335: 563-564); and transactivation domain 1 and/or 2 of the p65 subunit of nuclear factor-KB (NFκB; Schmitz et al. (1995) J. Biol. Chem. 270: 15576-15584; Schmitz and Baeuerle (1991) EMBO J. 10(12):3805-17) in human (SEQ ID NO: 91) or in mouse (SEQ ID NO: 92). Such zinc finger activator proteins of the invention are useful in upregulating the expression of wild-type gene products that are under (or not) expressed in a pathogenic condition.

Furthermore, for a useful therapeutic or diagnostic effect, in accordance with the invention, it is desirable to down-regulate or repress the expression of the pathogenic genes associated with expanded GGGGCC-hexanucleotide repeat sequences that are a focus of the present invention. Therefore, effector domains that effect repression or silencing of target gene expression are particularly beneficial. In particular, the peptides of the invention suitably comprise effector domains that cause repression or silencing of target pathogenic genes when the zinc finger nucleic acid binding domain of the protein directly binds with expanded GGGGCC-repeat sequences associated with the target gene.

In embodiments, the transcriptional repression domain is the Kruppel-associated box (KRAB) domain, which is a powerful repressor of gene activity. In some preferred embodiments, therefore, zinc finger repressor proteins or frameworks of the invention comprise the zinc finger peptides of the invention fused to the KRAB repressor domain from the human Kox-1 protein in order to repress a target gene activity (e.g. see Thiesen et al. (1990) New Biologist 2: 363-374). Fragments of the Kox-1 protein comprising the KRAB domain, up to and including full-length Kox protein may be used as transcriptional repression domains, as described in Abrink et al. (2001) Proc. Natl. Acad. Sci. USA, 98: 1422-1426. A useful human Kox-1 domain sequence for inhibition of target genes in humans is shown in Table 9 (SEQ ID NO: 151). A useful mouse KRAB repressor domain sequence for inhibition of target genes in mice is the mouse analogue of human Kox-1, i.e. the KRAB domain from mouse ZF87 (SEQ ID NO: 152). Other transcriptional repressor domains known in the art may alternatively be used according to the desired result and the intended host, such as the engrailed domain, the snag domain, and the transcriptional repression domain of v-erbA.

All known methods of conjugating an effector domain to a peptide sequence are incorporated. The term ‘conjugate’ is used in its broadest sense to encompass all methods of attachment or joining that are known in the art, and is used interchangeably with the terms such as ‘linked’, ‘bound’, ‘associated’ or ‘attached’. The effector domain(s) can be covalently or non-covalently attached to the binding domain: for example, where the effector domain is a polypeptide, it may be directly linked to a zinc finger peptide (e.g. at the C-terminus) by any suitable flexible or structured amino acid (linker) sequence (encoded by the corresponding nucleic acid molecule).

Non-limiting suitable linker sequences for joining an effector domain to the C-terminus of a zinc finger peptide are illustrated in Table 9 (e.g. LRQKDGGGGSGGGGSGGGGSQLVSS, SEQ ID NO: 153; LRQKDGGGGSGGGGSS, SEQ ID NO: 154; LRQKDGGGSGGGGS, SEQ ID NO: 155; and LRQKDGGGGSGGGGS, SEQ ID NO: 95). Alternatively, a synthetic non-amino acid or chemical linker may be used, such as polyethylene glycol, a maleimide-thiol linkage (useful for linking nucleic acids to amino acids), or a disulphide link. Synthetic linkers are commercially available, and methods of chemical conjugation are known in the art. A preferred linker for conjugating the human kox-1 domain to a zinc finger peptide of the invention is the peptide of SEQ ID NO: 154. A preferred linker for conjugating the mouse ZF87 domain to a zinc finger peptide of the invention is the peptide of SEQ ID NO: 155. It will be appreciated, however, that the amino acid sequences of such long, flexible linkers may not be critical and, for example, the number of G and/or S repeats may be varied as desired, provided the resultant linker does not interfere with the activities of any associated effector domains.

Non-covalent linkages between a zinc finger peptide and an effector domain can be formed using, for example, leucine zipper/coiled coil domains, or other naturally occurring or synthetic dimerisation domains (Luscher & Larsson (1999) Oncogene 18: 2955-2966; and Gouldson et al. (2000) Neuropsychopharm. 23: S60-S77. Other non-covalent means of conjugation may include a biotin-(strept)avidin link or the like. In some cases, antibody (or antibody fragment)-antigen interactions may also be suitably employed, such as the fluorescein-antifluorescein interaction.

To cause a desired biological effect via modulation of gene expression, zinc finger peptides or their corresponding fusion peptides are allowed to interact with, and bind to, one or more target nucleotide sequence associated with the target gene, either in vivo or in vitro depending to the application. Beneficially, therefore, a nuclear localisation domain is attached to the DNA binding domain to direct the protein to the nucleus. One useful nuclear localisation sequence is the SV40 NLS (PKKKRKV, SEQ ID NO: 148). Desirably, however, the nuclear localisation sequence is a host-derived sequence, such as the NLS from human protein KIAA2022 NLS (PKKRRKVT; NP_001008537.1, SEQ ID NO: 149) for use in humans; or the NLS from mouse primase p58 (RIRKKLR; GenBank: BAA04203.1, SEQ ID NO: 150) for use in mice.

Thus, preferred zinc finger-containing polypeptides of the invention include a nuclear localisation sequence (NLS), a poly-zinc finger peptide sequence and a transcriptional repressor (e.g. KRAB domain) or a transcriptional activator (e.g. p65-RelA activation domain). Particularly preferred poly-zinc finger peptide sequences of the disclosure include SEQ ID NOs: 166 to 180, which in embodiments are beneficially operable linked to one or more nuclear localisation sequence (NLS), a transcriptional repressor(e.g. KRAB domain) or a transcriptional activator (e.g. p65-RelA activation domain) domain and optionally signal peptide sequences as described herein.

In some embodiments, it may be advantageous to include more than one NLS as described herein; for example, between 2 and 5 NLSs; suitably 2 or 3 NLSs; preferably 2. When more than one NLS is provided, said NLSs may suitably be arranged in tandem. NLS sequences generally provide a net positive charge, and arranging more than one NLS (e.g. 2, 3, 4 or 5) in tandem can enhance cell-penetration of the zinc finger-containing polypeptide by providing a concentration of positively charged amino acid residues.

In accordance with some preferred embodiments, as described elsewhere, the zinc finger polypeptides of the invention may further include one or more protein secretion signal (SS) or signal peptide (SP) for promoting secretion of zinc finger polypeptides from the cell in which they are produced. A suitable protein secretion signal for use in human cells is the human BMP10 protein secretion signal, MGSLVLTLCALFCLAAYLVSG (SEQ ID NO: 156). In some such embodiments a nucleic acid or polypeptide cleavage site may be incorporated between the signal peptide and the zinc finger peptide sequence of the encoded zinc finger polypeptide, for example, so that the signal peptides of some expressed polypeptides may be separated from the transcription factor portion of the zinc finger polypeptide before it is secreted. In this way, at least some expressed zinc finger polypeptide remains inside the cell in which it was expressed. Suitably, the cleavage sequence is the RIRR peptide cleavage site (SEQ ID NO: 85).

DNA regions from which to effect the up- or down-regulation of specific genes may include promoters, enhancers or locus control regions (LCRs). In accordance with the invention, preferred target sequences for repression of pathogenic genes are GGGGCC-hexanucleotide repeat sequences comprising more than 30 repeats; while preferred target sequences for activation of wild-type genes are GGGGCC-hexanucleotide repeat sequences comprising 30 or less repeats.

### Nucleic Acids and Peptide Expression

The zinc finger peptides according to the invention and, where appropriate, the zinc finger peptide modulators (conjugate/effector molecules) of the invention may be produced by recombinant DNA technology and standard protein expression and purification procedures. Thus, the invention further provides nucleic acid molecules that encode the zinc finger peptides of the invention as well as their derivatives; and nucleic acid constructs, such as expression vectors that comprise nucleic acid encoding peptides and derivatives according to the invention.

For instance, the DNA encoding the relevant peptide can be inserted into a suitable expression vector (e.g. pGEM®, Promega Corp., USA), where it is operably linked to appropriate expression sequences, and transformed into a suitable host cell for protein expression according to conventional techniques (Sambrook J. et al., Molecular Cloning: a Laboratory Manual, Cold Spring Harbor Press, Cold Spring Harbor, NY). Suitable host cells are those that can be grown in culture and are amenable to transformation with exogenous DNA, including bacteria, fungal cells and cells of higher eukaryotic origin, preferably mammalian cells (e.g. particularly mice or human).

To aid in purification, the zinc finger peptides (and corresponding nucleic acids) of the invention may include a purification sequence, such as a His-tag. In addition, or alternatively, the zinc finger peptides may, for example, be grown in fusion with another protein and purified as insoluble inclusion bodies from bacterial cells. This is particularly convenient when the zinc finger peptide or effector moiety may be toxic to the host cell in which it is to be expressed. Alternatively, peptides of the invention may be synthesised in vitro using a suitable in vitro (transcription and) translation system (e.g. the E. coli S30 extract system, Promega corp., USA). The present invention is particularly directed to the expression of zinc finger-containing peptides of the invention in host cells in vivo or in host cell for ex vivo applications, to modulate the expression of endogenous genes. Preferred peptides of the invention may therefore be devoid of such sequences (e.g. His-tags) that are intended for purification or other in vitro based manipulations.

The term ‘operably linked’, when applied to DNA sequences, for example in an expression vector or construct, indicates that the sequences are arranged so that they function cooperatively in order to achieve their intended purposes, i.e. a promoter sequence allows for initiation of transcription that proceeds through a linked coding sequence as far as the termination sequence.

It will be appreciated that, depending on the application, the zinc finger peptide or fusion protein of the invention may comprise an additional peptide sequence or sequences at the N- and/or C-terminus for ease of protein expression, cloning, and/or peptide or RNA stability, without changing the sequence of any zinc finger domain. For example, suitable N-terminal leader peptide sequences for incorporation into peptides of the invention are MA or MG and ERP. Nuclear localisation sequences (one or more) may be suitably incorporated at the N-terminus of the peptides of the invention to create an N-terminal leader sequence. A useful N-terminal leader sequence for expression and nuclear targeting in human cells is MGPKKRRKVTGERP (SEQ ID NO: 157) or MGPKKRRKVTLAERP (SEQ ID NO: 158), and a useful N-terminal leader sequence for expression and nuclear targeting in mouse cells is MGRIRKKLRLAERP (SEQ ID NO: 159). Another particularly useful nuclear localisation sequence is the SV40 sequence PKKKRKV (SEQ ID NO: 148), which may be used in tandem (e.g. SEQ ID NO: 160) to enhance cellular uptake (as well as nuclear localisation).

In some applications it may be desirable to control the expression of zinc finger (fusion) polypeptides of the invention by tissue specific promoter sequences or inducible promoters, which may provide the benefits of organ or tissue specific and/or inducible expression of polypeptides of the invention. These systems may be particularly advantageous for in vivo applications and gene therapy in vivo or ex vivo. Examples of tissue-specific promoters include the human CD2 promoter (for T-cells and thymocytes, Zhumabekov et al. (1995) J. Immunological Methods 185: 133-140); the alpha-calcium-calmodulin dependent kinase II promoter (for hippocampus and neocortex cells, Tsien et al. (1996) Cell 87: 1327-1338); the whey acidic protein promoter (mammary gland, Wagner et al. (1997) Nucleic Acids Res. 25: 4323-4330); the mouse myogenin promoter (skeletal muscle, Grieshammer et al. (1998) Dev. Biol. 197: 234-247); and many other tissue specific promoters that are known in the art.

It is particularly desirable to express the zinc finger peptides and other zinc finger constructs of the invention, such as zinc finger repressor or zinc finger activator proteins, from vectors suitable for use in vivo or ex vivo, e.g. for therapeutic applications (gene therapy). Where the therapy involves use of zinc finger nucleic acid constructs for expression of protein in vivo, the expression system selected should be capable of expressing protein in the appropriate tissue/cells where the therapy is to take effect. Desirably an expression system for use in accordance with the invention is also capable of targeting the nucleic acid constructs or peptides of the invention to the appropriate region, tissue or cells of the body in which the treatment is intended. A particularly suitable expression and targeting system is based on recombinant adeno-associated virus (AAV), e.g. the AAV2/1 subtype.

For ALS and/or FTD disease gene therapy it is desirable to infect particular parts of the brain (e.g. the striatum), central nervous system (e.g. motor neurons) and/or muscle with therapeutic viral vectors. In some embodiments, AAV2/1 subtype vectors (see e.g. Molecular Therapy (2004) 10: 302-317) are ideal for this purpose. Such vectors can be used with a strong AAV promoter or a weak promoter according to preference—for example, a strong AAV vector would be used in conjunction with a zinc finger repressor protein of the invention (to provide relatively large quantities of weaker binding extended poly-zinc finger-containing proteins of the invention), whereas a weak promotor may be used in conjunction with a zinc finger activator protein of the invention (to provide relatively small quantities of stronger binding poly-zinc finger-containing proteins of the invention).

Instead or in addition to AAV2/1 subtype vectors, other AAV subtype vectors may be used, such as AAV2/9 subtype vectors. The AAV2/1 tropism is more specific for infecting neurons, whereas AAV2/9 infects more widely (Expert Opin Biol Ther. 2012 June; 12(6): 757-766.) and certain variants can even be applied intravenously (Nature Biotech 34(2): 204-209). Therefore, using the AAV2/9 subtype (alone or in combination with AAV2/1) advantageously allows targeting of a wider variety of cell types. In the context of ALS and/or FTD, this allows targeting of other (non-neuron) cell types in the brain that may also play a role in disease, such as glia. Additionally, this may advantageously allow targeting to peripheral tissues, such as the heart, muscle or liver which may be advantageous in some embodiments and therapeutic applications.

A promoter for use in AAV2/1 viral vectors and that is suitable for use in humans and mice is the pCAG promoter (CMV early enhancer element and the chicken β-actin promoter). Another useful sequence for inclusion in AAV vectors is the Woodchuck hepatitis virus posttranscriptional regulatory element (WPRE; Garg et al., (2004) J. Immunol., 173: 550-558). More suitably, other promoters that may be advantageous for sustained expression in human and mice/rats in vivo include: (i) the pNSE promoter (neuron-specific promoter of the enolase gene), as described in Xu et al. (2001), Gene Ther., 8:1323-32 (rat: NCBI NC_005103.4; human: NCBI NC_000012.12); (ii) the pHsp90ab1 promoter, as described in WO 2017/077329 (mouse: NCBI 15516 NC_000083.6; human: NCBI 3326 NC_000006.12); (iii) the CBh promoter (including the CMV enhancer, chicken b-actin promoter and hybrid intron), as described in Gray et al., (2011), Human Gene Therapy (2011), 22(9):1143-1153; (iv) the human EF1α-1 promoter, as described in Zheng and Baum (2014), Int. J. Med. Sci., 11(5):404-408); and (v) the human synapsin promoter, as described in Kugler et al. (2003), Gene Ther., 10(4):337-47).

Furthermore, endogenous promoters such as pNSE and pHSP90AB1 are expressed in neurons and ubiquitously, respectively. NSE is ‘very strong’ promoter, while HSP90AB1 is a ‘strong’ promoter. These promoters are typically used for the high-level expression of zinc finger repressor proteins in accordance with the invention. In this regard, the present inventors have previously designed synthetic mouse and human pNSE promoter-enhancers (see e.g. WO 2017/077329, Example 17) comprising a portion of sequence upstream and downstream of the transcription start site of the enolase gene from human and rat: such sequences are explicitly incorporated herein as promoter-enhancer regions, which are minimal where no flanking sequences are also included. Of course, however, any other suitable endogenous promoter sequence may alternatively be used. As the skilled person will appreciate, the selection of an appropriate endogenous promoter may suitably be construct- and/or application-dependent; e.g. according to the desired expression level of the zinc finger polypeptide concerned. Thus, the selection of endogenous promoter can be used to tune the expression level of the zinc finger polypeptide as desired. Flanking restriction sites may be added to the sequence for cloning into an appropriate vector. Since the pNSE promoter is neuron-specific, it is particularly advantageously used in combination with AAV2/1 or other neuron-specific vectors.

A promoter that may be suitable for use with AAV2/9 viral vectors is the pHSP promoter (promoter of the ubiquitously expressed Hsp90ab1 gene). This promotor may also be suitable for use in humans and mice. Again, as disclosed in the inventors earlier patent application (WO 2017/077329, Example 17), it was found that a synthetic promoter-enhancer design comprising a portion of the sequence upstream and downstream of the transcription start site of the mouse or human Hsp90ab1 gene could be advantageously used to obtain sustained expression of a transgene, such as the zinc finger peptides of the invention. In particular, a 1.7 kb region upstream of the transcription start site of the Hsp90ab1 gene that comprises multiple enhancers and can be advantageously used as a minimal hsp90ab1 constitutive promoter, in combination with a portion of exon 1 of the gene. The sequences of the mouse and human minimal promoters with flanking restriction sites for cloning into a vector are explicitly incorporated herein by reference. Mouse and human minimal promoters without flanking restriction sites are also explicitly incorporated herein by reference. These promoter-enhancer sequences may be operably associated with/linked to nucleic acid sequences encoding the zinc finger peptides and modulators of the invention; and the use/methods of using such constructs for sustained expression of (zinc finger) peptides in vivo. Particularly appropriate in vivo systems are human and mouse. The present invention therefore encompasses expression constructs and vectors (e.g. AAV2/1 or AAV2/9 viral vectors) comprising these sequences, as well as the use of such promotor sequences for expression of zinc finger repressor and/or activator peptides of the invention.

Suitable medical uses and methods of therapy may, in accordance with the invention, encompass the combined use—either separate, sequential or simultaneous—of the viral vectors AAV2/1 and AAV2/9. In some such embodiments, at least the AAV2/9 vector may comprise a hsp90ab1 constitutive promoter according to Example 17 of WO 2017/077329. Suitably, these medical uses and methods of therapy further comprise such vectors encoding one or more zinc finger peptide/modulator of the invention. Most suitably the medical uses and methods of therapy are directed to the treatment of ALS and/or FTD in a subject, such as a human; or the study of ALS and/or FTD in a subject, such as a mouse.

As the person skilled in the art would understand, strict compliance to the sequences provided is not necessary for the function of the promoter, provided that functional elements, e.g. enhancers, and their spatial relationships are essentially maintained. In particular, the promoter sequences provided comprise flanking restriction sites for cloning into a vector. The person skilled in the art would know to adapt these restriction sites to the particular cloning system used, as well as to make any point mutations that may be required in the sequence of the promoter to remove e.g. a cryptic restriction site (see e.g. Example 17 of WO 2017/077329).

Suitable inducible systems may use small molecule induction, such as the tetracycline-controlled systems (tet-on and tet-off), the radiation-inducible early growth response gene-1 (EGR1) promoter, and any other appropriate inducible system known in the art.

### Differential Expression of and Target Gene Regulation by Zinc Finger Effectors:

In aspects and embodiments of the invention, for example, in therapeutic applications, it may be desirable to increase the expression of a wild-type protein in order to address a haploinsufficiency, such as in the case of ALS and/or FTD. In such diseases, the wild-type C9Orf72 gene, which has a wild-type number of GGGGCC-repeat sequences (i.e. less than 30 repeats) may be underexpressed, leading to a loss of function phenotype; whereas expression of the pathogenic gene construct, which has over 30 GGGGCC repeats (generally over 100 repeats), causes pathogenesis.

This presents a practical problem for gene therapy treatments and other therapeutic applications based on gene regulation, because a designer transcriptional activator peptide of the invention for targeting relatively short hexa- or trinucleotide repeats of such wild-type genes will find a greater number of target sites associated with a pathogenic gene and so, presumably, would preferentially activate the pathogenic gene.

The present inventors have addressed this problem by ‘tuning’ respective zinc finger repressor and activator proteins to provide a beneficial balance between activation of the wild-type gene and repression of the mutant allele.

As described above, therefore, zinc finger repressor proteins of the first aspects and embodiments of the invention are optimised with novel binding-destabilising mutations to target binding to repetitive GGGGCC sequences of at least 30 repeats (Lancet Neurol. (2012); 11: 323-30) and, beneficially bind with increasing strength as the number of GGGGCC repeats increases, e.g. to over 100 repeats (patients diagnosed with disease typically have 700-1,600 GGGGCC repeats, whereas healthy individuals have between about 2 and 23 repeats; Neuron (2011) 72, 245-56).

Conversely, such long, binding-destabilised zinc finger peptides bind relatively weakly to the short, wild-type gene sequences. Accordingly, the short WT allele should not be bound (or is bound comparatively weakly) by the extended poly-zinc finger repressor proteins of the invention in view of the specifically designed binding-destabilising mutations within the zinc finger recognition sequences, as discussed herein above, and/or in the linker sequences between adjacent zinc finger domains (or adjacent zinc finger domain pairs). In other words, the zinc finger repressor proteins of the invention may be expressed under the control of a strong promoter sequence (as described here), and preferential binding to expanded, pathogenic nucleotide repeat target sequences is achieved by use of weakened DNA-binding interfaces that favour long DNA-targets and/or specially designed destabilising linkers for use between zinc finger domains or domain pairs. By adding more of these destabilising mutations, an increased number of hexanucleotide repeats, and a higher zinc finger repressor protein concentration are needed to achieve repression of the pathogenic target gene. Furthermore, without wishing to be bound by theory, the inventors have postulated that zinc finger binding to dsDNA (for example) slightly unwinds the DNA, favouring subsequent adjacent zinc finger peptide binding; this leads to cooperativity, also favouring the preferential binding of extended zinc finger repressor protein arrays to long expanded GGGGCC repeat target sequences.

Thus, as described elsewhere herein, the long allele zinc finger repressor proteins of the invention comprise a tandem array of at least 6 zinc finger domains, and typically from 8 to 32 zinc finger domains. Suitably, the repressor proteins of the invention have from 8 to 18 zinc finger domains arranged in tandem; more suitably between 10 and 12 zinc finger domains; and preferably 11 zinc finger domains (along with e.g. a KRAB repression domain, such as mouse Zfp87 for use in a mouse host, or human Kox-1 for use in a human host).

In conjunction with the above zinc finger repressor proteins of the invention, the methods and therapies of the invention may advantageously comprise designed poly-zinc finger activator proteins to upregulate/activate the expression of the WT allele to help to overcome haploinsufficiency. As in the case for zinc finger repressor proteins and their intended gene target, the zinc finger activator proteins of the invention are tuned to preferentially activate the wild-type gene (associated with a relatively short nucleotide repeat sequence); i.e. wild-type C9Orf72, by adjusting the affinity and/or concentration of zinc finger activator proteins within a target cell or system. In principle, of course, a zinc finger activator protein could within the same cell (if not suitably tuned) simultaneously activate both wild-type and pathogenic alleles to an extent. However, by simultaneous/separate or sequential administration or expression of a zinc finger repressor protein of the invention, the potentially toxic gain of function may advantageously be dominantly repressed by the longer (lower affinity) extended poly-zinc finger repressor proteins of the invention, whose affinity and concentration are tuned to repress the longer mutant allele preferentially. Beneficially, a higher expression concentration of the longer repressor protein may also help to outcompete the activator protein at the longer pathogenic gene sequences.

So as to avoid introducing a bias for binding to long hexanucleotide repeat sequences, the wild-type (short) allele-targeting zinc finger activator proteins of the invention comprise a tandem array of at most 8 zinc finger domains, and typically at most 6 or 7 zinc finger domains. Suitably, the zinc finger activator peptides of the invention has only 5, 6 or 7 zinc finger domains, and preferably have 6 zinc finger domains (along with a transactivation domain such as p65-RelA (human/mouse; EMBO J. (1991) 10(12):3805-17); VP16 or VP64 (Herpes simplex) for use in mouse or human hosts).

Moreover, the inventors have found that it can be advantageous to use the high-affinity (shorter) zinc finger activator proteins of the invention at a lower concentration (within a target cell or system) than the lower affinity extended poly-zinc finger repressor protein variants for targeting the long mutant allele. In this way, length discrimination of target genes can be maximised and enable selective activation or repression of short/long gene alleles, respectively.

Many systems are known and available to the skilled person to allow for differential expression levels of co-expressed exogenous genes, such as the zinc finger activator and zinc finger repressor proteins of the invention. For example, in embodiments, the concentration of a desired peptide may be tuned by the design of promoter-enhancer constructs, 5-UTRs and/or start codon sequence.

As discussed above, for neuronal and/or ubiquitously gene expression, respectively, NSE is considered to be a very strong promotor, while HSPAB1 is considered to be a strong promoter.

As described herein, weaker expression of the high-affinity zinc finger activator proteins of the invention compared to the lower-affinity repressor proteins of the invention is desired for therapeutic applications. In embodiments, relatively lower expression of zinc finger activator proteins of the invention may be achieved using a weak (or weaker) promoter compared aot HSPAB1 or NSE. However, as the skilled person would appreciate, reduced gene expression can also be achieved in other manners, for example, using weaker/lower-efficiency start codons. Thus, in embodiments, alternative weaker-efficiency start codons are used in zinc finger activator expression constructs of the invention. For example, in mammalian cells, protein expression from a gene sequence beginning at a CTG codon is approx. 20% of the level that would be expected using a normal ATG start condon; whereas expression from a GTG codon is about 10% of the ATG codon level; and expression from a TTG codon is only approx. 2% of the level of an ATG codon (PNAS (2010) 107: 18056-18060; Genes & Dev. (2017) 31: 1717-1731).

Accordingly, in embodiments of the invention, a zinc finger repressor protein of the invention may be expressed using pNSE or pHSP90AB1 promoter sequences in conjunction with a convention ATG start codon. In some beneficial embodiments, however, a zinc finger activator protein may be expressed from the same promotor constructs, but in conjunction with a non-ATG start codon as noted above. Suitably, the non-ATG start codon is CTG, such that the expression of a zinc finger activator protein of the invention is about 20% of the level of the repressor protein; although of course, other combinations of modified ‘starting’ codon are possible.

According to other embodiments of the invention, it is also possible to ‘tune’ (or down-regulate) expression of zinc finger activator proteins of the invention by adding RNA hairpins in the 5′-UTR region, upstream of the start codon (Synthetic Biology (2018) 3(1): ysy019). These and any other measures for regulating gene expression can be used in isolation or in conjunction with any other method for modifying gene expression levels, as described herein and/or as known to the person skilled in the art.

### Therapeutic Compositions

A zinc finger peptide or chimeric modulator of the invention may be incorporated into a pharmaceutical composition for use in treating an animal; preferably a human. A therapeutic peptide of the invention (or derivative thereof) may be used to treat one or more diseases or infections, depending on which binding site the zinc finger peptide is selected or designed to recognise. Alternatively, a nucleic acid encoding the therapeutic peptide may be inserted into an expression construct/vector and incorporated into pharmaceutical formulations/medicaments for the same purpose.

As will be understood by the person of skill in the art, potential therapeutic molecules, such as zinc finger peptides and modulators of the invention may be tested in an animal model, such as a mouse, before they can be approved for use in human subjects. Accordingly, zinc finger peptide or chimeric modulator proteins of the invention may be expressed in vivo in mice or ex vivo in mouse cells as well as in humans. In accordance with the invention, appropriate expression cassettes and expression constructs/vectors may be designed for each animal system specifically.

Zinc finger peptides and chimeric modulators of the invention typically contain naturally occurring amino acid residues, but in some cases non-naturally occurring amino acid residues may also be present. Therefore, so-called ‘peptide mimetics’ and ‘peptide analogues’, which may include non-amino acid chemical structures that mimic the structure of a particular amino acid or peptide, may also be used within the context of the invention. Such mimetics or analogues are characterised generally as exhibiting similar physical characteristics such as size, charge or hydrophobicity, and the appropriate spatial orientation that is found in their natural peptide counterparts. A specific example of a peptide mimetic compound is a compound in which the amide bond between one or more of the amino acids is replaced by, for example, a carbon-carbon bond or other non-amide bond, as is well known in the art (see, for example Sawyer, in Peptide Based Drug Design, pp. 378-422, ACS, Washington D.C. 1995). Such modifications may be particularly advantageous for increasing the stability of zinc finger peptide therapeutics and/or for improving or modifying solubility, bioavailability and delivery characteristics (e.g. for in vivo applications) when a peptide is to be administered as the therapeutic molecule.

The therapeutic peptides and nucleic acids of the invention may be particularly suitable for the treatment of diseases, conditions and/or infections that can be targeted (and treated) intracellularly, for example, by targeting genetic sequences within an animal cell; and also for in vitro and ex vivo applications. As used herein, the terms ‘therapeutic agent’ and ‘active agent’ encompass both peptides and the nucleic acids that encode a therapeutic zinc finger peptide of the invention. Therapeutic nucleic acids include vectors, viral genomes and modified viruses, such as AAV, which comprise nucleic acid sequences encoding zinc finger peptides and fusion proteins of the invention.

Therapeutic uses and applications for the zinc finger peptides and nucleic acids include any disease, disorder or other medical condition that may be treatable by modulating the expression of a target gene or nucleic acid.

In accordance with first aspects and embodiments of the present invention, diseases of hexanucleotide repeat expansion are a particular target of the present therapies based on poly-zinc finger therapeutic molecules, for example: Amyotrophic lateral sclerosis (ALS) and familial Frontotemporal dementia (FTD), both of which are associated with expanded GGGGCC polynucleotide repeat sequences. Zinc finger peptides of the invention are particularly adapted to target and bind to GGG-GCC-repeat sequences within human or animal genomes. A preferred target gene is C90RF72, which is known to be susceptible to expansion of the wild-type short GGGGCC repeat sequence. In this example, a wild-type gene is typically associated with less than 30 GGGGCC repeat sequences, and generally between 2 and 23 such repeats. On the other hand, abnormal, pathogenic C9ORF72 genes comprise at least 30, and typically in the range of 700 to 1,600 GGGGCC repeat sequences.

One or more additional pharmaceutically acceptable carrier (such as diluents, adjuvants, excipients or vehicles) may be combined with the therapeutic peptide(s) of the invention in a pharmaceutical composition. Suitable pharmaceutical carriers are described in “Remington's Pharmaceutical Sciences” by E. W. Martin. Pharmaceutical formulations and compositions of the invention are formulated to conform to regulatory standards and can be administered orally, intravenously, topically, or via other standard routes.

In accordance with the invention, the therapeutic peptides or nucleic acids may be manufactured into medicaments or may be formulated into pharmaceutical compositions. When administered to a subject, a therapeutic agent is suitably administered as a component of a composition that comprises a pharmaceutically acceptable vehicle. The molecules, compounds and compositions of the invention may be administered by any convenient route, for example, intradermal, intramuscular, intraperitoneal, intravenous, subcutaneous, intranasal, epidural, oral, sublingual, intranasal, intravaginal, transdermal, rectally, by inhalation, or topically to the skin. Administration can be systemic or local. Delivery systems that are known also include, for example, encapsulation in microgels, liposomes, microparticles, microcapsules, capsules, etc., and any of these may be used in some embodiments to administer the compounds of the invention. Any other suitable delivery systems known in the art are also envisaged in use of the present invention.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle particularly when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

In some embodiments the therapeutic compositions or medicaments of the invention are formulated in accordance with routine procedures as a pharmaceutical composition adapted for oral administration (more suitably for human beings). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. One skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the therapeutic agent or nucleic acid (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the peptide or nucleic acid or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the invention is via pulmonary or nasal delivery.

Additives may be included to enhance cellular uptake of the therapeutic peptide (or derivative) or nucleic acid of the invention, such as the fatty acids, oleic acid, linoleic acid and linolenic acid.

In one exemplary pharmaceutical composition of the invention, one or more zinc finger peptide or nucleic acid of the invention (and optionally any associated non-zinc finger moiety, e.g. a modulator of gene expression and/or targeting moiety) may be mixed with a population of liposomes (i.e. a lipid vesicle or other artificial membrane-encapsulated compartment), to create a therapeutic population of liposomes that contain the therapeutic agent and optionally the modulator or effector moiety. The therapeutic population of liposomes can then be administered to a patient by any suitable means, such as by intravenous injection. Where it is necessary for the therapeutic liposome composition to target specifically a particular cell-type, such as a particular microbial species or an infected or abnormal cell, the liposome composition may additionally be formulated with an appropriate antibody domain or the like (e.g. Fab, F(ab)₂, scFv etc.) or alternative targeting moiety, which naturally or has been adapted to recognise the target cell-type. Such methods are known to the person of skill in the art.

The therapeutic peptides or nucleic acids of the invention may also be formulated into compositions for topical application to the skin of a subject.

In embodiments of the invention the therapeutic compositions may include only one therapeutic peptide/protein or nucleic acid of the invention; or may include two or more e.g. two complementary therapeutic peptides/proteins or nucleic acids of the invention. For example, a poly-zinc finger repressor protein of the invention may be used alone, or in combination with another zinc-finger peptide or therapeutic agent, e.g. to downregulate expression of a pathogenic gene target. In other embodiments, two therapeutic zinc finger peptides of the invention may be used in concert; e.g. a zinc finger repressor protein for downregulating expression of a target pathogenic gene (e.g. associated with causing ALS and/or FTD) may be used in combination with a zinc finger activator protein for upregulating expression of an associated target wild-type gene, thereby to address haploinsufficiency in an affected subject. When two (or more) therapeutic zinc finger peptides are contemplated, the different zinc finger peptides or encoding nucleic acid constructs or viral vectors may be incorporated into the same pharmaceutical composition, or may be manufactured separately. Where two (or more) pharmaceutical compositions are manufactured for administration to the same individual, it will be appreciated that the compositions may be administered simultaneously, sequentially, or separately, as directed/required.

Zinc finger peptides and nucleic acids of the invention may also be useful in non-pharmaceutical applications, such as in diagnostic tests, imaging, as affinity reagents for purification and as delivery vehicles.

### Gene Therapy

One aspect of the invention relates to gene therapy treatments utilising zinc finger peptides of the invention for treating diseases.

Gene therapy relates to the use of heterologous genes in a subject, such as the insertion of genes into an individual's cell (e.g. animal or human) and biological tissues to treat disease, for example: by replacing deleterious mutant alleles with functional/corrected versions, by inactivated mutant alleles by removing all or part of the mutant allele, or by inserting an expression cassette for sustained expression of a therapeutic zinc finger construct according to the invention. The most promising target diseases to date are those that are caused by single-gene defects, such as cystic fibrosis, haemophilia, muscular dystrophy, sickle cell anaemia, Huntington's disease (HD), ALS, FTD, FXTAS and FXS. Other common gene therapy targets are aimed at cancer and hereditary diseases linked to a genetic defect, such as expanded nucleotide repeats. The present invention is concerned with the treatment of genes associated with expanded polynucleotide repeats, and in particular, with expanded repeats of the hexanucleotide sequence GGGGCC or variants thereof (such as GGGCCG, GGCCGG, GCCGGG and CCGGGG).

Gene therapy is classified into two types: germ line gene therapy, in which germ cells, (i.e. sperm or eggs), are modified by the introduction of therapeutic genes, which are typically integrated into the genome and have the capacity to be heritable (i.e. passed on to later generations); and somatic gene therapy, in which the therapeutic genes are transferred into somatic cells of a patient, meaning that they may be localised and are not inherited by future generations.

Gene therapy treatments require delivery of the therapeutic gene (or DNA or RNA molecule) into target cells. There are two categories of delivery systems, either viral-based delivery mechanisms or non-viral mechanisms, and both mechanisms are envisaged for use with the present invention.

Viral systems may be based on any suitable virus, such as: retroviruses, which carry RNA (e.g. influenza, SIV, HIV, lentivirus, and Moloney murine leukaemia); adenoviruses, which carry dsDNA; adeno-associated viruses (AAV), which carry ssDNA; herpes simplex virus (HSV), which carries dsDNA; and chimeric viruses (e.g. where the envelop of the virus has been modified using envelop proteins from another virus).

A particularly preferred viral delivery system is AAV. AAV is a small virus of the parvovirus family with a genome of single stranded DNA. A key characteristic of wild-type AAV is that it almost invariably inserts its genetic material at a specific site on human chromosome 19. However, recombinant AAV, which contains a therapeutic gene in place of its normal viral genes, may not integrate into the animal genome, and instead may form circular episomal DNA, which is likely to be the primary cause of long-term gene expression. Advantages of AAV-based gene therapy vectors include: that the virus is non-pathogenic to humans (and is already carried by most people); most people treated with AAV will not build an immune response to remove either the virus or the cells that have been successfully infected with it (in the absence or heterologous gene expression); it will infect dividing as well as non-dividing (quiescent) cells; and it shows particular promise for gene therapy treatments of muscle, eye, and brain. AAV vectors have been used for first- and second-phase clinical trials for the treatment of cystic fibrosis; and first-phase clinical trials have been carried out for the treatment of haemophilia. There have also been encouraging results from phase I clinical trials for Parkinson's disease, which provides hope for treatments requiring delivery to the central nervous system. Gene therapy trials using AAV have also been reported for treatment of Canavan disease, muscular dystrophy and late infantile neuronal ceroid lipofuscinosis. HSV, which naturally infects nerve cells in humans, may also offer advantages for gene therapy of diseases involving the nervous system.

Suitably, in accordance with the invention, zinc finger encoding nucleic acid constructs (as described herein) are inserted into an adeno-associated virus (AAV) vector, particularly the AAV2/1 subtype (see e.g. Molecular Therapy (2004) 10: 302-317). This vector is particularly suitable for injection into and infection of the striatum, in the brain, where the therapeutics of the invention may be particularly useful. Alternatively, the vector can be injected intrathecally or directly into the cisterna magna or brain. Intrathecally is a preferred mode route for administration of AAV2/1 therapeutics of the present invention. In this way, the zinc finger encoding nucleic acid constructs of the invention can be delivered to desired target cells, and the zinc finger peptides expressed in order to repress the expression of pathogenic genes associated with GGGGCC repeat sequences, such as mutant C9ORF72 genes.

In embodiments, viral vectors with a wider tropism are used instead, or in addition to, vectors with a more specific tropism. For example, the neuron specific AAV2/1 subtype may be used in combination with the AAV2/9 subtype. This may advantageously allow targeting of both neurons and other types of cells present in the brain, such as glial cells. Ubiquitous/promiscuous viral vectors, such as AAV2/9, may also be used alone, for example, where the therapy is targeted at peripheral tissues. In addition, AAV2/9 can beneficially be used systemically and intravenously, and/or delivered to different organs of a subject, e.g. by intramuscular injection. Again, however, intrathecal administration of AAV2/9 therapeutics may be preferred.

Although ALS and FTD are primarily considered to be neurological diseases, the effects of the diseases are far-reaching throughout the body. Therefore, targeting of tissues other than the central nervous system with the zinc finger peptides/modulators of the invention may prove beneficial. In such applications use of a promiscuous vector (such as AAV2/9) or an organ/tissue specific vector may be particularly useful.

In embodiments, the tropism of the viral vector and the specificity of the promoter used for expression of the therapeutic construct can be tailored for targeting of specific populations of cells. For example, neuron-specific viral vectors may be used in combination with neuron-specific promoters. Conversely, promiscuous vectors may be used in combinations with ubiquitous promoters (or tissue specific promoters as desired).

In specific embodiments, AAV2/1 viruses may be used in combination with a synthetic pNSE promoter, as described above (see also WO 2017/077329). In other embodiments, AAV2/9 viruses may be used in combination with a synthetic pHSP vector, also as described above (see also WO 2017/077329). In embodiments, combinations of these two types of constructs may be used in order to simultaneously target multiple cell types, e.g. for the treatment of ALS and/or FTD.

For some applications non-viral based approaches for gene therapy can provide advantages over viral methods, for example, in view of the simple large-scale production and low host immunogenicity. Types of non-viral mechanism include: naked DNA (e.g. plasmids); oligonucleotides (e.g. antisense, siRNA, decoy ds oligodeoxynucleotides, and ssDNA oligonucleotides); lipoplexes (complexes of nucleic acids and liposomes); polyplexes (complexes of nucleic acids and polymers); and dendrimers (highly branched, roughly spherical macromolecules).

Accordingly, the zinc finger-encoding nucleic acids of the invention may be used in methods of treating diseases by gene therapy. As already explained, particularly suitable diseases are those of the nervous system (especially motor neurons); and preferably those associated with GGGGCC repeat sequences, such as ALS and FTD.

Accordingly, the gene therapy therapeutics and regimes of the invention may provide for the expression of therapeutic zinc fingers in target cells in vivo or in ex vivo applications for repressing the expression of target genes, such as those having non-wild-type expanded GGGGCC-repeat sequences, and especially the mutant C9ORF72 gene.

Zinc finger nucleases of the invention (e.g. as fusion proteins with Fok-1 nuclease domain) may also be useful in gene therapy treatments for gene cutting or directing the site of integration of therapeutic genes to specific chromosomal sites, as previously reported by Durai et al. (2005) Nucleic Acids Res. 33, 18: 5978-5990.

### Amyotrophic Lateral Sclerosis (ALS) and Frontotemporal Dementia (FTD)

As in many other neurological disorders, such as Alzheimer's and Parkinson's diseases, Amyotrophic lateral sclerosis (ALS; or motor neurone disease) and familial Frontotemporal dementia (FTD) have complex disease pathologies.

ALS is a neurodegenerative syndrome characterised by adult-onset progressive loss of motor neurons with a focal onset of progressive paresis and muscle wasting (Brooks (1994) J. Neurol. Sci. 124, Suppl: 96-107). Less than 10% of cases are reported to have a familial predisposition (fALS), and the remaining cases are considered to be sporadic ALS (sALS). Mutations in approx. 37 genes have been reported to predispose an individual to ALS; but the most commonly reported mutation is the GGGGCC repeat-expansion in the intron of the C9orf72 gene (C9orf72HRE), which is identified in about 10% of patients with ALS. Missense, substitution or deletion mutations in the genes encoding superoxide dismutase-1 (SOD1), TAR-DNA-binding protein 43 (TDP-43), fused in sarcoma (FUS) and kinesin heavy chain isoform 5A (KIF5A) are also commonly found in ALS.

Carriers of C9orf72HRE, FUS, VCP and TBK1 genetic mutations may also develop frontotemporal dementia (FTD); sometimes even without showing obvious signs of ALS.

The presence of neuronal inclusions containing aggregated SOD1 protein is a recognised pathological hallmark of ALS, which can be caused by SOD1 mutations in patients and in transgenic (Tg) animal models overexpressing mutant human (h) SOD1 (Jonsson et al., (2004) Brain, 127: 73-88; Bruijn et al. (1998) Science, 281: 1851-1854). Indeed, there is increasing evidence that misfolded SOD1 is neurotoxic and may be a trigger for ALS pathogenesis generally—not only in patients carrying mutations in SOD1 (Forsberg et al. (2019) Journal of Neurology, Neurosurgery & Psychiatry, 16 Apr. 2019). Therefore, although new therapies targeting SOD1 are ongoing or are being planned, simply targeting a mutant SOD1 gene with tailored genetic inhibition therapy, for example, may not be sufficient to halt disease progression.

Accordingly, there is a need for new therapies for ALS and/or FTD, for example, which target genes other than SOD1, but which may have a positive effect on appropriate SOD1 folding and/or wild-type SOD1 expression. The present inventors have thus hypothesised that reversing or alleviating the haploinsufficiency that can result from pathogenic GGGGCC repeats in the C9ORF72 gene may provide potential new therapeutic treatments for ALS and/or FTD.

### Host Organism Toxicity and Immunogenicity

It was proposed that toxicity and immunogenicity (immunotoxicity) of heterologous peptides when expressed in host organisms might be reduced by optimising the primary peptide sequence to match the primary peptide sequence of natural host peptides.

As previously described (Garriga et al., 2012 and in WO 2017/077329), zinc finger peptides based on a generic/universal zinc finger peptide framework, and particularly on the peptide framework of Zif268, which is a natural zinc finger protein having homologues in both mice and humans can be beneficial for reducing host immune reactions. However, in general, the recognition sequences of a zinc finger domain should be based on the perceived best match for the target nucleic acid sequences (i.e. the recognition code for zinc finger-dsDNA interactions) and on binding optimisation studies. Such designs according to the prior art have no regard to the target host organism in which the zinc finger peptides would be ultimately expressed (e.g. mouse or human). Similarly, effector domains, such as transcriptional activator and repressor domains and other effector functions, such as nuclear localisation and purification tags have been previously selected without regard to the host organism. This has been shown to be a potential reason for failure to express exogenous, therapeutic peptides over the long term in a host organism. The inventors' previous work (WO 2017/077329) addressed this problem in the art, and the present invention follows those important teachings.

Thus, zinc finger peptides and modulator peptides of the invention have greater than 50%, greater than 60%, greater than 70% or even greater than 75% identity to endogenous/natural protein sequences in the target, host organism in which they are intended to be expressed for therapeutic use. More suitably, the peptides of the invention have at least 80%, 81%, 82%, 83%, 84% or at least 85% identity to endogenous/natural proteins in the target organism. In some cases, it is desirable to have still greater identity to peptide sequences of the target/host organism, such as between approximately 75% and 98% identity, between 78% and 95% identity, between 80% and 90% identity. At the same time, it will be appreciated that the peptides of the invention are different to known peptide sequences. Thus, the peptides may be up to 50%, up to 40%, up to 30% or up to 25% non-identical to endogenous/natural peptide sequences found in the host organism and/or previously known. It will be appreciated that by ‘up to x %’, in this context, means greater than 0% and less than x %. Preferably, the peptides of the invention are up to 20%, 19%, 18%, 17%, 16%, 15%, 14%, 13%, 12%, 11% or 10% non-identical to endogenous/natural peptide sequences found in the host organism; for example, the peptides of the invention may be between approximately 1% and 25%, between approximately 3% and 20% or between approximately 5% and 15% non-identical to an endogenous peptide sequence of the host organism.

Sequence identity can be assessed in any way known to the person of skill in the art, such as using the algorithm described by Lipman & Pearson (1985), Science 227, pp1435; or by sequence alignment.

As used herein, ‘percent identity’ means that, when aligned, that percentage of amino acid residues (or bases in the context of nucleic acid sequences) are the same when comparing the two sequences. Amino acid sequences are not identical, where an amino acid is substituted, deleted, or added compared to the reference sequence. In the context of the present invention, since the subject proteins may be considered to be modular, i.e. comprising several different domains or effector and auxiliary sequences (such as NLS sequences, expression peptides, zinc finger modules/domains, and effector domains (e.g. repressor peptides)), sequence identity may conveniently be assessed separately for each domain/module of the peptide relative to any homologous endogenous or natural peptide domain/module known in the host organism. This is considered to be an acceptable approach since relatively short peptide fragments (epitopes) of any host-expressed peptides may be responsible for determining immunogenicity through recognition or otherwise of self/non-self peptides when expressed in a host organism in vivo. By way of example, a peptide sequence of 100 amino acids comprising a host zinc finger domain directly fused to a host repressor domain wherein neither sequence has been modified by mutation would be considered to be 100% identical to host peptide sequences. It does not matter for this assessment whether such zinc finger domain(s) or non-zinc finger domain, e.g. repressor domain, is only a fragment from a natural, larger protein expressed in the host. If one of 100 amino acids has been modified from the natural sequence, however, the modified sequence would be considered 99% identical to natural protein sequences of the host; whilst if the same zinc finger domain were linked to the same repressor domain by a linker sequence of 10 amino acids and that linker sequence is not naturally found in that context in the host organism, then the resultant sequence would be (10/110) ×100% non-identical to host sequences.

Thus, the degree of sequence identity between a query sequence and a reference sequence may, in some embodiments be determined by: (1) aligning the two sequences by any suitable alignment program using the default scoring matrix and default gap penalty; (2) identifying the number of exact matches, where an exact match is where the alignment program has identified an identical amino acid or nucleotide in the two aligned sequences on a given position in the alignment; and (3) dividing the number of exact matches with the length of the reference sequence. In other embodiments, step (3) may involve dividing the number of exact matches with the length of the longest of the two sequences; and in other embodiments, step (3) may involve dividing the number of exact matches with the ‘alignment length’, where the alignment length is the length of the entire alignment including gaps and overhanging parts of the sequences. As explained above, in this context, the alignment length is the accumulative amino acid length of all peptide domains, modules or fragments that have been used as reference sequences for each respective domain or module of the query peptide.

Sequence identity comparisons can be conducted by eye, or more usually, with the aid of readily available sequence comparison programs. Commercially available computer programs may use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms may include one or more and typically all of: (i) assignment of a penalty score each time a gap is inserted (gap penalty score); (ii) assignment of a penalty score each time an existing gap is extended with an extra position (extension penalty score); (iii) assignment of high scores upon alignment of identical amino acids; and (iv) assignment of variable scores upon alignment of non-identical amino acids. Most alignment programs allow the gap penalties to be modified. However, it is preferred to use the default values when using such software for sequence comparisons.

In some algorithms, the scores given for alignment of non-identical amino acids are assigned according to a scoring matrix, which may also be called a substitution matrix. The scores provided in such substitution matrices may reflect the fact that the likelihood of one amino acid being substituted with another during evolution varies and depends on the physical/chemical nature of the amino acid to be substituted. For example, the likelihood of a polar amino acid being substituted with another polar amino acid is higher compared to the likelihood that the same amino acid would be substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are perhaps the PAM matrices (Dayhoff et al. (1978), Jones et al. (1992)), the BLOSUM matrices (Henikoff & Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins DG & Sharp PM (1988), Higgins et al. (1992), Thompson et al. (1994), Larkin et al. (2007). A selection of different alignment tools is available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/and which was firstly described in Altschul et al. (1990), J. Mol. Biol. 215; pp 403-410. Examples of programs that perform global alignments are those based on the Needleman-Wunsch algorithm, e.g. the EMBOSS Needle and EMBOSS Stretcher programs. In one embodiment, it is preferred to use the ClustalW software for performing sequence alignments. ClustalW2 is for example made available on the internet by the European Bioinformatics Institute at the EMBL-EBI webpage www.ebi.ac.uk under tools-sequence analysis-ClustalW2.

Once an appropriate software program has produced an alignment or a group of alignments, it is possible to calculate % similarity and % sequence identity. The software typically does this as part of the sequence comparison and generates a numerical result. In a preferred embodiment of the present invention, the alignment is run over domain stretches rather than by performing a global alignment to attempt to optimise the alignment over the full-length of a sequence. Therefore, in preferred embodiments, whilst an alignment program may be used for ease of reference and consistency, since sequence lengths are relatively short and peptides of the invention may contain domains derived from several different proteins, sequence identity is most simply carried out by visual inspection of aligned full or partial sequences and manual calculation of identity.

The present inventors have designed a series of zinc finger peptides and zinc finger peptide effectors based in part on their intended optimal binding-mode and functionality and partly which are adapted to increase their compatibility with the host organism in which they are to be expressed, e.g. mouse or human. These so-called ‘mousified’ and ‘humanised’ zinc finger peptides have been found to substantially reduce potential immunogenicity and toxicity effects in vivo in this and earlier studies (e.g. WO 2017/077329).

The aim of ‘humanisation’ or ‘mousification’ is to minimise the amino acid sequence differences between an artificial zinc finger design, chosen to bind poly-GGGGCC, and a naturally-occurring zinc finger repeat, Zif268 (which has human and mouse homologues, and which naturally binds the sequence GCG-TGG-GCG; Pavletich, 1991). In practice, ‘humanisation’ or ‘mousification’, has the intention of reducing the potential for foreign epitopes in the zinc finger peptide sequences of the invention. These changes must be carried out within the constraints of achieving effective targeting of and binding to GGGGCC-repeat sequences within a desired range of binding affinity according to the length of the zinc finger array and the intended effect (repression or activation).

Importantly, since Zif268 has homologues in mouse and human cells, and the zinc finger scaffold framework of Zif268 is almost identical in mice and humans (see SEQ ID NO: 164; SEQ ID NO: 165, respectively), the inventors have previously shown that a single appropriately modified host-optimised zinc finger peptide sequence of the invention may be suitable for use in both mouse and human cells without resulting in adverse immunogenic effects: thus, a single host optimised zinc finger design for binding poly-GGGGCC can be useful in both species. Desirably, the sequence identity of a peptide of the invention to each of native mouse and human sequences is at least about 75%, at least about 80% or at least about 85%; such as between about 75% and 95%, or between about 80% and 90%. It will of course be appreciated that the sequence identity cannot reach 100% because the zinc finger peptides of the invention are specifically designed for binding particular identified pathogenic or therapeutic DNA target sequences which are not identical to the target sequence of Zif268. Therefore, as regards percentage indentity of the peptides of the invention, ‘at least x %’ must always be lower than 100% (e.g. at most about 99% identity).

In order to improve sequence identity, the KRAB repressor domain, Kox-1, which was suitable for and ‘host-matched’ for use in humans, is replaced by the mouse analogue KRAB domain from ZF87, also called MZF22 (Abrink et al., 2001) for mouse studies. To further improve host optimisation, nuclear localisation signals were selected from human (KIAA2022) and mouse (p58 protein) sequences for expression in humans or mice, respectively.

In addition, improved host-optimisation can be achieved by modifying the originally designed recognition helices and zinc finger linkers in order to match them as closely as possible to the human (or mouse respectively) Zif268 transcription factor sequences. Thus, for example, the first zinc finger recognition sequence in a zinc finger array may have the amino acid sequence LT in the +4 and +5 positions, respectively, of the alpha-helix, rather than the amino acid sequence RK, which is found in the third recognition sequence of Zif268.

As used herein, a short-hand nomenclature of a ‘humanised’ zinc finger peptide of the invention (e.g. having 11 zinc fingers) is termed herein, ‘hZF . . . ’, whereas a ‘mousified’ version of the zinc finger peptode is termed ‘mZF . . . ’.

Particular differences between the mouse and human variants of the zinc finger peptides of the invention lie in the repressor domain, which is the ZF87 KRAB domain for mouse and the Kox-1 KRAB domain for humans; and the nuclear localisation signal (NLS), which may suitably be derived from a human variant peptide for use in humans (Human protein KIAA2022 NLS), and a mouse peptide for use in mouse, as described elsewhere herein. Similarly, the activation domain of zinc finger activator peptides of the invention may be the p65 RelA activation domain derived from the human variant for use in humans or from the mouse variant for use in mice (EMBO J. (1991) 10(12):3805-17), or VP16/VP64 activation domains may be used as appropriate.

It has thus been found that several design variants of zinc finger peptide sequences can be synthesised to retain desired poly-GGGGCC binding characteristics, while improving/maximising host matching properties and minimising toxicity in vivo. Surprisingly, such design variants can include a relatively high number of modifications within zinc finger alpha-helical recognition sequences and within zinc finger linker sequences, both of which might be expected to affect (e.g. reduce) target nucleic acid binding affinity and specificity, without adversely affecting the efficacy of the potential therapeutic for use in vivo. Moreover, by beneficially reducing immunogenicity and toxicity effects in vivo, mid to long-term activity of the therapeutic peptides of the invention are significantly increased.

### Active Delivery of Therapeutic Zinc Finger Peptides

Efficient long-term delivery of gene regulatory factors to somatic cells has great potential in medicine: especially for cases where one wishes to reprogram genetic networks or to control gene expression at will.

In recent years, there have been reported in the art many examples of designer gene-specific transcription factors being used to up- or down-regulate target disease genes. However, in most cases long-term treatment (from a single therapeutic administration) is impossible. Against this background, the inventors have developed a universal method for enhanced control of gene expression in vitro and, advantageously, in vivo with artificial gene-regulatory transcription factors, such as zinc finger peptides. This new method provides a means for significantly increasing the ability to artificially control somatic gene expression, based on the concept of ‘active delivery’ of therapeutic peptides, such as transcription factors (e.g. zinc finger peptides), to cells. The process of active delivery involves the general steps of: expression of a therapeutic peptide in a first cell; secretion of the therapeutic peptide from the first cell; diffusion of the therapeutic peptide from the first cell to a neighbouring (second) cell; cell-penetration of the neighbouring cell by the secreted therapeutic peptide; and therapeutic peptide targeting, such that the therapeutic peptide delivers its therapeutic effect to a desired location within the neighbouring cell. The therapeutic peptide is desirably a designer transcription factor, such as one or more of the zinc finger peptides described herein.

Thus, the present disclosure also relates to methods and peptide/nucleic acid constructs for prolonged and/or enhanced therapy. In this regard, the inventors have surprisingly discovered that ‘active delivery’ of therapeutic zinc finger peptides to diseased cells can be achieved in vitro and in vivo, and that such active delivery can improve the efficacy of a therapeutic treatment. In particular, active delivery of therapeutic peptides to pathogenic cells which have not been directly contacted with or transduced by a gene therapy vector (such as an AAV vector) can enhance a single therapeutic treatment, by delivering therapeutic peptides to diseased cells that would otherwise be unaffected by the treatment. In addition, active delivery of therapeutic peptides can continue to deliver therapeutic peptides to diseased cells which previously had been treated with a gene therapy or therapeutic peptide, in circumstances where the gene therapy has been silenced or has otherwise become ineffective.

Indeed, the inventors have previously shown that ZFP therapies are currently limited by long-term expression efficiency: for example, for treatment of Huntingtin's disease, despite that long term expression of therapeutic ZFP transcription factors was achieved by, inter alia, host-matching of therapeutic peptide sequences; target gene repression was limited to approximately 25% in the whole brain after 6 months (Agustin-Pavón et al. (2016) Mol. Neurodegener., 11(1):64). Therefore, while expression of a therapeutic peptide in a proportion of target cells may be effective for a short time period, the therapeutic benefit to the host organism may be rapidly diminished due to the initial failure to deliver the therapeutic transgene into every desirable target cell, followed by the loss of expression of therapeutic transgenes in cells that were initially successfully targeted. Having regard to the prior art, a transgene expression profile after 6 months of 25% of target cells is currently a positive result, but this significantly reduces the effectiveness of any therapy such that further treatments will be necessary to maintain a therapeutic effect in the mid- to long-term.

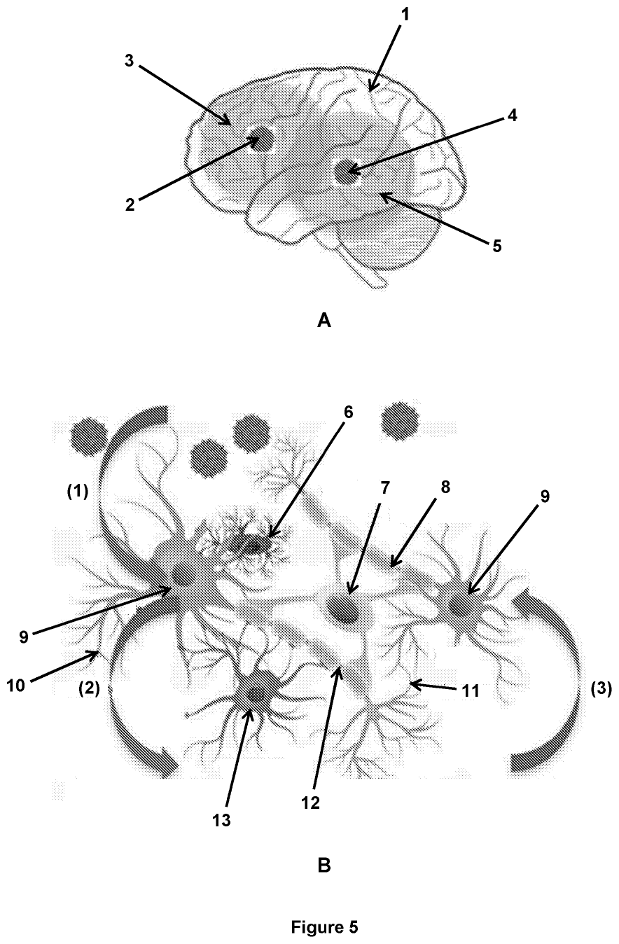

The inventors have now shown that active delivery constructs can improve long-term therapeutic effects by continuing to provide (e.g. to ‘drip-feed’) secreted cell-penetrating therapeutic zinc finger transcription factors to bystander/neighbouring cells in the brain and other tissues, which would not otherwise be exposed to the therapeutic molecules (see FIGS. 5A and 5B).

As exemplified in FIG. 5A, therapeutic delivery agents, e.g. viral vectors (or other delivery systems, such as naked nucleic acids) may conveniently be used to deliver nucleic acid expression constructs to target cells within a host organ(ism). Direct injection of the therapeutic delivery agent is one convenient means for delivering the agent to a desired region of a subject organism. However, whilst such therapeutic delivery agents may infect/enter a plurality of target cells, complete delivery of agent to every target cell is impossible and, even if the delivery were complete or almost complete, it is known that the effectiveness of a gene therapy treatment (e.g. by expression of an exogenous therapeutic peptide agent), is typically limited by gene silencing or vector/transgene loss within the short- or medium-term (e.g. between a few days and a few months). As shown in FIG. 5A, a (first) population of target cells at sites of administration/injection A and B receive a therapeutic transgene (in this example from a viral vector delivery agent), and successfully express the therapeutic peptide. Expressed therapeutic peptides are adapted to be secretable from targeted cells by way of an expressed protein secretion signal (SS) or signal peptide (SP), which causes at least a proportion of the expressed therapeutic peptide to be secreted from the targeted cells that express the peptide. Secreted therapeutic peptides may then diffuse away from the cell in which they were expressed into a ‘diffusion volume’ (e.g. a surrounding region within the host organism), and may come into contact with a multitude more cells of similar type (i.e. a second population of target cells) within the diffusion volume. For example, as depicted in FIG. 5B, infected neuronal cells may express and secret therapeutic peptides, which diffuse away from the cell in which they were expressed and come into contact with non-treated cells, such as astrocytes and other neuronal cells. Furthermore, the secreted therapeutic peptides are advantageously adapted for cell penetration, for example, by way of one or more expressed nuclear localisation signal (NLS), which provides a net positive charge, enhancing the ability of the peptide to penetrate cells. Once inside a ‘neighbouring’ cell, the therapeutic peptide may be targeted to the nucleus (for example), in order to provide a beneficial therapeutic effect in the new cell.

In this way, less than total delivery and expression of a trans/exogenous gene in target cells can be supplemented by exposure of neighbouring cells to the resultant, expressed therapeutic peptide. Such a mechanism can greatly increase the effectiveness of a therapeutic treatment by increasing both the proportion of target cells that receive therapeutic agents and the length of time over which target cells are exposed to therapeutic peptides/agents.

This novel approach is particularly beneficial in conjunction with the zinc finger peptides described elsewhere herein, because the process of cell penetration positively exploits the intrinsic cell penetrating properties of zinc finger peptides (Gaj et al., (2012) Nat. Methods, 9, 805-7; Gaj et al., (2014) ACS Chem. Biol., 9, 1662-7; Liu et al., (2015) Mol. Ther. Nucleic Acids, 4, e232; Mino et al., (2013) PLoS One, 8, e56633). These cell-penetration properties have not been coupled before to secretion in vivo, nor to gene therapy processes based on delivery of an agent with AAVs.

Active delivery can be achieved within a population of cells in vitro or, more advantageously, in vivo: for example, in mouse or humans, using AAV-based vectors to deliver expression constructs encoding therapeutic peptides cabable of secretion from and penetration into target cells. It will be appreciated, however, than any other suitable delivery agent/virus could be used, as could any other appropriately modified therapeutic peptide/agent.

It is generally desired that a delivery vector for use in ‘active delivery’ should be capable of cell/tissue-type specific expression and/or long-term expression and/or strong expression of therapeutic peptides. Thus, delivery vectors according to this disclosure may beneficially comprise a promoter/enhancer sequence such as pCMV, pNSE, pHsp90, CBh, EF1α-1, synapsin or pCAG, which may also be depending on the target organism (e.g. human, mouse, rat etc.). Preferred promoter/enhancer sequences are pNSE, pHsp90, CBh, EF1α-1 and synapsin; especially pNSE and pHsp90, as described herein.

As explained above, a therapeutic peptide for ‘active delivery’ (at least in vivo) must be capable of secretion from the cell in which it is expressed. Multiple cell secretion methods are known to the person skilled in the art and may potentially be employed in accordance with the invention. In particular, cell secretion peptide signal sequences are known and are convenient for use in conjunction with an expressed peptide therapeutic. Thus, the therapeutic peptide may suitably comprise at least one protein secretion signal (SS) or signal peptide (SP), which is expressed as a fusion with the therapeutic peptide. A convenient protein secretion signal is the sequence from human BMP10 protein, which has the sequence MGSLVLTLCALFCLAAYLVSG (SEQ ID NO: 156). However, any secretion signal with downstream cleavage site may alternatively be used (see e.g. Hegde et al. (2006) Trends Biochem Sci., 31(10), 563-71; http://www.signalpeptide.de for examples of possible sequences). Preferably, the SS/SP is host-matched: e.g. human signals would preferably be used for use in humans. Following cell secretion, the therapeutic peptide must be capable of penetrating a cell, and, if the therapeutic peptide is a transcription factor or other DNA-interacting molecule, targeting the nucleus of a cell. Thus, it is convenient that the therapeutic peptide further comprises at least one nuclear localisation sequence (NLS). A suitable NLS sequence is the SV40 NLS (PKKKRKV, SEQ ID NO: 148). However, the nuclear localisation sequence could be a host-derived sequence, such as the NLS from human protein KIAA2022 NLS (PKKRRKVT; NP_001008537.1, SEQ ID NO: 149) for use in humans; or the NLS from mouse primase p58 (RIRKKLR; GenBank: BAA04203.1, SEQ ID NO: 150) for use in mice. In other embodiments, any other suitable NLS known to the person of skill in the art could also be used; e.g. human or mouse NLSs from NLSdb (Nair et al. (2003) Nucleic Acids Res. 31(1): 397-399). In any of these embodiments, in order to enhance cellular uptake, it may be advantageous to combine more than one NLS sequence in tandem; for example, up to 6 NLS, such as 2 (SEQ ID NO: 160), 3, 4 or 5.

The expression construct may further be designed/adapted to place a peptide cleavage site between the SS or SP sequence and the therapeutic peptide effector domain (e.g. such as a zinc finger peptide). Peptide cleavage at the cleavage site separates the therapeutic peptide sequence from the SS or SP sequence and, hence, cleaved therapeutic peptide sequences may remain inside the cell in which they were expressed (or may remain inside the cell in which it eventually penetrates), such that a therapeutic effect may be experienced in the cell that expressed the therapeutic peptide, or the cell in which the therapeutic peptide is delivered to. In preferred embodiments, the gene encoding the therapeutic peptide for active delivery may be constructed such that the NLS sequence or sequences are N-terminal to the therapeutic peptide/zinc finger peptide sequence when expressed. Suitably, also, the secretion signal (SS) or signal peptide (SP) may be arranged N-terminal to the zinc finger peptide sequence. In some particularly beneficial embodiments, the SS or SP sequence is N-terminal to the one or more NLS. Accordingly, cleaved therapeutic peptide advantageously retains the NLS in combination with the therapeutic effector molecule and, thus, the ability to target the nucleus via the NLS or NLSs. It will be appreciated that any suitable peptide cleavage sequence may be employed in conjunction with the invention. One convenient cleavage site is the RIRR peptidase cleavage site. In alternative embodiments, where the therapeutic effect is to be delivered by targeting an organelle other than the nucleus, it will be appreciated that the therapeutic peptide may not comprise an NLS; and may instead include an alternative, appropriate, targeting/cell localisation sequence.

In summary, a therapeutic peptide or designer transcription factor secretion/cell-penetration system according to the invention may advantageously enable bystander cells (neighbouring cells that have not been directly transduced by the therapeutic peptide/transcription factor construct) to receive a steady flow of freshly-expressed therapeutic protein/transcription factor, which may significantly enhance the percentage of a target tissue/organ that can be treated (e.g. by gene regulation). For example, if only 25% of cells would continue expressing a non-secreted therapeutic peptide/artificial transcription factor at 6 months after transduction, then such a treatment could only have a maximum efficacy of 25%. By contrast, if that first population of 25% of the target cells continue to express the therapeutic peptide and the expressed peptide is capable or secretion and subsequent cell-penetration, those 25% of expressing cells may deliver the therapeutic agent to a second population of the target cells, and thereby produce a much more effective functional signal to a much higher percentage of target cells (see FIG. 5B).

Any suitable ‘therapeutic agent’ may be used in conjunction with the ‘active delivery’ platform of the invention, such as zinc finger peptides, TALE transcription factors, CRISPR transcription factors, RNAi etc. However, in some embodiments, therapeutic peptides comprising zinc finger transcription factors may be preferred as an alternative to CRISPR transcription factors, RNAi and TALE transcription factors because: (1) zinc finger peptides are naturally cell-penetrating with high efficiency; (2) zinc finger peptides can be redesigned to target virtually any desired gene; and (3) zinc finger peptides are mammalian in origin, whereas CRISPR/Cas and TALE systems are bacterial—zinc finger peptides therefore have immunological advantages for long-term expression in in vivo systems; and, in addition, (4) zinc finger transcription factors are not based on a nuclease approach—genomic DNA is not cut by zinc finger transcription factors, reducing the risk of undesirable mutagenic effects.

The active delivery platform of the invention is particularly beneficial in conjunction with gene expression construct delivery in patients, and is amenable for a variety of monogenic diseases where targeted genes need to be switched on or off. The approach is especially amenable to direct, injectable therapies.

### EXAMPLES

The invention will now be further illustrated by way of the following non-limiting examples.

Unless otherwise indicated, commercially available reagents and standard techniques in molecular biological and biochemistry were used.

### Materials and Methods

The following procedures used by the Applicant are described in Sambrook, J. et al., 1989 supra.: analysis of restriction enzyme digestion products on agarose gels and preparation of phosphate buffered saline. General purpose reagents, oligonucleotides, chemicals and solvents were purchased from Sigma-Aldrich Quimica S A (Madrid, Spain). Enzymes and polymerases were obtained from New England Biolabs (NEB Inc.; c/o IZASA, S. A. Barcelona, Spain).

### Vector and Zinc Finger Peptide (ZFP) Construction for Binding GGGGCC Repeats

To build a zinc finger peptide (ZFP) framework that recognises GGGCCC repeat DNA sequences (which are found within expanded GGGGCC-repeats), a zinc finger scaffold based on the wild-type backbone sequence of the zinc finger region of wild-type human Zif268 was selected. Amino acid residues responsible for DNA target recognition (i.e. the ‘recognition sequence’, which essentially corresponds to the α-helical region of the framework) were first designed having regard to known zinc finger amino acid-nucleic acid recognition codes (e.g. Isalan et al. (1998) Biochemistry 37(35): 12026-12033; (WO 2012/049332)). Since in first aspects and embodiments of the invention it is intended for the zinc finger peptides to bind generally to contiguous 6 nucleotide sequences of GGGGCC, zinc finger peptides were designed to include pairs of adjacent zinc finger domains to target the GGG and GCC triplets.

1A. To bind the GGG triplet with the most N-terminal zinc finger domain, a selection of α-helical amino acid sequences (recognition sequences) were tested based around an initially designed RSDHLTR sequence (SEQ ID NO: 75).

1B. To bind the GCC triplet with the most N-terminal zinc finger domain, a selection of α-helical amino acid sequences (recognition sequences) were tested based around an initially designed DSSVLTR sequence (SEQ ID NO: 13).

2A. To bind the GCC triplet with a non-N-terminal zinc finger domain, a selection of α-helical amino acid sequences (recognition sequences) were tested based around an initially designed DSSVRKR sequence (SEQ ID NO: 14).

2B. To bind the GGG triplet with a non-N-terminal zinc finger domain, a selection of α-helical amino acid sequences (recognition sequences) were tested based around an initially designed RSDHLTR sequence (SEQ ID NO: 75).

By linking together an N-terminal most zinc finger domain sequence with a non-N-terminal zinc finger domain sequence selected from the pairs 1A and 2A or 1B and 2B first pairs of zinc finger domains forming fingers 1 and 2 of a zinc finger peptide are produced. The third and each subsequent zinc finger domain can then be designed based on alternating recognition sequences taken from 2A or 2B. In this way, poly-zinc finger peptides are produced to bind to repetitive 5′-GGGGCC . . . -3′ or 5′-GCCGGG . . . -3′ sequences.

The zinc finger peptides can be assembled according to any of Structures I to V (described above) to bind to contiguous GGGGCC repeat binding sites. Alternatively, as also described above, pairs of zinc finger sub-arrays can be linked by a long, flexible linker to form a longer zinc finger array peptide, and such adjacent pairs of zinc finger sub-arrays are capable of binding to discontinuous binding sites. In this way, a zinc finger sub-array targeting the binding site 5′-GGGGCC . . . -3′ can, for example, be joined to a zinc finger sub-array targeting the binding site 5′-GCCGGG . . . -3′ or vice versa.

Initially, poly-zinc finger peptides having 5, 6 and 11 zinc finger domains were produced and cloned ino a pUC57 vector (Genscript Corporation (Piscataway, NJ), with the names and sequences indicated in Table 6 below. This vector also included a T7 promoter, an N-terminal NLS (PKKKRKV for use in human cells, SEQ ID NO: 148; and RIRKKLR for use in mouse cells, SEQ ID NO: 150). Subcloning was performed similarly to that previously described in WO 2012/049332.

The zinc finger peptides were then subcloned into the mammalian expression vector pTarget (Promega); a 3xFLAG tag sequence was introduced by PCR at the N-terminus, and: for the 11-zinc finger peptide either the Kox-1 (human) or KRAB (mouse) transcription repression domain coding sequence was introduced at the C-terminus; or for the 5- and 6-zinc finger peptides the p65 ReIA activation domain of either the human or mouse coding sequence was introduced at the C-terminus. In all cases, a peptide linker sequence based on G and S amino acids was placed between the zinc finger peptide and the effector domain as described in WO 2012/049332.

### Design of ‘Mousified’ and ‘Humanised’ Zinc Finger Peptides

For in vivo experiments, in order to optimise the zinc finger repressor and activator peptides of the invention for use in mouse or human cells, respectively, the viral SV40 nuclear localisation signal (NLS; PKKKRKV, SEQ ID NO: 148) was replaced with a mouse primase p58 NLS (RIRKKLR; GenBank: BAA04203.1; SEQ ID NO: 150) or a human protein KIAA2022 NLS (PKKRRKVT; GenBank: NP_001008537.1; SEQ ID NO: 149) using native adjacent residues as linkers. In addition, the triple FLAG-tag reporter from ZF-Kox-1 was removed.

Zinc finger linker peptides were modified to make them as close as possible to canonical zinc finger linkers (e.g. TGEKP, TGQKP, SEQ ID NOs: 112 and 114), while retaining non-wild-type canonical-like linkers (e.g. TGSQKP, SEQ ID NO: 123) after every 2 fingers. Such an arrangement has been shown to be important for function of long zinc finger arrays (Moore et al. (2001), Proc. Natl. Acad. Sci. USA, 98: 1437-1441). Likewise, long, flexible linkers were introduced at appropriate spacings, i.e. after finger 5 (for the 11-finger construct) and after the last finger (e.g. finger 11 of the 11-finger construct) between the zinc finger domain and the repressor domain. These linkers can be reduced in length as much as possible while retaining functional separation of the respective domains in order to further reduce the amount of non-host sequence. Similarly, non-native functional residues in the zinc finger alpha helices (recognition sequences) were minimised by rational design in order to further reduce the amount of non-host sequence.

In addition, for human constructs, human Kox-1 was used in repressor proteins and, for mouse constructs the mouse KRAB repression domain from ZF87 (SEQ ID NO: 152; a.k.a. MZF22 (Abrink et al. (2001), Proc. Natl. Acad. Sci. USA, 98: 1422-1426.); refSeq_NM_133228.3) was used. The 1-76 amino acid KRAB-domain fragment of ZF87, when fused to Gal4 DNA-binding domain, has been previously reported to achieve similar levels of repression compared to Gal4-Kox-1 (Abrink et al. (2001), Proc. Natl. Acad. Sci. USA, 98: 1422-1426.) in mice.

### Tuning of Zinc Finger Peptides for Binding to GGGGCC Repeat Sequences

As described above, the zinc finger recognition sequences can be varied to ‘tune’ the peptides to bind to their target sites with an appropriate, desired specificity and affinity.

Accordingly, the initially designed/optimised recognition sequences of RSDHLTR (SEQ ID NO: 75), DSSVLTR (SEQ ID NO: 13), and DSSVRKR (SEQ ID NO: 14) were varied according to the sequences defined by SEQ ID NOs: 1 to 12, 133, 134, 135, 136, 184 to 186 for the 11-zinc finger peptide. More specifically, the generic formulae defined by SEQ ID NOs: 1, 2, 4 to 11 and 184 to 186 were used as a guide to design variants of the GCC-binding zinc finger domains, and the generic formulae defined by SEQ ID NOs: 3 and 12, 133, 134, 135, 136 were used as a guide to design variants of the GGG-binding zinc finger domains.

With respect to the 5- and 6-zinc finger peptides, the sequences were varied between SEQ ID NOs: 107 and 108 as a guide to design variants of the GCC-binding zinc finger domains, and the generic formulae defined by SEQ ID NOs: 109 and 110 were used as a guide to design variants of the GGG-binding zinc finger domains.

Phage ELISA experiments as previously described (Isalan et al. (2001), Nat. Biotechnol. 19: 656-660), were performed to guide the alpha-helix recognition sequence design to ensure that the modified sequences retained an appropriate binding strength and selectivity to GGGGCC or GCCGGG hexanucleotide repeat sequences.

### In Vitro Gel Shift Assays

Based on the pUC57 vector zinc finger constructs, appropriate forward and reverse primers were used to generate PCR products for in vitro expression of the ZFP, using the TNT T7 Quick PCR DNA kit (Promega). Double stranded DNA probes with different numbers of CAG repeats were produced by Klenow fill-in as described in WO 2012/049332. 100 ng of double stranded DNA was used in a DIG-labeling reaction using Gel Shift kit, 2^{nd} generation (Roche), following the manufacturer's instructions. For gel shift assays, 0.005 pmol of DIG-labelled probe were incubated with increasing amounts of TNT-expressed protein in a 20 μl reaction containing 0.1 mg/ml BSA, 0.1 μg/ml polydl:dC, 5% glycerol, 20 mM Bis-Tris Propane, 100 mM NaCl, 5 mM MgCI₂, 50 mg/ml ZnCl₂, 0.1% NonidetP40 and 5 mM DTT for 1 hour at 25° C. Binding reactions were separated in a 7% non-denaturing acrylamide gel for 1 hour at 100 V, transferred to a nylon membrane for 30 min at 400 mA, and visualisation was performed following manufacturer's instructions.

### Cell Culture and Gene Delivery

The cell line HEK-293T (ATCC) was cultured in 5% CO₂ at 37° C. in DMEM (Gibco) supplemented with 10% FBS (Gibco). Qiagen purified DNA was transfected into cells using Lipofectamine 2000 (Invitrogen) according to the manufacturer's instructions. Briefly, cells were plated onto 10 mm wells to a density of 50% and 70 ng of reporter plasmid, 330 ng of ZFP expression plasmid and 2 μl of Lipofectamine 2000 were mixed and added to the cells. Cells were harvested for analysis 48 hours later.

STHdh+/Hdh+ and STHdhQ111/Hdh 111 cells (gift from M.E. MacDonald) were cultured in 5% CO₂ at 33° C. in DMEM supplemented with 10% FBS (Gibco) and 400 μg/ml G418 (PAA). Cells were infected with retroviral particles using the pRetroX system (Clontech) according to the manufacturer's instructions.

### Flow Cytometry Analysis

Cells were harvested 48 hours post-transfection and analysed in a BD FACS Canto Flow cytometer using BD FACSDiva software.

### Western Blot

293T cells were harvested 48 hours post-transfection in 100 μl of 2xSDS loading dye with Complete protease inhibitor (Roche). 20 μl of sample was separated in 4-15% Criterion Tris-HCl ready gels (BioRad) for 2 hours at 100V, transferred to Hybond-C membrane (GE Healthcare) for 1 hour at 100V. Proteins were detected with either the primary antibody anti β-actin (Sigma A1978) at 1:3000 dilution or anti-EGFP (Roche) at 1:1500 dilution and with a peroxidase-conjugated donkey anti-mouse secondary antibody (Jackson ImmunoResearch) at 1:10000 dilution. Visualisation was performed with ECL system (GE Healthcare) using a LAS-3000 imaging system (Fujifilm). STHdh cells were trypsinised and harvested in PBS containing Complete protease inhibitor (Roche). Cells were resuspended in RIPA buffer (1% TritonX-100, 1% sodium deoxycholate, 40 mM Tris-HCl, 150 mM NaCl, 0.2% SDS, Complete), incubated in ice for 15 min, and were centrifuged at 13000 rpm for 15 min. The supernatant was collected and protein concentration was determined using BioRad's Dc protein assay. 60 μg of protein was separated in a 5% Criterion Tris-HCl ready gel (BioRad) for 2 hours at 100V, transferred using iBlot Dry Blotting System (Invitrogen) for 8 min and endogenous Htt protein was detected with anti-Huntingtin primary antibody (Millipore MAB2166) at a 1:1000 dilution.

### Production of Adeno-Associated Viral Vector

rAAV2/1 vectors containing zinc finger peptides/effectors of the invention as described in WO 2017/077329, e.g. containing a pCAG promoter (CMV early enhancer element and the chicken beta-actin promoter) and WPRE (Woodchuck post-translational regulatory element), can be produced, for example, at the Centre for Animal Biotechnology and Gene Therapy of the Universitat Autonoma of Barcelona (CBATEG-UAB; see also Salvetti et al. (1998) Hum. Gene Ther. 9: 695-706). Recombinant virus can be purified by precipitation with PEG8000 followed by iodixanol gradient ultracentrifugation with a final titre of approx 10¹² genome copies/ml.

### Animals—C9-500 Transgenic Mice

For this study we used the transgenic expansion repeat model and wild-type (WT) mice. For example, for C9orf72 we used C9-500 mice (Jackson) which have approx. 500 GGGGCC expansion repeats. Hemizygotes display neurodegeneration, RAN protein and both sense and antisense RNA foci which are all characteristic pathological markers of both ALS and FTD.

Alternative models include the C919 BAC transgenic mouse line (C9B77) (Jackson) with approx. 90/450 repeat alleles. In practice, any suitable C9orf72 expansion model may be used.

All animal experiments were conducted in accordance with Directive 86/609/EU of the European Commission, the Animals (Scientific Procedures) 1986 Act of the United Kingdom, and following protocols approved by the Ethical Committee of the Barcelona Biomedical Research Park and the Animal Welfare and Ethical Review Body of Imperial College London. The predicted number of mice for each experiment is given in Table 5 based on HD ZFP studies.

TABLE 5
Summary of number of mice injected with lead ZF-1 and ZF-2
(bind GGGGCC expansion repeats, up- and down-regulating
the targets, respectively), GFP or PBS.

| | | | Weeks post- | |
|---|---|---|---|---|
| Experiment | Treatment | Genotype | injection | n |
| Histology | ZF-1 | WT | 4 | 4 |
| analysis: | | | 6 | 4 |
| inflammatory | ZF-2 | | 4 | 4 |
| responses and | | | 6 | 4 |
| neuronal loss | GFP | | 4 | 4 |
| | | | 6 | 4 |
| | PBS | | 4 | 3 |
| | | | 6 | 3 |
| Gene | ZF-1 | C9-500 | 2 | 3 |
| expression | | | 4 | 3 |
| analysis | | | 6 | 3 |
| | ZF-2 | C9-500 | 2 | 6 |
| | | | 4 | 7 |
| | | | 6 | 7 |

### Stereotaxic Surgery

Briefly, mice are anesthetised with isofluorane for any surgical application and fixed on a stereotaxic frame if necessary. Buprenorphine is injected at 8 μg/kg to provide analgesia.

AAVs are injected bilaterally or unilaterally (depending on the study) into various brain regions using a 10 μl Hamilton syringe at a rate of 0.25 μl/min controlled by an Ultramicropump (World Precision Instruments). For each injection, a total volume of 1.5 to 3 μl (approx. 2×10⁹ genomic particles) or 1.5 μl PBS is injected. For example, a two-step administration may be performed as follows: 1.5 μl are injected at −3.0 mm DV, the needle is let to stand for 3 minutes in position, and then the other half is injected at −2.5 mm DV, as in case of intra-striatal injections.

In some studies, mice are injected only in one hemisphere with AAV expressing the test protein (either zinc finger or GFP control protein), or with PBS as a negative control.

Mice are sacrificed at different ages for posterior analysis by RT-PCR, immunohistochemistry or western blot; typically at 2, 4 or 6 weeks after administration of agent.

### Animal Behavioral Tests

Behavioural monitoring typically commences at 4 weeks of age and tests take place bimonthly until 11 weeks of age. All the experiments are performed double-blind with respect to the genotype and treatment of the mice.

Examples of Behavioural Tests that May be Performed:

Clasping behaviour is checked by suspending the animal by the tail for 20 seconds. Mice clasping their hindlimbs are given a score of 1, and mice that do not clasp are given a score of 0.

Grip strength is measured by allowing the mice to secure to a grip strength meter, then pulling gently by the tail. The test is repeated three times and the mean and maximum strength recorded.

For the accelerating rotarod test, mice are trained at 4 weeks of age to stay on the rod at a constant speed of 4 rpm until they reach a criterion of 3 consecutive minutes on the rod. In the testing phase, mice are put on the rotarod at 4 rpm and the speed is constantly increased for 2 minutes until 40 rpm is reached. The assay is repeated twice and the maximum and average latency taken to fall from the rod is recorded.

For the open field test, mice are put in the centre of a white methacrylate squared open field (70×70 cm), illuminated by a dim light (70 lux) to avoid aversion, and their distance travelled, speed and position is automatically measured with a video tracking software (SMART system, Panlab, Spain). Other activities, such as rearing, leaning, grooming and number of faeces are monitored de visu.

For the paw print test, mice hindpaws are painted with a non-toxic dye and mice are allowed to walk through a small tunnel (10×10×70 cm) with a clean sheet of white paper on the floor. Footsteps are analysed for three step cycles and three parameters measured: (1) stride length—the average distance between one step to the next; (2) hind-base width—the average distance between left and right hind footprints; and (3) splay length—the diagonal distance between contralateral hindpaws as the animal walks.

### Examples of Molecular Analysis:

qRT-PCR

For studies of target gene expression in vivo, mice are humanely killed by cervical dislocation. As rapidly as possible, they are decapitated and various brain regions are dissected on ice and immediately frozen in liquid nitrogen for later RNA extraction.

RNA is prepared with an RNeasy kit (Qiagen) and reversed transcribed with Superscript Ill (Invitrogen). Real Time PCR is performed in a LightCycler® 480 Instrument (Roche) using LightCycler® 480 Taqman master mix (Roche). A specific set of primers and probes is used to assess molecular readouts of disease progression.

### Immunohistochemistry

Mice are transcardially perfused with PBS followed by formalin 4% (v/v). Brains are removed and post-fixed overnight at 4° C. in formalin 4% (v/v). Brains are then cryoprotected in a solution of sucrose 30% (w/v), at 4° C., until they sink. Brains are then frozen and sliced with a freezing microtome in six parallel coronal series of 40 μm (distance between slices in each parallel series: 240 μm). The indirect ABC procedure is employed for the detection of the neuronal marker Neu-N(1:100, MAB377 Millipore) in the first series; the reactive astroglial marker GFAP (1:500, Dako) in the second series; and the microglial marker Iba1 (1:1000, Wako) in the third series. Briefly, sections are blocked with 2% (v/v) Normal Goat Serum (NGS, Vector Laboratories) in PBS-Triton 100 0.3% (v/v) and endogenous peroxidase activity blocked with 1% (v/v) hydrogen peroxide (H₂O₂) in PBS for 30 minutes at room temperature. This is based on similar approach used to assess the therapeutic effect of ZFP on disease progression in Huntington's Disease (HD).

Subsequently, sections are incubated for 30 minutes at room temperature in: (i) primary antibody (at the concentration indicated above) in PBS with 0.3% (v/v) Triton X100 and 2% (v/v) NGS; (ii) biotinylated secondary antibody in the same buffer; and (iii) avidin-biotin-peroxidase complex (ABC Elite kit Vector Laboratories) in PBS-Triton X-100 0.3% (v/v). Sections are washed for 3×10 min in PBS and peroxidase activity is revealed with SIGMAFAST-DAB (3,3′-Diaminobenzidine tetrahydrochloride, Sigma-Aldrich) in PBS for 5 min. Sections are rinsed and mounted onto slides, cleared with Histoclear (Fisher Scientific) and cover-slipped with Eukitt (Fluka).

The fourth GFP-injected series is mounted onto slides and covered with Mowiol (Sigma-Aldrich) for fluorescence analysis.

### Image Analysis

### Determination of the Volume of Injection:

Five coronal slices per GFP-injected hemisphere from bregma 1.5 mm levels, separated by 240 μm, are photographed with a digital camera attached to a macrozoom microscope (Leica). The contours around the GFP-expressing area and dorsal striatum are manually defined and the area is measured with ImageJ software (National Institute of Health, USA). Volume is calculated as area per distance between slices, according to the Cavalieri principle (Oorschot (1996), J. Comp. Neurol.; 366: 580-599).

### Determination of 0.D. For GFAP and Iba1 Stainings:

Four coronal slices per mouse and hemisphere covering the striatum from bregma 1.5 mm levels are selected, and a region of interest of 670×897 μm² in the middle of the dorsal striatum is captured with a 10× objective using a digital camera attached to a microscope (Leica DMIRBE). The O.D. of the areas is measured with ImageJ, the mean density per hemisphere calculated, and O.D. for GFAP and Iba1 of control hemispheres is subtracted from the injected hemisphere.

### Determination of the Neuronal Density of the Different Brain Region on Striata as an Example:

Cell density is calculated using an adaptation of the unbiased fractionator method (Oorschot (1996), J. Comp. Neurol.; 366: 580-599). Four coronal slices per mouse and hemisphere covering the striatum from bregma 1.5 mm levels are selected, and a region of interest of 447×598 μm² in the middle of the dorsal striatum is captured with a 15× objective, using a digital camera attached to a microscope (Leica DMIRBE). A grid image leaving 16 squares of 35×35 μm² is superimposed onto the pictures, and a person (blinded to sample treatment) counts the number of stained nuclei.

### Statistical Analysis

Data are analysed using the StatPlus package for Excel (Microsoft) and IBM SPSS Statistics 22. To test the inflammatory response, the difference of O.D. of the injected hemisphere versus the control hemisphere is calculated, and a Student's t test is performed against the no difference value (0).

For neuronal density, a paired Student's t test of neuronal density in the injected hemisphere, versus the control hemisphere, is performed. Neuronal density is analysed across contralateral hemispheres with ANOVA, followed by post-hoc comparisons with the contralateral hemispheres of the PBS samples. To test repression, the percentage of mutant gene of interest in the injected brain is calculated with respect to the control hemisphere, and a one sample Student's t test against the no repression value (100%) is performed. To ensure a fair comparison between injected and contralateral hemispheres, only mice with <1% ZF expression in the contralateral hemisphere, relative to the injected hemisphere, are used for statistical analyses. To test the correlation between RNA levels of the different genes and ZF expression, a linear regression test is applied. To test expression levels across different times post-injection, a one-way ANOVA is performed. All significance values may, for example, be set at p=0.05.

### Example 1

### Design of Zinc Finger Peptide (ZFP) Arrays to Bind GGGGCC or GCCGGG Repeats

It is known that zinc finger domains can be concatenated to form multi-finger (e.g. 6-finger) chains (Moore et al. (2001) Proc. Natl. Acad. Sci. USA 98(4): 1437-1441; and Kim & Pabo (1998) Proc. Natl. Acad. Sci. USA 95(6): 2812-2817). Our previous study, see WO 2012/049332, was the first to report on the systematic exploration of the binding modes of different-length ZFP to long repetitive DNA tracts. In this earlier study, rational design was used to construct a zinc finger domain (ZF×Hunt) that would bind the 5′- GC(A/T) -3′ sequence in double stranded DNA.

In contrast to this earlier study, the poly-zinc finger peptides of this invention are adapted to bind to hexanucleotide repeat sequences. Therefore, this earlier teaching of how to produce extended arrays of poly-zinc finger peptides was adapted to provide extended arrays of zinc finger binding pairs, to bind the hexanucleotide repeat sequences 5′-GGG GCC-3′ or 5′-GCC GGG-3′ (see Materials and Methods above and FIGS. 1, 2A and 2B).

Both 5′-GGG GCC-3′ and 5′-GCC GGG-3′ repeat sequences were targeted as part of the zinc finger peptide ‘tuning’ process to understand and manipulate binding interactions between different zinc finger peptides and their respective target sites.

To try to avoid the zinc finger peptides of the invention losing their register with cognate DNA (after 3 or more adjacent fingers and 9 contiguous base pairs of double helical DNA), the linker sequences were carefully designed. In particular, the length of the linkers between adjacent zinc fingers in the arrays was modulated. In this way, the register between the longer arrays of zinc finger peptides, especially on binding to dsDNA, could be optimised. Using structural considerations, it was decided to periodically modify the standard canonical linker sequences in the arrays. Therefore, canonical-like linker sequences containing an extra Gly (or Ser) residue were included in the long zinc finger array after every 2-zinc fingers, and flexible (up to 29-residue) linker sequences were included in the long zinc finger array after every 5-/6-fingers. In this way, different numbers of zinc fingers could be tested for optimal length-dependent discrimination. Sequences of the various zinc finger peptides having 5-, 6- and 11-zinc finger domains arranged in tandem are indicated in the table below. 5- and 6- zinc finger peptides are designed for use as transcriptional activators in order to increase expression of a wild-type gene sequence; whereas 11-zinc finger peptides are designed for use as transcriptional repressors in order to reduce expression of mutant target genes. 11-zinc finger peptides are ‘tuned’ in order to disrupt optimal binding interaction with the target mutant nucleic acid sequence in order to reduce off/non-target interactions of the repressor protein—e.g. with the wild-type gene sequence.

TABLE 6
Zinc finger peptide framework amino acid sequences of
humanised or mousified 5-, 6- and 11-zinc finger domains of
the invention for binding to 5-GGG-GCC-3′ repeat nucleic
acid sequences. Nucleic acid-binding recognition sequences
are underlined and linker sequencesare shown in bold.

```
ZF11xALS1 amino acid sequence (SEQ ID NO: 166):
YACPVESCDRRFS DSSVLTR HIRIH TGSQKP
    FQCRICMRNFS RSDHLTR HIRTH TGEKP
    FACDICGRKFA DSSVRKR HTKIH TGSQKP
    FQCRICMRNFS RSDHLTR HIRTH TGEKP
    FACDICGRKFA DSSVRKR HTKIH LRQKDGGGGSGGGGSGGGGSQKP
    FQCRICMRNFS RSDHLTR HIRTH TGEKP
    FACDICGRKFA DSSVRKR HTKIH TGSQKP
    FQCRICMRNFS RSDHLTR HIRTH TGEKP
    FACDICGRKFA DSSVRKR HTKIH TGSQKP
    FQCRICMRNFS RSDHLTR HIRTH TGEKP
    FACDICGRKFA DSSVRKR HTKIH
ZF11xALS2 amino acid sequence (SEQ ID NO: 167):
YACPVESCDRRFS DNRDLTR HIRIH TGSQKP
    FQCRICMRNFS RSDHLTR HIRTH TGEKP
YACPVESCDRRFS DNRDLTR HIRIH TGSQKP
    FQCRICMRNFS RSDHLTR HIRTH TGEKP
YACPVESCDRRFS DNRDLTR HIRIH TGQKPGGGGSGGGGSGGGGSQKP
    FQCRICMRNFS RSDHLTR HIRTH TGEKP
YACPVESCDRRFS DNRDLTR HIRIH TGSQKP
    FQCRICMRNFS RSDHLTR HIRTH TGEKP
YACPVESCDRRFS DNRDLTR HIRIH TGSQKP
    FQCRICMRNFS RSDHLTR HIRTH TGEKP
YACPVESCDRRFS DNRDLTR HIRIH
ZF11xALS3 amino acid sequence (SEQ ID NO: 168):
YACPVESCDRRFS TREDLTR HIRIH TGSQKP
    FQCRICMRNFS RSDHLTR HIRTH TGEKP
    FACDICGRKFA TREDRKR HTKIH TGSQKP
    FQCRICMRNFS RSDHLTR HIRTH TGEKP
    FACDICGRKFA TREDRKR HTKIH LRQKDGGGGSGGGGSGGGGSQKP
    FQCRICMRNFS RSDHLTR HIRTH TGEKP
    FACDICGRKFA TREDRKR HTKIH TGSQKP
    FQCRICMRNFS RSDHLTR HIRTH TGEKP
    FACDICGRKFA TREDRKR HTKIH TGSQKP
    FQCRICMRNFS RSDHLTR HIRTH TGEKP
    FACDICGRKFA TREDRKR HTKIH
ZF5xALS1 amino acid sequence (SEQ ID NO: 169):
YACPVESCDRRFS DSSVLTR HIRIH TGSQKP
    FQCRICMRNFS RSDHLTR HIRTH TGEKP
    FACDICGRKFA DSSVRKR HTKIH TGSQKP
    FQCRICMRNFS RSDHLTR HIRTH TGEKP
    FACDICGRKFA DSSVRKR HTKIH
ZF6xALS1 amino acid sequence (SEQ ID NO: 170):
FQCRICMRNFS RSDHLTR HIRTH TGEKP
FACDICGRKFA DSSVRKR HTKIH TGSQKP
FQCRICMRNFS RSDHLTR HIRTH TGEKP
FACDICGRKFA DSSVRKR HTKIH TGSQKP
FQCRICMRNFS RSDHLTR HIRTH TGEKP
FACDICGRKFA DSSVRKR HTKIH
```

### Example 2

### Binding of Zinc Finger Peptides to DNA Target Sequences In Vitro

To show that the zinc finger peptides of Example 1 are capable of binding to GGGGCC repeat sequences, in vitro gel shift assays can be carried out as follows.

The zinc finger peptide arrays containing 5-, 6- and 11-zinc finger domains of Example 1 were constructed and tested in gel shift assays for binding to double-stranded GGGGCC repeat sequence probes.

All zinc finger peptides of Example 1 demonstrated the ability to bind poly 5′-GGGGCC-3′ DNA probes in vitro (data not shown). Furthermore, it is expected that the longer zinc finger peptides having 11-fingers and designed for optimal binding interactions with the target sites bind most specifically and efficiently to the longer repeat sequence target sites; whereas the shorter zinc finger peptides having 5- or 6-fingers exhibit less preference for the length of the target site.

‘Tuning’ of the optimally designed 11-zinc finger peptides by substitution of an optimal amino acid residue at one or more of positions −1, 3 and 6 of the zinc finger recognition helices of the peptides with Gly residues was demonstrated to weaken the binding affinity of the 11-finger peptides for their target 5′-GGGGCC-3′ DNA probes. Therefore, it is shown that Gly (or Ala) substitution of appropriately selected amino acid binding residues with Gly or Ala can be used to (de)tune the binding affinity of a poly-zinc finger peptide and, thus, to desirably control the strength of the binding interaction according to preference. Accordingly, incremental increases in the number of Gly and/or Ala substitutions, which are expected not to contribute to (or otherwise to weaken) the binding interaction between a zinc finger domain and a target nucleic acid sequence, are expected to incrementally weaken the binding affinity of a zinc finger peptide for its target sequence.

In this way, the binding affinity of an 11-zinc finger peptide according to the present invention can be reduced so as not to out-compete a shorter (e.g. 5- or 6-zinc finger peptide) for the same target binding site.

### Example 3

### Repression of Reporter Genes In Vivo

The intracellular activity of the zinc finger peptides of Example 1 having 6- and 11-zinc finger domains can be tested in vivo using reporter vectors with different numbers of 5′-GGGGCC-3′ repeats in frame with EGFP. To assess whether there were any non-specific effects caused by the zinc finger proteins, an HcRed reporter is cloned in a different region of the same vector, under an independent promoter.

HEK293T cells were transiently cotransfected with the indicated reporter and zinc finger peptide expression vectors, in which zinc finger expression was driven by CMV promoters. Three sets of assays can be carried out to test reporter expression levels: quantifying EGFP and HcRed fluorescent cells using Fluorescence-Activated Cell Sorting (FACS); EGFP protein levels in Western blots; and EGFP and HcRed mRNA levels in qRT-PCR.

To test the potential for even stronger repression, the KRAB repression domain Kox-1 (Groner et al. PLoS Genet 6(3): e1000869) was fused to the C-terminus of each zinc finger protein (Human Kox-1 domain amino acid sequence: SEQ ID NO: 151; Mouse KRAB domain amino acid sequence from ZF87: SEQ ID NO: 152), and reporter gene repression is expected to be significantly stronger than without the dedicated repressor domain. Repression is also expected to be proportional to zinc finger peptide and nucleotide-repeat number, favouring gene repression with respect to extended poly-zinc finger peptides of 11 zinc finger domains targeted against expanded GGGGCC-repeat sequences that are associated with pathogenic genes.

Suitable zinc finger-effector domain amino acid linker sequences may, for example, be selected from the sequences of SEQ ID NO: 153, 154 and 155.

### Example 4

### Competition Binding Assays for Repression of Long GGGGCC-Repeats

For human therapeutic use, ZFPs should preferentially repress long mutant GGGGCC-alleles, but have less effect on short wt alleles (e.g. 2- to 23-repeats; the length of wt C9ORF72 repeats varies in the human population, but is usually in this range). Therefore, a competition assay can be developed to measure length-preference directly. HEK293T cells can be cotransfected with three plasmids: (1) an EGFP reporter vector containing a GGGGCC repeat sequence, (2) an mCherry reporter vector containing a GGGGCC repeat sequence, together with (3) various zinc finger peptide expressing vectors according to the invention, which express one of the zinc finger peptides of Example 1.

The relative expression of the two reporters can be measured by FACS (EGFP or mCherry positive cells).

All constructs are expected to demonstrate active repression of the longer GGGGCC-repeat reporters. It is also expected that the results will demonstrate that longer GGGGCC-repeats are preferentially targeted and repressed by the extended poly-zinc finger peptide of 11 finger domains.

As the inventors have discussed with respect to their previous work (e.g. WO 2012/049332; WO 2017/077329), it is possible that the selective inhibition of longer target sequences may be at least partly due to a mass action effect (i.e. longer GGGGCC-repeats contain more potential binding sites for the zinc finger peptides). However, it is also possible that in the case of longer arrays of zinc fingers and shorter GGGGCC-repeat sequences, the peptides may compete with each other for the binding site, and as a consequence, the longer arrays of zinc fingers may bind more transiently or more weakly (e.g. to partial or sub-optimal recognition sequences).

### Example 5

### Zinc Finger Recognition Sequence Designs for GCCGGG- or GGGGCC-Repeat Binding Affinity ‘Tuning’

The DSSVLTR (SEQ ID NO: 13) and RSDHLTR (SEQ ID NO: 75) zinc finger recognition helix sequences were rationally designed, as described elsewhere in this document, in order to provide optimal binding interactions to the GCCGGG hexanucleotide repeat sequence in double-stranded DNA, so as to provide poly-zinc finger peptides that bind with high affinity and specificity to pathogenic GGGGCC-repeat sequences in genomic DNA. In this way, it is possible to provide zinc finger repressor proteins for specific targeting and downregulation of pathogenic genes associated with diseases such as ALS and FTD.

However, GGGGCC-repeat sequences are also associated with wild-type gene sequences—in particular C9ORF72—albeit in much fewer repeat lengths; and it is thought that haploinsufficiency of C9ORF72 gene expression and the loss of function of the C9ORF72 gene product may in fact also contribute to disease pathology. Therefore, the inventors consider it desirable to reduce, minimise or eliminate any unintended repression of wild-type gene expression, which may undesirably further contribute to the predicted haploinsufficiency.

As discussed, the inventors have hypothesised that wild-type C9ORF72 gene expression may be upregulated using relatively short poly-zinc finger activator peptides (e.g. from 4 to 8 zinc fingers, and more suitably 5, 6 or 7 zinc fingers) having transcriptional activation domains associated therewith, which are capable of binding with high affinity to wild-type GGGGCC repeat sequences of less than 30 repeats, but which show little or no preference for the length of the GGGGCC repeat sequence length. In this way, the desirable gene product may be selectively increased while not over-proportionally increasing the expression levels of the pathogenic gene product. In conjunction with this, the inventors further hypothesised that the unintentional upregulation of the pathogenic gene through undesirable binding of the relatively short zinc finger activator peptides (e.g. having 3 to 8, 4 to 7, 5, 6 or 7 fingers) to pathogenic expanded GGGGCC-repeat sequences could be mitigated against by providing, in conjunction with the activator peptide of the invention, extended poly-zinc finger repressor peptides (having from 8 to 32 zinc fingers, such as from 8 to 18, e.g. 10, 11 or 12 zinc finger domains), which preferentially target and bind to the expanded GGGGCC-repeat sequences of the pathogenic genes. In this way, pathogenic genes are preferentially targeted by extended poly-zinc finger repressor peptides of 8 or more zinc finger domains (preferably 11 or 12 zinc finger domains), and poly-zinc finger activator peptides of 8 or less zinc finger domains (preferably 5 or 6 zinc finger domains), which are outcompeted at pathogenic sites, preferentially target wild-type gene sequences.

It is further considered that unintentional repression of wild-type gene expression can be reduced, minimised or eliminated through a combination of: (i) the length of the extended poly-zinc finger repressor proteins, which preferentially target longer, expanded GGGGCC repeat sequences (e.g. for steric reasons); and (ii) by reducing the binding strength of the zinc finger recognition sequences of the extended poly-zinc finger repressor proteins for each GGGGCC (or GCCGGG) target site.

The inventors have previously shown that it is possible to vary zinc finger domain backbone residues and zinc finger linker sequences without adversely affecting useful properties, such as viral packaging (WO 2017/077329, Example 10) and nucleic acid binding capability, while at the same time reducing host immunogenicity (WO 2017/077329, Example 11). This Example describes recognition sequence variations to selectively reduce the strength of the binding interaction between zinc finger repressor proteins of the invention and GGGGCC-repeat sequences of 23 of less repeats, without adversely affecting zinc finger specificity and gene targeting.

As previously described, in order to improve host cell expression, longevity and generally to reduce toxicity and immunogenicity in host organisms, it is desirable to minimise the number of non-wild-type peptide sequences that result from the incorporation of sequence variability and differences in peptide sequence compared to endogenous protein sequences. In particular, the number of potentially ‘foreign’ epitopes that may be detected by an animal body following administration of expression constructs of the invention, such as AAV vectors, should be reduced.

One strategy, therefore, where possible, is to focus the redesign of recognition sequences on alpha-helix positions that already vary from the wild-type sequence, as indicated in the below.

In a first set of experiments, the zinc finger pair for binding the sequence 5′-GGG GCC-3′ was varied as indicated below:

| | |
|---|---|
| Binding Site: | C C G G G G |
| Finger Number: | F1 F2 etc. |
| Optimal Sequence: | DSSVLTR RSDHLTR |
| Variants: | A D K A S K |
| | G E G G A G |
| | A |
| | G |

Alpha-helix positions −1, 2, 3 and 6 (shown in bold above) are already altered from the endogenous gene sequence in order to target the GGGGCC repeat. The potential variability of this embodiment is defined by SEQ ID NO: 4: (D/A/G)SS(V/D/E/A/G)LT(R/K/G) for finger 1, and SEQ ID NO: 3: RS(D/A/G)HL(T/S/A)(R/K/G) for finger 2.

Similar tests were performed based around the potential recognition sequence of SEQ ID NO: 6 (D/A/G)SS(V/D/E/A/G)RK(R/K/G) and SEQ ID NO: 3 respectively for finger pairs 3 and 4, 5 and 6 etc. of a poly-zinc finger peptide.

In a second set of experiments, the zinc finger pair for binding the sequence 5′-GGG GCC-3′ was varied as indicated below:

| | |
|---|---|
| Binding Site: | C C G G G G |
| Finger Number: | F1 F2 etc. |
| Optimal Sequence: | DSSVLTR RSDHLTR |
| Variants: | ANRD K A SK |
| | GREE G G AG |
| | T AA |
| | G GG |
| | S I |
| | L |
| | S |
| | T |

The potential variability of this embodiments is defined by SEQ ID NO: 5: (D/A/G/T/V/S)(S/N/R)(S/R/E/A/G)(V/D/E/A/G/I/L/S/T)LT(R/K/G) for finger 1, and SEQ ID NO: 3: RS(D/A/G)HL(T/S/A)(R/K/G) for finger 2.

Similar tests were performed based around the potential recognition sequence of SEQ ID NO: 7 (D/A/G/T/V/S)(S/N/R)(S/R/E/A/G)(V/D/E/A/G/I/L/S/T)RK(R/K/G) and SEQ ID NO: 3 respectfully, for finger pairs 3 and 4, 5 and 6 etc. of a poly-zinc finger peptide.

According to the above formulae, a series of poly-zinc finger peptides (e.g. having 8, 11 or 12 zinc finger domains) were created to test how the sequence changes from the perceived ‘optimal’ sequence would affect factors such as binding affinity, specificity and binding competition with shorter poly-zinc finger peptides designed to bind to the same target sequences through the originally designed, more ‘optimal’ recognition sequences based on the expected nucleic acid to amino acid side chain interactions.

As the number of zinc finger domains increases, the number of G residues may be increased to reduce the binding strength of the zinc finger peptides against shorter nucleic acid target sites.

Thus, in one set of zinc finger peptide variants, the D residue in the 2 position of each even-numbered zinc finger domain (F2, F4 etc.) is replaced with G to weaken the binding interaction of the zinc finger peptide. V and A residues may also be used in this position.

In another set of zinc finger peptide variants, the D residue in the −1 position of each odd-numbered zinc finger domain (F1, F3 etc.) is replaced with A to weaken the binding interaction of the zinc finger peptide. V and G residues may also be used in this position in alternatives. At the same time, or separately, in the even-numbered zinc finger domains, the V residue at the 3 position may be varied—e.g. to an E residue, in order to balance the overall charge of the two-finger peptide recognition sequence.

In another set of zinc finger peptide variants, separately or in conjunction with the above variants, the R residue at the 6 position in each even-numbered (and/or in each odd-numbered) finger may be varied to K to slightly weaken the binding interaction with a guanine base of the target sequence. In other variants a G amino acid may alternatively be used to further reduce the binding strength of the zinc finger peptide.

The inventors have hypothesised that the weaker the binding mode of the poly-zinc finger peptides of the invention against the intended target site, the higher will be the necessary zinc finger protein concentration in vivo to cause the desired effector function (repression), but also the longer the GGGGCC expansion that will be required to ensure effective binding and repression by the variant zinc finger peptide. Thus, the effectiveness of the zinc finger (repressor) proteins of the invention can be ‘tuned’ by a combination of binding strength reduction and protein expression level in order to generate the desired technical response.

Exemplary zinc finger peptide sequence variants—especially for use in zinc finger repressor proteins—are illustrated in the table below.

TABLE 7
'Tuned' zinc finger peptide framework amino acid sequences
of humanised or mousified 11-zinc finger domains of the
invention for binding to mutant 5-GGG-GCC-3' repeat nucleic
acid sequences. Nucleic acid-binding recognition sequences
are underlined and linker sequences are shown in bold.
'Tuned' residues to deliberately alter binding affinity to
target sequences are shown in bold and underlined. In any
of the above tuned recognition sequences
A residues may be replaced with G residues and vice versa.

```
ZF11xALS1-TV1 amino acid sequence (SEQ ID NO: 171):
YACPVESCDRRFS DSSVLTR HIRIH TGSQKP
    FQCRICMRNFS RSGHLTR HIRTH TGEKP
    FACDICGRKFA DSSVRKR HTKIH TGSQKP
    FQCRICMRNFS RSGHLTR HIRTH TGEKP
    FACDICFGKFA DSSVRKR HTKIH LRQKDGGGGSGGGGGGGGSQKP
    FQCRICMRNFS RSGHLTR HIRTH TGEKP
    FACDICGRKFA DSSVRKR HTKIH TGSQKP
    FQCRICMRNFS RSGHLTR HIRTH TGEKP
    FACDICGRKFA DSSVRKR HTKIH TGSQKP
    FQCRICMRNFS RSGHLTR HIRTH TGEKP
    FACDICGRKFA DSSVRKR HTKIH
ZF11xALS1-TV2 amino acid sequence (SEQ ID NO: 172):
YACPVESCDRRFS ASSELTR HIRIH TGSQKP
    FQCRICMRNFS RSGHLTR HIRTH TGEKP
    FACDICGRKFA ASSERKR HTKIH TGSQKP
    FQCRICMRNFS RSGHLTR HIRTH TGEKP
    FACDICGRKFA ASSERKR HTKIH LRQKDGGGGSGGGGSGGGGSQKP
    FQCRICMRNFS RSGHLTR HIRTH TGEKP
    FACDICGRKFA ASSERKR HTKIH TGSQKP
    FQCRICMRNFS RSGHLTR HIRTH TGEKP
    FACDICGRKFA ASSERKR HTKIH TGSQKP
    FQCRICMRNFS RSGHLTR HIRTH TGEKP
    FACDICGRKFA ASSERKR HTKIH
  ZF11xALS1-TV3 amino acid sequence (SEQ ID NO: 173):
YACPVESCDRRFS ASSELTR HIRIH TGSQKP
    FQCRICMRNFS RSGHLTK HIRTH TGEKP
    FACDICGRKFA ASSERKR HTKIH TGSQKP
    FQCRICMRNFS RSGHLTK HIRTH TGEKP
    FACDICGRKFA ASSERKR HTKIH LRQKDGGGGSGGGGSGGGGSQKP
    FQCRICMRNFS RSGHLTK HIRTH TGEKP
    FACDICGRKFA ASSERKR HTKIH TGSQKP
    FQCRICMRNFS RSGHLTK HIRTH TGEKP
    FACDICGRKFA ASSERKR HTKIH TGSQKP
    FQCRICMRNFS RSGHLTK HIRTH TGEKP
    FACDICGRKFA ASSERKR HTKIH
ZF11xALS1-TV4 amino acid sequence (SEQ ID NO: 174):
YACPVESCDRRFS DSGDLTR HIRIH TGSQKP
    FQCRICMRNFS RSGHLTK HIRTH TGEKP
    FACDICGRKFA DSGDRKR HTKIH TGSQKP
    FQCRICMRNFS RSGHLTK HIRTH TGEKP
    FACDICGRKFA DSGDRKR HTKIH LRQKDGGGGSGGGGSGGGGSQKP
    FQCRICMRNFS RSGHLTK HIRTH TGEKP
    FACDICGRKFA DSGDRKR HTKIH TGSQKP
    FQCRICMRNFS RSGHLTK HIRTH TGEKP
    FACDICGRKFA DSGDRKR HTKIH TGSQKP
    FQCRICMRNFS RSGHLTK HIRTH TGEKP
    FACDICGRKFA DSGDRKR HTKIH
ZF11xALS1-TV5 amino acid sequence (SEQ ID NO: 175):
YACPVESCDRRFS DSGELTR HIRIH TGSQKP
    FQCRICMRNFS RSGHLTG HIRTH TGEKP
    FACDICGRKFA DSGERKR HTKIH TGSQKP
    FQCRICMRNFS RSGHLTG HIRTH TGEKP
    FACDICGRKFA DSGERKR HTKIH LRQKDGGGGSGGGGSGGGGSQKP
    FQCRICMRNFS RSGHLTG HIRTH TGEKP
    FACDICGRKFA DSGERKR HTKIH TGSQKP
    FQCRICMRNFS RSGLTKG HIRTH TGEKP
    FACDICGRKFA DSGERKR HTKIH TGSQKP
    FQCRICMRNFS RSGHLTG HIRTH TGEKP
    FACDICGRKFA DSGERKR HTKIH
ZF11xALS1-TV6 amino acid sequence (SEQ ID NO: 176):
YACPVESCDRRFS GSSELTR HIRIH TGSQKP
    FQCRICMRNFS RSDHLTR HIRTH TGEKP
    FACDICGRKFA GSSERKR HTKIH TGSQKP
    FQCRICMRNFS RSDHLTR HIRTH TGEKP
    FACDICGRKFA GSSERKR HTKIH LRQKDGGGGSGGGGSGGGGSQKP
    FQCRICMRNFS RSDHLTR HIRTH TGEKP
    FACDICGRKFA GSSERKR HTKIH TGSQKP
    FQCRICMRNFS RSDHLTR HIRTH TGEKP
    FACDICGRKFA GSSERKR HTKIH TGSQKP
    FQCRICMRNFS RSDHLTR HIRTH TGEKP
    FACDICGRKFA GSSERKR HTKIH
ZF11xALS1-TV7 amino acid sequence (SEQ ID NO: 177):
YACPVESCDRRFS GSSELTK HIRIH TGSQKP
    FQCRICMRNFS RSGHLTK HIRTH TGEKP
    FACDICGRKFA GSSELTK HTKIH TGSQKP
    FQCRICMRNFS RSGHLTK HIRTH TGEKP
    FACDICGRKFA GSSELTK HTKIH LRQKDGGGGSGGGGSGGGGSQKP
    FQCRICMRNFS RSGHLTK HIRTH TGEKP
    FACDICGRKFA GSSELTK HTKIH TGSQKP
    FQCRICMRNFS RSGHLTK HIRTH TGEKP
    FACDICGRKFA GSSELTK HTKIH TGSQKP
    FQCRICMRNFS RSGHLTK HIRTH TGEKP
    FACDICGRKFA GSSELTK HTKIH
ZF11xALS-TV8 amino acid sequence (SEQ ID NO: 178):
YACPVESCDRRFS DNGDLTR HIRIH TGSQKP
    FQCRICMRNFS RSAHLTR HIRTH TGEKP
YACPVESCDRRFS DNGDLTR HIRIH TGSQKP
    FQCRICMRNFS RSAHLTR HIRTH TGEKP
YACPVESCDRRFS DNGDLTR HIRIH TGQKPGGGGSGGGGSGGGGSQKP
    FQCRICMRNFS RSAHLTR HIRTH TGEKP
YACPVESCDRRFS DNGDLTR HIRIH TGSQKP
    FQCRICMRNFS RSAHLTR HIRTH TGEKP
YACPVESCDRRFS DNGDLTR HIRIH TGSQKP
    FQCRICMRNFS RSAHLTR HIRTH TGEKP
YACPVESCDRRFS DNGDLTR HIRIH
ZF11xALS-TV9 amino acid sequence (SEQ ID NO: 179):
YACPVESCDRRFS DGADLTR HIRIH TGSQKP
    FQCRICMRNFS RSAHLTR HIRTH TGEKP
YACPVESCDRRFS DGADLTR HIRIH TGSQKP
    FQCRICMRNFS RSAHLTR HIRTH TGEKP
YACPVESCDRRFS DGADLTR HIRIH TGQKPGGGGSGGGGSGGGGSQKP
    FQCRICMRNFS RSAHLTR HIRTH TGEKP
YACPVESCDRRFS DGADLTR HIRIH TGSQKP
    FQCRICMRNFS RSAHLTR HIRTH TGEKP
YACPVESCDRRFS DGADLTR HIRIH TGSQKP
    FQCRICMRNFS RSAHLTR HIRTH TGEKP
YACPVESCDRRFS DGADLTR HIRIH
ZF11xALS-TV10 amino acid sequence (SEQ ID NO: 180):
YACPVESCDRRFS AGADLTR HIRIH TGSQKP
    FQCRICMRNFS RSAHLTR HIRTH TGEKP
YACPVESCDRRFS AGADLTR HIRIH TGSQKP
    FQCRICMRNFS RSAHLTR HIRTH TGEKP
YACPVESCDRRFS AGADLTR HIRIH TGQKPGGGGSGGGGSGGGGSQKP
    FQCRICMRNFS RSAHLTR HIRTH TGEKP
YACPVESCDRRFS AGADLTR HIRIH TGSQKP
    FQCRICMRNFS RSAHLTR HIRTH TGEKP
YACPVESCDRRFS AGADLTR HIRIH TGSQKP
    FQCRICMRNFS RSAHLTR HIRTH TGEKP
YACPVESCDRRFS AGADLTR HIRIH
```

The binding strength and affinity of the zinc finger peptide variants above were tested to assess the affects of these sequence adjustments on the overall binding interaction with the GGGGCC target sequence. Binding affinity and competition assays were carried out, and the extended poly-zinc finger peptide variants were found to exhibit the expected results.

### Example 6

### Chromosomal Repression of Mutant C9ORF72

Repression of mutant C9ORF72 can be assessed using primary human B lymphocytes isolated from various C9ORF72 mutant carriers (a collection of 70 cell lines is available from the Coriell Institute, US). In addition, there are numbers of transgenic mouse lines that may be used for testing the efficacy of ZFP repression of mutant C9ORF72 locus, either in vivo or in vitro, using primary cultures including MEFs (Mouse embryonic Fibroblasts) or neurons.

FVB/NJ-Tg(C9orf72)500Lpwr/J (Jax Stock No: 030581) is also known as: C9¹⁹ BAC transgenic mouse line (C9B77). The C9¹⁹ BAC transgenic mouse line (C9B77) expresses multiple copies of a truncated human C9orf72 gene, modified in intron 1a to have hexanucleotide repeat expansions (GGGGCC). Individual transgene copies express C9orf72 with approx. 90 hexanucleotide repeats, or C9orf72 with approx. 450 hexanucleotide repeats.

The C9-500 BAC transgenic mouse line (Jax stock no Stock No: 029099) expresses a human C9orf72 gene with approx. 500 hexanucleotide repeats. Hemizygous mice develop age-dependent paralysis, anxiety-like behavior, decreased survival and widespread neurodegeneration of the brain and spinal cord, accompanied by accumulation of sense/antisense RNA foci and aggregation of RAN protein and TDP43. C9-500 mice allow study of both an acute, rapidly progressive disease as well as a slow progressive disease.

The effects of the zinc finger repressor peptides, the 11-finger peptide, on chromosomal C9ORF72 genes can be tested by qRT-PCR or protein level measurements.

### Example 7

### Cell Toxicity Assay

Since it would be advantageous for a ZFP-repressor therapy to have low toxicity, dye-labelling cell viability assays were performed to test the (non-specific) toxicity of the zinc finger peptides.

HEK-293T cells can be transfected with 400 ng of the indicated vector constructs using Lipofectamine2000 and harvested 48 hours after transfection. As a control Lipofectamine2000-only or non-transfected cells (negative) may be used. Cytotoxicity can be analysed using the Guava Cell Toxicity (PCA) Assay according to the manufacturer's instructions, and the results presented as the percentage of dead, mid-apoptotic and viable cells.

It is expected that the data will show that no statistically significant toxicity effects are produced in cells expressing zinc finger peptides of the invention, as compared to control experiments. It is expected that the repressor properties of the zinc finger peptides of the invention, and their potential for stable expression, will confirm that the peptides of the invention have significant potential for gene therapeutic applications.

### Example 8

### Long-Term Repression of Mut C9orf72/GGGGCC-Repeat Target In Vivo

Two C9orf72/GGGGCC mouse models, as described in Example 6, are used to assay long term repression. Furthermore, a similar molecular and behavioural approach was used as was used for ZFP efficacy in HD mouse models (Garriga-Canut et al. (2012), Proc. Natl. Acad. Sci., 109, E3136-3145); Agustin-Pavón et al. (2016) Mol. Neurodegener., 11(1):64). Briefly, rAAV2/1-ZFs are injected into appropriate mouse models. Test injections are either performed only in one hemisphere (so that the contralateral hemisphere is left untreated for the purpose of having a baseline comparison) or in whole brains to monitor overall efficiency (Molecular Neurodegeneration 11(1):64 (2016)). Brain samples from sacrificed animals are taken at 2, 4, 6 and 24 weeks post-injection, and RNA levels are analysed via quantitative real-time PCR (Garriga-Canut et al. (2012), Proc. Natl. Acad. Sci., 109, E3136-3145; Agustin-Pavón et al. (2016) Mol. Neurodegener., 11(1):64).

### Example 9

### Specific Repression of Pathogenic C9orf72 by Zinc Finger Repressor

We use a similar approach to that previously used and described to assess the efficacy of ZFP repression of the mutant HTT gene (Garriga-Canut et al. (2012), Proc. Natl. Acad. Sci., 109, E3136-3145); Agustin-Pavón et al. (2016) Mol. Neurodegener., 11(1):64). Thus, to assess C9orf72 repression, we compare repression by the anti-C9orf72 ZF against other DNA repeats in the mammalian genome, such as polyQ expansions. For example, since the mouse genome contains seven potential polyQ expansion genes (Garriga-Canut et al. (2012), Proc. Natl. Acad. Sci.;109, E3136-3145), it is important to understand whether the transcriptional repression is specific or whether the test repressor proteins might also affect one or more of the other potential polyCAG-targets. Thus, the effects of ZFs on the expression of four of these genes (wild-type wt HTT, ATN1, ATXN2, TBP; Table 8), are tested.

| | | | HTT (4, 7) | | ATN1 (3, | | ATXN2 (6, | | TBP (3, |
|---|---|---|---|---|---|---|---|---|---|
| Treatment | Time¹ | LR² | % | LR² | 10) % | LR² | 10) % | LR² | 13) % |
| ZF | 2 | R² = 0.53 | 98.3 ± 3.3 | R² = 0.02 | 109.4 ± 10.6 | R² = 0.00 | 103.3 ± 11.8 | R² = 0.08 | 99.1 ± 7.9 |
| | 4 | R² = 0.14 | 88.4 ± 6.7 | R² = 0.19 | 88.6 ± 7.3 | R² = 0.36 | 89.6 ± 5.4 | R² = 0.43 | 90.5 ± 10.4 |
| | 6 | R² = 0.43 | 92.5 ± 11.35 | R² = 0.16 | 91.4 ± 6.5 | R² = 0.00 | 96.7 ± 4.4 | R² = 0.07 | 94.7 ± 4.8 |

¹Weeks post-injection
²LR = Linear Regression

Table 8: Expression of mouse endogenous CAG-containing genes after treatment with a designed ZF (Agustin-Pavón et al. (2016) Mol. Neurodegener., 11(1):64). The first number (in brackets after the name of the gene) represents the number of CAG repeats, the second the number of glutamines in the coding stretch (CAG+CAA). Values are given as the percentage expression of the gene of interest, with respect to the average values in the control hemispheres. In bold: § P<0.1; *P<0.05. ATN1: atrophin 1; ATXN2: ataxin 2; HTT: huntingtin (mouse); TBP: TATA binding protein.

The results of this study show that the RNA levels of the four tested genes were not negatively correlated with the expression of the designed zinc finger construct. Therefore, several design variants—as discussed above—are possible to bind the DNA repeats to which they are designed and avoid other genomic repeats.

### Example 10

Zinc Finger Repression of the c9orf72 Locus in Various Cell Lines

To further demonstrate that the designed zinc finger transcription factors of this invention can control target gene expression at suitable endogenous genomic loci, in cell lines derived from human patients with repeat expansion diseases, the following experiments were carried out.

In this study, the zinc finger repressor peptides of SEQ ID NOs: 96 to 101 for targeting the c9orf72 locus comprising 5′-GGGGCC-3′ repeat sequences were cloned into appropriate expression vectors (see below), and expressed in target cells so as to repress the chosen target loci. Each of the zinc finger repressor peptides included the human KOX-1 repression domain. Activation can be similarly achieved using any appropriate activation domain, such as VP16, VP64, p65-RELA-AD, or any other activation domain (AD) suitable for gene activation in human cells.

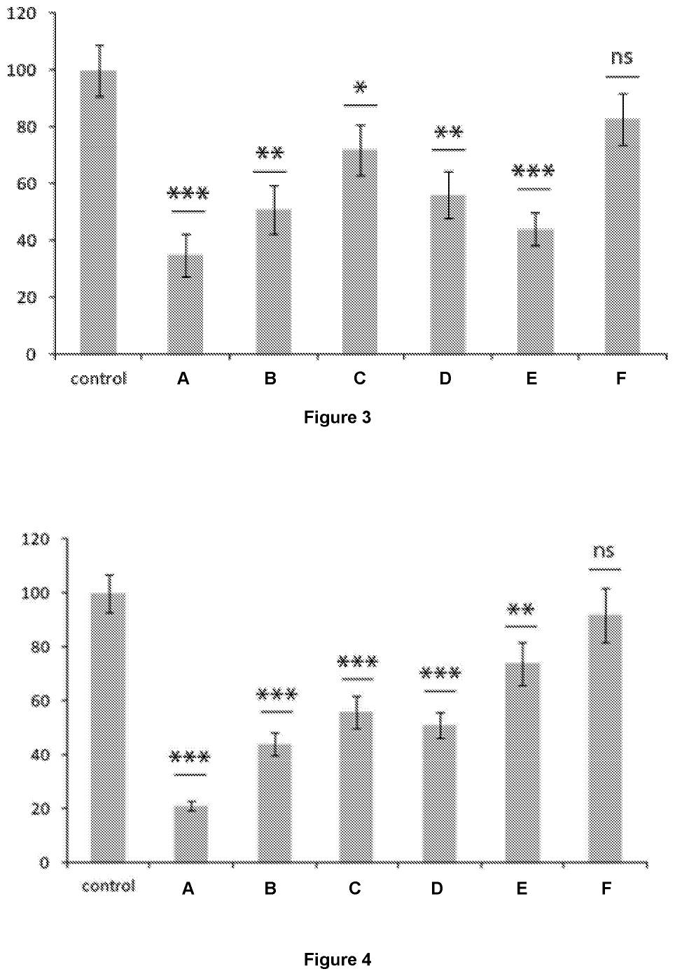

The zinc finger constructs were transiently transfected into the chosen cell lines and target gene expression, in the presence or absence of zinc finger repressor protein expression, was measured by qRT-PCR. The anti-ALS zinc fingers repressor proteins designed to bind at the mutant c9orf72 locus (i.e. ZF11xALS1-Kox, ZF11xALS2-Kox, ZF11xALS3-Kox, ZF11xALS-TV8-Kox, ZF11 xALS-TV9-Kox and ZF11xALS-TV10-Kox), repressed the mutant c9orf72 locus both in the lymphoblastoid cell line (LCL; ND10966) see FIG. 3, and in the human induced pluripotent stem cell line (hiPSC; RCFB60c7, RCi177) see FIG. 4.

As demonstrated, the various different designs of ‘tuned’ zinc finger repressor peptides have desirably different gene regulation activities, enabling tuning of target locus expression, as desired, depending on whether it is desired to achieve a stronger or a weaker repression of the target gene.

### Cloning:

All zinc finger (ZF) constructs were synthesised by Genscript and cloned into the pUC57 vector.

All mammalian expression plasmids were prepared as follows. Briefly, the KOX fragment was fused in frame to the zinc finger nucleotide sequence using Gibson assembly. The entire ZF-KOX cassette was then amplified by PCR and cloned into pcDNA3.1 vector using the TOPO system (Invitrogen). The expression of all ZF-KOX fragments was driven by the CMV promoter for these assays although alternative promoter-enhancers are possible, as described elsewhere herein. General purpose reagents, oligonucleotides, chemicals and solvents were purchased from Sigma-Aldrich, Eurofins and ThermoFisher. Enzymes and polymerases were obtained from New England Biolabs.

### Cell Culture and Transient Transfections:

The lymphoblastoid cell line (LCL) derived from a carrier of the c9orf72 ALS mutation (ND10966) was purchased from the Coriell Institute and cultured in RPMI 1640 medium, supplemented with 15% fetal bovine serum (FBS, Life Technologies). Cells were kept in suspension in tissue culture T75 flasks (NUNC, Thermo Scientific) at 37° C. in a 5% CO₂ incubator and maintained between 2×10⁵ and 8×10⁵ cells/ml.

For transfection, cells were passaged at 3.5×10⁵ cells, 48 hours and 24 hours before transfection. A total of 5×10⁶ ND10966 cells were transfected with 1.5 μg of pcDNA 3.1-ZF-KOX plasmid or empty pcDNA3.1 plasmid. GFP control cells received 1.5 μg of GFP plasmid, while negative control cells received transfection reagents only. Transfections were conducted with the Lipofectamine LTX kit according to the manufacturer's instructions (Invitrogen). After transfection, cells were suspended in medium and incubated overnight under normal cell culture conditions, and then replaced with fresh medium. The cells were pelleted 96 hours post-transfection, washed twice with ice-cold PBS, resuspended in the TRIzol reagent (Ambion) and stored at −80° C. for further analysis.

The human induced pluripotent stem cell (hiPSC) line was derived from a carrier of c9orf72 ALS mutation and was purchased from Public Health England. The hiPSC cell line (RCFB60c7, RCi177) was derived from human fibroblasts. The cells were grown on the 6-well plates covered with Matri-gel™ Matrix (BD Bioscience) in Essential medium 8 (Invitrogen). The medium was refreshed daily, and cells were passaged using an enzyme-free dissociation method based on EDTA. For transfection, cells were passaged at 5×10⁴ cells, at 24 hours before transfection. Cells were transfected with 1 μg of pcDNA 3.1-ZF-KOX plasmid or empty pcDNA3.1 plasmid. GFP control cells received 1 μg of GFP plasmid, while negative control cells received transfection reagents only. Transfections were conducted with the Lipofectamine 3000 kit, according to the manufacturer's instructions (Invitrogen). After transfection, cells were suspended in medium and incubated overnight under normal cell culture conditions, and then replaced with fresh medium. The cells were pelleted 96 hours post-transfection, washed twice with ice-cold PBS, resuspended in the TRIzol reagent (Ambion) and stored at −80° C. for further analysis.

### RNA Extraction and Taqman Real-Time PCR Expression Analysis:

Total RNA from cells was extracted with the mini-RNA kit (Qiagen, UK), according to the manufacturer's instructions. The reverse transcription reaction was performed using MMLV superscript reverse transcriptase (Invitrogen) and random hexamers (Invitrogen). All qPCR reactions were performed with a LightCycler® 480 Instrument (Roche). The qPCR reaction was carried out using 2× Taqman Master Mix buffer (Roche). mRNA copy number was determined in triplicate for each RNA sample by comparison with the geometric mean of three endogenous housekeeping genes: Gapdh, 18S and Hprt (Primer Design, UK). The c9orf72 transcripts (NM_145005) were detected with the following primers and probe set (Applied biosystems): Fw: 5′- CGGAAAGGAAGAATATGGATGC -3′; Rw: 5′- CCATTACAGGAATCACTTCTCCA -3′; Probe: 5′- AGCATTGGAATAATACTCTGACCCTGATCTTC -3′. The frataxin transcripts were detected using pre-designed primers and probe mix from Applied biosystems.

### Statistical Analysis:

Quantitative real time PCR analysis was carried out using the 2(−ΔΔC(T)) method. Values were presented as mean±SEM. Statistical analysis was performed using paired Student t tests (Excel). A p-value of 0.05 was considered as a significant difference.

### Example 11

Active Delivery of ZFs In Vivo Enhances Gene Regulation when Compared with Standard Delivery

The inventors have previously shown that zinc finger peptide (ZFP) therapies are currently limited by long-term expression efficiency: for the treatment of Huntington's disease, it was found that target mutant gene repression by zinc finger transcription factors was limited to only approx. 25% in the whole brain after 6 months (Agustin-Pavón et al. (2016) Mol. Neurodegener., 11(1):64). The concept of ‘active delivery’ could improve this situation by continuing to ‘drip-feed’ secreted cell-penetrating factors to neighbouring/bystander cells in the brain and other tissues (FIG. 5A, 5B).

In this Example, therefore, the inventors establish and demonstrate a universal method for achieving enhanced control of gene expression in vivo in mouse and human cells with artificial gene-regulatory transcription factors, which method is based on ‘active delivery’ of zinc finger peptides (ZFPs) by active gene expression, secretion and cell-penetration of designer transcription factors such as ZFPs. Beneficially, this approach exploits the intrinsic cell penetrating properties of ZFPs (Gaj et al. (2012), Nat. Methods, 9(8):805-807; Gaj et al. (2014), ACS Chem. Bio., 9(8):1662-1667; Liu et al. (2015), Mol. Ther. Nucleic Acids, 10;4:e232; and Lee et al. (1997), Virus Research, 52(1):97-108. These cell-penetration properties have not been coupled before to secretion in vivo, nor delivery with AAVs.

The artificial gene-regulatory transcription factor of this example was an 11-zinc finger peptide that demonstrates preferential binding to mutant CAG trinucleotide repeat sequences (e.g. as found in Huntington's Disease) in comparison with wild-type CAG trinucleotide repeat sequences (WO 2012/049332).

### Method Steps:

1. In the first step, expression cassettes were engineered to contain (in 5′ to 3′/N- to C-direction): the constitutive promoter/enhancer CMV; a protein secretion signal (SS) from human BMP10 protein (also known as a signal peptide (SP); SEQ ID NOs: 156 (prt) and 84 (dna)); a tandem array of two Nuclear Localisation Signals (NLSs; PKKKRKVPKKKRKV (SEQ ID NO: 160); SEQ ID NO: 87 (dna)) to enhance cell-penetration by providing a net positive charge; an 11-zinc finger peptide fused to a KRAB repressor domain (from KOX-1). The pCMV-IRES-GFP vector backbone (Clontech) was used as the template for the construct, where the GFP can be used to monitor transfection efficiency. In this construct an RIRR (SEQ ID NO: 85 (prt); SEQ ID NO: 86 (dna)) peptide cleavage site was placed between the SP and the NLS. Three 11-zinc finger peptides were tested, one previously shown by the inventors to successfully target the CAG-trinucleotide repeat associated with Huntington's disease gene sequences (SEQ ID NO: 102); one shown herein to target the GGGGCC-hexanucleotide repeat sequences associated with ALS disease gene sequences (SEQ ID NO: 103) and one designed to target the GCG-trinucleotide repeat sequences associated with FXTAS disease gene sequences (SEQ ID NO: 104).

2. Hela cells were grown in Dulbecco's modified Eagle's medium (DMEM)+1 g/L D-glucose and pyruvate supplemented with 10% (v/v) foetal bovine serum (FBS; Life Technologies, UK) without antibiotics, at sub-confluent cell density, in an incubator at 5% CO₂ and 37° C. Cells were passaged every two days, using 0.05% trypsin-EDTA (Life Technologies, UK). Cells were transfected at 50-60% confluency, using 5 μl of Lipofectamine LTX (Invitrogen) and 1 μg of plasmid DNA (pCMV-SS-2NLS-ZFP-KOX-IRES-GFP or pCMV-IRES-GFP) per 10 cm plate using the manufacturer's protocol. 24 hours post transfection, transfection efficiency was checked using a fluorescence microscope and cells reached on average 90% transfection efficiency. Next, medium was replaced with fresh serum-free culture medium. Cells were cultured for a further 96 hours without medium replacement. Next, enriched medium containing secreted ZFP was harvested and centrifuged for 5 minutes at 800×g at 4° C. in order to remove cell debris. The supernatant fraction was retained. 3. The following cell lines were used as ZFP receivers: (a) HEK293 stably expressing 25Q-Exon-1-GFP or 103Q-Exon-1-GFP under a CMV promoter; (b) human HD fibroblasts from the Cornell Institute depository collection—these cells contained one allele with a 67 CAG-trinucleotide repeat expansion, while the second allele contained 21 CAG-trinucleotide repeat sequence within the HTT gene; (c) primary human B lymphocytes isolated from C9ORF72 mutant carriers (Corriell ND06751, Control: ND08616); (d) C9B77 mouse cells (C9orf72 ˜450/90 GGGGCC repeats); (e) primary human B lymphocytes isolated from mutant FXTAS carrier (Corriell GM20233, ˜117 CGG repeats). Cell lines were grown in Dulbecco's modified Eagle's medium (DMEM)+1 g/L D-glucose and pyruvate supplemented with 10% (v/v) foetal bovine serum (FBS) (Life Technologies, UK) without antibiotics, at sub-confluent cell density, in an incubator at 5% CO₂ and 37° C.

4. SF medium containing secreted ZFP from Step 2 was diluted in fresh medium to provide 0%, 50% or 100% v/v mixtures of ZFP medium to fresh medium; and this was added to separate samples of cell receivers from Step 3 and incubated for 96h. Next, all three sample lines were washed with PBS and harvested by a direct application of 1 ml of TRIZOL reagent (Invitrogen). Cell lysates were immediately frozen and stored at −80° C. The next day, cell lysates were incubated at 37° C. for 2-3 minutes and placed on ice. 200 μl of chloroform was applied per 1 ml of cell lysate following by centrifugation at 8,000×g at 4° C. for 15 minutes. The upper aqueous fraction was then transferred into new tubes (approximately 400 μl) and an RNeasy Mini Kit (QIAGEN, UK) was used to extract total RNA following the manufacturer's instructions.

5. RNA samples (1 μg of total RNA) were treated with RNase-free DNase I (Promega, US) at 37° C. for 1h, followed by deactivation at 65° C. for 20 min. 1 μg of total RNA sample was reverse-transcribed using SuperScript Ill First—Strand Synthesis Kit (Invitrogen) according to manufacturer's instructions.

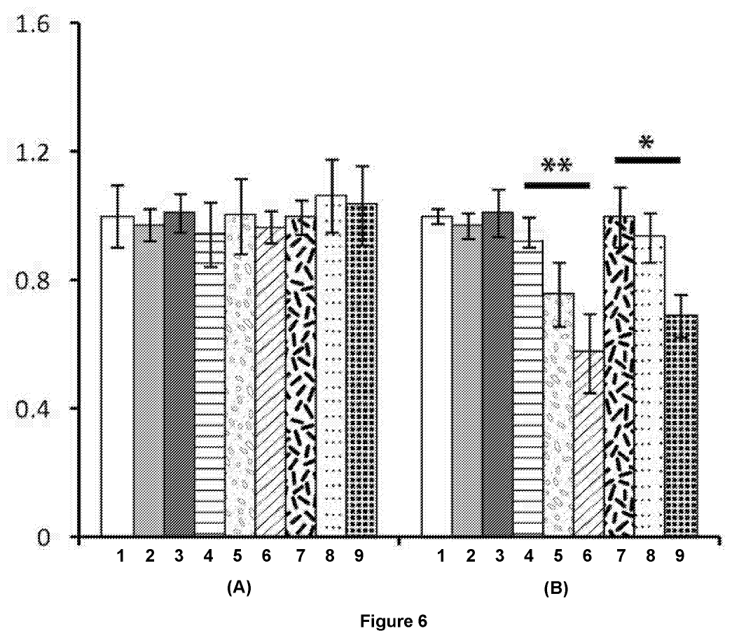

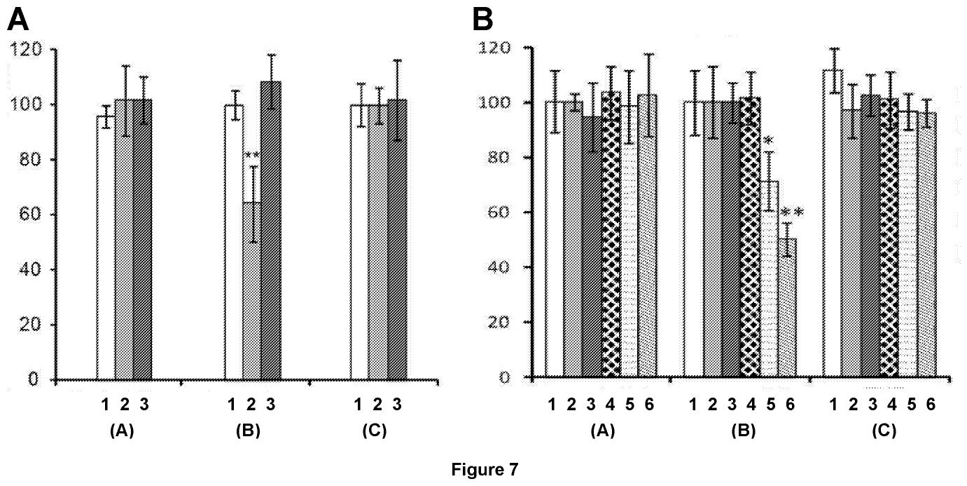

6. RT and Taqman qPCR: All qPCR reactions were performed using Light Cycler 480 Real Time Thermal Block Cycler in 384-well plates (Roche). Typically, 3 μl of approximately 5 ng/μl cDNA were used per reaction. For each biological replicate, three technical replicates were used. Sigma water was used as a negative control. qPCR cycling parameters were as follows: denaturation at 95° C. for 20s, followed by 45 cycles of amplification at 95° C. for 1 min, and subsequently cooling at 40° C. for 30s. Double Delta CT (cycle threshold) analysis was used for relative quantification, according to the equation Expression fold change=2\hat{ }(−ΔΔCt). Typical results are shown in FIGS. 6 and 7.

Wild-type and mutant target mRNAs were analysed by Taqman qPCR. Values were normalized to the housekeeping gene human 18S. Error bars are SEM (n=3). Student's t-test: *p<0.05; **p<0.01. ZF secretion leading to cell penetration and target gene repression are thus demonstrated in vitro in mouse and human cells.

The data of FIG. 6 and FIG. 7 clearly show that ZFP supernatant from HeLa cells (i.e. cell medium including secreted 11-zinc finger transcriptional repression peptide) can specifically repress mutant but not wild-type targets (as expected), in two different cell lines, in vitro (FIG. 6), and in vivo in mice (FIG. 7). The data also show that target gene repression level is proportional to the concentration of ZFP in the medium to which the the target cells are exposed. Repression is demonstrated in both whole brain and peripheral tissue (muscle). Similar results were obtained for each ZFP repressor protein against its target pathogenic sequence, showing in all cases that the zinc finger transcriptional repressor peptides were able to specifically downregulate target disease gene sequences while leaving non-target gene expression essentially at normal, expected levels.

7. For active delivery in vivo, the desired gene construct or constructs is/are subcloned into a suitable vector (e.g. SEQ ID NO: 88) together with a suitable promoter-enhancer. For mouse brain transduction, a recombinant AAV2/1 or AAV2/9 viral vector was used, as previously described (Agustin-Pavón et al. (2016) Mol. Neurodegener., 11(1):64). Delivery of viral vector was achieved by standard injection methods, including stereotaxis (2 μl viral preparation per hemisphere) and intrathecal injection (100 μl viral preparation) as previously described.

### Discussion

In these Examples, zinc finger peptides have been designed that are able to recognise and bind GGGGCC hexanucleotide repeats; and it has been shown that such proteins are able to induce transcription repression of target genes both in vitro and in vivo.

Fusing the Kox-1 or ZF87 KRAB repression domain to the zinc finger peptides of the invention was found to enhance the repression of targeted genes. Similarly, fusing the p65-ReIA activation domain to the poly-zinc finger peptides of the invention was found to increase the expression of targeted genes.

The zinc finger repressor peptides described herein (e.g. having 11-zinc finger domains arranged in tandem) are able to repress a target gene (in vitro) with expanded GGGGCC-repeat sequences (e.g. 100 or more repeats) preferentially over shorter repeat sequences (e.g. 23 of fewer repeats), thus demonstrating the therapeutic potential of zinc finger repressor proteins of the invention in downregulating expression of pathogenic genes associated with GGGGCC-repeat sequences.

Using expression cassettes developed by the inventors in their earlier reported work (e.g. WO 2017/077329), long-term, stable expression of zinc finger peptides of the invention can be achieved in model cell lines targeting pathogenic genes containing GGGGCC-repeat sequences. Repression of target gene expression can thus be demonstrated both at the protein and the RNA levels; and the expression of ‘wild-type’ genes having shorter genomic GGGGCC-repeat sequences remains broadly unaffected.

Thus, the extended poly-zinc finger peptides (especially having 11-zinc finger domains) were able to target the expanded GGGGCC repeats associated with the mutant C9Orf72 gene in preference to the normal GGGGCC repeats associated with the wild-type C9Orf72 gene. Similarly, beneficial effects are expected with the other zinc finger modulator peptides disclosed herein, which may contain, for example, 8, 10, 11, 12 or 18 adjacent zinc finger domains.

Likewise, poly-zinc finger peptides of the invention developed for optimal binding to short, wild-type GGGGCC-repeat sequences (i.e. peptides have 8 or less; most suitably 5 or 6 zinc finger domains) have been shown to bind with desirable, strong affinity to GGGGCC-repeat sequences containing less than 30 hexanucleotide repeats.

In addition, binding competition experiments demonstrate that higher concentrations of extended poly-zinc finger peptides according to the invention (e.g. having 11 zinc finger domains arranged in tandem) are able to out-compete shorter poly-zinc finger peptides (e.g. having 5 or 6 zinc finger domains arranged in tandem) for binding to expanded GGGGCC nucleic acid repeat sequences (e.g. of 100 or more repeats) more effectively than against short GGGGCC repeat sequences (e.g. of 2 to 23 repeats).

Toxicity effects of therapeutic molecules, especially for use in gene therapy and other similar strategies that require mid- or long-term expression of a heterologous protein, is a particular issue. Indeed, studies have previously shown that non-self proteins can elicit immune responses in vivo that are severe enough to cause widespread cell death.

In order to improve the mid- to long-term effects of zinc finger peptide expression in target organisms, especially in the brain, the inventors have previously developed strategies to reduce the toxicity and immunogenicity of the potentially therapeutic zinc finger peptides and repressor proteins of the invention (WO 2017/077329). Thus, in first aspects and embodiments, the present disclosure also provides zinc finger peptides and nucleic acid sequences that are suitable for repression of mutant C9Orf72 and/or activation of wild-type C9Orf72 in vivo and ex vivo in both mouse and human cells. Likewise, the zinc finger peptides disclosed herein are suitable for the targeting and modulation of other genes—especially those containing long GGGGCC-hexanucleotide repeat sequences.

Using a competition assay, it has been shown that the extended poly-zinc finger peptides of the invention (e.g. having 11 zinc finger domains) preferentially repress the expression of reporter genes containing over 30 GGGGCC repeats, which suggests that they hold significant promise for a therapeutic strategy to reduce the levels of mutant C9ORF72 protein in heterozygous patients.

Gene therapy is an attractive therapeutic strategy for various neurodegenerative diseases. For example, lentiviral vectors have been used to mediate the widespread and long-term expression of transgenes in non-dividing cells such as mature neurons (Dreyer, Methods Mol. Biol. 614: 3-35). Additionally, further benefits are associated with the use of the ubiquitous promoter, pHSP (based on Hsp90) characterised in our earlier patent application, WO 2017/077329. In particular, these benefits of the invention are enhanced when the promoter is used in combination with rAAV2/9 vectors, based on a virus that infects a wide variety of cell types. Alternatively, the neuron-specific promoter (pNSE) has been shown to provide similar results. Similar effects can be expected in animal (human) subjects using either the mouse promoter or the human equivalent of the synthetic pHSP promoter used in some of these studies.

The benefits of the zinc finger repressor peptides of the invention, and the zinc finger repressor/activator pairings of the invention may be further enhanced when used in combination with the ‘active delivery’ system disclosed herein. In this regard, by creating zinc finger peptide constructs that comprise a combination of secretion and cell-penetration signal sequences/peptides, therapeutic peptides are created that are capable of directing its own secretion from the cell in which it was expressed, and its subsequent penetration of a neighbouring cell which it comes into contact with, e.g. by diffusion. Once inside such a neighbouring cell, the zinc finger peptide of the invention may be targeted to the cell nucleus (e.g. byway of a nuclear localisation sequence) so that it can deliver its intended therapeutic effect within that neighbouring cell.

Accordingly, the active delivery system of the invention may provide one or both of prolonged therapeutic activity—by potentially continuing to deliver therapeutic peptides to cells that previously expressed but no longer express the therapeutic peptide (for example, a result of of gene silencing); and broader/enhanced therapeutic effect—by delivery of active, therapeutic peptides to cells that were not initially infected/transduced with the therapeutic construct.

Notably, the active delivery system of the present disclosure is not only suitable for use in conjunction with the therapeutic zinc finger peptides of the invention, but may also be used in conjunction with any other therapeutic agent (in particular a polypeptide) that may be expressed in a cell in vivo or in vitro.

### CONCLUSION

This study demonstrates that extended poly-zinc finger repressor proteins can be designed and contructed to reduce pathogenic gene expression of target gene sequences both in vitro and in vivo. Such zinc finger repressor proteins, suitably at least 8 zinc fingers (and preferably more than 8 zinc fingers) in length, may be useful for the downregulation of pathogenic genes associated with expanded GGGGCC-repeat sequences, such as for the potential treatment of Amyotrophic lateral sclerosis (ALS) and familial Frontotemporal dementia (FTD).

In addition, it has been demonstrated that shorter poly-zinc finger activator proteins of no more than 8 zinc fingers (and more suitably from 5 to 7 zinc finger domains) can be designed to bind effectively to and activate gene expression of wild-type gene constructs, e.g. having less than 30 GGGGCC-repeat sequences. Such zinc finger activator peptides are particularly suited for addressing haploinsufficiency wherein the desired wild-type gene product is underexpressed against a background of pathogenesis in the same disease state.

In particular, by combining the zinc finger repressor and zinc finger activator proteins of the invention, a particularly effective strategy for treating diseases such as ALS and FTD may be achieved. In this regard, it has also been postulated that the therapeutic effects/treatments of the invention may be enhanced by: (i) reducing the amount/concentration of the zinc finger activator peptide that is administered when compared to the amount/concentration of zinc finger repressor protein of the invention (e.g. to reduce the possibility of the zinc finger activator protein competing for and binding to pathogenic sequence sites); and (ii) reducing the binding strength of the longer zinc finger repressor proteins of the invention for their target nucleotide sequence to favour binding of the zinc finger repressor proteins of the invention to the expanded, pathogenic nucleotide repeat target sites.

Moreover, it has been demonstrated that long-term gene therapy treatments involving down-regulation of pathogenic genes and/or upregulation of wild-type genes is enhanced through ‘active delivery’ of therapeutic agents to non-transduced target cells; i.e. by delivery of therapeutic peptides from cells in which they are expressed to neighbouring cells in which they are not expressed. In this way, despite a reduction in the proportion of cells in a target cell population that express therapeutic peptide over time, a relatively enhanced therapeutic effect can be maintained by secretion and cell penetration of therapeutic peptides from expressing cells into neighbouring, non-expressing target cells. By adapting the therapeutic zinc finger peptides of the invention for active delivery, as described herein, it is believed that long-term (over 6 months) effective gene therapy treatment can be achieved in vivo from a single treatment/administration.

TABLE 9
Sequences
Peptide and Nucleic Acid Sequences.

| SEQ | | |
|---|---|---|
| ID | | |
| NO: | Sequence Type | Sequence |
| 1 | Recognition (prt) | (D/A/G)SS(V/D/E/A/G)(L/R)(T/K)(R/K/G) |
| 2 | Recognition (prt) | (D/A/G/T/V/S)(S/N/R/A/G)(S/R/E/A/G)(V/D/E/A/G/I/L/S/T) |
| | | (L/R)(T/K)(R/K/G) |
| 3 | Recognition (prt) | RS(D/A/G)HL(T/S/A)(R/K/G) |
| 133 | Recognition (prt) | (R/G)S(D/G)HL(T/S/A)(R/K/G) |
| 134 | Recognition (prt) | (R/G)G(D/S/G)HR(K/I/A)(R/K/G) |
| 4 | Recognition (prt) | (D/A/G)SS(V/D/E/A/G)LT(R/K/G) |
| 5 | Recognition (prt) | (D/A/G/T/V/S)(S/N/R)(S/R/E/A/G)(V/D/E/A/G/I/L/S/T)LT |
| | | (R/K/G) |
| 6 | Recognition (prt) | (D/A/G)SS(V/D/E/A/G)RK(R/K/G) |
| 7 | Recognition (prt) | (D/A/G/T/V/S)(S/N/R)(S/R/E/A/G)(V/D/E/A/G/I/L/S/T)RK |
| | | (R/K/G) |
| 8 | Recognition (prt) | (D/A)SS(V/E)LT(R/K) |
| 9 | Recognition (prt) | (D/A/T)(S/N/R)(S/R/E)(V/E/D)LTR |
| 10 | Recognition (prt) | (D/A)SS(V/E)RK(R/K) |
| 11 | Recognition (prt) | (D/A/T)(S/N/R)(S/R/E)(V/E/D)RKR |
| 181 | Recognition (prt) | (G/D)S(S/G)(E/D)(L/R)(T/K)(R/K) |
| 182 | Recognition (prt) | (G/D)S(S/G)(E/D)LT(R/K) |
| 183 | Recognition (prt) | (G/D)S(S/G)(E/D)RK(R/K) |
| 184 | Recognition (prt) | (D/A)(N/G)(G/A)(V/D)(L/R)(T/K)(R/K) |
| 185 | Recognition (prt) | (D/A)(N/G)(G/A)(V/D)LT(R/K) |
| 186 | Recognition (prt) | (D/A)(N/G)(G/A)(V/D)RK(R/K) |
| 12 | Recognition (prt) | RS(D/G)HLT(R/K) |
| 135 | Recognition (prt) | (R/G)SDHLT(R/K) |
| 136 | Recognition (prt) | RG(D/S)HRK(R/K) |
| 13 | Recognition (prt) | DSSVLTR |
| 137 | Recognition (prt) | DSSVLTG |
| 14 | Recognition (prt) | DSSVRKR |
| 138 | Recognition (prt) | DSSVRKG |
| 187 | Recognition (prt) | DSGDLTR |
| 188 | Recognition (prt) | DSGDRKR |
| 189 | Recognition (prt) | DSGELTR |
| 190 | Recognition (prt) | DSGERKR |
| 191 | Recognition (prt) | GSSELTK |
| 15 | Recognition (prt) | ASSVLTR |
| 16 | Recognition (prt) | ASSVRKR |
| 17 | Recognition (prt) | DSSELTR |
| 139 | Recognition (prt) | DSSELTG |
| 18 | Recognition (prt) | DSSERKR |
| 140 | Recognition (prt) | DSSERKG |
| 19 | Recognition (prt) | ASSELTR |
| 20 | Recognition (prt) | ASSERKR |
| 21 | Recognition (prt) | GSSVLTR |
| 22 | Recognition (prt) | GSSVRKR |
| 23 | Recognition (prt) | GSSELTR |
| 24 | Recognition (prt) | GSSERKR |
| 25 | Recognition (prt) | DSSGLTR |
| 26 | Recognition (prt) | DSSGRKR |
| 27 | Recognition (prt) | ASSGLTR |
| 28 | Recognition (prt) | ASSGRKR |
| 29 | Recognition (prt) | GSSGLTR |
| 30 | Recognition (prt) | GSSGRKR |
| 31 | Recognition (prt) | DNRDLTR |
| 32 | Recognition (prt) | DNRDRKR |
| 33 | Recognition (prt) | TREDLTR |
| 34 | Recognition (prt) | TREDRKR |
| 35 | Recognition (prt) | DNRELTR |
| 36 | Recognition (prt) | DNRERKR |
| 37 | Recognition (prt) | ANRELTR |
| 38 | Recognition (prt) | ANRERKR |
| 39 | Recognition (prt) | DREELTR |
| 40 | Recognition (prt) | DREERKR |
| 41 | Recognition (prt) | AREELTR |
| 42 | Recognition (prt) | AREERKR |
| 43 | Recognition (prt) | TNRELTR |
| 44 | Recognition (prt) | TNRERKR |
| 45 | Recognition (prt) | TREELTR |
| 46 | Recognition (prt) | TREERKR |
| 47 | Recognition (prt) | GNRELTR |
| 48 | Recognition (prt) | GNRERKR |
| 49 | Recognition (prt) | GREELTR |
| 50 | Recognition (prt) | GREERKR |
| 51 | Recognition (prt) | DNRGLTR |
| 52 | Recognition (prt) | DNRGRKR |
| 53 | Recognition (prt) | ANRGLTR |
| 54 | Recognition (prt) | ANRGRKR |
| 55 | Recognition (prt) | DREGLTR |
| 56 | Recognition (prt) | DREGRKR |
| 57 | Recognition (prt) | AREGLTR |
| 58 | Recognition (prt) | AREGRKR |
| 59 | Recognition (prt) | TNRGLTR |
| 60 | Recognition (prt) | TNRGRKR |
| 61 | Recognition (prt) | TREGLTR |
| 62 | Recognition (prt) | TREGRKR |
| 63 | Recognition (prt) | DNRELTG |
| 64 | Recognition (prt) | DNRERKG |
| 65 | Recognition (prt) | TREELTG |
| 66 | Recognition (prt) | TREERKG |
| 67 | Recognition (prt) | GNRDLTR |
| 68 | Recognition (prt) | GNRDRKR |
| 69 | Recognition (prt) | GREDLTR |
| 70 | Recognition (prt) | GREDRKR |
| 71 | Recognition (prt) | GNRGLTR |
| 72 | Recognition (prt) | GNRGRKR |
| 73 | Recognition (prt) | GREGLTR |
| 74 | Recognition (prt) | GREGRKR |
| 75 | Recognition (prt) | RSDHLTR |
| 141 | Recognition (prt) | RGDHLTR |
| 142 | Recognition (prt) | GSDHLTR |
| 76 | Recognition (prt) | RSDHLTK |
| 143 | Recognition (prt) | RGDHLTK |
| 144 | Recognition (prt) | GSDHLTK |
| 77 | Recognition (prt) | RSDHLTG |
| 78 | Recognition (prt) | RSAHLTR |
| 79 | Recognition (prt) | RSAHLTK |
| 80 | Recognition (prt) | RSAHLTG |
| 81 | Recognition (prt) | RSGHLTR |
| 82 | Recognition (prt) | RSGHLTK |
| 83 | Recognition (prt) | RSGHLTG |
| 107 | Recognition (prt) | (D/E/T/V/S)(S/N/R)(S/R/E)(V/D/E/I/L/S/T)(L/R)(T/K)(R/K) |
| 108 | Recognition (prt) | DSSVL(T/S/A)R |
| 109 | Recognition (prt) | RSDH(L/R)(T/K)(R/K) |
| 110 | Recognition (prt) | RSDHL(T/S/A)(R/K) |
| 111 | Recognition (prt) | RSDHRKR |
| 145 | Recognition (prt) | DNGDLTR |
| 146 | Recognition (prt) | DGADLTR |
| 147 | Recognition (prt) | AGADLTR |
| 112 | Linker (prt) | TGEKP |
| 113 | Linker (prt) | TG(E/Q)(K/R)P |
| 114 | Linker (prt) | TGQKP |
| 115 | Linker (prt) | TG(G/S)(E/Q)(K/R)P |
| 116 | Linker (prt) | TGGERP |
| 117 | Linker (prt) | TGSERP |
| 118 | Linker (prt) | TGGQRP |
| 119 | Linker (prt) | TGSQRP |
| 120 | Linker (prt) | TGGEKP |
| 121 | Linker (prt) | TGSEKP |
| 122 | Linker (prt) | TGGQKP |
| 123 | Linker (prt) | TGSQKP |
| 124 | Linker (prt) | TG(G/S)₀₋₂(E/Q)(K/R)P |
| 125 | Linker (prt) | T(G/S)₀₋₂G(E/Q)(K/R)P |
| 126 | Linker (prt) | TG(G/S)₃(E/Q)(K/R)P |
| 127 | Linker (prt) | T(G/S)₃G(E/Q)(K/R)P |
| 128 | Linker (prt) | LRQKD(GGGGS)₁₋₄QLVGTAERP |
| 129 | Linker (prt) | LRQKD(GGGGS)₁₋₄QKP |
| 130 | Linker (prt) | LRQKDGGGGSGGGGSGGGGSQLVGTAERP |
| 131 | Linker (prt) | LRQKDGGGGSGGGGSGGGGSQKP |
| 132 | Linker (prt) | TGERP |
| 148 | SV40 NLS (prt) | PKKKRKV |
| 149 | Human | PKKRRKVT |
| | KIAA2022 NLS | |
| | (prt) | |
| 150 | mouse primase | RIRKKLR |
| | p58 NLS9 (prt) | |
| 151 | Human Kox-1 | LSPQHSAVTQGSIIKNKEGMDAKSLTAWSRTLVTFKDVFVDFTREEWKLLDTAQQIV |
| | KRAB domain | YRNVMLENYKNLVSLGYQLTKPDVILRLEKGEEPWLVEREIHQETHPDSETAFEIKS |
| | (prt) | SV |
| 152 | Mouse ZF87 | EEMLSFRDVAIDFSAEEWECLEPAQWNLYRDVMLENYSHLVFLGLASCKPYLVTFLE |
| | KRAB domain | QRQEPSVVKRPAAATVHP |
| | (prt) | |
| 153 | Linker (prt) | LRQKDGGGGSGGGGSGGGGSQLVSS |
| 154 | Linker (prt) | LRQKDGGGGSGGGGSS |
| 155 | Linker (prt) | LRQKDGGGSGGGGS |
| 156 | BMP10 | MGSLVLTLCALFCLAAYLVSG |
| | secretion signal | |
| | (prt) | |
| 157 | Human N- | MGPKKRRKVTGERP |
| | terminal leader | |
| | (prt) | |
| 158 | Human N- | MGPKKRRKVTLAERP |
| | terminal leader | |
| | (prt) | |
| 159 | Mouse N- | MGRIRKKLRLAERP |
| | terminal leader | |
| | (prt) | |
| 160 | Double SV40 | PKKKRKVPKKKRKV |
| | NLS (prt) | |
| 164 | Mouse Zif268 | ERPYACPVESCDRRFSRSDELTRHIRIHTGQKPFQCRICMRNFSRSDHLTTHIRTHT |
| | (prt) | GEKPFACDICGRKFARSDERKRHTKIHLRQKD |
| 165 | Human Zif 268 | ERPYACPVESCDRRFSRSDELTRHIRIHTGQKPFQCRICMRNFSRSDHLTTHIRTHT |
| | (prt) | GEKPFACDICGRKFARSDERKGHTKIHLRQKD |
| 166 | 11-zinc finger | YACPVESCDRRFSDSSVLTRHIRIHTGSQKPFQCRICMRNFSRSDHLTRHIRTHTGE |
| | peptide-ALS- | KPFACDICGRKFADSSVRKRHTKIHTGSQKPFQCRICMRNFSRSDHLTRHIRTHTGE |
| | binding 1 (prt) | KPFACDICGRKFADSSVRKRHTKIHLRQKDGGGGSGGGGSGGGGSQKPFQCRICMRN |
| | | FSRSDHLTRHIRTHTGEKPFACDICGRKFADSSVRKRHTKIHTGSQKPFQCRICMRN |
| | | FSRSDHLTRHIRTHTGEKPFACDICGRKFADSSVRKRHTKIHTGSQKPFQCRICMRN |
| | | FSRSDHLTRHIRTHTGEKPFACDICGRKFADSSVRKRHTKIH |
| 167 | 11-zinc finger | YACPVESCDRRESDNRDLTRHIRIHTGSQKPFQCRICMRNFSRSDHLTRHIRTHTGE |
| | peptide-ALS- | KPYACPVESCDRRFSDNRDLTRHIRIHTGSQKPFQCRICMRNFSRSDHLTRHIRTHT |
| | binding 2 (prt) | GEKPYACPVESCDRRFSDNRDLTRHIRIHTGQKPGGGGSGGGGSGGGGSQKPFQCRI |
| | | CMRNFSRSDHLTRHIRTHTGEKPYACPVESCDRRFSDNRDLTRHIRIHTGSQKPFQC |
| | | RICMRNFSRSDHLTRHIRTHTGEKPYACPVESCDRRFSDNRDLTRHIRIHTGSQKPF |
| | | QCRICMRNFSRSDHLTRHIRTHTGEKPYACPVESCDRRFSDNRDLTRHIRIH |
| 168 | 11-zinc finger | YACPVESCDRRFSTREDLTRHIRIHTGSQKPFQCRICMRNFSRSDHLTRHIRTHTGE |
| | peptide-ALS- | KPFACDICGRKFATREDRKRHTKIHTGSQKPFQCRICMRNFSRSDHLTRHIRTHTGE |
| | binding 3 (prt) | KPFACDICGRKFATREDRKRHTKIHLRQKDGGGGSGGGGSGGGGSQKPFQCRICMRN |
| | | FSRSDHLTRHIRTHTGEKPFACDICGRKFATREDRKRHTKIHTGSQKPFQCRICMRN |
| | | FSRSDHLTRHIRTHTGEKPFACDICGRKFATREDRKRHTKIHTGSQKPFQCRICMRN |
| | | FSRSDHLTRHIRTHTGEKPFACDICGRKFATREDRKRHTKIH |
| 169 | 5-zinc finger | YACPVESCDRRFSDSSVLTRHIRIHTGSQKPFQCRICMRNFSRSDHLTRHIRTHTGE |
| | peptide-ALS- | KPFACDICGRKFADSSVRKRHTKIHTGSQKPFQCRICMRNFSRSDHLTRHIRTHTGE |
| | binding 1 (prt) | KPFACDICGRKFADSSVRKRHTKIH |
| 170 | 6-zinc finger | FQCRICMRNFSRSDHLTRHIRTHTGEKPFACDICGRKFADSSVRKRHTKIHTGSQKP |
| | peptide-ALS- | FQCRICMRNFSRSDHLTRHIRTHTGEKPFACDICGRKFADSSVRKRHTKIHTGSQKP |
| | binding 1 (prt) | FQCRICMRNFSRSDHLTRHIRTHTGEKPFACDICGRKFADSSVRKRHTKIH |
| 171 | 11-zinc finger | YACPVESCDRRESDSSVLTRHIRIHTGSQKPFQCRICMRNFSRSGHLTRHIRTHTGE |
| | peptide-ALS- | KPFACDICGRKFADSSVRKRHTKIHTGSQKPFQCRICMRNFSRSGHLTRHIRTHTGE |
| | binding variant 1 | KPFACDICGRKFADSSVRKRHTKIHLRQKDGGGGSGGGGSGGGGSQKPFQCRICMRN |
| | (TV1) (prt) | FSRSGHLTRHIRTHTGEKPFACDICGRKFADSSVRKRHTKIHTGSQKPFQCRICMRN |
| | | FSRSGHLTRHIRTHTGEKPFACDICGRKFADSSVRKRHTKIHTGSQKPFQCRICMRN |
| | | FSRSGHLTRHIRTHTGEKPFACDICGRKFADSSVRKRHTKIH |
| 172 | 11-zinc finger | YACPVESCDRRFSASSELTRHIRIHTGSQKPFQCRICMRNFSRSGHLTRHIRTHTGE |
| | peptide-ALS- | KPFACDICGRKFAASSERKRHTKIHTGSQKPFQCRICMRNFSRSGHLTRHIRTHTGE |
| | binding variant 2 | KPFACDICGRKFAASSERKRHTKIHLRQKDGGGGSGGGGSGGGGSQKPFQCRICMRN |
| | (TV2) (prt) | FSRSGHLTRHIRTHTGEKPFACDICGRKFAASSERKRHTKIHTGSQKPFQCRICMRN |
| | | FSRSGHLTRHIRTHTGEKPFACDICGRKFAASSERKRHTKIHTGSQKPFQCRICMRN |
| | | FSRSGHLTRHIRTHTGEKPFACDICGRKFAASSERKRHTKIH |
| 173 | 11-zinc finger | YACPVESCDRRFSASSELTRHIRIHTGSQKPFQCRICMRNFSRSGHLTKHIRTHTGE |
| | peptide-ALS- | KPFACDICGRKFAASSERKRHTKIHTGSQKPFQCRICMRNFSRSGHLTKHIRTHTGE |
| | binding variant 3 | KPFACDICGRKFAASSERKRHTKIHLRQKDGGGGSGGGGSGGGGSQKPFQCRICMRN |
| | (TV3) (prt) | FSRSGHLTKHIRTHTGEKPFACDICGRKFAASSERKRHTKIHTGSQKPFQCRICMRN |
| | | FSRSGHLTKHIRTHTGEKPFACDICGRKFAASSERKRHTKIHTGSQKPFQCRICMRN |
| | | FSRSGHLTKHIRTHTGEKPFACDICGRKFAASSERKRHTKIH |
| 174 | 11-zinc finger | YACPVESCDRRFSDSGDLTRHIRIHTGSQKPFQCRICMRNFSRSGHLTKHIRTHTGE |
| | peptide-ALS- | KPFACDICGRKFADSGDRKRHTKIHTGSQKPFQCRICMRNFSRSGHLTKHIRTHTGE |
| | binding variant 4 | KPFACDICGRKFADSGDRKRHTKIHLRQKDGGGGSGGGGSGGGGSQKPFQCRICMRN |
| | (TV4) (prt) | FSRSGHLTKHIRTHTGEKPFACDICGRKFADSGDRKRHTKIHTGSQKPFQCRICMRN |
| | | FSRSGHLTKHIRTHTGEKPFACDICGRKFADSGDRKRHTKIHTGSQKPFQCRICMRN |
| | | FSRSGHLTKHIRTHTGEKPFACDICGRKFADSGDRKRHTKIH |
| 175 | 11-zinc finger | YACPVESCDRRFSDSGELTRHIRIHTGSQKPFQCRICMRNFSRSGHLTGHIRTHTGE |
| | peptide-ALS- | KPFACDICGRKFADSGERKRHTKIHTGSQKPFQCRICMRNFSRSGHLTGHIRTHTGE |
| | binding variant 5 | KPFACDICGRKFADSGERKRHTKIHLRQKDGGGGSGGGGSGGGGSQKPFQCRICMRN |
| | (TV5) (prt) | FSRSGHLTGHIRTHTGEKPFACDICGRKFADSGERKRHTKIHTGSQKPFQCRICMRN |
| | | FSRSGLTKGHIRTHTGEKPFACDICGRKFADSGERKRHTKIHTGSQKPFQCRICMRN |
| | | FSRSGHLTGHIRTHTGEKPFACDICGRKFADSGERKRHTKIH |
| 176 | 11-zinc finger | YACPVESCDRRFSGSSELTRHIRIHTGSQKPFQCRICMRNFSRSDHLTRHIRTHTGE |
| | peptide-ALS- | KPFACDICGRKFAGSSERKRHTKIHTGSQKPFQCRICMRNFSRSDHLTRHIRTHTGE |
| | binding variant 6 | KPFACDICGRKFAGSSERKRHTKIHLRQKDGGGGSGGGGSGGGGSQKPFQCRICMRN |
| | (TV6) (prt) | FSRSDHLTRHIRTHTGEKPFACDICGRKFAGSSERKRHTKIHTGSQKPFQCRICMRN |
| | | FSRSDHLTRHIRTHTGEKPFACDICGRKFAGSSERKRHTKIHTGSQKPFQCRICMRN |
| | | FSRSDHLTRHIRTHTGEKPFACDICGRKFAGSSERKRHTKIH |
| 177 | 11-zinc finger | YACPVESCDRRFSGSSELTKHIRIHTGSQKPFQCRICMRNFSRSGHLTKHIRTHTGE |
| | peptide-ALS- | KPFACDICGRKFAGSSELTKHTKIHTGSQKPFQCRICMRNFSRSGHLTKHIRTHTGE |
| | binding variant 7 | KPFACDICGRKFAGSSELTKHTKIHLRQKDGGGGSGGGGSGGGGSQKPFQCRICMRN |
| | (TV7) (prt) | FSRSGHLTKHIRTHTGEKPFACDICGRKFAGSSELTKHTKIHTGSQKPFQCRICMRN |
| | | FSRSGHLTKHIRTHTGEKPFACDICGRKFAGSSELTKHTKIHTGSQKPFQCRICMRN |
| | | FSRSGHLTKHIRTHTGEKPFACDICGRKFAGSSELTKHTKIH |
| 178 | 11-zinc finger | YACPVESCDRRFSDNGDLTRHIRIHTGSQKPFQCRICMRNFSRSAHLTRHIRTHTGE |
| | peptide-ALS- | KPYACPVESCDRRFSDNGDLTRHIRIHTGSQKPFQCRICMRNFSRSAHLTRHIRTHT |
| | binding variant 8 | GEKPYACPVESCDRRFSDNGDLTRHIRIHTGQKPGGGGSGGGGSGGGGSQKPFQCRI |
| | (TV8) (prt) | CMRNFSRSAHLTRHIRTHTGEKPYACPVESCDRRFSDNGDLTRHIRIHTGSQKPFQC |
| | | RICMRNFSRSAHLTRHIRTHTGEKPYACPVESCDRRFSDNGDLTRHIRIHTGSQKPF |
| | | QCRICMRNFSRSAHLTRHIRTHTGEKPYACPVESCDRRFSDNGDLTRHIRIH |
| 179 | 11-zinc finger | YACPVESCDRRFSDGADLTRHIRIHTGSQKPFQCRICMRNFSRSAHLTRHIRTHTGE |
| | peptide-ALS- | KPYACPVESCDRRFSDGADLTRHIRIHTGSQKPFQCRICMRNFSRSAHLTRHIRTHT |
| | binding variant 9 | GEKPYACPVESCDRRFSDGADLTRHIRIHTGQKPGGGGSGGGGSGGGGSQKPFQCRI |
| | (TV9) (prt) | CMRNFSRSAHLTRHIRTHTGEKPYACPVESCDRRESDGADLTRHIRIHTGSQKPFQC |
| | | RICMRNFSRSAHLTRHIRTHTGEKPYACPVESCDRRESDGADLTRHIRIHTGSQKPF |
| | | QCRICMRNFSRSAHLTRHIRTHTGEKPYACPVESCDRRFSDGADLTRHIRIH |
| 180 | 11-zinc finger | YACPVESCDRRFSAGADLTRHIRIHTGSQKPFQCRICMRNFSRSAHLTRHIRTHTGE |
| | peptide-ALS- | KPYACPVESCDRRFSAGADLTRHIRIHTGSQKPFQCRICMRNFSRSAHLTRHIRTHT |
| | binding variant | GEKPYACPVESCDRRFSAGADLTRHIRIHTGQKPGGGGSGGGGSGGGGSQKPFQCRI |
| | 10 (TV10) (prt) | CMRNFSRSAHLTRHIRTHTGEKPYACPVESCDRRFSAGADLTRHIRIHTGSQKPFQC |
| | | RICMRNFSRSAHLTRHIRTHTGEKPYACPVESCDRRFSAGADLTRHIRIHTGSQKPF |
| | | QCRICMRNFSRSAHLTRHIRTHTGEKPYACPVESCDRRFSAGADLTRHIRIH |
| 84 | BMP10 | ATGGGCTCTCTGGTCCTGACACTGTGCGCTCTTTTCTGCCTGGCAGCTTACTTGGTT |
| | secretion signal | TCTGGC |
| | (dna) | |
| 85 | Peptide | RIRR |
| | cleavage | |
| | sequence (prt) | |
| 86 | Peptide | CGAATCAGAAGG |
| | cleavage | |
| | sequence (dna) | |
| 87 | Double-NLS | CCGAAGAAAAAACGTAAAGTGCCGAAGAAAAAACGTAAAGTG |
| | (dna) | |
| 88 | Active delivery | ATGGGCTCTCTGGTCCTGACACTGTGCGCTCTTTTCTGCCTGGCAGCTTACTTGGTT |
| | construct (dna): | TCTGGCCGAATCAGAAGGGATATGGGACCGAAGAAAAAACGTAAAGTGCCGAAGAAA |
| | pCMV-SS- | AAACGTAAAGTGGGCGAAAGACCATACGCATGTCCCGTCGAAAGTTGCGATAGAAGG |
| | 2xNLS-ZFP- | TTTAGTCAGTCTGGCGACCTGACCAGGCACATCCGCATCCACACAGGCTCCCAGAAG |
| | KOX-IRES-GFP | CCATTCCAGTGCAGGATCTGTATGCGCAACTTTTCTCAGAGCGGCGATCTGACCCGG |
| | | CACATCAGAACCCACACAGGCGAGAAGCCCTTCGCCTGCGACATCTGTGGCAGGAAG |
| | | TTTGCCCAGTCCGGCGATCGGAAGAGACACACCAAGATCCACACAGGCTCTCAGAAG |
| | | CCTTTCCAGTGCCGGATCTGTATGAGAAATTTTTCCCAGTCTGGCGACCTGACTAGA |
| | | CACATCCGCACTCATACAGGCGAGAAGCCATTCGCCTGTGATATCTGTGGCCGGAAG |
| | | TTTGCCCAGAGCGGCGATAGGAAGCGCCACACAAAGATCCACCTGAGACAGAAGGAT |
| | | GGAGGAGGAGGCTCTGGAGGAGGAGGCAGCGGAGGAGGAGGCTCCCAGAAGCCCTTT |
| | | CAGTGTAGAATTTGTATGCGCAACTTTAGCCAGTCTGGCGATCTGACTAGACATATT |
| | | AGGACTCATACCGGCGAGAAGCCTTTCGCCTGTGATATTTGTGGCCGGAAATTCGCC |
| | | CAGAGTGGCGATCGGAAAAGGCATACTAAAATTCATACCGGCTCCCAGAAACCATTT |
| | | CAGTGTAGAATCTGCATGAGAAATTTTTCTCAGAGCGGCGACCTGACTCGCCACATC |
| | | AGGACTCATACTGGAGAAAAACCCTTCGCCTGTGACATCTGTGGCAGAAAGTTTGCC |
| | | CAGTCTGGCGATAGGAAAAGACATACTAAGATCCACACAGGCAGCCAGAAACCATTC |
| | | CAGTGTAGAATCTGTATGAGAAACTTTTCCCAGTCCGGCGATCTGACTAGACACATT |
| | | AGGACTCACACCGGAGAGAAGCCATTCGCCTGCGACATCTGCGGCAGGAAATTCGCT |
| | | CAGAGCGGCGATCGGAAAAGGCACACCAAAATCCACCTGCGCCAGAAAGATGGAGGA |
| | | GGAGGATCCGGCGGAGGAGGCAGCTCCCTGAGCCCCCAGCACTCCGCCGTGACCCAG |
| | | GGCTCTATCATCAAGAACAAGGAGGGCATGGATGCCAAGTCTCTGACAGCCTGGAGC |
| | | AGGACCCTGGTGACATTCAAGGACGTGTTCGTGGACTTCACCCGGGAGGAGTGGAAG |
| | | CTGCTGGACACAGCCCAGCAGATCGTGTACAGAAATGTGATGCTGGAGAACTATAAG |
| | | AATCTGGTGAGCCTGGGCTACCAGCTGACCAAGCCCGATGTGATCCTGCGGCTGGAG |
| | | AAGGGCGAGGAGCCTTGGCTGGTGGAGAGAGAGATTCATCAGGAAACTCATCCCGAT |
| | | AGCGAAACCGCATTCGAGATTAAGTCATCCGTGTGA |
| 89 | Mouse ZFP | MGRIRKKLRLAERP |
| | leader sequence | |
| | (prt) | |
| 90 | Human ZFP | MGPKKRRKVTGERP |
| | leader sequence | |
| | (prt) | |
| 91 | Human p65- | PLGAPGLPNGLLSGDEDESSIADMDFSALLSQISS |
| | RelA activation | |
| | domain (prt) | |
| 92 | Mouse p65- | PLGTSGLPNGLSGDEDFSSIADMDFSALLSQISS |
| | RelA activation | |
| | domain (prt) | |
| 93 | Herpes simplex | PADALDDFDLDMLGDGDSP |
| | virus VP16 | |
| | domain (prt) | |
| 94 | VP64 domain | TSDALDDFDLDMLGSDALDDEDLDMLGSDALDDFDLDMLGSDALDDFDLDMLSSQLS |
| | (prt) | Q |
| 95 | Linker (prt) | LRQKDGGGGSGGGGS |
| 96 | ZF11xALS1-Kox | MGRIRKKLRLAERPYACPVESCDRRFSDSSVLTRHIRIHTGSQKPFQCRICMRNFSR |
| | | SDHLTRHIRTHTGEKPFACDICGRKFADSSVRKRHTKIHTGSQKPFQCRICMRNFSR |
| | | SDHLTRHIRTHTGEKPFACDICGRKFADSSVRKRHTKIHLRQKDGGGGSGGGGSGGG |
| | | GSQKPFQCRICMRNFSRSDHLTRHIRTHTGEKPFACDICGRKFADSSVRKRHTKIHT |
| | | GSQKPFQCRICMRNFSRSDHLTRHIRTHTGEKPFACDICGRKFADSSVRKRHTKIHT |
| | | GSQKPFQCRICMRNFSRSDHLTRHIRTHTGEKPFACDICGRKFADSSVRKRHTKIHL |
| | | RQKDGGGSGGGGSSLSPQHSAVTQGSIIKNKEGMDAKSLTAWSRTLVTFKDVFVDFT |
| | | REEWKLLDTAQQIVYRNVMLENYKNLVSLGYQLTKPDVILRLEKGEEPWLVEREIHQ |
| | | ETHPDSETAFEIKSSV |
| 97 | ZF11xALS2-Kox | MGRIRKKLRLAERPYACPVESCDRRFSDNRDLTRHIRIHTGSQKPFQCRICMRNFSR |
| | | SDHLTRHIRTHTGEKPYACPVESCDRRFSDNRDLTRHIRIHTGSQKPFQCRICMRNE |
| | | SRSDHLTRHIRTHTGEKPYACPVESCDRRFSDNRDLTRHIRIHTGQKPGGGGSGGGG |
| | | SGGGGSQKPFQCRICMRNFSRSDHLTRHIRTHTGEKPYACPVESCDRRFSDNRDLTR |
| | | HIRIHTGSQKPFQCRICMRNFSRSDHLTRHIRTHTGEKPYACPVESCDRRFSDNRDL |
| | | TRHIRIHTGSQKPFQCRICMRNFSRSDHLTRHIRTHTGEKPYACPVESCDRRESDNR |
| | | DLTRHIRIHTGQKPGGGSGGGGSSLSPQHSAVTQGSIIKNKEGMDAKSLTAWSRTLV |
| | | TFKDVFVDFTREEWKLLDTAQQIVYRNVMLENYKNLVSLGYQLTKPDVILRLEKGEE |
| | | PWLVEREIHQETHPDSETAFEIKSSV |
| 98 | ZF11xALS3-Kox | MGRIRKKLRLAERPYACPVESCDRRFSTREDLTRHIRIHTGSQKPFQCRICMRNFSR |
| | | SDHLTRHIRTHTGEKPFACDICGRKFATREDRKRHTKIHTGSQKPFQCRICMRNFSR |
| | | SDHLTRHIRTHTGEKPFACDICGRKFATREDRKRHTKIHLRQKDGGGGSGGGGSGGG |
| | | GSQKPFQCRICMRNFSRSDHLTRHIRTHTGEKPFACDICGRKFATREDRKRHTKIHT |
| | | GSQKPFQCRICMRNFSRSDHLTRHIRTHTGEKPFACDICGRKFATREDRKRHTKIHT |
| | | GSQKPFQCRICMRNFSRSDHLTRHIRTHTGEKPFACDICGRKFATREDRKRHTKIHL |
| | | RQKDLRQKDGGGSGGGGSSLSPQHSAVTQGSIIKNKEGMDAKSLTAWSRTLVTFKDV |
| | | FVDFTREEWKLLDTAQQIVYRNVMLENYKNLVSLGYQLTKPDVILRLEKGEEPWLVE |
| | | REIHQETHPDSETAFEIKSSV |
| 99 | ZF11xALS-TV8- | MGRIRKKLRLAERPYACPVESCDRRFSDNGDLTRHIRIHTGSQKPFQCRICMRNFSR |
| | Kox | SAHLTRHIRTHTGEKPYACPVESCDRRFSDNGDLTRHIRIHTGSQKPFQCRICMRNF |
| | | SRSAHLTRHIRTHTGEKPYACPVESCDRRFSDNGDLTRHIRIHTGQKPGGGGSGGGG |
| | | SGGGGSQKPFQCRICMRNFSRSAHLTRHIRTHTGEKPYACPVESCDRRFSDNGDLTR |
| | | HIRIHTGSQKPFQCRICMRNFSRSAHLTRHIRTHTGEKPYACPVESCDRRFSDNGDL |
| | | TRHIRIHTGSQKPFQCRICMRNFSRSAHLTRHIRTHTGEKPYACPVESCDRRESDNG |
| | | DLTRHIRIHTGQKPGGGSGGGGSSLSPQHSAVTQGSIIKNKEGMDAKSLTAWSRTLV |
| | | TFKDVFVDFTREEWKLLDTAQQIVYRNVMLENYKNLVSLGYQLTKPDVILRLEKGEE |
| | | PWLVEREIHQETHPDSETAFEIKSSV |
| 100 | ZF11xALS-TV9- | MGRIRKKLRLAERPYACPVESCDRRFSDGADLTRHIRIHTGSQKPFQCRICMRNFSR |
| | Kox | SAHLTRHIRTHTGEKPYACPVESCDRRESDGADLTRHIRIHTGSQKPFQCRICMRNF |
| | | SRSAHLTRHIRTHTGEKPYACPVESCDRRFSDGADLTRHIRIHTGQKPGGGGSGGGG |
| | | SGGGGSQKPFQCRICMRNFSRSAHLTRHIRTHTGEKPYACPVESCDRRFSDGADLTR |
| | | HIRIHTGSQKPFQCRICMRNFSRSAHLTRHIRTHTGEKPYACPVESCDRRFSDGADL |
| | | TRHIRIHTGSQKPFQCRICMRNFSRSAHLTRHIRTHTGEKPYACPVESCDRRESDGA |
| | | DLTRHIRIHTGQKPGGGSGGGGSSLSPQHSAVTQGSIIKNKEGMDAKSLTAWSRTLV |
| | | TFKDVFVDFTREEWKLLDTAQQIVYRNVMLENYKNLVSLGYQLTKPDVILRLEKGEE |
| | | PWLVEREIHQETHPDSETAFEIKSSV |
| 101 | ZF11xALS- | MGRIRKKLRLAERPYACPVESCDRRFSAGADLTRHIRIHTGSQKPFQCRICMRNFSR |
| | TV10-Kox | SAHLTRHIRTHTGEKPYACPVESCDRRFSAGADLTRHIRIHTGSQKPFQCRICMRNF |
| | | SRSAHLTRHIRTHTGEKPYACPVESCDRRFSAGADLTRHIRIHTGQKPGGGGSGGGG |
| | | SGGGGSQKPFQCRICMRNFSRSAHLTRHIRTHTGEKPYACPVESCDRRFSAGADLTR |
| | | HIRIHTGSQKPFQCRICMRNFSRSAHLTRHIRTHTGEKPYACPVESCDRRFSAGADL |
| | | TRHIRIHTGSQKPFQCRICMRNFSRSAHLTRHIRTHTGEKPYACPVESCDRRFSAGA |
| | | DLTRHIRIHTGQKPGGGSGGGGSSLSPQHSAVTQGSIIKNKEGMDAKSLTAWSRTLV |
| | | TFKDVFVDFTREEWKLLDTAQQIVYRNVMLENYKNLVSLGYQLTKPDVILRLEKGEE |
| | | PWLVEREIHQETHPDSETAFEIKSSV |
| 102 | Active delivery | MGSLVLTLCALFCLAAYLVSGRIRRDMGPKKKRKVPKKKRKVGERPYACPVESCDRR |
| | construct (prt): | FSQSGDLTRHIRIHTGSQKPFQCRICMRNFSQSGDLTRHIRTHTGEKPFACDICGRK |
| | SS-2xNLS-ZFP- | FAQSGDRKRHTKIHTGSQKPFQCRICMRNFSQSGDLTRHIRTHTGEKPFACDICGRK |
| | KOX | FAQSGDRKRHTKIHLRQKDGGGGSGGGGSGGGGSQKPFQCRICMRNFSQSGDLTRHI |
| | | RTHTGEKPFACDICGRKFAQSGDRKRHTKIHTGSQKPFQCRICMRNFSQSGDLTRHI |
| | | RTHTGEKPFACDICGRKFAQSGDRKRHTKIHTGSQKPFQCRICMRNFSQSGDLTRHI |
| | | RTHTGEKPFACDICGRKFAQSGDRKRHTKIHLRQKDGGGGSGGGGSSLSPQHSAVTQ |
| | | GSIIKNKEGMDAKSLTAWSRTLVTFKDVFVDFTREEWKLLDTAQQIVYRNVMLENYK |
| | | NLVSLGYQLTKPDVILRLEKGEEPWLVEREIHQETHPDSETAFEIKSSV |
| 103 | Active delivery | MGSLVLTLCALFCLAAYLVSGRIRRDMGPKKKRKVPKKKRKVGERPYACPVESCDRR |
| | construct (prt): | FSDSSVLTRHIRIHTGSQKPFQCRICMRNFSRSDHLTRHIRTHTGEKPFACDICGRK |
| | SS-2xNLS-ZFP- | FADSSVRKRHTKIHTGSQKPFQCRICMRNFSRSDHLTRHIRTHTGEKPFACDICGRK |
| | ALS1-KOX | FADSSVRKRHTKIHLRQKDGGGGSGGGGSGGGGSQKPFQCRICMRNFSRSDHLTRHI |
| | | RTHTGEKPFACDICGRKFADSSVRKRHTKIHTGSQKPFQCRICMRNFSRSDHLTRHI |
| | | RTHTGEKPFACDICGRKFADSSVRKRHTKIHTGSQKPFQCRICMRNFSRSDHLTRHI |
| | | RTHTGEKPFACDICGRKFADSSVRKRHTKIHLRQKDGGGGSGGGGSSLSPQHSAVTQ |
| | | GSIIKNKEGMDAKSLTAWSRTLVTFKDVFVDFTREEWKLLDTAQQIVYRNVMLENYK |
| | | NLVSLGYQLTKPDVILRLEKGEEPWLVEREIHQETHPDSETAFEIKSSV |
| 104 | Active delivery | MGSLVLTLCALFCLAAYLVSGRIRRDMGPKKKRKVPKKKRKVGERPYACPVESCDRR |
| | construct (prt): | FSRSDELTRHIRIHTGSQKPFQCRICMRNFSRSDELTRHIRTHTGEKPFACDICGRK |
| | SS-2xNLS-ZFP- | FARSDERKRHTKIHTGSQKPFQCRICMRNFSRSDELTRHIRTHTGEKPFACDICGRK |
| | FXTAS1-KOX | FARSDERKRHTKIHLRQKDGGGGSGGGGSGGGGSQKPFQCRICMRNFSRSDELTRHI |
| | | RTHTGEKPFACDICGRKFARSDERKRHTKIHTGSQKPFQCRICMRNFSRSDELTRHI |
| | | RTHTGEKPFACDICGRKFARSDERKRHTKIHTGSQKPFQCRICMRNFSRSDELTRHI |
| | | RTHTGEKPFACDICGRKFARSDERKRHTKIHLRQKDGGGGSGGGGSSLSPQHSAVTQ |
| | | GSIIKNKEGMDAKSLTAWSRTLVTFKDVFVDFTREEWKLLDTAQQIVYRNVMLENYK |
| | | NLVSLGYQLTKPDVILRLEKGEEPWLVEREIHQETHPDSETAFEIKSSV |
| 105 | CMV promoter | TCAATATTGGCCATTAGCCATATTATTCATTGGTTATATAGCATAAATCAATATTGG |
| | (ATG start | CTATTGGCCATTGCATACGTTGTATCTATATCATAATATGTACATTTATATTGGCTC |
| | codon - | ATGTCCAATATGACCGCCATGTTGGCATTGATTATTGACTAGTTATTAATAGTAATC |
| | underlined) | AATTACGGGGTCATTAGTTCATAGCCCATATATGGAGTTCCGCGTTACATAACTTAC |
| | | GGTAAATGGCCCGCCTGGCTGACCGCCCAACGACCCCCGCCCATTGACGTCAATAAT |
| | | GACGTATGTTCCCATAGTAACGCCAATAGGGACTTTCCATTGACGTCAATGGGTGGA |
| | | GTATTTACGGTAAACTGCCCACTTGGCAGTACATCAAGTGTATCATATGCCAAGTCC |
| | | GCCCCCTATTGACGTCAATGACGGTAAATGGCCCGCCTGGCATTATGCCCAGTACAT |
| | | GACCTTACGGGACTTTCCTACTTGGCAGTACATCTACGTATTAGTCATCGCTATTAC |
| | | CATGGTGATGCGGTTTTGGCAGTACACCAATGGGCGTGGATAGCGGTTTGACTCACG |
| | | GGGATTTCCAAGTCTCCACCCCATTGACGTCAATGGGAGTTTGTTTTGGCACCAAAA |
| | | TCAACGGGACTTTCCAAAATGTCGTAACAACTGCGATCGCCCGCCCCGTTGACGCAA |
| | | ATGGGCGGTAGGCGTGTACGGTGGGAGGTCTATATAAGCAGAGCTCGTTTAGTGAAC |
| | | CGTCAGATCACTAGAAGCTTTATTGCGGTAGTTTATCACAGTTAAATTGCTAACGCA |
| | | GTCAGTGCTTCTGACACAACAGTCTCGAACTTAAGCTGCAGTGACTCTCTTAAGGTA |
| | | GCCTTGCAGAAGTTGGTCGTGAGGCACTGGGCAGGTAAGTATCAAGGTTACAAGACA |
| | | GGTTTAAGGAGACCAATAGAAACTGGGCTTGTCGAGACAGAGAAGACTCTTGCGTTT |
| | | CTGATAGGCACCTATTGGTCTTACTGACATCCACTTTGCCTTTCTCTCCACAGGTGT |
| | | CCACTCCCAGTTCAATTACAGCTCTTAAAAATTGGATCTCCATTCGCCATTCAGGCT |
| | | GCGCAACTGCTGGGAAGGACGATCAGAGCGGGCCTCTTCGCTATTACGCCAGCTGGC |
| | | GAAAGGGACGTGGCAAGCAAGGCGATTAAGTTGAGTTACGCCAGGATTTTCCCAGTC |
| | | ACGACGTTGTAAAACGACGGCCAGAGAATTATAATACGACTCACTATAGGGCGAATT |
| | | CGGATCCTTGCTAGCCTCGAGACGCGTGATTCACCATG |
| 106 | CBh promoter | gcggccgcacgcgtcgttacataacttacggtaaatggcccgcctggctgaccgccc |
| | (NotI, gcggccgc; | aacgacccccgcccattgacgtcaatagtaacgccaatagggactttccattgacgt |
| | and BamHI, | caatgggtggagtatttacggtaaactgcccacttggcagtacatcaagtgtatcat |
| | ggattc restriction | atgccaagtacgccccctattgacgtcaatgacggtaaatggcccgcctggcattgt |
| | sites-bold) | gcccagtacatgaccttatgggactttcctacttggcagtacatctacgtattagtc |
| | | atcgctattaccatggtcgaggtgagccccacgttctgcttcactctccccatctcc |
| | | cccccctccccacccccaattttgtatttatttattttttaattattttgtgcagcg |
| | | atgggggcggggggggggggggggcgcgcgccaggcggggcggggcggggcgagggg |
| | | cggggcggggcgaggcggagaggtgcggcggcagccaatcagagcggcgcgctccga |
| | | aagtttccttttatggcgaggcggcggcggcggcggccctataaaaagcgaagcgcg |
| | | cggcgggcgggagtcgctgcgacgctgccttcgccccgtgccccgctccgccgccgc |
| | | ctcgcgccgcccgccccggctctgactgaccgcgttactcccacaggtgagcgggcg |
| | | ggacggcccttctcctccgggctgtaattagctgagcaagaggtaagggtttaaggg |
| | | atggttggttggtggggtattaatgtttaattacctggagcacctgcctgaaatcac |
| | | tttttttcaggttggggatcc |
| 161 | Human EF1a-1 | gaggctccggtgcccgtcagtgggcagagcgcacatcgcccacagtccccgagaagt |
| | promoter - | tggggggaggggtcggcaattgaaccggtgcctagagaaggtggcgcggggtaaact |
| | 1188bp | gggaaagtgatgtcgtgtactggctccgcctttttcccgagggtgggggagaaccgt |
| | (GenBank: | atataagtgcagtagtcgccgtgaacgttctttttcgcaacgggtttgccgccagaa |
| | J04617.1) | cacaggtaagtgccgtgtgtggttcccgcgggcctggcctctttacgggttatggcc |
| | | cttgcgtgccttgaattacttccacgcccctggctgcagtacgtgattcttgatccc |
| | | gagcttcgggttggaagtgggtgggagagttcgaggccttgcgcttaaggagcccct |
| | | tcgcctcgtgcttgagttgaggcctggcctgggcgctggggccgccgcgtgcgaatc |
| | | tggtggcaccttcgcgcctgtctcgctgctttcgataagtctctagccatttaaaat |
| | | ttttgatgacctgctgcgacgctttttttctggcaagatagtcttgtaaatgcgggc |
| | | caagatctgcacactggtatttcggtttttggggccgcgggcggcgacggggcccgt |
| | | gcgtcccagcgcacatgttcggcgaggcggggcctgcgagcgcggccaccgagaatc |
| | | ggacgggggtagtctcaagctggccggcctgctctggtgcctggcctcgcgccgccg |
| | | tgtatcgccccgccctgggcggcaaggctggcccggtcggcaccagttgcgtgagcg |
| | | gaaagatggccgcttcccggccctgctgcagggagctcaaaatggaggacgcggcgc |
| | | tcgggagagcgggcgggtgagtcacccacacaaaggaaaagggcctttccgtcctca |
| | | gccgtcgcttcatgtgactccacggagtaccgggcgccgtccaggcacctcgattag |
| | | ttctcgagcttttggagtacgtcgtctttaggttggggggaggggttttatgcgatg |
| | | gagtttccccacactgagtgggtggagactgaagttaggccagcttggcacttgatg |
| | | taattctccttggaatttgccctttttgagtttggatcttggttcattctcaagcct |
| | | cagacagtggttcaaagtttttttcttccatttcaggtgtcgtgaaaa |
| 162 | Human | Gtgtctagactgcagagggccctgcgtatgagtgcaagtgggttttaggaccaggat |
| | synapsin | gaggcggggtgggggtgcctacctgacgaccgaccccgacccactggacaagcaccc |
| | promoter | aacccccattccccaaattgcgcatcccctatcagagagggggaggggaaacaggat |
| | (XbaI, gtgtct; | gcggcgaggcgcgtgcgcactgccagcttcagcaccgcggacagtgccttcgccccc |
| | and NcoI, | gcctggcggcgcgcgccaccgccgcctcagcactgaaggcgcgctgacgtcactcgc |
| | ccatgg | cggtcccccgcaaactccccttcccggccaccttggtcgcgtccgcgccgccgccgg |
| | restriction sites- | cccagccggaccgcaccacgcgaggcgcgagataggggggcacgggcgcgaccatct |
| | bold; ATG | gcgctgcggcgccggcgactcagcgctgcctcagtctgcggtgggcagcggaggagt |
| | start codon- | cgtgtcgtgcctgagagcgcagtcgagaaggtaccggatccgccaccatgg |
| | underlined) | |
| 163 | pCAG-promoter | TCGACATTGATTATTGACTAGTTATTAATAGTAATCAATTACGGGGTCATTAGTTCA |
| | (NheI restriction | TAGCCCATATATGGAGTTCCGCGTTACATAACTTACGGTAAATGGCCCGCCTGGCTG |
| | site, gctagc- | ACCGCCCAACGACCCCCGCCCATTGACGTCAATAATGACGTATGTTCCCATAGTAAC |
| | bold underlined; | GCCAATAGGGACTTTCCATTGACGTCAATGGGTGGAGTATTTACGGTAAACTGCCCA |
| | transcribed | CTTGGCAGTACATCAAGTGTATCATATGCCAAGTACGCCCCCTATTGACGTCAATGA |
| | region-bold; | CGGTAAATGGCCCGCCTGGCATTATGCCCAGTACATGACCTTATGGGACTTTCCTAC |
| | ATG start codon- | TTGGCAGTACATCTACGTATTAGTCATCGCTATTACCATGGTCGAGGTGAGCCCCAC |
| | bold, | GTTCTGCTTCACTCTCCCCATCTCCCCCCCCTCCCCACCCCCAATTTTGTATTTATT |
| | underlined) | TATTTTTTAATTATTTTGTGCAGCGATGGGGGCGGGGGGGGGGGGGGGGCGCGCGCC |
| | | AGGCGGGGCGGGGCGGGGCGAGGGGCGGGGCGGGGCGAGGCGGAGAGGTGCGGCGGC |
| | | AGCCAATCAGAGCGGCGCGCTCCGAAAGTTTCCTTTTATGGCGAGGCGGCGGCGGCG |
| | | GCGGCCCTATAAAAAGCGAAGCGCGCGGCGGGCGGGAGTCGCTGCGTTGCCTTCGCC |
| | | CCGTGCCCCGCTCCGCGCCGCCTCGCGCCGCCCGCCCCGGCTCTGACTGACCGCGTT |
| | | ACTCCCACAGGTGAGCGGGCGGGACGGCCCTTCTCCTCCGGGCTGTAATTAGCGCTT |
| | | GGTTTAATGACGGCTTGTTTCTTTTCTGTGGCTGCGTGAAAGCCTTGAGGGGCTCCG |
| | | GGAGGGCCCTTTGTGCGGGGGGAGCGGCTCGGGGGGTGCGTGCGTGTGTGTGTGCGT |
| | | GGGGAGCGCCGCGTGCGGCTCCGCGCTGCCCGGCGGCTGTGAGCGCTGCGGGCGCGG |
| | | CGCGGGGCTTTGTGCGCTCCGCAGTGTGCGCGAGGGGAGCGCGGCCGGGGGCGGTGC |
| | | CCCGCGGTGCGGGGGGCTGCGAGGGGAACAAAGGCTGCGTGCGGGGTGTGTGCGTGG |
| | | GGGGGTGAGCAGGGGGTGTGGGCGCGTCGGTCGGGCTGCAACCCCCCCTGCACCCCC |
| | | CTCCCCGAGTTGCTGAGCACGGCCCGGCTTCGGGTGCGGGGCTCCGTACGGGGCGTG |
| | | GCGCGGGGCTCGCCGTGCCGGGCGGGGGGTGGCGGCAGGTGGGGGTGCCGGGCGGGG |
| | | CGGGGCCGCCTCGGGCCGGGGAGGGCTCGGGGGAGGGGCGCGGCGGCCCCCGGAGCG |
| | | CCGGCGGCTGTCGAGGCGCGGCGAGCCGCAGCCATTGCCTTTTATGGTAATCGTGCG |
| | | AGAGGGCGCAGGGACTTCCTTTGTCCCAAATCTGTGCGGAGCCGAAATCTGGGAGGC |
| | | GCCGCCGCACCCCCTCTAGCGGGCGCGGGGCGAAGCGGTGCGGCGCCGGCAGGAAGG |
| | | AAATGGGCGGGGAGGGCCTTCGTGCGTCGCCGCGCCGCCGTCCCCTTCTCCCTCTCC |
| | | AGCCTCGGGGCTGTCCGCGGGGGGACGGCTGCCTTCGGGGGGGACGGGGCAGGGCGG |
| | | GGTTCGGCTTCTGGCGTGTGACCGGCGGCTCTAGAGCCTCTGCTAACCATGTTCATG |
| | | CCTTCTTCTTTTTCCTACAGCTCCTGGGCAACGTGCTGGTTATTGTGCTGTCTCATC |
| | | ATTTTGGCAAAGAATTGATTAATTCGAGCGAACGCGTCGAGTCGCTCGGTACGATTT |
| | | AAATTGaattggcctcgagcgcaagcttgagctagcctcgagaccATG |

### CLAUSES

Alternative expressions of the inventive concept are set out in each of the following numbered clauses.

A1. A polypeptide comprising a zinc finger peptide having from 8 to 32 zinc finger domains (F1 to F32) according to Formula 2: X₀₋₂ C X₁₋₅ C X₂₋₇ X⁻¹ X⁺¹ X⁺² X⁺³ X⁺⁴ X⁺⁵ X⁺⁶ H X₃₋₆ H/c where X is any amino acid, the numbers in subscript indicate the possible numbers of residues represented by X, and the numbers in superscript indicate the position of the amino acid in the α-helix;
wherein the polypeptide binds to a 5′-GGGGCC-3′ nucleic acid repeat sequence; and at least 8 adjacent zinc finger domains, F1 to F8, have a recognition sequence X⁻¹ X⁺¹ X⁺² X⁺³ X⁺⁴ X⁺⁵ X⁺⁶ according to the following pattern:

| | | F2, F4, F6, | F3, F5, F7, |
|---|---|---|---|
| | F1 | F8, F10 etc | F9, F11 etc |
| ZFP I: | SEQ ID NO: 8 | SEQ ID NO: 3 | SEQ ID NO: 8 |
| ZFP J: | SEQ ID NO: 8 | SEQ ID NO: 3 | SEQ ID NO: 10 |
| ZFP K: | SEQ ID NO: 10 | SEQ ID NO: 3 | SEQ ID NO: 10 |
| ZFP L: | SEQ ID NO: 9 | SEQ ID NO: 3 | SEQ ID NO: 9 |
| ZFP M: | SEQ ID NO: 9 | SEQ ID NO: 3 | SEQ ID NO: 11 |
| ZFP N: | SEQ ID NO: 11 | SEQ ID NO: 3 | SEQ ID NO: 11 |
| ZFP W: | SEQ ID NO: 8 | SEQ ID NO: 12 | SEQ ID NO: 8 |
| ZFP X: | SEQ ID NO: 8 | SEQ ID NO: 12 | SEQ ID NO: 10 |
| ZFP Y: | SEQ ID NO: 10 | SEQ ID NO: 12 | SEQ ID NO: 10 |
| ZFP Z: | SEQ ID NO: 9 | SEQ ID NO: 12 | SEQ ID NO: 9 |
| ZFP AA: | SEQ ID NO: 9 | SEQ ID NO: 12 | SEQ ID NO: 11 |
| ZFP AB: | SEQ ID NO: 11 | SEQ ID NO: 12 | SEQ ID NO: 11 |
| ZFP AI: | SEQ ID NO: 3 | SEQ ID NO: 8 | SEQ ID NO: 3 |
| ZFP AJ: | SEQ ID NO: 3 | SEQ ID NO: 9 | SEQ ID NO: 3 |
| ZFP AK: | SEQ ID NO: 3 | SEQ ID NO: 10 | SEQ ID NO: 3 |
| ZFP AL: | SEQ ID NO: 3 | SEQ ID NO: 11 | SEQ ID NO: 3 |
| ZFP AS: | SEQ ID NO: 12 | SEQ ID NO: 8 | SEQ ID NO: 12 |
| ZFP AT: | SEQ ID NO: 12 | SEQ ID NO: 9 | SEQ ID NO: 12 |
| ZFP AU: | SEQ ID NO: 12 | SEQ ID NO: 10 | SEQ ID NO: 12 |
| ZFP AV: | SEQ ID NO: 12 | SEQ ID NO: 11 | SEQ ID NO: 12 |
| ZFP JX: | SEQ ID NO: 181 | SEQ ID NO: 12 | SEQ ID NO: 181 |
| ZFP JY: | SEQ ID NO: 182 | SEQ ID NO: 12 | SEQ ID NO: 183 |
| ZFP JZ: | SEQ ID NO: 182 | SEQ ID NO: 12 | SEQ ID NO: 183 |
| ZFP KA: | SEQ ID NO: 181 | SEQ ID NO: 133 | SEQ ID NO: 181 |
| ZFP KB: | SEQ ID NO: 182 | SEQ ID NO: 133 | SEQ ID NO: 182 |
| ZFP KC: | SEQ ID NO: 182 | SEQ ID NO: 133 | SEQ ID NO: 183 |
| ZFP KD: | SEQ ID NO: 181 | SEQ ID NO: 134 | SEQ ID NO: 181 |
| ZFP KE: | SEQ ID NO: 182 | SEQ ID NO: 134 | SEQ ID NO: 182 |
| ZFP KF: | SEQ ID NO: 182 | SEQ ID NO: 134 | SEQ ID NO: 183 |
| ZFP KG: | SEQ ID NO: 12 | SEQ ID NO: 181 | SEQ ID NO: 134 |
| ZFP KH: | SEQ ID NO: 12 | SEQ ID NO: 182 | SEQ ID NO: 134 |
| ZFP KI: | SEQ ID NO: 12 | SEQ ID NO: 183 | SEQ ID NO: 134 |
| ZFP KJ: | SEQ ID NO: 133 | SEQ ID NO: 181 | SEQ ID NO: 134 |
| ZFP KK: | SEQ ID NO: 133 | SEQ ID NO: 182 | SEQ ID NO: 134 |
| ZFP KL: | SEQ ID NO: 133 | SEQ ID NO: 183 | SEQ ID NO: 134 |
| ZFP LP: | SEQ ID NO: 184 | SEQ ID NO: 3 | SEQ ID NO: 184 |
| ZFP LQ: | SEQ ID NO: 185 | SEQ ID NO: 3 | SEQ ID NO: 185 |
| ZFP LR: | SEQ ID NO: 186 | SEQ ID NO: 3 | SEQ ID NO: 186 |
| ZFP LS: | SEQ ID NO: 184 | SEQ ID NO: 12 | SEQ ID NO: 184 |
| ZFP LT: | SEQ ID NO: 185 | SEQ ID NO: 12 | SEQ ID NO: 185 |
| ZFP LU: | SEQ ID NO: 186 | SEQ ID NO: 12 | SEQ ID NO: 186. |

A2. The polypeptide according to Clause A1, which is selected from ZFP I, J, L, M, W, X, Z or AA.

A3. The polypeptide according to Clause A1 or Clause A2, wherein at least 8 adjacent zinc finger domains, F1 to F8, have a recognition sequence X⁻¹ X⁺¹ X⁺² X⁺³ X⁺⁴ X⁺⁵ X⁺⁶ according to the pattern of ZFP W or ZFP X, and wherein:
(i) SEQ ID NO: 8 is selected from: DSSVLTR (SEQ ID NO: 13) and ASSELTR (SEQ ID NO: 19);
SEQ ID NO: 12 is selected from: RSDHLTR (SEQ ID NO: 75) and RSGHLTR (SEQ ID NO: 81); and
SEQ ID NO: 10 is selected from: DSSVRKR (SEQ ID NO: 14) and ASSERKR (SEQ ID NO: 20);
(ii) SEQ ID NO: 8 is selected from: DSSVLTR (SEQ ID NO: 13) and ASSELTR (SEQ ID NO: 19)
SEQ ID NO: 12 is selected from: RSDHLTK (SEQ ID NO: 76) and RSGHLTK (SEQ ID NO: 82); and
SEQ ID NO: 10 is selected from: DSSVRKR (SEQ ID NO: 14) and ASSERKR (SEQ ID NO: 20);
(iii) SEQ ID NO: 9 is selected from: DNRDLTR (SEQ ID NO: 31) and TREDLTR (SEQ ID NO: 33)
SEQ ID NO: 12 is selected from: RSDHLTR (SEQ ID NO: 75) and RSGHLTR (SEQ ID NO: 81); and
SEQ ID NO: 11 is selected from: DNRDRKR (SEQ ID NO: 32) and TREDRKR (SEQ ID NO: 34); or
(iv) SEQ ID NO: 9 is selected from: DNRDLTR (SEQ ID NO: 31) and TREDLTR (SEQ ID NO: 33)
SEQ ID NO: 12 is selected from: RSDHLTK (SEQ ID NO: 76) and RSGHLTK (SEQ ID NO: 82); and
SEQ ID NO: 11 is selected from: DNRDRKR (SEQ ID NO: 32) and TREDRKR (SEQ ID NO: 34); or
(v) SEQ ID NO: 184 is selected from: DNGDLTR (SEQ ID NO: 145) and DGADLTR (SEQ ID NO: 146), and
SEQ ID NO: 12 is selected from: RSDHLTK (SEQ ID NO: 76) and RSGHLTK (SEQ ID NO: 82); or
(vi) SEQ ID NO: 185 is selected from: DGADLTR (SEQ ID NO: 146) and AGADLTR (SEQ ID NO: 147), and
SEQ ID NO: 12 is selected from: RSDHLTK (SEQ ID NO: 76) and RSGHLTK (SEQ ID NO: 82).

A4. The polypeptide according to any of Clauses A1 to A3, wherein the polypeptide has the pattern of ZFP X, and wherein:

(i)
SEQ ID NO: 8 is
(SEQ ID NO: 13)
DSSVLTR,
SEQ ID NO: 12 is
(SEQ ID NO: 75)
RSDHLTR
and
SEQ ID NO: 10 is
(SEQ ID NO: 14)
DSSVRKR;
(ii)
SEQ ID NO: 8 is
(SEQ ID NO: 13)
DSSVLTR,
SEQ ID NO: 12 is
(SEQ ID NO: 81)
RSGHLTR
and
SEQ ID NO: 10 is
(SEQ ID NO: 14)
DSSVRKR;
(iii)
SEQ ID NO: 8 is
(SEQ ID NO: 19)
ASSELTR,
SEQ ID NO: 12 is
(SEQ ID NO: 81)
RSGHLTR
and
SEQ ID NO: 10 is
(SEQ ID NO: 20)
ASSERKR;
or
(iv)
SEQ ID NO: 8 is
(SEQ ID NO: 19)
ASSELTR,
SEQ ID NO: 12 is
(SEQ ID NO: 82)
RSGHLTK
and
SEQ ID NO: 10 is
(SEQ ID NO: 20)
ASSERKR.

A5. The polypeptide according to any of Clauses A1 to A3, wherein the polypeptide has the pattern of ZFP Z, and wherein:

(i)
SEQ ID NO: 9 is
(SEQ ID NO: 31)
DNRDLTR,
SEQ ID NO: 12 is
(SEQ ID NO: 75)
RSDHLTR
and
SEQ ID NO: 9 is
(SEQ ID NO: 31)
DNRDLTR;
(ii)
SEQ ID NO: 9 is
(SEQ ID NO: 31)
DNRDLTR,
SEQ ID NO: 12 is
(SEQ ID NO: 81)
RSGHLTR
and
SEQ ID NO: 9 is
(SEQ ID NO: 31)
DNRDLTR;
(iii)
SEQ ID NO: 9 is
(SEQ ID NO: 33)
TREDLTR,
SEQ ID NO: 12 is
(SEQ ID NO: 75)
RSDHLTR
and
SEQ ID NO: 9 is
(SEQ ID NO: 33)
TREDLTR;
or
(iv)
SEQ ID NO: 9 is
(SEQ ID NO: 33)
TREDLTR,
SEQ ID NO: 12 is
(SEQ ID NO: 81)
RSGHLTR
and
SEQ ID NO: 9 is
(SEQ ID NO: 33)
TREDLTR.

A6. The polypeptide according to any of Clauses A1 to A3, wherein the polypeptide has the pattern of ZFP AA, and wherein:

(i)
SEQ ID NO: 9 is
(SEQ ID NO: 31)
DNRDLTR,
SEQ ID NO: 12 is
(SEQ ID NO: 75)
RSDHLTR
and
SEQ ID NO: 11 is
(SEQ ID NO: 32)
DNRDRKR;
(ii)
SEQ ID NO: 9 is
(SEQ ID NO: 31)
DNRDLTR,
SEQ ID NO: 12 is
(SEQ ID NO: 81)
RSGHLTR
and
SEQ ID NO: 11 is
(SEQ ID NO: 32)
DNRDRKR;
(iii)
SEQ ID NO: 9 is
(SEQ ID NO: 33)
TREDLTR,
SEQ ID NO: 12 is
(SEQ ID NO: 75)
RSDHLTR
and
SEQ ID NO: 11 is
(SEQ ID NO: 34)
TREDRKR;
or
(iv)
SEQ ID NO: 9 is
(SEQ ID NO: 33)
TREDLTR,
SEQ ID NO: 12 is
(SEQ ID NO: 81)
RSGHLTR
and
SEQ ID NO: 11 is
(SEQ ID NO: 34)
TREDRKR.

A7. The polypeptide according to any of Clauses A1 to A6, which:
(i) has 10, 11, 12 or 18 zinc finger domains;
(ii) has 11 zinc finger domains;
(ii) has from 10 to 18 zinc finger domains and all of the zinc finger domains of the polypeptide are defined according to the pattern of ZFP I, ZFP J, ZFP L, ZFP M, ZFP W, ZFP X, ZFP Z or ZFP AA;
(iv) is selected from ZFP KM, KN, KO, KP, KQ, KR, KS, KT, KU, KV, KW, KX, KY, KZ, LA, LB and LP to LU; and/or
(v) comprises from 10 to 18 zinc finger domains, wherein at least 10 to 18 adjacent zinc finger domains comprise recognition sequences selected from SEQ ID NOs: 145, 146 or 147 which alternate with recognition sequences selected from 78, 82 or 75.

A8. The polypeptide according to any of Clauses A1 to A7, which comprises the sequence of any of SEQ ID NOs: 166 to 180; or a sequence having at least 90%, at least 95%, or at least 98% identity thereto.

A9. The polypeptide according to any of Clauses A1 to A8, comprising a zinc finger peptide according to the sequence:
N′-[(Formula 4)-L₃]ₙ₀-{[(Formula 6)-L₂-(Formula 6)-L₃]ₙ₁-[(Formula 6)-L₂-(Formula 6)-X_{L}])}ₙ₂-[(Formula 4)-L₂-(Formula 6)-L₃]ₙ₃-[(Formula 6)-L₂-(Formula 6)]-[L₃-(Formula 6)-]ₙ₄ —C′,
wherein n0 is 0 or 1, n1 is from 1 to 4, n2 is 1 or 2, n3 is from 1 to 4, n4 is 0 or 1, L₂ is the linker sequence -TGE/QK/RP- (SEQ ID NO: 113), L₃ is the linker sequence -TGG/SE/QK/RP-(SEQ ID NO: 115), and X_{L} is a linker sequence of between 8 and 50 amino acids; Formula 4 is a zinc finger domain of the sequence X₂ C X_{2,4} C X₅ X⁻¹ X⁺¹ X⁺² X⁺³ X⁺⁴ X⁺⁵ X⁺⁶ H X_{3,4,5} H/C and Formula 6 is a zinc finger domain of the sequence X₂ C X₂ C X₅ X⁻¹ X⁺¹ X⁺² X⁺³ X⁺⁴ X⁺⁵ X⁺⁶ H X₃ H.

A10. The polypeptide according to Clause A9, wherein:
(i) L3 is selected from the group consisting of -TGSERP- (SEQ ID NO: 117) and -TGSQKP-(SEQ ID NO: 123); and/or
(ii) L2 is selected from the group consisting of -TGEKP- (SEQ ID NO: 112) and -TGQKP- (SEQ ID NO: 114); or
(iii) L2 is -TGEKP- (SEQ ID NO: 112) and L3 is TGQKP- (SEQ ID NO: 114); and/or
(iv) X_{L} is selected from the group consisting of SEQ ID NOs: 126 to 131; preferably, wherein X_{L} is SEQ ID NO: 131.

A11. The polypeptide according to any of Clauses A1 to A10, wherein the polypeptide comprises a repression domain from the human KRAB repressor from Kox-1 or a repression domain from the mouse KRAB repressor from ZF87; optionally, wherein the repression domain from the human KRAB repressor comprises the sequence according to SEQ ID NO: 151, or the repression domain from the mouse KRAB repressor comprises the sequence according to SEQ ID NO: 152; preferably wherein the repressor domain is attached to the C-terminal end of the zinc finger peptide.

A12. The polypeptide according to Clause A11, wherein the repression domain is attached to the C-terminus of the zinc finger peptide; optionally via the linker sequence of SEQ ID NO: 153, 154 or155.

A13. The polypeptide according to any of Clauses A1 to A12, wherein the polypeptide comprises a nuclear localisation signal (NLS) sequence; optionally, wherein the nuclear localisation signal comprises the nuclear localisation signal from SV40, mouse primase p58, or human protein KIAA2022; preferably, wherein the nuclear localisation signal is the mouse primase p58 NLS according to SEQ ID NO: 150 or the human protein KIAA2022 NLS according to SEQ ID NO: 149.

A14. The polypeptide of any of Clauses A1 to A13, which binds to an expanded GGGGCC-hexanucleotide repeat sequences containing at least 30 at least 100 or at least 200-hexanucleotide repeats, with a binding affinity stronger than about 1 μM, stronger than about 100 nM, stronger than about 10 nM, or stronger than about 1 nM.

A15. An isolated nucleic acid encoding the polypeptide of any of Clauses A1 to A14.

A16. A vector comprising the nucleic acid of Clause A15.

A17. The vector according to Clause A16, which is a viral vector derived from retroviruses, such as influenza, SIV, HIV, lentivirus, and Moloney murine leukaemia; adenoviruses; adeno-associated viruses (AAV); herpes simplex virus (HSV); and chimeric viruses.

A18. The vector according to Clause A17, which is an adeno-associated virus (AAV) vector; optionally wherein the AAV vector is an AAV2/1 subtype vector; or an AAV2/9 subtype vector; preferably wherein the AAV vector is an AAV2/1 subtype vector.

A19. A polypeptide comprising a zinc finger peptide having from 5 to 7 zinc finger domains (F1 to F7) according to Formula 2: X0-2 C X1-5 C X2-7 X⁻¹ X⁺¹ X⁺² X⁺³ X⁺⁴ X⁺⁵ X⁺⁶ H X3-6 H/C where X is any amino acid, the numbers in subscript indicate the possible numbers of residues represented by X, and the numbers in superscript indicate the position of the amino acid in the α-helix;
wherein the polypeptide binds to a 5′-GGGGCC-3′ nucleic acid repeat sequence; and
the zinc finger domains have a recognition sequence X−1 X+1 X+2 X+3 X+4 X+5 X+6 according to the following pattern:

| | F1 | F2, F4, F6 | F3, F5, F7 |
|---|---|---|---|
| ZFP GO: | SEQ ID NO: 109 | SEQ ID NO: 108 | SEQ ID NO: 109 |
| ZFP GN: | SEQ ID NO: 109 | SEQ ID NO: 108 | SEQ ID NO: 110 |
| ZFP FW: | SEQ ID NO: 107 | SEQ ID NO: 109 | SEQ ID NO: 107 |
| ZFP FV: | SEQ ID NO: 107 | SEQ ID NO: 109 | SEQ ID NO: 108. |

A20. The polypeptide according to Clause A19, wherein the zinc finger domains of the zinc finger peptide are arranged according to a zinc finger array of Table 3 or Table 4.

A21. The polypeptide according to Clause A19 or Clause A20, wherein:
(i) the zinc finger peptide has 6 adjacent zinc finger domains, F1 to F6, according to ZFP GO, i.e. wherein:

SEQ ID NO: 109 is
(SEQ ID NO: 75)
RSDHLTR;
and
SEQ ID NO: 108 is
(SEQ ID NO: 14)
DSSVRKR;

or
(ii) the zinc finger peptide has 5 adjacent zinc finger domains, F1 to F5, according to ZFP FW, i.e. wherein:

SEQ ID NO: 107 is
(SEQ ID NO: 13)
DSSVLTR;
and
SEQ ID NO: 109 is
(SEQ ID NO: 75)
RSDHLTR.

A22. The polypeptide according to any of Clauses A19 to A21, wherein the 5′-GGGGCC-3′ nucleic acid repeat sequence-binding portion consists essentially of 5, 6 or 7 zinc finger domains; or wherein the 5′-GGGGCC-3′ nucleic acid repeat sequence-binding portion has no more than 5, 6 or 7 zinc finger domains; or wherein the 5′-GGGGCC-3′ nucleic acid repeat sequence-binding portion has between 5 and 7 zinc finger domains.

A23. The polypeptide according to any of Clauses A19 to A22, which comprises the sequence of SEQ ID NOs: 169 or 170; or a sequence having at least 90%, at least 95%, or at least 98% identity thereto.

A24. The polypeptide according to any of Clauses A19 to A23, wherein the polypeptide comprises an activation domain selected from the VP64 domain (SEQ ID NO: 94), the herpes simplex virus (HSV) VP16 domain (SEQ ID NO: 93), or the p65-ReIA activation domain; preferably wherein the activation domain is the human p65-ReIA activation domain (SEQ ID NO: 201) or the mouse p65-ReIA activation domain (SEQ ID NO: 92); preferably wherein the activation domain is attached to the C-terminal end of the zinc finger peptide.

A25. The polypeptide according to Clause A24, wherein the activation domain is attached to the C-terminus of the zinc finger peptide via the linker sequence of SEQ ID NO: 153, 154, 155 or 95.

A26. The polypeptide according to any of Clauses A19 to A25, wherein the polypeptide comprises a nuclear localisation signal (NLS) sequence; optionally, wherein the nuclear localisation signal comprises the nuclear localisation signal from SV40, mouse primase p58, or human protein KIAA2022; preferably, wherein the nuclear localisation signal is the mouse primase p58 NLS (SEQ ID NO: 150) or the human protein KIAA2022 NLS (SEQ ID NO: 149).

A27. The polypeptide of any of Clauses A19 to A26, which binds to an expanded GGGGCC-hexanucleotide repeat sequences containing less than 100 less than 30 or less than 15-hexanucleotide repeats, with a binding affinity stronger than about 1 μM, stronger than about 100 nM, stronger than about 10 nM, or stronger than about 1 nM.

A28. An isolated nucleic acid encoding the polypeptide according to any of Clauses A19 to A27.

A29. A vector comprising the nucleic acid of Clause A28.

A30. The vector according to Clause A29, which is a viral vector derived from retroviruses, such as influenza, SIV, HIV, lentivirus, and Moloney murine leukaemia; adenoviruses; adeno-associated viruses (AAV); herpes simplex virus (HSV); and chimeric viruses.

A31. The vector according to Clause A30, which is an adeno-associated virus (AAV) vector; optionally wherein the AAV vector is an AAV2/1 subtype vector; or an AAV2/9 subtype vector; preferably wherein the AAV vector is an AAV2/1 subtype vector.

### Combination Therapy

A32. An isolated nucleic acid encoding the polypeptide of any of Clauses A1 to A14 and the polypeptide of any of Clauses A19 to A27.

A33. An isolated nucleic acid according to Clause A32, comprising a nucleic acid sequence encoding at least one sequence selected from SEQ ID NOs: 166 to 168 or 171 to 185 or a sequence having at least 90%, at least 95%, or at least 98% identity thereto and at least one sequence selected from SEQ ID NOs: 169 and 170 or a sequence having at least 90%, at least 95%, or at least 98% identity thereto.

A34. A vector comprising the nucleic acid of Clause A32 or Clause A33.

A35. The vector according to Clause A34, which is a viral vector derived from retroviruses, such as influenza, SIV, HIV, lentivirus, and Moloney murine leukaemia; adenoviruses; adeno-associated viruses (AAV); herpes simplex virus (HSV); and chimeric viruses.

A36. The vector according to Clause A35, which is an adeno-associated virus (AAV) vector; optionally wherein the AAV vector is an AAV2/1 subtype vector; or an AAV2/9 subtype vector; preferably wherein the AAV vector is an AAV2/1 subtype vector.

A37. In combination:
(i) a polypeptide according to any of Clauses A1 to A14 and a polypeptide according to any of Clauses A19 to A27; or
(ii) a nucleic acid according to Clause A15 and a nucleic acid according to Clause A28; or
(iii) a vector according to any of Clauses A16 to A18 and a vector according to any of Clauses A29 to A31.

A38. A polypeptide according to any of Clauses A1 to A14, a nucleic acid according to Clause A15, and/or a vector according to any of Clauses A16 to A18, for use in medicine.

A39. A polypeptide according to any of Clauses A19 to A27, a nucleic acid according to Clause A28, and/or a vector according to any of Clauses A29 to A31, for use in medicine.

A40. A nucleic acid according to Clause A32 or A33 and/or a vector according to any of Clauses A34 to A36 for use in medicine.

A41. The combination according to Clause A37 for use in medicine.

A42. The polypeptide, nucleic acid, vector or combination for use according to any of Clauses A38 to A41, wherein the use is in a method for treating a disease associated with expanded GGGGCC-hexanucleotide repeat sequences; optionally wherein the disease is a motor neuron disease or dementia; preferably wherein the use is in a method for treating Amyotrophic lateral sclerosis (ALS) and/or Frontotemporal dementia (FTD).

A43. The polypeptide, nucleic acid, vector or combination for use in a method according to Clause A42 in combination with an additional therapeutic agent.

A44. The polypeptide, nucleic acid or vector for use according to any of Clauses A38 to A42, wherein the method comprises:
(a) administering to a subject the polypeptide, nucleic acid or vector according to Clause 38, such that the polypeptide of Clauses A1 to A14 is expressed in or delivered to target cells of the subject; and
(b) administering to the subject the polypeptide, nucleic acid or vector according to Clause A39, such that the polypeptide of Clauses A19 to A27 is expressed in or delivered to target cells of the subject; wherein
step (b) is performed simultaneously, sequentially or separately from step (a) and wherein both the polypeptide of Clauses A1 to A14 and the polypeptide of Clauses A19 to A27 are simultaneously expressed in or delivered to the same target cells of the subject.

A45. The polypeptide, nucleic acid or vector for use according to Clause A44, wherein the polypeptide of Clauses A19 to A27 is delivered to or expressed in cells at a lower concentration than the polypeptide of Clauses A1 to A14; preferably, at a concentration of less than 50%, less than 25%, or less than 10% of the concentration of the polypeptide of Clauses A1 to A14.

A46. A method of treating a disease in a subject in need thereof, the method comprising administering to the subject a polypeptide according to any of Clauses A1 to A14 and/or a polypeptide according to any of Clauses A19 to A27; or administering to the subject a nucleic acid or vector according to any of Clauses A15 to A18 and/or a nucleic acid or vector according to any of Clauses A28 to A31 and causing the polypeptide to be delivered to and/or expressed in target cells of the subject.

A47. A method of treating a disease in a subject in need thereof according to Clause A46, which comprises administering to the subject:
(i) a polypeptide according to any of Clauses A1 to A14 in combination with a polypeptide according to any of Clauses A19 to A27;
(ii) a nucleic acid or vector according to any of Clauses A15 to A18 in combination with a nucleic acid or vector according to any of Clauses A28 to A31;
(iii) a polypeptide according to any of Clauses A1 to A14 in combination with a nucleic acid or vector according to any of Clauses A28 to A31; or
(iv) a polypeptide according to any of Clauses A19 to A27 in combination with a nucleic acid or vector according to any of Clauses A15 to A18.

A48. A gene therapy method comprising administering to a subject in need thereof a vector according to any of Clauses A16 to A18, or A29 to A31.

A49. The method according to any of Clauses A46 to A48, wherein the method is for treating a disease associated with expanded GGGGCC-hexanucleotide repeat sequences; optionally wherein the disease is a motor neuron disease or dementia; preferably wherein the method is for treating a patient suffering from Amyotrophic lateral sclerosis (ALS) and/or Frontotemporal dementia (FTD).

A50. A pharmaceutical composition comprising the polypeptide according to any of Clauses A1 to A14 and/or Clauses A19 to A27; a nucleic acid according to Clause A15 and/or Clause A28, and/or Clause A32 or Clause A33; or a vector according to any of Clauses A16 to A18 and/or Clauses A29 to Clause A31 and/or Clauses A34 to A36.

A51. The pharmaceutical composition according to Clause A50, comprising a polypeptide according to any of Clauses A1 to A14 in combination with a polypeptide according to any of Clauses A19 to A27; or one or more nucleic acid or vector for expressing a polypeptide according to any of Clauses A1 to A14 in combination with a polypeptide according to any of Clauses A19 to A27.

A52. The pharmaceutical composition according to Clause A50 or Clause A51 for use in a method of treating a disease associated with expanded GGGGCC-hexanucleotide repeat sequences, such as a motor neuron disease or dementia; and preferably wherein the disease is Amyotrophic lateral sclerosis (ALS) and/or Frontotemporal dementia (FTD).

A53. The polypeptide, nucleic acid or vector for use according to any of Clauses A38 to A45, the method of any of Clauses A46 to A49, or the pharmaceutical composition for use according to Clause A52, wherein the use or method is in combination with one or more additional therapeutic agent; optionally wherein the use or method is in a combination therapy which comprises the sequential, simultaneous or separate administration of the additional therapeutic agent.

A54. The polypeptide, nucleic acid, or vector for use according to Clause A38 or Clause A39, or the combination for use according to Clause A41, wherein the use in is a method which comprises: causing the polypeptide of any of Clauses A1 to A14 to be expressed in cells of the subject in combination with causing the polypeptide of any of Clauses A19 to A27 to be expressed in cells of the subject.

A55. The polypeptide, nucleic acid, or vector for use according to Clause A38 or Clause A39 or the combination for use according to Clause A41, wherein the use in is a method which comprises: administering to a subject a first AAV2/1 subtype adeno-associated virus (AAV) vector optionally in combination with a first AAV2/9 subtype adeno-associated virus (AAV) vector, wherein the first AAV2/1 and first AAV2/9 vector are capable of expressing the polypeptide of any of Clauses A1 to A14 in cells of the subject; in combination with a second AAV2/1 subtype adeno-associated virus (AAV) vector optionally in combination with a second AAV2/9 subtype adeno-associated virus (AAV) vector, wherein the second AAV2/1 and second AAV2/9 vector are capable of expressing the polypeptide of any of Clauses A19 to A27 in cells of the subject; and wherein the administering of the first AAV2/1 subtype vector and optional first AAV2/9 subtype vector is simultaneous, separate or sequential with the administering of the second AAV2/1 and optional second AAV2/9 subtype vector.

A56. A method for treating a disease in a subject in need thereof, wherein the method comprises administering to the subject an AAV2/1 and/or AAV2/9 subtype adeno-associated virus (AAV) vector according to Clause A18, in combination with an AAV2/1 and/or AAV2/9 subtype adeno-associated virus (AAV) vector according to Clause A31, wherein the administering is simultaneous, separate or sequential, and wherein a polypeptide according to any of Clauses A1 to A14 is co-expressed with a polypeptide according to any of Clauses A19 to A27 in the same target cells of the subject.

A57. The method of Clause A56, wherein the polypeptide according to any of Clauses A19 to A27 is expressed in the target cells at a concentration that is less than the concentration of the polypeptide according to any of Clauses A1 to A14; preferably, wherein the concentration is less than 50%, less than 25%, or less than 10% of the concentration of the polypeptide according to any of Clauses A1 to A14.

A58. The method of Clause A56 or Clause A57, wherein the disease is associated with expanded GGGGCC-hexanucleotide repeat sequences, such as a motor neuron disease or dementia; preferably wherein the disease is Amyotrophic lateral sclerosis (ALS) and/or Frontotemporal dementia (FTD).

B1. An isolated polynucleotide encoding a polypeptide for delivery of an effector peptide to a cell different to the cell in which it was expressed; the polynucleotide comprising:
(a) sequence encoding a polypeptide, the polypeptide comprising:
   (i) the effector peptide sequence;
   (ii) a cell secretion peptide sequence operably linked to the effector peptide sequence;
   (iii) a cell penetration peptide sequence operably linked to the effector peptide sequence; and
(b) a polypeptide expression element operable to cause the polypeptide to be expressed in a target cell in vivo.

B2. The polynucleotide of Clause B1, wherein the cell secretion peptide sequence comprises a protein secretion signal (SS) from human BMP10 protein.

B3. The polynucleotide of Clause B1 or Clause B2, wherein the cell penetration peptide sequence comprises one or more nuclear localisation signals (NLS); optionally wherein the cell penetration peptide sequence has 2, 3, 4 or 5 NLSs arranged in tandem.

B4. The polynucleotide of any of Clauses B1 to B3, wherein the cell penetration peptide sequence comprises:
(i) the nuclear localisation sequence from SV40 virus (PKKKRKV, SEQ ID NO: 148)
(ii) the nuclear localisation sequence from human protein KIAA2022 (PKKRRKVT; NP_001008537.1, SEQ ID NO: 149); or
(iii) the nuclear localisation sequence from mouse primase p58 (RIRKKLR; GenBank: BAA04203.1, SEQ ID NO: 150).

B5. The polynucleotide of any of Clauses B1 to B4, wherein the effector peptide comprises a transcription factor.

B6. The polynucleotide of any of Clauses B1 to B5, wherein the effector peptide comprises a zinc finger peptide, TALE transcription factor or CRISPR transcription factor; preferably wherein the transcription factor is a zinc finger peptide.

B7. The polynucleotide of any of Clauses B1 to B6, wherein the effector peptide comprises a KRAB repression domain from Kox-1.

B8. The polynucleotide of any of Clauses B1 to B7, wherein the polypeptide expression element comprises a strong endogenous constitutive promoter and/or enhancer; preferably, wherein the polypeptide expression element comprises a constitutive promoter/enhancer sequence selected from the group consisting of: CMV, pNSE, PHSP90ab1, Cbh, human EF1α-1, human synapsin promoter and pCAG-promoter.

B9. The polynucleotide of any of Clauses B1 to B8, wherein the polynucleotide encodes a polypeptide comprising the cell secretion peptide arranged N-terminal to the cell penetration peptide, and the cell penetration peptide arranged N-terminal to the effector peptide.

B10. The polynucleotide of Clause B9, which encodes a peptide cleavage sequence arranged between the cell secretion peptide and the cell penetration peptide.

B111. The polynucleotide of Clause B10, wherein the peptide cleavage sequence comprises the RIRR amino acid cleavage site.

B12. The polynucleotide of any of Clauses B1 to B12, wherein the cell secretion peptide comprises the amino acid sequence of MGSLVLTLCALFCLAAYLVSG (SEQ ID NO: 156) B13. The polynucleotide of any of Clauses B1 to B12, wherein the cell penetration peptide comprises the amino acid sequence of PKKKRKVPKKKRKV (SEQ ID NO: 160).

B14. The polynucleotide of any of Clauses B1 to B13, wherein:
(i) the polynucleotide encoding the cell penetration peptide comprises the nucleic acid sequence of CCGAAGAAAAAACGTAAAGTGCCGAAGAAAAAACGTAAAGTG (SEQ ID NO: 87);
(ii) the polynucleotide encoding the cell secretion peptide comprises the nucleic acid sequence of ATGGGCTCTCTGGTCCTGACACTGTGCGCTCTTTTCTGCCTGGCAGCTTACTTGGTTTCT GGC (SEQ ID NO: 84); and/or
(iii) the polynucleotide encoding the RIRR amino acid cleavage site comprises the nucleic acid sequence of CGAATCAGAAGG (SEQ ID NO: 86).

B15. The polynucleotide of any of Clauses B1 to B14, wherein the effector peptide comprises a peptide according to any of Clauses A1 to A14 and/or A19 to A27.

B16. The polynucleotide according to any of Clauses B1 to B15, which encodes a polypeptide comprising the sequence of any of SEQ ID NOs: 166 to 180, 96 to 101 or 102 to 104 or a sequence having at least 90%, at least 95%, or at least 98% identity thereto.

B17. A vector comprising the nucleic acid of any of Clauses B1 to B16.

B18. The vector according to Clause B17, which is a viral vector derived from retroviruses, such as influenza, SIV, HIV, lentivirus, and Moloney murine leukaemia; adenoviruses; adeno-associated viruses (AAV); herpes simplex virus (HSV); and chimeric viruses.

B19. The vector according to Clause B18, which is an adeno-associated virus (AAV) vector; optionally wherein the AAV vector is an AAV2/1 subtype vector; or an AAV2/9 subtype vector; preferably wherein the AAV vector is an AAV2/1 subtype vector.

B20. A polypeptide encoded by the polynucleotide or vector of any of Clauses B1 to B19.

B21. A polypeptide having a sequence according to SEQ ID Nos: 102, 103 or 104 or a sequence having at least 90%, at least 95%, or at least 98% identity thereto.

B22. A method for delivery of a biological effector moiety to a target cell in which it was not expressed (or which cell does not comprise a nucleic acid expression sequence for the biological effector moiety), the method comprising:
(i) providing a nucleic acid expression construct encoding an expressible biological effector peptide, the biological effector peptide adapted for cell secretion from a first target cell and cell penetration of a second target cell, wherein the first and second target cells may be of the same type or of different types;
(ii) delivering the nucleic acid expression construct to the first target cell;
(iii) expressing the expressible biological effector peptide in the first target cell and allowing it to be secreted from the first target cell;
(iv) bringing the secreted biological effector peptide into contact with a second target cell under conditions that allow the biological effector peptide to penetrate the second target cell;
   thereby to deliver the biological effector moiety to the target cell.

B23. The method of Clause B22, wherein the method is performed in vivo or in vitro.

B24. The method of Clause B22 or Clause B23, wherein the biological effector moiety comprises a polypeptide as defined in Clause B20 or Clause B21.

1. A polypeptide comprising a zinc finger peptide having from 8 to 32 zinc finger domains (F1 to F32) according to Formula 2: X₀₋₂ C X₁₋₅ C X₂₋₇ X⁻¹ X⁺¹ X⁺² X⁺³ X⁺⁴ X⁺⁵ X⁺⁶ H X₃₋₆ H/c where X is any amino acid, the numbers in subscript indicate the number of residues represented by X, and the numbers in superscript indicate a position of the amino acid in the α-helix;
wherein the polypeptide binds to a 5′-GGGGCC-3′ nucleic acid repeat sequence; and at least 8 adjacent zinc finger domains, F1 to F8, have a recognition sequence X⁻¹ X⁺¹ X⁺² X⁺³ X⁺⁴ X⁺⁵ X⁺⁶ according to the following pattern:

| | | F2, F4, F6, F8, | F3, F5, F7, F9, |
|---|---|---|---|
| | F1 | F10, . . . F32 | F11, . . . F31 |
| ZFP I: | SEQ ID NO: 8 | SEQ ID NO: 3 | SEQ ID NO: 8 |
| ZFP J: | SEQ ID NO: 8 | SEQ ID NO: 3 | SEQ ID NO: 10 |
| ZFP K: | SEQ ID NO: 10 | SEQ ID NO: 3 | SEQ ID NO: 10 |
| ZFP L: | SEQ ID NO: 9 | SEQ ID NO: 3 | SEQ ID NO: 9 |
| ZFP M: | SEQ ID NO: 9 | SEQ ID NO: 3 | SEQ ID NO: 11 |
| ZFP N: | SEQ ID NO: 11 | SEQ ID NO: 3 | SEQ ID NO: 11 |
| ZFP W: | SEQ ID NO: 8 | SEQ ID NO: 12 | SEQ ID NO: 8 |
| ZFP X: | SEQ ID NO: 8 | SEQ ID NO: 12 | SEQ ID NO: 10 |
| ZFP Y: | SEQ ID NO: 10 | SEQ ID NO: 12 | SEQ ID NO: 10 |
| ZFP Z: | SEQ ID NO: 9 | SEQ ID NO: 12 | SEQ ID NO: 9 |
| ZFP AA: | SEQ ID NO: 9 | SEQ ID NO: 12 | SEQ ID NO: 11 |
| ZFP AB: | SEQ ID NO: 11 | SEQ ID NO: 12 | SEQ ID NO: 11 |
| ZFP AI: | SEQ ID NO: 3 | SEQ ID NO: 8 | SEQ ID NO: 3 |
| ZFP AJ: | SEQ ID NO: 3 | SEQ ID NO: 9 | SEQ ID NO: 3 |
| ZFP AK: | SEQ ID NO: 3 | SEQ ID NO: 10 | SEQ ID NO: 3 |
| ZFP AL: | SEQ ID NO: 3 | SEQ ID NO: 11 | SEQ ID NO: 3 |
| ZFP AS: | SEQ ID NO: 12 | SEQ ID NO: 8 | SEQ ID NO: 12 |
| ZFP AT: | SEQ ID NO: 12 | SEQ ID NO: 9 | SEQ ID NO: 12 |
| ZFP AU: | SEQ ID NO: 12 | SEQ ID NO: 10 | SEQ ID NO: 12 |
| ZFP AV: | SEQ ID NO: 12 | SEQ ID NO: 11 | SEQ ID NO: 12 |
| ZFP JX: | SEQ ID NO: 181 | SEQ ID NO: 12 | SEQ ID NO: 181 |
| ZFP JY: | SEQ ID NO: 182 | SEQ ID NO: 12 | SEQ ID NO: 183 |
| ZFP JZ: | SEQ ID NO: 182 | SEQ ID NO: 12 | SEQ ID NO: 183 |
| ZFP KA: | SEQ ID NO: 181 | SEQ ID NO: 133 | SEQ ID NO: 181 |
| ZFP KB: | SEQ ID NO: 182 | SEQ ID NO: 133 | SEQ ID NO: 182 |
| ZFP KC: | SEQ ID NO: 182 | SEQ ID NO: 133 | SEQ ID NO: 183 |
| ZFP KD: | SEQ ID NO: 181 | SEQ ID NO: 134 | SEQ ID NO: 181 |
| ZFP KE: | SEQ ID NO: 182 | SEQ ID NO: 134 | SEQ ID NO: 182 |
| ZFP KF: | SEQ ID NO: 182 | SEQ ID NO: 134 | SEQ ID NO: 183 |
| ZFP KG: | SEQ ID NO: 12 | SEQ ID NO: 181 | SEQ ID NO: 134 |
| ZFP KH: | SEQ ID NO: 12 | SEQ ID NO: 182 | SEQ ID NO: 134 |
| ZFP KI: | SEQ ID NO: 12 | SEQ ID NO: 183 | SEQ ID NO: 134 |
| ZFP KJ: | SEQ ID NO: 133 | SEQ ID NO: 181 | SEQ ID NO: 134 |
| ZFP KK: | SEQ ID NO: 133 | SEQ ID NO: 182 | SEQ ID NO: 134 |
| ZFP KL: | SEQ ID NO: 133 | SEQ ID NO: 183 | SEQ ID NO: 134 |
| ZFP LP: | SEQ ID NO: 184 | SEQ ID NO: 3 | SEQ ID NO: 184 |
| ZFP LQ: | SEQ ID NO: 185 | SEQ ID NO: 3 | SEQ ID NO: 185 |
| ZFP LR: | SEQ ID NO: 186 | SEQ ID NO: 3 | SEQ ID NO: 186 |
| ZFP LS: | SEQ ID NO: 184 | SEQ ID NO: 12 | SEQ ID NO: 184 |
| ZFP LT: | SEQ ID NO: 185 | SEQ ID NO: 12 | SEQ ID NO: 185 |
| ZFP LU: | SEQ ID NO: 186 | SEQ ID NO: 12 | SEQ ID NO: 186. |

2. The polypeptide according to claim 1, which:
(i) has 10, 11, 12 or 18 zinc finger domains;
(ii) has 11 zinc finger domains;
(ii) has from 10 to 18 zinc finger domains and all of the zinc finger domains of the polypeptide are defined according to the pattern of ZFP I, ZFP J, ZFP L, ZFP M, ZFP W, ZFP X, ZFP Z or ZFP AA;
(iv) has from 10 to 18 zinc finger domains arranged according to a zinc finger array of Table 2; or
(v) has 11 zinc finger domains arranged according to the pattern of any one of ZFP KM to LB of Table 2; or
(vi) has from 10 to 18 zinc finger domains arranged according to the pattern of ZFP LP, ZFP LQ, ZFP LR, ZFP LS, ZFP LT, or ZFP LU.

3. The polypeptide according to claim 1, wherein:
(i)
SEQ ID NO: 184 is selected from:
(SEQ ID NO: 145)
DNGDLTR
and
(SEQ ID NO: 146)
DGADLTR,
and
SEQ ID NO: 12 is selected from:
(SEQ ID NO: 76)
RSDHLTK
and
(SEQ ID NO: 82)
RSGHLTK;
or
(ii)
SEQ ID NO: 185 is selected from:
(SEQ ID NO: 146)
DGADLTR
and
(SEQ ID NO: 147)
AGADLTR,
and
SEQ ID NO: 12 is selected from:
(SEQ ID NO: 76)
RSDHLTK
and
(SEQ ID NO: 82)
RSGHLTK.

4. The polypeptide according to claim 1, wherein the polypeptide comprises a repression domain from the human KRAB repressor from Kox-1 or a repression domain from the mouse KRAB repressor from ZF87.

5. A polypeptide comprising a zinc finger peptide having from 5 to 7 zinc finger domains (F1 to F7) according to Formula 2: X₀₋₂ C X₁₋₅ C X₂₋₇ X⁻¹ X⁺¹ X⁺² X⁺³ X⁺⁴ X⁺⁵ X⁺⁶ H X₃₋₆ H/C where X is any amino acid, the numbers in subscript indicate the number of residues represented by X, and numbers in superscript indicate a position of the amino acid in the α-helix;
wherein the polypeptide binds to a 5′-GGGGCC-3′ nucleic acid repeat sequence; and
the zinc finger domains have a recognition sequence X⁻¹ X⁺¹ X⁺² X⁺³ X⁺⁴ X⁺⁵ X⁺⁶ according to the following pattern:

| | F1 | F2, F4, F6 | F3, F5, F7 |
|---|---|---|---|
| ZFP GO: | SEQ ID NO: 109 | SEQ ID NO: 108 | SEQ ID NO: 109 |
| ZFP GN: | SEQ ID NO: 109 | SEQ ID NO: 108 | SEQ ID NO: 110 |
| ZFP FW: | SEQ ID NO: 107 | SEQ ID NO: 109 | SEQ ID NO: 107 |
| ZFP FV: | SEQ ID NO: 107 | SEQ ID NO: 109 | SEQ ID NO: 108. |

6. The polypeptide according to claim 5, wherein:
(i) the zinc finger peptide has 6 adjacent zinc finger domains, F1 to F6, according to ZFP GO, wherein:
SEQ ID NO: 109 is
(SEQ ID NO: 75)
RSDHLTR;
and
SEQ ID NO: 108 is
(SEQ ID NO: 14)
DSSVRKR;
or
(ii) the zinc finger peptide has 5 adjacent zinc finger domains, F1 to F5, according to ZFP FW, wherein:
SEQ ID NO: 107 is
(SEQ ID NO: 13)
DSSVLTR;
and
SEQ ID NO: 109 is
(SEQ ID NO: 75)
RSDHLTR.

7. The polypeptide according to claim 5, wherein the polypeptide comprises an activation domain selected from a VP64 domain, a herpes simplex virus (HSV) VP16 domain, or a p65-RelA activation domain.

8. The polypeptide according to claim 1, wherein the polypeptide comprises a nuclear localization signal (NLS) sequence; optionally, wherein the nuclear localization signal comprises a nuclear localization signal from SV40, mouse primase p58, or human protein KIAA2022.

9. An isolated nucleic acid encoding the polypeptide of claim 1.

10. A vector comprising the nucleic acid of claim 9, wherein the vector is a viral vector derived from retroviruses; adenoviruses; adeno-associated viruses (AAV); herpes simplex virus (HSV); or chimeric viruses.

11. (canceled)

12. (canceled)

13. A method for treating a disease associated with expanded GGGGCC-hexanucleotide repeat sequences in a subject in need thereof, the method comprising administering to the subject an effective amount of the polypeptide according to claim 1.

14. The method according to claim 13, wherein:
(a) the polypeptide of is expressed in or delivered to a target cell of the subject
(b) administering to the subject an effective amount of a second polypeptide comprising a zinc finger peptide having from 5 to 7 zinc finger domains (F1 to F7) according to Formula 2: X₀₋₂ C X₁₋₅ C X₂₋₇ X⁻¹ X⁺¹ X⁺² X⁺³ X⁺⁴ X⁺⁵ X⁺⁶ H X₃₋₆ H/C where X is any amino acid, the numbers in subscript indicate the number of residues represented by X, and the numbers in superscript indicate the position of the amino acid in the α-helix;
wherein the second polypeptide binds to a 5′-GGGGCC-3′ nucleic acid repeat sequence; and
the zinc finger domains of the second polypeptide have a recognition sequence X⁻¹ X⁺¹ X⁺² X⁺³ X⁺⁴ X⁺⁵ X⁺⁶ according to the following pattern:

| | F1 | F2, F4, F6 | F3, F5, F7 |
|---|---|---|---|
| ZFP GO: | SEQ ID NO: 109 | SEQ ID NO: 108 | SEQ ID NO: 109 |
| ZFP GN: | SEQ ID NO: 109 | SEQ ID NO: 108 | SEQ ID NO: 110 |
| ZFP FW: | SEQ ID NO: 107 | SEQ ID NO: 109 | SEQ ID NO: 107 |
| ZFP FV: | SEQ ID NO: 107 | SEQ ID NO: 109 | SEQ ID NO: 108; |

wherein the polypeptide and the second polypeptide are administered simultaneously, sequentially, or separately and are simultaneously expressed in or delivered to the same target cell of the subject.

15. The method according to claim 14, wherein the second polypeptide is delivered to or expressed in the target cell at a lower concentration than the polypeptide.

16. method according to claim 14, wherein the method comprises: administering to a subject a first AAV2/1 subtype adeno-associated virus (AAV) vector, wherein the first AAV2/1 vector is capable of expressing the polypeptide in combination with a second AAV2/1 subtype adeno-associated virus (AAV) vector, wherein the second AAV2/1 vector is capable of expressing the second polypeptide in cells of the subject; and wherein the administering of the first AAV2/1 subtype vector is simultaneous, separate or sequential with the administering of the second AAV2/1 subtype vector.

17. An isolated nucleic acid encoding the polypeptide of claim 5.

18. A vector comprising the nucleic acid of claim 17, wherein the vector is a viral vector derived from retroviruses, adeno-associated viruses (AAV), herpes simplex virus (HSV), or chimeric viruses.

19. The method according to claim 13, wherein the disease associated with expanded GGGGCC-hexanucleotide repeat sequences is Amyotrophic lateral sclerosis (ALS) and/or Frontotemporal dementia (FTD).

20. The polypeptide according to claim 4, wherein the repression domain from the human KRAB repressor comprises the sequence according to SEQ ID NO: 151, or the repression domain from the mouse KRAB repressor comprises the sequence according to SEQ ID NO: 152;
wherein the repressor domain is attached to the C-terminal end of the zinc finger peptide.

21. The polypeptide according to claim 7, wherein the activation domain is a human p65-RelA activation domain according to SEQ ID NO: 91 or a mouse p65-RelA activation domain according to SEQ ID NO: 92; wherein the activation domain is attached to the C-terminal end of the zinc finger peptide.

22. The polypeptide according to claim 8, wherein the nuclear localization signal is a mouse primase p58 NLS according to SEQ ID NO: 150 or a human protein KIAA2022 NLS according to SEQ ID NO: 149.
